(19) United States
(12) Patent Application Publication
(10) Pub. No.: US 2025/0163119 A1
(43) Pub. Date: May 22, 2025

(54) COMPOUNDS FOR THE TREATMENT OF DIABETES OR OBESITY

(71) Applicant: Eli Lilly and Company, Indianapolis, IN (US)

(72) Inventors: Milata Mary ABRAHAM, Indianapolis, IN (US); Jorge ALSINA-FERNANDEZ, Indianapolis, IN (US); Darryl Wayne HILLIARD, Fishers, IN (US); Samantha Grace Lyons KEYSER, Carmel, IN (US); Wen LIU, Carmel, IN (US); Hongchang QU, Carmel, IN (US); Kristi Lynn SLUKA, Sharpsville, IN (US)

(21) Appl. No.: 18/949,045

(22) Filed: Nov. 15, 2024

Related U.S. Application Data
(60) Provisional application No. 63/600,468, filed on Nov. 17, 2023.

(51) Int. Cl. C07K 14/575; A61K 38/26; A61K 47/54; A61P 3/04; A61P 3/10

(52) U.S. Cl. CPC C07K 14/57509; A61K 38/26; A61K 47/542; A61P 3/04; A61P 3/10

(57) ABSTRACT

The present disclosure relates to novel urocortin-2 polypeptides, having activity at the corticotropin-releasing hormone receptor-2 (CRHR2), pharmaceutical compositions comprising the polypeptides, and methods of using the polypeptides to treat disorders associated with CRHR2.

### SEQUENCE LISTING

The present application is being filed along with a Sequence Listing in ST.26 XML format. The Sequence Listing is provided as a file titled “30698_WO_SL” created 31 Oct. 2024 and is 2.48 megabytes in size. The Sequence Listing information in the ST.26 XML format is incorporated herein by reference in its entirety.

### FIELD OF INVENTION

The present disclosure relates to polypeptides with activity at the corticotropin-releasing hormone receptor-2 (CRHR2), pharmaceutical compositions comprising the polypeptides, and methods of using the polypeptides to treat disorders associated therewith.

### BACKGROUND

Diabetes, obesity, and chronic kidney disease (CKD) are prevalent diseases worldwide with significant health challenges associated therewith. While they are separate diseases, they often occur together as co-morbidities.

Diabetes is a metabolic disorder that occurs either when the pancreas does not produce enough insulin or when the body needs more insulin than it produces, leading to hyperglycemia, or high blood glucose. In 2021, the U.S. Centers for Disease Control and Prevention (CDC) estimated that among the U.S. population, approximately 29.7 million people, representing 8.9% of the U.S. population, had diagnosed diabetes. Even more alarming, among U.S. adults aged 18 years or older, approximately 38.0% of the population is estimated to have prediabetes. One of the main risk factors diabetes is obesity.

Obesity is a medical disorder resulting in excessive accumulation of adipose tissue mass. Today obesity is a global public health concern that the CDC approximates impacts 40% of the adult population. Furthermore, obesity is associated with high costs and serious, undesired health outcomes and morbidities. Some patients with obesity may be further characterized as having a subset of obesity, e.g., sarcopenic obesity, a condition that involves high body fat and low muscle mass. Both obesity and sarcopenic obesity can lead to metabolic problems such as Type 2 diabetes mellitus (T2DM), high blood pressure, and cardiovascular diseases. Therefore, for all patients with obesity, quality weight loss is important, i.e., the loss of adipose tissue without or with minimal impact to lean tissue.

Furthermore, both diabetes and obesity are risk factors for chronic kidney disease (CKD), a condition characterized by the progressive loss of kidney function. Over time, individuals who have CKD experience an increase in albuminuria, proteinuria, serum creatine, and/or renal histopathological lesions. For many patients, CKD eventually develops into end stage renal disease (ESRD), requiring either dialysis or kidney transplant.

Challenges associated with addressing diabetes, obesity, and/or chronic kidney disease includes the need to address one or more of the underlying factors contributing to each patient's particular disease (e.g., high blood sugar, excess weight, the presence of excess fat mass compared to lean mass).

At present, patients with diabetes, obesity, and/or chronic kidney disease have a panoply of treatment choices, including incretin agonists (e.g., liraglutide, tirzepatide, semaglutide), amylin agonists (e.g., pramlintide), DPP-4 inhibitors, metformin, insulin, and many others. However, presently available treatments may present a number of challenges in some patients, including short half-lives, requiring frequent dosing, unpleasant side effects, such as gastrointestinal upset, vomiting, and/or hypoglycemia reactions, immunogenicity reactions, and/or the inability to effectively treat one or more co-factors associated with the disease state (e.g., high blood sugar, excess weight, the presence of excess fat mass compared to lean mass).

Human urocortin-2 (UCN2) is a thirty-eight amino acid endogenous peptide (SEQ ID NO:53). It is one of three known endogenous urocortins (UCN1 and UCN3 are the others) found in mammals and is part of the corticotropin releasing hormone (CRH) family. Urocortin peptides are short-acting peptides that act through CRH receptors known as CRHR1 and/or CRHR2. UCN2 has been reported to improve insulin sensitivity and glucose tolerance through its action as a ligand for the G protein coupled receptor corticotropin releasing hormone receptor 2 (CRHR2) and thus polypeptides with activity at the CRHR2 receptor have been explored as treatments for diabetes and related diseases. Chen et. al 16580-16585 (2006). However, while urocortin analogs are known in the art (see, e.g., U.S. Pat. No. 10,894,817 (Alsina-Fernandez, Eli Lilly) WO 2023/285334 (Li, Novo Nordisk), WO 2023/161229 (Janiak, Corteria Pharmaceuticals), no urocortin analogs have been authorized for medical use to date.

In view thereof, there is a need for polypeptides with activity at the CRH2 receptor. In particular, there is a need for therapeutic compounds which can treat obesity, diabetes, and/or CKD with the preservation of lean mass. Further, there is a need for compounds including one or more of the following properties: a favorable in vitro immunogenicity profile, selective potency for the CHRH2 receptor, improved PK (half-life) compared to endogenous human urocortin, quality weight loss in which a preservation of lean mass is seen compared to fat mass, and suitable developability properties (e.g., association state).

### SUMMARY

Provided herein are polypeptides that have activity at the CRH2 receptor for the treatment of diabetes, obesity, and/or CKD that also address one or more of the above challenges. According to an embodiment of the present disclosure, provided herein are polypeptides, or pharmaceutically acceptable salts thereof, of Formula I comprising:

(SEQ ID NO: 68)
X₁X₂X₃X₄X₅X₆IVTSX₁₁DX₁₃PX₁₅X₁₆X₁₇LX₁₉X₂₀X₂₁
X₂₂EQEX₂₆X₂₇EKX₃₀X₃₁QQAX₃₅EX₃₇X₃₈EILAQV,

wherein
X₁ is Pyr, E, γE, or absent,
X₂ is G or absent,
X₃ is G, S, or absent,
X₄ is S, P, G, or absent,
X₅ is S, P, or absent,
X₆ is G, S, P, or absent,
X₁₁ is L or αMeL,
X₁₃ is V or D-Val,
X₁₅ is T or I,
X₁₆ is Aib or G,
X₁₇ is L or αMeL,
X₁₉ is Q or E,
X₂₀ is K or I,
X₂₁ is I, K, Aib, or L,
X₂₂ is I or L,
X₂₆ is R or K,
X₂₇ is A or Q,
X₃₀ is A or E,
X₃₁ is R or K,
X₃₅ is K or T,
X₃₇ is A or N,
X₃₈ is A or T,
   wherein the N-term amino acid is optionally N-acylated, N-acetylated or N-methylated, and wherein the C-term amino acid is optionally amidated.

In some embodiments of Formula I, the V at position 44 is the C-term amino acid. The C-term may comprise an amide, carboxylic acid, or other modifications. The N-term amino acid depends on the embodiment but will always be the first amino acid present in the sequence which indicates the start of the polypeptide. Therefore, in embodiments of Formula I, the N-term amino acid may be at position X₁, X₂, X₃, X₄, X₅, X₆, or the I at position 7. Further, in some embodiments of Formula I, the N-term may comprise modifications (e.g., acetylation, Ac; methylation, Me; acylation, or other modifications). Further, if any amino acids at positions X₁—X₆ are indicated as “absent”, the next available amino acid will be conjugated to the previously available amino acid. Thus, various embodiments of Formula I result in polypeptides comprising varying lengths, e.g., 38 to 44 amino acids.

Further, in addition to the sequences described herein, the polypeptides described herein may include one or more conservative amino acid substitutions, provided, however, that the polypeptides remain capable of having activity at the CRHR2.

In some embodiments, the polypeptide, or a pharmaceutically acceptable salt thereof, further comprises a means for increasing the half-life of the polypeptide. In some embodiments, the polypeptide, or a pharmaceutically acceptable salt thereof, further comprises a means for increasing the half-life of a polypeptide via any amino acid on the polypeptide with a suitable functional group for conjugation. In some embodiments, the amino acid with a suitable functional group for conjugation is γE, E, or K. In some embodiments, the functional group available for conjugation is conjugated to a phosphonate, tetrazole, sulfonate, bifurcated fatty acid, peptide, protein (such as a VHH, Fc, or monoclonal antibody), or protein fragment (such as, Fab, scFv, Fv, or scFab (single chain Fab). In some embodiments, the fatty acid, linker, protein, or protein fragment may further act as an albumin binder and provide a potential to further generate long-acting compounds.

In some embodiments, the means is a fatty acid.

In some embodiments wherein the amino acid with a functional group for conjugation is K, the conjugation is to an epsilon-amino group of the K. In some embodiments, the polypeptide further comprises a fatty acid conjugated to K at X₃₅, wherein the fatty acid is conjugated to the epsilon-amino group of K via a direct bond or via a linker between K and the fatty acid. In some embodiments wherein the amino acid with a functional group available for conjugation is γE at X₁, the conjugation is via an amide bond between the alpha amino group of the γE and the carboxylic group of the fatty acid. In some embodiments, the means for increasing the half-life of the polypeptide is a fatty acid, wherein the fatty acid is conjugated to the polypeptide at an amino acid with a functional group available for conjugation, via a direct bond or via a linker between the amino acid and the fatty acid.

In some embodiments, the N-term amino acid is N-acylated via a fatty acid conjugated to the N-term amino acid, via a direct bond or via a linker between the amino acid and the fatty acid. In some such embodiments, the linker-fatty acid is conjugated to the peptide after synthesis of the peptide. In other such embodiments, the linker-fatty acid is conjugated to an amino acid (for example, in some embodiments to an E or YE) prior to addition of the addition to the peptide.

In some embodiments, the polypeptide is conjugated to a fatty acid optionally via a linker between the amino acid and the fatty acid. Thus, in some embodiments, the polypeptide is conjugated to a fatty acid via a direct bond between the amino acid and the fatty acid. In other embodiments, the polypeptide is conjugated to a fatty acid via a linker between the amino acid and the fatty acid. In some embodiments, the fatty acid is a C₁₆-C₂₂ fatty acid. It some embodiments, the conjugation of a linker to a C₁₆-C₂₂ fatty acid is referred to as a linker-fatty acid.

Examples of saturated C₁₆-C₂₂ fatty acids for use herein include, but are not limited to, palmitic acid (hexadecenoic acid)(C₁₆ monoacid), hexadecanedioic acid (C₁₆ diacid), margaric acid (heptadecanoic acid) (C₁₇ monoacid), heptadecanedioic acid (C₁₇ diacid), stearic acid (C₁₈ monoacid), octadecanedioic acid (C₁₈ diacid), nonadecylic acid (nonadecanoic acid) (C₁₉ monoacid), nonadecanedioic acid (C₁₉ diacid), arachidic acid (eicosanoic acid) (C₂₀ monoacid), eicosanedioic acid (C₂₀ diacid), heneicosylic acid (heneicosanoic acid) (C₂₁ monoacid), henicosanedioic acid (C₂₁ diacid), behenic acid (docosanoic acid)(C₂₂ monoacid), docosanedioic acid (C₂₂ diacid), including branched and substituted derivatives thereof.

In certain instances, the polypeptide comprises a linker. The linker can have one or more (2-[2-(2-amino-ethoxy)-ethoxy]-acetyl moieties or εK, optionally in combination with one or more amino acids. In instances where the linker includes at least one amino acid, the amino acid can be E or γE amino acid residues. In some instances, the linker can include one or two or three or four or five E or γE amino acid residues, including the D-forms thereof. For example, the linker can include either one or two or three or four γE amino acid residues. Alternatively, the linker can include one to five amino acid residues (e.g., E or γE amino acids) used in combination with one to five (2-[2-(2-amino-ethoxy)-ethoxy]-acetyl) (“AEEA”) or one to five εK moieties. Specifically, the linker can be combinations of one to five E or γE amino acids and one to five (2-[2-(2-amino-ethoxy)-ethoxy]-acetyl) (“AEEA”) or one to five εK moieties, or one to five E or γE amino acids and one to five εK moieties. In some instances, the linker can be combinations of one or two or three γE amino acids and one or two (2-[2-(2-amino-ethoxy)-ethoxy]-acetyl) or εK moieties.

In specific embodiments, the polypeptide, or pharmaceutically acceptable salt thereof, further comprises a linker-fatty acid, wherein the linker-fatty acid is selected from the group consisting of: (2-[2-(2-amino-ethoxy)-ethoxy]-acetyl)-PKE-γE-CO—(CH₂)₁₈—CO₂H, Ahx-(2-[2-(2-amino-ethoxy)-ethoxy]-acetyl)-γE-CO—(CH₂)₁₈—CO₂H, γE-(2-[2-(2-amino-ethoxy)-ethoxy]-acetyl)-Ahx-CO—(CH₂)₁₈—CO₂H, γE-GKEKEKE-CO—(CH₂)₁₈—CO₂H, G-(βA)₃-γE-CO—(CH₂)₁₈—CO₂H, G-(βA)₄-γE-CO—(CH₂)₁₈—CO₂H, (2-[2-(2-amino-ethoxy)-ethoxy]-acetyl)-KEKEKE-γE-CO—(CH₂)₁₈—CO₂H, βA-S-βA-S-γE-CO—(CH₂)₁₈—CO₂H, γE-PKE-Ahx-CO—(CH₂)₁₈—CO₂H, γE-PKE-εK—CO—(CH₂)₁₈—CO₂H, γE-PPP—εK—CO—(CH₂)₁₈—CO₂H, γE-PPPPPP-CO—(CH₂)₁₈—CO₂H, γE-PEPEPE-CO—(CH₂)₁₈—CO₂H, γE-(2-[2-(2-amino-ethoxy)-ethoxy]-acetyl)₂-εK—CO—(CH₂)₁₈—CO₂H, γE-εK-γE-εK—CO—(CH₂)₁₈—CO₂H, γE-εK-(2-[2-(2-amino-ethoxy)-ethoxy]-acetyl)-εK—CO—(CH₂)₁₈—CO₂H, γE-(2-[2-(2-amino-ethoxy)-ethoxy]-acetyl)-εK—CO—(CH₂)₁₆—CO₂H, 2-[2-(2-amino-ethoxy)-ethoxy]-acetyl)-εK—CO—(CH₂)₁₈—CO₂H, (2-[2-(2-amino-ethoxy)-ethoxy]-acetyl)-K—CO—(CH₂)₁₈—CO₂H, (2-[2-(2-amino-ethoxy)-ethoxy]-acetyl)-εK—CO—(CH₂)₁₆—CO₂H, (2-[2-(2-amino-ethoxy)-ethoxy]-acetyl)-Dap-CO—(CH₂)₁₈—CO₂H, (2-[2-(2-amino-ethoxy)-ethoxy]-acetyl)₂-γE-CO—(CH₂)₁₈—CO₂H, γE-(2-[2-(2-amino-ethoxy)-ethoxy]-acetyl)-γE-CO—(CH₂)₁₈—CO₂H, γE₂-(2-[2-(2-amino-ethoxy)-ethoxy]-acetyl)-CO—(CH₂)₁₈—CO₂H, 2-[2-(2-amino-ethoxy)-ethoxy]-acetyl)-εK-γE-CO—(CH₂)₁₈—CO₂, 2-[2-(2-amino-ethoxy)-ethoxy]-acetyl)-γE-εK—CO—(CH₂)₁₈—CO₂H, γK-(2-[2-(2-amino-ethoxy)-ethoxy]-acetyl)-γE-CO—(CH₂)₁₈—CO₂H, εK-γE-(2-[2-(2-amino-ethoxy)-ethoxy]-acetyl)-CO—(CH₂)₁₈—CO₂H, γE-εK-(2-[2-(2-amino-ethoxy)-ethoxy]-acetyl)-CO—(CH₂)₁₈—CO₂H, γE-γE-εK—CO—(CH₂)₁₈—CO₂H, γE-εK-γE-CO—(CH₂)₁₈—CO₂H, εK-γE-γE-CO—(CH₂)₁₈—CO₂H, K(GKEKEKE-CO—(CH₂)₁₈—CO₂H), 2-[2-(2-amino-ethoxy)-ethoxy]-acetyl)-γE₂-CO—(CH₂)₁₈—CO₂H, γE-(2-[2-(2-amino-ethoxy)-ethoxy]-acetyl)-εK—CO—(CH₂)₁₈—CO₂H, 2-[2-(2-amino-ethoxy)-ethoxy]-acetyl)₂γE-C20-OH, and γE-(2-[2-(2-amino-ethoxy)-ethoxy]-acetyl)-εK—CO—(CH₂)₁₈—CO₂H.

In some embodiments, the N-term amino acid is N-acetylated. In some embodiments, the N-term amino acid is N-methylated. In some embodiments, the C-term amino acid is amidated.

In one embodiment of Formula I, X₁ is absent, X₂ is absent, X₃ is G, X₄ is S, X₅ is P, X₆ is S, X₁₁ is αMeL, X₁₃ is V, X₁₅ is T, X₁₆ is Aib, X₁₇ is αMeL, X₁₉ is Q, X₂₀ is K, X₂₁ is I or L, X₂₂ is L, X₂₆ is R, X₂₇ is A, X₃₀ is A, X₃₁ is R, X₃₅ is K, X₃₇ is A, and X₃₈ is A. In such embodiments, the polypeptide comprises a 42 amino acid sequence.

Thus, in some embodiments, provided herein is a polypeptide, or a pharmaceutically acceptable salt thereof, of Formula Ia comprising:

(SEQ ID NO: 69)
GSPSIVTS αMeL DVPT Aib αMeL
LQKX₂₁LEQERAEKARQQAKEAAEILAQV,

wherein
X₂₁ is I or L [wherein when X₁ and X₂ are absent, X₂₁ is at position 19 of the polypeptide],
wherein the N-term amino acid is optionally N-acetylated, and wherein the C-term amino acid is optionally amidated.

In some embodiments of Formula Ia, X₂₁ is I. In some embodiments, the polypeptide comprises SEQ ID NO:20. In another embodiment, the polypeptide is SEQ ID NO:20. Alternatively, the polypeptide can have at least about 90 to 97% sequence similarity to SEQ ID NO:20. Alternatively, the polypeptide can have at least 90%, 91%, 92%, 93%, 94%, 95%, 96%, or 97% sequence similarity to SEQ ID NO:20.

In a specific embodiment of Formula Ia comprising the polypeptide, or a pharmaceutically acceptable salt thereof, X₂₁ is I and the polypeptide further comprises a linker-fatty acid. In a particular embodiment, the linker fatty acid is γE-(2-[2-(2-amino-ethoxy)-ethoxy]-acetyl)-εK—CO—(CH₂)₁₈—CO₂H. In a specific embodiment of Formula Ia comprising the polypeptide, or a pharmaceutically acceptable salt thereof, the K at X₃₅ is conjugated to the linker-fatty acid. In another embodiment, the polypeptide comprises SEQ ID NO:1. In another embodiment, the polypeptide is SEQ ID NO:1. Alternatively, the polypeptide can have at least about 90 to 97% sequence similarity to SEQ ID NO: 1. Alternatively, the polypeptide can have at least 90%, 91%, 92%, 93%, 94%, 95%, 96%, or 97% sequence similarity to SEQ ID NO:1.

In a specific embodiment of Formula Ia comprising the polypeptide, or a pharmaceutically acceptable salt thereof, X₂₁ is L and the polypeptide further comprises a linker-fatty acid. In a particular embodiment, the linker-fatty acid is γE-(2-[2-(2-amino-ethoxy)-ethoxy]-acetyl)-εK—CO—(CH₂)₁₈—CO₂H. In a specific embodiment of Formula Ia comprising the polypeptide, or a pharmaceutically acceptable salt thereof, the K at X₃₅ is conjugated to the linker-fatty acid. In another embodiment, the polypeptide comprises SEQ ID NO:2. In another embodiment, the polypeptide is SEQ ID NO:2. Alternatively, the polypeptide can have at least about 90 to 97% sequence similarity to SEQ ID NO:2. Alternatively, the polypeptide can have at least 90%, 91%, 92%, 93%, 94%, 95%, 96%, or 97% sequence similarity to SEQ ID NO:2.

In a specific embodiment of Formula Ia comprising the polypeptide, or a pharmaceutically acceptable salt thereof, X₂₁ is I and the polypeptide further comprises a linker-fatty acid. In a particular embodiment, the linker-fatty acid is 2-[2-(2-amino-ethoxy)-ethoxy]-acetyl)-PKE-γE-CO—(CH₂)₁₈—CO₂H. In a specific embodiment of Formula Ia comprising the polypeptide, or a pharmaceutically acceptable salt thereof, the K at X₃₅ is conjugated to the linker-fatty acid. In another embodiment, the polypeptide comprises SEQ ID NO:3. In another embodiment, the polypeptide is SEQ ID NO:3. Alternatively, the polypeptide can have at least about 90 to 97% sequence similarity to SEQ ID NO:3. Alternatively, the polypeptide can have at least 90%, 91%, 92%, 93%, 94%, 95%, 96%, or 97% sequence similarity to SEQ ID NO:3.

In a specific embodiment of Formula Ia comprising the polypeptide, or a pharmaceutically acceptable salt thereof, X₂₁ is I and the polypeptide further comprises a linker-fatty acid. In a particular embodiment, the linker-fatty acid is Ahx-(2-[2-(2-amino-ethoxy)-ethoxy]-acetyl)-γE-CO—(CH₂)₁₈—CO₂H. In a specific embodiment of Formula Ia comprising the polypeptide, or a pharmaceutically acceptable salt thereof, the K at X₃₅ is conjugated to the linker-fatty acid. In another embodiment, the polypeptide comprises SEQ ID NO:4. In another embodiment, the polypeptide is SEQ ID NO:4. Alternatively, the polypeptide can have at least about 90 to 97% sequence similarity to SEQ ID NO:4. Alternatively, the polypeptide can have at least 90%, 91%, 92%, 93%, 94%, 95%, 96%, or 97% sequence similarity to SEQ ID NO:4.

In a specific embodiment of Formula Ia comprising the polypeptide, or a pharmaceutically acceptable salt thereof, X₂₁ is I and the polypeptide further comprises a linker-fatty acid. In a particular embodiment, the linker-fatty-acid is γE-(2-[2-(2-amino-ethoxy)-ethoxy]-acetyl)-Ahx-CO—(CH₂)₁₈—CO₂H. In a specific embodiment of Formula Ia comprising the polypeptide, or a pharmaceutically acceptable salt thereof, the K at X₃₅ is conjugated to the linker-fatty acid. In another embodiment, the polypeptide comprises SEQ ID NO:5. In another embodiment, the polypeptide is SEQ ID NO:5. Alternatively, the polypeptide can have at least about 90 to 97% sequence similarity to SEQ ID NO:5. Alternatively, the polypeptide can have at least 90%, 91%, 92%, 93%, 94%, 95%, 96%, or 97% sequence similarity to SEQ ID NO:5.

In a specific embodiment of Formula Ia comprising the polypeptide, or a pharmaceutically acceptable salt thereof, X₂₁ is I and the polypeptide further comprises a linker-fatty acid. In a particular embodiment, the linker-fatty acid is γE-GKEKEKE-CO—(CH₂)₁₈—CO₂H. In a specific embodiment of Formula Ia comprising polypeptide or a pharmaceutically acceptable salt thereof, the K at X₃₅ is conjugated to the linker-fatty acid. In another embodiment, the polypeptide comprises SEQ ID NO:6. In another embodiment, the polypeptide is SEQ ID NO:6. Alternatively, the polypeptide can have at least about 90 to 97% sequence similarity to SEQ ID NO:6. Alternatively, the polypeptide can have at least 90%, 91%, 92%, 93%, 94%, 95%, 96%, or 97% sequence similarity to SEQ ID NO:6.

In a specific embodiment of Formula Ia comprising the polypeptide, or a pharmaceutically acceptable salt thereof, X₂₁ is I and the polypeptide further comprises a linker-fatty acid. In a particular embodiment, the linker-fatty acid is G-(βA)₃-γE-CO—(CH₂)₁₈—CO₂H. In a specific embodiment of Formula Ia comprising the polypeptide, or a pharmaceutically acceptable salt thereof, the K at X₃₅ is conjugated to the linker-fatty acid. In another embodiment, the polypeptide comprises SEQ ID NO:7. In another embodiment, the polypeptide is SEQ ID NO:7. Alternatively, the polypeptide can have at least about 90 to 97% sequence similarity to SEQ ID NO:7. Alternatively, the polypeptide can have at least 90%, 91%, 92%, 93%, 94%, 95%, 96%, or 97% sequence similarity to SEQ ID NO:7.

In a specific embodiment of Formula Ia comprising the polypeptide, or a pharmaceutically acceptable salt thereof, X₂₁ is I and the polypeptide further comprises a linker-fatty acid. In a particular embodiment, the linker-fatty acid is G-(βA)₄-γE-CO—(CH₂)₁₈—CO₂H. In a specific embodiment of Formula Ia comprising the polypeptide, or a pharmaceutically acceptable salt thereof, the K at X₃₅ is conjugated to the linker-fatty acid. In another embodiment, the polypeptide comprises SEQ ID NO:8. In another embodiment the polypeptide is SEQ ID NO:8. Alternatively, the polypeptide can have at least about 90 to 97% sequence similarity to SEQ ID NO:8. Alternatively, the polypeptide can have at least 90%, 91%, 92%, 93%, 94%, 95%, 96%, or 97% sequence similarity to SEQ ID NO:8.

In a specific embodiment of Formula Ia comprising the polypeptide, or a pharmaceutically acceptable salt thereof, X₂₁ is I and the polypeptide further comprises a linker-fatty acid. In a particular embodiment, the linker-fatty acid is 2-[2-(2-amino-ethoxy)-ethoxy]-acetyl)-KEKEKE-γE-CO—(CH₂)₁₈—CO₂H. In a specific embodiment of Formula Ia comprising the polypeptide, or a pharmaceutically acceptable salt thereof, the K at X₃₅ is conjugated to the linker-fatty acid. In another embodiment, the polypeptide comprises SEQ ID NO:9. In another embodiment, the polypeptide is SEQ ID NO:9. Alternatively, the polypeptide can have at least about 90 to 97% sequence similarity to SEQ ID NO:9. Alternatively, the polypeptide can have at least 90%, 91%, 92%, 93%, 94%, 95%, 96%, or 97% sequence similarity to SEQ ID NO:9.

In a specific embodiment of Formula Ia comprising the polypeptide, or a pharmaceutically acceptable salt thereof, X₂₁ is I and the polypeptide further comprises a linker-fatty acid. In a particular embodiment, the linker-fatty acid is βA-S-βA-S-γE-CO—(CH₂)₁₈—CO₂H. In a specific embodiment of Formula Ia comprising the polypeptide, or a pharmaceutically acceptable salt thereof, the K at X₃₅ is conjugated to the linker-fatty acid. In another embodiment, the polypeptide comprises SEQ ID NO:10. In another embodiment, the polypeptide is SEQ ID NO:10. Alternatively, the polypeptide can have at least about 90 to 97% sequence similarity to SEQ ID NO:10. Alternatively, the polypeptide can have at least 90%, 91%, 92%, 93%, 94%, 95%, 96%, or 97% sequence similarity to SEQ ID NO:10.

In a specific embodiment of Formula Ia comprising the polypeptide, or a pharmaceutically acceptable salt thereof, X₂₁ is I and the polypeptide further comprises a linker-fatty acid. In a particular embodiment, the linker-fatty acid is γE-PKE-Ahx-CO—(CH₂)₁₈—CO₂H. In a specific embodiment of Formula Ia comprising the polypeptide, or a pharmaceutically acceptable salt thereof, the K at X₃₅ is conjugated to the linker-fatty acid. In another embodiment, the polypeptide comprises SEQ ID NO:11. In another embodiment, the polypeptide is SEQ ID NO:11. Alternatively, the polypeptide can have at least about 90 to 97% sequence similarity to SEQ ID NO:11. Alternatively, the polypeptide can have at least 90%, 91%, 92%, 93%, 94%, 95%, 96%, or 97% sequence similarity to SEQ ID NO:11.

In a specific embodiment of Formula Ia comprising the polypeptide, or a pharmaceutically acceptable salt thereof, X₂₁ is L and the polypeptide further comprises a linker-fatty acid. In a particular embodiment, the linker-fatty acid is γE-(2-[2-(2-amino-ethoxy)-ethoxy]-acetyl)-εK—CO—(CH₂)₁₆—CO₂H. In a specific embodiment of Formula Ia comprising the polypeptide, or a pharmaceutically acceptable salt thereof, the K at X₃₅ is conjugated to the linker-fatty acid. In another embodiment, the polypeptide comprises SEQ ID NO:12. In another embodiment, the polypeptide is SEQ ID NO:12. Alternatively, the polypeptide can have at least about 90 to 97% sequence similarity to SEQ ID NO: 12. Alternatively, the polypeptide can have at least 90%, 91%, 92%, 93%, 94%, 95%, 96%, or 97% sequence similarity to SEQ ID NO:12.

In a specific embodiment of Formula Ia comprising the polypeptide, or a pharmaceutically acceptable salt thereof, X₂₁ is I and the polypeptide further comprises a linker-fatty acid. In a particular embodiment, the linker-fatty acid is γE-PKE-εK—CO—(CH₂)₁₈—CO₂H. In a specific embodiment of Formula Ia comprising the polypeptide, or a pharmaceutically acceptable salt thereof, the K at X₃₅ is conjugated to the linker-fatty acid. Alternatively, the polypeptide can have at least about 90 to 97% sequence similarity to SEQ ID NO:13. Alternatively, the polypeptide can have at least 90%, 91%, 92%, 93%, 94%, 95%, 96%, or 97% sequence similarity to SEQ ID NO: 13.

In a specific embodiment of Formula Ia comprising the polypeptide, or a pharmaceutically acceptable salt thereof, X₂₁ is I and the polypeptide further comprises a linker-fatty acid. In a particular embodiment, the linker-fatty acid is γE-PPP—εK—CO—(CH₂)₁₈—CO₂H. In a specific embodiment of Formula Ia comprising the polypeptide, or a pharmaceutically acceptable salt thereof, the K at X₃₅ is conjugated to the linker-fatty acid. In another embodiment, the polypeptide comprises SEQ ID NO:14. In another embodiment, the polypeptide is SEQ ID NO:14. Alternatively, the polypeptide can have at least about 90 to 97% sequence similarity to SEQ ID NO:14. Alternatively, the polypeptide can have at least 90%, 91%, 92%, 93%, 94%, 95%, 96%, or 97% sequence similarity to SEQ ID NO:14.

In a specific embodiment of Formula Ia comprising the polypeptide, or a pharmaceutically acceptable salt thereof, X₂₁ is I and the polypeptide further comprises a linker-fatty acid. In a particular embodiment, the linker-fatty acid is γE-PPPPPP-CO—(CH₂)₁₈—CO₂H. In a specific embodiment of the Formula Ia comprising the polypeptide, or a pharmaceutically acceptable salt thereof, the K at X₃₅ is conjugated to the linker-fatty acid. In another embodiment the polypeptide comprises SEQ ID NO:15. In another embodiment, the polypeptide is SEQ ID NO:15. Alternatively, the polypeptide can have at least about 90 to 97% sequence similarity to SEQ ID NO:15. Alternatively, the polypeptide can have at least 90%, 91%, 92%, 93%, 94%, 95%, 96%, or 97% sequence similarity to SEQ ID NO:15.

In a specific embodiment of Formula Ia comprising the polypeptide, or a pharmaceutically acceptable salt thereof, X₂₁ is I and the polypeptide further comprises a linker-fatty acid. In a particular embodiment, the linker-fatty acid is γE-PEPEPE-CO—(CH₂)₁₈—CO₂H. In a specific embodiment of Formula Ia comprising the polypeptide, or a pharmaceutically acceptable salt thereof, the K at X₃₅ is conjugated to the linker-fatty acid. In another embodiment, the polypeptide comprises SEQ ID NO:16. In another embodiment, the polypeptide is SEQ ID NO:16. Alternatively, the polypeptide can have at least about 90 to 97% sequence similarity to SEQ ID NO:16. Alternatively, the polypeptide can have at least 90%, 91%, 92%, 93%, 94%, 95%, 96%, or 97% sequence similarity to SEQ ID NO:16.

In a specific embodiment of Formula Ia comprising the polypeptide, or a pharmaceutically acceptable salt thereof, X₂₁ is I and the polypeptide further comprises a linker-fatty acid. In a particular embodiment, the linker-fatty acid is γE-(2-[2-(2-amino-ethoxy)-ethoxy]-acetyl)₂-εK—CO—(CH₂)₁₈—CO₂H. In a specific embodiment of Formula Ia comprising the polypeptide, or a pharmaceutically acceptable salt thereof, the K at X₃₅ is conjugated to the linker-fatty acid. In another embodiment, the polypeptide comprises SEQ ID NO:17. In another embodiment, the polypeptide is SEQ ID NO:17. Alternatively, the polypeptide can have at least about 90 to 97% sequence similarity to SEQ ID NO: 17. Alternatively, the polypeptide can have at least 90%, 91%, 92%, 93%, 94%, 95%, 96%, or 97% sequence similarity to SEQ ID NO:17.

In a specific embodiment of Formula Ia comprising the polypeptide, or a pharmaceutically acceptable salt thereof, X₂₁ is I and the polypeptide further comprises a linker-fatty acid. In a particular embodiment, the linker-fatty acid is γE-εK-γE-εK—CO—(CH₂)₁₈—CO₂H. In a specific embodiment of Formula Ia comprising the polypeptide, or a pharmaceutically acceptable salt thereof, the K at X₃₅ is conjugated to the linker-fatty acid. Alternatively, the polypeptide can have at least about 90 to 97% sequence similarity to SEQ ID NO:18. Alternatively, the polypeptide can have at least 90%, 91%, 92%, 93%, 94%, 95%, 96%, or 97% sequence similarity to SEQ ID NO:18.

In a specific embodiment of Formula Ia comprising the polypeptide, or a pharmaceutically acceptable salt thereof, X₂₁ is I and the polypeptide further comprises a linker-fatty acid. In a particular embodiment, the linker-fatty acid is γE-εK-(2-[2-(2-amino-ethoxy)-ethoxy]-acetyl)-εK—CO—(CH₂)₁₈—CO₂H. In a specific embodiment of Formula Ia comprising the polypeptide, or a pharmaceutically acceptable salt thereof, the K at X₃₅ is conjugated to the linker-fatty acid. In another embodiment, the polypeptide comprises SEQ ID NO:19. In another embodiment, the polypeptide is SEQ ID NO:19. Alternatively, the polypeptide can have at least about 90 to 97% sequence similarity to SEQ ID NO:19. Alternatively, the polypeptide can have at least 90%, 91%, 92%, 93%, 94%, 95%, 96%, or 97% sequence similarity to SEQ ID NO: 19.

In a specific embodiment of Formula Ia comprising the polypeptide, or a pharmaceutically acceptable salt thereof, X₂₁ is I and the polypeptide further comprises a linker-fatty acid. In a particular embodiment, the linker-fatty acid is γE-(2-[2-(2-amino-ethoxy)-ethoxy]-acetyl)-εK—CO—(CH₂)₁₆—CO₂H. In a specific embodiment of Formula Ia comprising the polypeptide, or a pharmaceutically acceptable salt thereof, the K at X₃₅ is conjugated to the linker-fatty acid. In another embodiment, the polypeptide comprises SEQ ID NO:21. In another embodiment, the polypeptide is SEQ ID NO:21. Alternatively, the polypeptide can have at least about 90 to 97% sequence similarity to SEQ ID NO:21. Alternatively, the polypeptide can have at least 90%, 91%, 92%, 93%, 94%, 95%, 96%, or 97% sequence similarity to SEQ ID NO:21.

In another embodiment of Formula I, X₁ is Pyr, X₂ is G, X₃ is S, X₄ is P, X₅ is S, X₆ is G, X₁₃ is αMeL, X₁₅ is V, X₁₆ is T, X₆ is Aib, X₁₇ is αMeL, X₁₉ is Q, X₂₀ is K, X₂₁ is I, X₂₂ is L, X₂₆ is R, X₂₇ is A, X₃₀ is A, X₃₁ is R, X₃₅ is K, X₃₇ is A, and X₃₈ is A. In such embodiments, the polypeptide comprises a 44 amino acid sequence.

Thus, in some embodiments, provided herein is a polypeptide, or a pharmaceutically acceptable salt thereof, of Formula Ib, comprising:

(SEQ ID NO: 70)
Pyr GSPSGIVTS αMeL DVPT Aib αMeL
LQKILEQERAEKARQQAKEAAEILAQV,

wherein the C-term amino acid is optionally amidated.

In another embodiment the polypeptide comprises SEQ ID NO:23. In another embodiment, the polypeptide is SEQ ID NO:23. Alternatively, the polypeptide can have at least about 90 to 97% sequence similarity to SEQ ID NO:23. Alternatively, the polypeptide can have at least 90%, 91%, 92%, 93%, 94%, 95%, 96%, or 97% sequence similarity to SEQ ID NO:23.

In a specific embodiment of Formula Ib comprising the polypeptide, or a pharmaceutically acceptable salt thereof, the polypeptide further comprises a linker-fatty acid. In a particular embodiment, the linker-fatty acid is γE-(2-[2-(2-amino-ethoxy)-ethoxy]-acetyl)-εK—CO—(CH₂)₁₈—CO₂H. In a specific embodiment of Formula Ib comprising the polypeptide, or a pharmaceutically acceptable salt thereof, the K at X₃₅ is conjugated to the linker-fatty acid. In another embodiment, the polypeptide comprises SEQ ID NO:22. In another embodiment, the polypeptide is SEQ ID NO:22. Alternatively, the polypeptide can have at least about 90 to 97% sequence similarity to SEQ ID NO:22. Alternatively, the polypeptide can have at least 90%, 91%, 92%, 93%, 94%, 95%, 96%, or 97% sequence similarity to SEQ ID NO:22.

In a specific embodiment of Formula Ib comprising the polypeptide, or a pharmaceutically acceptable salt thereof, the polypeptide further comprises a linker-fatty acid. In a particular embodiment, the linker-fatty acid is γE-(2-[2-(2-amino-ethoxy)-ethoxy]-acetyl)-εK—CO—(CH₂)₁₆—CO₂H. In a specific embodiment of Formula Ib comprising the polypeptide, or a pharmaceutically acceptable salt thereof, the K at X₃₅ is conjugated to the linker-fatty acid. In another embodiment, the polypeptide comprises SEQ ID NO:24. In another embodiment the polypeptide is SEQ ID NO:24. Alternatively, the polypeptide can have at least about 90 to 97% sequence similarity to SEQ ID NO:24. Alternatively, the polypeptide can have at least 90%, 91%, 92%, 93%, 94%, 95%, 96%, or 97% sequence similarity to SEQ ID NO:24.

In another embodiment of Formula I, X₁ is γE, E, or absent, X₂ is absent, X₃ is G, X₄ is S, X₅ is P, X₆ is S, X₁₁ is αMeL, X₁₃ is V, X₁₅ is T, X₁₆ is Aib, X₁₇ is αMeL, X₁₉ is Q or E, X₂₀ is K, X₂₁ is I, X₂₂ is L, X₂₆ is R, X₂₇ is A, X₃₀ is A, X₃₁ is R, X₃₅ is T, X₃₇ is A, and X₃₈ is A.

In another embodiment of Formula I, X₁ is absent, X₂ is absent, X₃ is G, X₄ is S, X₅ is P, X₆ is S, X₁₁ is αMeL, X₁₃ is V, X₁₅ is T, X₁₆ is Aib, X₁₇ is αMeL, X₁₉ is Q, X₂₀ is K, X₂₁ is I, X₂₂ is L, X₂₆ is R, X₂₇ is A, X₃₀ is A, X₃₁ is R, X₃₅ is T, X₃₇ is A, and X₃₈ is A. In such embodiments, the polypeptide comprises a 42 amino acid sequence.

In another embodiment, the polypeptide comprises SEQ ID NO:27. In another embodiment, the polypeptide is SEQ ID NO:27. Alternatively, the polypeptide can have at least about 90 to 97% sequence similarity to SEQ ID NO:27. Alternatively, the polypeptide can have at least 90%, 91%, 92%, 93%, 94%, 95%, 96%, or 97% sequence similarity to SEQ ID NO:27.

In another embodiment of Formula I, X₁ is γE or E, X₂ is absent, X₃ is G, X₄ is S, X₅ is P, X₆ is S, X₁₁ is αMeL, X₁₃ is V, X₁₅ is T, X₁₆ is Aib, X₁₇ is αMeL, X₁₉ is Q or E, X₂₀ is K, X₂₁ is I, X₂₂ is L, X₂₆ is R, X₂₇ is A, X₃₀ is A, X₃₁ is R, X₃₅ is T, X₃₇ is A, and X₃₈ is A. In such embodiments, the polypeptide comprises a 43 amino acid sequence.

Thus, in some embodiments, provided herein is a polypeptide, or a pharmaceutically acceptable salt thereof, of Formula Ic, comprising:

(SEQ ID NO: 71)
X₁GSPSIVTS αMeL DVPT Aib αMeLLX₁₉
KILEQERAEKARQQATEAAEILAQV,

wherein
X₁ is γE or E,
X₁₉ is Q or E, [wherein when X₂ is absent, X₁₉ is at position 18 of the polypeptide] wherein the C-term amino acid is optionally amidated.

In a specific embodiment of Formula Ic comprising the polypeptide, or a pharmaceutically acceptable salt thereof, X₁ is γE, X₁₉ is Q, and the linker-fatty acid is (2-[2-(2-amino-ethoxy)-ethoxy]-acetyl)-εK—CO—(CH₂)₁₈—CO₂H. In another embodiment, the polypeptide comprises SEQ ID NO:25. In another embodiment, the polypeptide is SEQ ID NO:25. Alternatively, the polypeptide can have at least about 90 to 97% sequence similarity to SEQ ID NO:25. Alternatively, the polypeptide can have at least 90%, 91%, 92%, 93%, 94%, 95%, 96%, or 97% sequence similarity to SEQ ID NO:25.

In a specific embodiment of Formula Ic comprising the polypeptide, or a pharmaceutically acceptable salt thereof, X₁ is γE, X₁₉ is Q, and the linker-fatty acid is (2-[2-(2-amino-ethoxy)-ethoxy]-acetyl)-K—CO—(CH₂)₁₈—CO₂H. In another embodiment, the polypeptide comprises SEQ ID NO:26. In another embodiment the polypeptide is SEQ ID NO:26. Alternatively, the polypeptide can have at least about 90 to 97% sequence similarity to SEQ ID NO:26. Alternatively, the polypeptide can have at least 90%, 91%, 92%, 93%, 94%, 95%, 96%, or 97% sequence similarity to SEQ ID NO:26.

In a specific embodiment of Formula Ic comprising the polypeptide, or a pharmaceutically acceptable salt thereof, X₁ is γE, X₁₉ is Q, and the linker-fatty acid is (2-[2-(2-amino-ethoxy)-ethoxy]-acetyl)-εK—CO—(CH₂)₁₆—CO₂H. In another embodiment, the polypeptide comprises SEQ ID NO:28. In another embodiment, the polypeptide is SEQ ID NO:28. Alternatively, the polypeptide can have at least about 90 to 97% sequence similarity to SEQ ID NO:28. Alternatively, the polypeptide can have at least 90%, 91%, 92%, 93%, 94%, 95%, 96%, or 97% sequence similarity to SEQ ID NO:28.

In a specific embodiment of Formula Ic comprising the polypeptide, or a pharmaceutically acceptable salt thereof, X₁ is γE, X₁₉ is E, and the linker-fatty acid is (2-[2-(2-amino-ethoxy)-ethoxy]-acetyl)-Dap-CO—(CH₂)₁₈—CO₂H. In another embodiment, the polypeptide comprises SEQ ID NO:52. In another embodiment, the polypeptide is SEQ ID NO:52. Alternatively, the polypeptide can have at least about 90 to 97% sequence similarity to SEQ ID NO:52. Alternatively, the polypeptide can have at least 90%, 91%, 92%, 93%, 94%, 95%, 96%, or 97% sequence similarity to SEQ ID NO:52.

In another embodiment of Formula I, X₁ is absent, X₂ is absent, X₃ is absent, X₄ is absent, X₅ is absent, X₆ is absent, X₁₁ is αMeL, X₁₃ is D-Val, X₁₅ is T or I, X₁₆ is Aib or G, X₁₇ is αMeL, X₁₉ is Q, X₂₀ is I, X₂₁ is K, X₂₂ is L, X₂₆ is R, X₂₇ is A, X₃₀ is A, X₃₁ is R, X₃₅ is K, X₃₇ is N, and X₃₈ is T. In such embodiments, the polypeptide comprises a 38 amino acid sequence.

Thus, in some embodiments, provided herein is a polypeptide, or a pharmaceutically acceptable salt thereof, of Formula Id, comprising

(SEQ ID NO: 72)
IVTS αMeL D D-Val PX₁₅X₁₆ αMeL L
QIKLEQERAEKARQQAKENTEILAQV,

wherein
X₁₅ is T or I [wherein when X₁—X₆ are absent, X₁₅ is at position 9 of the polypeptide],
X₁₆ is G or Aib [wherein when X₁—X₆ are absent, X₁₆ is at position 10 of the polypeptide],
wherein the N-term is amino acid is optionally N-acetylated, and wherein the C-term amino acid is optionally amidated.

In a specific embodiment of Formula Id comprising the polypeptide, or a pharmaceutically acceptable salt thereof, X₁₅ is T, X₁₆ is G, and the polypeptide further comprises a linker-fatty acid. In a particular embodiment, the linker-fatty acid is 2-[2-(2-amino-ethoxy)-ethoxy]-acetyl)₂-γE-CO—(CH₂)₁₈—CO₂H. In a specific embodiment of Formula Id comprising the polypeptide, or a pharmaceutically acceptable salt thereof, the K at X₃₅ is conjugated to the linker-fatty acid. In another embodiment, the polypeptide is SEQ ID NO:29. Alternatively, the polypeptide can have at least about 90 to 97% sequence similarity to SEQ ID NO:29. Alternatively, the polypeptide can have at least 90%, 91%, 92%, 93%, 94%, 95%, 96%, or 97% sequence similarity to SEQ ID NO:29.

In a specific embodiment of Formula Id comprising the polypeptide, or a pharmaceutically acceptable salt thereof, X₁₅ is T, X₁₆ is G, and the polypeptide further comprises a linker-fatty acid. In a particular embodiment, the linker-fatty acid is γE-(2-[2-(2-amino-ethoxy)-ethoxy]-acetyl)-γE-CO—(CH₂)₁₈—CO₂H. In a specific embodiment of Formula Id comprising the polypeptide, or a pharmaceutically acceptable salt thereof, the K at X₃₅ is conjugated to the linker-fatty acid. In another embodiment, the polypeptide comprises SEQ ID NO:30. In another embodiment, the polypeptide is SEQ ID NO:30. Alternatively, the polypeptide can have at least about 90 to 97% sequence similarity to SEQ ID NO:30. Alternatively, the polypeptide can have at least 90%, 91%, 92%, 93%, 94%, 95%, 96%, 97% sequence similarity to SEQ ID NO:30.

In a specific embodiment of Formula Id comprising the polypeptide, or a pharmaceutically acceptable salt thereof, X₁₅ is T, X₁₆ is G, and the polypeptide further comprises a linker-fatty acid. In a particular embodiment, the linker-fatty acid is γE₂-(2-[2-(2-amino-ethoxy)-ethoxy]-acetyl)-CO—(CH₂)₁₈—CO₂H. In a specific embodiment of Formula Id comprising the polypeptide, or pharmaceutically acceptable salt thereof, the K at X₃₅ is conjugated to the linker-fatty acid. In another embodiment, the polypeptide comprises SEQ ID NO:31. In another embodiment, the polypeptide is SEQ ID NO:31. Alternatively, the polypeptide can have at least about 90 to 99% sequence similarity to SEQ ID NO:31. Alternatively, the polypeptide can have at least 90%, 91%, 92%, 93%, 94%, 95%, 96%, 97%, 98% or 99% sequence similarity to SEQ ID NO:31.

In a specific embodiment of Formula Id comprising the polypeptide, or a pharmaceutically acceptable salt thereof, X₁₅ is T, X₁₆ is G, and the polypeptide further comprises a linker-fatty acid. In a particular embodiment, the linker-fatty acid is 2-[2-(2-amino-ethoxy)-ethoxy]-acetyl)-εK-γE-CO—(CH₂)₁₈—CO₂. In a specific embodiment of Formula Id comprising the polypeptide, or pharmaceutically acceptable salt thereof, the K at X₃₅ is conjugated to the linker-fatty acid. In another embodiment, the polypeptide comprises SEQ ID NO:32. In another embodiment, the polypeptide is SEQ ID NO:32. Alternatively, the polypeptide can have at least about 90 to 97% sequence similarity to SEQ ID NO:32. Alternatively, the polypeptide can have at least 90%, 91%, 92%, 93%, 94%, 95%, 96%, or 97% sequence similarity to SEQ ID NO:32.

In a specific embodiment of Formula Id comprising the polypeptide, or a pharmaceutically acceptable salt thereof, X₁₅ is T, X₁₆ is G, and the polypeptide further comprises a linker-fatty acid. In a particular embodiment, linker-fatty acid is 2-[2-(2-amino-ethoxy)-ethoxy]-acetyl)-γE-εK—CO—(CH₂)₁₈—CO₂H. In a specific embodiment of the Formula Id comprising the polypeptide, or a pharmaceutically acceptable salt thereof, the K at X₃₅ is conjugated to the linker-fatty acid. In another embodiment, the polypeptide comprises SEQ ID NO:33. In another embodiment, the polypeptide is SEQ ID NO:33. Alternatively, the polypeptide can have at least about 90 to 97% sequence similarity to SEQ ID NO:33. Alternatively, the polypeptide can have at least 90%, 91%, 92%, 93%, 94%, 95%, 96%, or 97% sequence similarity to SEQ ID NO:33.

In a specific embodiment of Formula Id comprising the polypeptide, or a pharmaceutically acceptable salt thereof, X₁₅ is T, X₁₆ is G, and the polypeptide further comprises a linker-fatty acid. In a particular embodiment, the linker-fatty acid is εK-(2-[2-(2-amino-ethoxy)-ethoxy]-acetyl)-γE-CO—(CH₂)₁₈—CO₂H. In a specific embodiment of the polypeptide, or a pharmaceutically acceptable salt thereof, the K at X₃₅ is conjugated to the linker-fatty acid. In another embodiment, the polypeptide comprises SEQ ID NO:34. In another embodiment, the polypeptide is SEQ ID NO:34. Alternatively, the polypeptide can have at least about 90 to 97% sequence similarity to SEQ ID NO:34. Alternatively, the polypeptide can have at least 90%, 91%, 92%, 93%, 94%, 95%, 96%, or 97% sequence similarity to SEQ ID NO:34.

In a specific embodiment of Formula Id comprising the polypeptide, or a pharmaceutically acceptable salt thereof, X₁₅ is T, X₁₆ is G, and the polypeptide further comprises a linker-fatty acid. In a particular embodiment, the linker-fatty acid is εK-γE-(2-[2-(2-amino-ethoxy)-ethoxy]-acetyl)-CO—(CH₂)₁₈—CO₂H. In a specific embodiment of Formula Id comprising the polypeptide, or a pharmaceutically acceptable salt thereof, the K at X₃₅ is conjugated to the linker-fatty acid. In another embodiment, the polypeptide comprises SEQ ID NO:35. In another embodiment, the polypeptide is SEQ ID NO:35. Alternatively, the polypeptide can have at least about 90 to 97% sequence similarity to SEQ ID NO:35. Alternatively, the polypeptide can have at least 90%, 91%, 92%, 93%, 94%, 95%, 96%, or 97% sequence similarity to SEQ ID NO:35.

In a specific embodiment of Formula Id comprising the polypeptide, or a pharmaceutically acceptable salt thereof, X₁₅ is T, X₁₆ is G, and the polypeptide further comprises a linker-fatty acid. In a particular embodiment, the linker-fatty acid is γE-(2-[2-(2-amino-ethoxy)-ethoxy]-acetyl)-εK—CO—(CH₂)₁₈—CO₂H. In a specific embodiment of Formula Id comprising the polypeptide, or a pharmaceutically acceptable salt thereof, the K at X₃₅ is conjugated to the linker-fatty acid. In another embodiment, the polypeptide comprises SEQ ID NO:36. In another embodiment, the polypeptide is SEQ ID NO:36. Alternatively, the polypeptide can have at least about 90 to 97% sequence similarity to SEQ ID NO:36. Alternatively, the polypeptide can have at least 90%, 91%, 92%, 93%, 94%, 95%, 96%, or 97% sequence similarity to SEQ ID NO:36.

In a specific embodiment of Formula Id comprising the polypeptide, or a pharmaceutically acceptable salt thereof, X₁₅ is T, X₁₆ is G, and the polypeptide further comprises a linker-fatty acid. In a particular embodiment, the linker-fatty acid is γE-εK-(2-[2-(2-amino-ethoxy)-ethoxy]-acetyl)-CO—(CH₂)₁₈—CO₂H. In a specific embodiment of Formula Id comprising the polypeptide, or a pharmaceutically acceptable salt thereof, the K at X₃₅ is conjugated to the linker-fatty acid. In another embodiment, the polypeptide comprises SEQ ID NO:37. In another embodiment the polypeptide is SEQ ID NO:37. Alternatively, the polypeptide can have at least about 90 to 97% sequence similarity to SEQ ID NO:37. Alternatively, the polypeptide can have at least 90%, 91%, 92%, 93%, 94%, 95%, 96%, or 97% sequence similarity to SEQ ID NO:37.

In a specific embodiment of Formula Id comprising the polypeptide, or a pharmaceutically acceptable salt thereof, X₁₅ is T, X₁₆ is G, and the polypeptide further comprises a linker-fatty acid. In a particular embodiment, the linker-fatty acid is γE-γE-εK—CO—(CH₂)₁₈—CO₂H. In a specific embodiment of Formula Id comprising the polypeptide, or a pharmaceutically acceptable salt thereof, the K at X₃₅ is conjugated to the linker-fatty acid. In another embodiment, the polypeptide comprises SEQ ID NO:38. In another embodiment, the polypeptide is SEQ ID NO:38. Alternatively, the polypeptide can have at least about 90 to 97% sequence similarity to SEQ ID NO:38. Alternatively, the polypeptide can have at least 90%, 91%, 92%, 93%, 94%, 95%, 96%, or 97% sequence similarity to SEQ ID NO:38.

In a specific embodiment of Formula Id comprising the polypeptide, or a pharmaceutically acceptable salt thereof, X₁₅ is T, X₁₆ is G, and the polypeptide further comprises a linker-fatty acid. In a particular embodiment, the linker-fatty acid is γE-εK-γE-CO—(CH₂)₁₈—CO₂H. In a specific embodiment of Formula Id comprising the polypeptide, or a pharmaceutically acceptable salt thereof, the K at X₃₅ is conjugated to the linker-fatty acid. In another embodiment, the polypeptide comprises SEQ ID NO:39. In another embodiment the polypeptide is SEQ ID NO:39. Alternatively, the polypeptide can have at least about 90 to 97% sequence similarity to SEQ ID NO:39. Alternatively, the polypeptide can have at least 90%, 91%, 92%, 93%, 94%, 95%, 96%, or 97% sequence similarity to SEQ ID NO:39.

In a specific embodiment of Formula Id comprising the polypeptide, or a pharmaceutically acceptable salt thereof, X₁₅ is T, X₁₆ is G, and the polypeptide further comprises a linker-fatty acid. In a particular embodiment, the linker-fatty acid is εK-γE-γE-CO—(CH₂)₁₈—CO₂H. In a specific embodiment of Formula Id comprising the polypeptide, or a pharmaceutically acceptable salt thereof, the K at X₃₅ is conjugated to the linker-fatty acid. In another embodiment, the polypeptide comprises SEQ ID NO:40. In another embodiment, the polypeptide is SEQ ID NO:40. Alternatively, the polypeptide can have at least about 90 to 97% sequence similarity to SEQ ID NO:40. Alternatively, the polypeptide can have at least 90%, 91%, 92%, 93%, 94%, 95%, 96%, or 97% sequence similarity to SEQ ID NO:40.

In a specific embodiment of Formula Id comprising the polypeptide, or a pharmaceutically acceptable salt thereof, X₁₅ is T, X₁₆ is Aib, and the polypeptide further comprises a linker-fatty acid. In a particular embodiment, the linker-fatty acid is 2-[2-(2-amino-ethoxy)-ethoxy]-acetyl)₂-γE-CO—(CH₂)₁₈—CO₂H. In a specific embodiment of Formula Id comprising the polypeptide, or a pharmaceutically acceptable salt thereof, the K at X₃₅ is conjugated to the linker-fatty acid. In another embodiment, the polypeptide comprises SEQ ID NO:41. In another embodiment, the polypeptide is SEQ ID NO:41. Alternatively, the polypeptide can have at least about 90 to 97% sequence similarity to SEQ ID NO:41. Alternatively, the polypeptide can have at least 90%, 91%, 92%, 93%, 94%, 95%, 96%, or 97% sequence similarity to SEQ ID NO:41.

In a specific embodiment of Formula Id comprising the polypeptide, or a pharmaceutically acceptable salt thereof, X₁₅ is I, X₁₆ is Aib, and the polypeptide further comprises a linker-fatty acid moiety. In a particular embodiment, the linker-fatty acid is 2-[2-(2-amino-ethoxy)-ethoxy]-acetyl)₂-γE-CO—(CH₂)₁₈—CO₂H. In a specific embodiment of Formula Id comprising the polypeptide, or pharmaceutically acceptable salt thereof, the K at X₃₅ is conjugated to the linker-fatty acid. In another embodiment, the polypeptide comprises SEQ ID NO:42. In another embodiment, the polypeptide is SEQ ID NO:42. Alternatively, the polypeptide can have at least about 90 to 97% sequence similarity to SEQ ID NO:42. Alternatively, the polypeptide can have at least 90%, 91%, 92%, 93%, 94%, 95%, 96%, or 97% sequence similarity to SEQ ID NO:42.

In a specific embodiment of Formula Id comprising the polypeptide, or a pharmaceutically acceptable salt thereof, X₁₅ is T, X₁₆ is G, and the polypeptide further comprises a linker-fatty acid. In a particular embodiment, the linker-fatty acid is GKEKEKE-CO—(CH₂)₁₈—CO₂H. In a specific embodiment of Formula Id comprising the polypeptide, or a pharmaceutically acceptable salt thereof, the K at X₃₅ is conjugated to the linker-fatty acid. In another embodiment, the polypeptide comprises SEQ ID NO:43. In another embodiment, the polypeptide is SEQ ID NO:43. Alternatively, the polypeptide can have at least about 90 to 97% sequence similarity to SEQ ID NO:43. Alternatively, the polypeptide can have at least 90%, 91%, 92%, 93%, 94%, 95%, 96%, or 97% sequence similarity to SEQ ID NO:43.

In a specific embodiment of Formula Id comprising the polypeptide, or a pharmaceutically acceptable salt thereof, X₁₅ is T, X₁₆ is G, and the polypeptide further comprises a linker-fatty acid. In a particular embodiment, the linker-fatty acid is 2-[2-(2-amino-ethoxy)-ethoxy]-acetyl)-γE₂-CO—(CH₂)₁₈—CO₂H. In a specific embodiment of Formula Id comprising the polypeptide, or a pharmaceutically acceptable salt thereof, the K at X₃₅ is conjugated to the linker-fatty acid. In another embodiment, the polypeptide comprises SEQ ID NO:44. In another embodiment, the polypeptide is SEQ ID NO:44. Alternatively, the polypeptide can have at least about 90 to 97% sequence similarity to SEQ ID NO:44. Alternatively, the polypeptide can have at least 90%, 91%, 92%, 93%, 94%, 95%, 96%, or 97% sequence similarity to SEQ ID NO:44.

In a specific embodiment of Formula Id comprising the polypeptide, or a pharmaceutically acceptable salt thereof, X₁₅ is I, X₁₆ is Aib, and the polypeptide further comprises a linker-fatty acid. In a particular embodiment, the linker-fatty acid is γE₂-CO—(CH₂)₁₈—CO₂H. In a specific embodiment of the Formula Id comprising the polypeptide, or a pharmaceutically acceptable salt thereof, the K at X₃₅ is conjugated to the linker-fatty acid. In another embodiment, the polypeptide comprises SEQ ID NO:51. In another embodiment, the polypeptide is SEQ ID NO:51. Alternatively, the polypeptide can have at least about 90 to 97% sequence similarity to SEQ ID NO:51. Alternatively, the polypeptide can have at least 90%, 91%, 92%, 93%, 94%, 95%, 96%, or 97% sequence similarity to SEQ ID NO:51.

In another embodiment of Formula I, X₁ is absent, X₂ is absent, X₃ is absent, X₄ is P, X₅ is P, X₆ is P, X₁₁ is αMeL, X₁₃ is V or D-Val, X₁₅ is T, X₁₆ is Aib, X₁₇ is αMeL, X₁₉ is Q, X₂₀ is K, X₂₁ is L or Aib, X₂₂ is L, X₂₆ is R, X₂₇ is A, X₃₀ is A, X₃₁ is R, X₃₅ is K, X₃₇ is A or N, X₃₈ is A or T. In such embodiments, the polypeptide comprises a 41 amino acid sequence.

Thus, in some embodiments, provided herein is a polypeptide, or a pharmaceutically acceptable salt thereof, of Formula Ie, comprising

(SEQ ID NO: 73)
PPPIVTS αMeL DX₁₃PTAib αMeL LQK
X₂₁LEQERAEKARQQAKEX₃₇X₃₈EILAQV,

wherein,
X₁₃ is V or D-Val [wherein when X₁—X₃ are absent, X₁₃ is at position 10 of the polypeptide],
X₂₁ is L or Aib [wherein when X₁—X₃ are absent, X₂₁ is at position 18 of the polypeptide],
X₃₇ is A or N [wherein when X₁—X₃ are absent, X₃₇ is at position 34 of the polypeptide],
X₃₈ is A or T [wherein when X₁—X₃ are absent, X₃₈ is at position 35 of the polypeptide], wherein the N-term amino acid is optionally N-acetylated, and wherein the C-term amino acid is optionally amidated.

In a specific embodiment of Formula Ie comprising the polypeptide, or a pharmaceutically acceptable salt thereof, X₁₃ is D-Val, X₂₁ is Aib, X₃₇ is N, X₃₈ is T, and the polypeptide further comprises a linker-fatty acid. In a particular embodiment, the linker-fatty acid is γE-(2-[2-(2-amino-ethoxy)-ethoxy]-acetyl)-εK—CO—(CH₂)₁₈—CO₂H. In a specific embodiment, the K at X₃₅ is conjugated to the linker-fatty acid. In some embodiments, the polypeptide comprises SEQ ID NO:46. In some embodiments, the polypeptide is SEQ ID NO:46. Alternatively, the polypeptide can have at least about 90 to 97% sequence similarity to SEQ ID NO:46. Alternatively, the polypeptide can have at least 90%, 91%, 92%, 93%, 94%, 95%, 96%, or 97% sequence similarity to SEQ ID NO:46.

In a specific embodiment of Formula Id comprising the polypeptide, or a pharmaceutically acceptable salt thereof, X₁₃ is D-Val, X₂₁ is L, X₃₇ is A, X₃₈ is A, and the polypeptide further comprises a linker-fatty acid. In a particular embodiment, the linker-fatty acid is γE-(2-[2-(2-amino-ethoxy)-ethoxy]-acetyl)-εK—CO—(CH₂)₁₈—CO₂H. In a specific embodiment of Formula Id comprising the polypeptide, or a pharmaceutically acceptable salt thereof, the K at X₃₅ is conjugated to the linker-fatty acid. In some embodiments, the polypeptide comprises SEQ ID NO:47. In some embodiments, the polypeptide is SEQ ID NO:47. Alternatively, the polypeptide can have at least about 90 to 97% sequence similarity to SEQ ID NO:47. Alternatively, the polypeptide can have at least 90%, 91%, 92%, 93%, 94%, 95%, 96%, or 97% sequence similarity to SEQ ID NO:47.

In a specific embodiment of Formula Id comprising the polypeptide, or a pharmaceutically acceptable salt thereof, X₁₃ is V, X₂₁ is L, X₃₇ is A, X₃₈ is A, and the polypeptide further comprises a linker-fatty acid. In a particular embodiment, the linker-fatty acid is (γE-(2-[2-(2-amino-ethoxy)-ethoxy]-acetyl)-εK—CO—(CH₂)₁₈—CO₂H. In a specific embodiment of Formula Id comprising the polypeptide, or a pharmaceutically acceptable salt thereof, the K at X₃₅ is conjugated to the linker-fatty acid. In some embodiments, the polypeptide comprises SEQ ID NO:48. In some embodiments, the polypeptide is SEQ ID NO:48. Alternatively, the polypeptide can have at least about 90 to 97% sequence similarity to SEQ ID NO:48. Alternatively, the polypeptide can have at least 90%, 91%, 92%, 93%, 94%, 95%, 96%, or 97% sequence similarity to SEQ ID NO:48.

In another embodiment of Formula I, X₁ is γE, X₂ is absent, X₃ is absent, X₄ is G, X₅ is P, X₆ is S, X₁₁ is αMeL, X₁₃ is D-Val, X₁₅ is I, X₁₆ is Aib, X₁₇ is αMeL, X₁₉ is Q, X₂₀ is I, X₂₁ is K, X₂₂ is L, X₂₆ is R, X₂₇ is A, X₃₀ is A, X₃₁ is R, X₃₅ is K, X₃₇ is N, X₃₈ is T. In a specific embodiment, the polypeptide further comprises a linker-fatty acid. In a particular embodiment, the linker fatty acid is (2-[2-(2-Amino-ethoxy)-ethoxy]-acetyl)₂-γE-CO—(CH₂)₁₈—CO₂H. In a specific embodiment comprising the polypeptide, or a pharmaceutically acceptable salt thereof, the K at X₃₅ is conjugated to the linker-fatty acid.

In some embodiments, the polypeptide comprises SEQ ID NO:45. In some embodiments, the polypeptide is SEQ ID NO:45. Alternatively, the polypeptide can have at least about 90 to 97% sequence similarity to SEQ ID NO:45. Alternatively, the polypeptide can have at least 90%, 91%, 92%, 93%, 94%, 95%, 96%, or 97% sequence similarity to SEQ ID NO:45.

In another embodiment of Formula I, X₁ is absent, X₂ is absent, X₃ is absent, X₄ is absent, X₅ is absent, X₆ is absent, X₁₁ is L, X₁₃ is V, X₁₅ is I, X₁₆ is G, X₁₇ is αMeL, X₁₉ is Q, X₂₀ is K, K₂₁ is L, X₂₂ is I, X₂₆ is K, X₂₇ is Q, X₃₀ is E, X₃₁ is K, X₃₅ is K, X₃₇ is N, X₃₈ is T.

In a specific embodiment of the polypeptide, or a pharmaceutically acceptable salt thereof, the polypeptide further comprises a linker-fatty acid. In a particular embodiment, the linker-fatty acid is (2-[2-(2-amino-ethoxy)-ethoxy]-acetyl)₂γE-C20-OH)). In a specific embodiment comprising the polypeptide, or a pharmaceutically acceptable salt thereof, the K at X₃₅ is conjugated to the linker-fatty acid.

In some embodiments, the polypeptide comprises SEQ ID NO:49. In some embodiments, the polypeptide is SEQ ID NO:49. Alternatively, the polypeptide can have at least about 90 to 97% sequence similarity to SEQ ID NO:49. Alternatively, the polypeptide can have at least 90%, 91%, 92%, 93%, 94%, 95%, 96%, or 97% sequence similarity to SEQ ID NO:49.

In another embodiment of Formula I, X₁ is absent, X₂ is absent, X₃ is absent, X₄ is absent, X₅ is absent, X₆ is absent, X₁₁ is L, X₁₃ is V, X₁₅ is I, X₁₆ is G, X₁₇ is αMeL, X₁₉ is Q, X₂₀ is K, K₂₁ is L, X₂₂ is L, X₂₆ is R, X₂₇ is A, X₃₀ is A, X₃₁ is R, X₃₅ is K, X₃₇ is A, X₃₈ is A.

In a specific embodiment of the polypeptide, or a pharmaceutically acceptable salt thereof, the polypeptide further comprises a linker-fatty acid. In a particular embodiment, the linker-fatty acid is 2-[2-(2-Amino-ethoxy)-ethoxy]-acetyl)₂-γE-CO—(CH₂)₁₈—CO₂H. In a specific embodiment comprising the polypeptide, or a pharmaceutically acceptable salt thereof, the K at X₃₅ is conjugated to the linker-fatty acid.

In some embodiments, the polypeptide comprises SEQ ID NO:50. In other embodiments, the polypeptide is SEQ ID NO:50. Alternatively, the polypeptide can have at least about 90 to 97% sequence similarity to SEQ ID NO:50. Alternatively, the polypeptide can have at least 90%, 91%, 92%, 93%, 94%, 95%, 96%, or 97% sequence similarity to SEQ ID NO:50.

In one aspect, provided herein are polypeptides, or pharmaceutically acceptable salts thereof, comprising sequences with at least eight amino acid modifications from human urocortin-2 (SEQ ID NO:53). In some embodiments, the polypeptides provided herein comprise sequences containing at least one amino acid modification at position: 3, 19, 22, 23, 26, 30, 33, or 37 from human urocortin-2 (SEQ ID NO:53). In some embodiments, the polypeptides provided herein comprise sequences containing at least one of the following amino acid modifications from human urocortin-2 (SEQ ID NO:53): L3T, A19E, R22E, A23K, E26Q, T30E, R33E, R37Q, or any combination thereof.

In another embodiment, provided herein is a polypeptide comprising any one of SEQ ID NOs:1-52, or a pharmaceutically acceptable salt thereof. Alternatively, the polypeptide can have at least about 90 to 99% sequence similarity to any one of SEQ ID NOs:1-52, or a pharmaceutically acceptable salt thereof. Alternatively, the polypeptide can have at least 90%, 91%, 92%, 93%, 94%, 95%, 96%, 97%, 98%, or 99% sequence similarity to any one of SEQ ID NOs:1-52.

In a specific embodiment, the polypeptide comprises SEQ ID NO:1, or a pharmaceutically acceptable salt thereof. In a specific embodiment, the polypeptide comprises SEQ ID NO:2, or a pharmaceutically acceptable salt thereof. In a specific embodiment, the polypeptide comprises SEQ ID NO:22, or a pharmaceutically acceptable salt thereof. In a specific embodiment, the polypeptide comprises SEQ ID NO:25, or a pharmaceutically acceptable salt thereof. In a specific embodiment, the polypeptide comprises SEQ ID NO:29, or a pharmaceutically acceptable salt thereof. In a specific embodiment, the polypeptide comprises SEQ ID NO:41, or a pharmaceutically acceptable salt thereof. In a specific embodiment, the polypeptide comprises SEQ ID NO:42, or a pharmaceutically acceptable salt thereof. In a specific embodiment, the polypeptide comprises SEQ ID NO:45, or a pharmaceutically acceptable salt thereof. In a specific embodiment, the polypeptide comprises SEQ ID NO:46, or a pharmaceutically acceptable salt thereof. In a specific embodiment, the polypeptide comprises SEQ ID NO:48, or a pharmaceutically acceptable salt thereof. In a specific embodiment, the polypeptide comprises SEQ ID NO:49, or a pharmaceutically acceptable salt thereof. In a specific embodiment, the polypeptide comprises SEQ ID NO:50, or a pharmaceutically acceptable salt thereof.

In a specific embodiment, the polypeptide, or a pharmaceutically acceptable salt thereof, is selected from the group consisting of SEQ ID NO:1, SEQ ID NO:2, SEQ ID NO:22, SEQ ID NO:25, SEQ ID NO:29, SEQ ID NO:41, SEQ ID NO:42, SEQ ID NO:45, SEQ ID NO:46, SEQ ID NO:48, SEQ ID NO:49, and SEQ ID NO:50.

In another embodiment, provided herein is a pharmaceutical composition comprising a polypeptide, or a pharmaceutically acceptable salt thereof, and at least one pharmaceutically acceptable carrier, diluent, or excipient.

In another embodiment, a method is provided of treating a disease or condition selected from the group consisting of diabetes mellitus, obesity, chronic weight management, non-alcoholic fatty liver disease (NAFLD), non-alcoholic steatohepatitis (NASH), dyslipidemia, metabolic syndrome, chronic kidney disease (CKD), osteoarthritis (OA), obesity-related sleep apnea (OSA), sarcopenia, cachexia, sarcopenic obesity (SO), and polycystic ovarian syndrome (PCOS), the method comprising administering to an individual in need thereof an effective amount of a polypeptide, or a pharmaceutically acceptable salt thereof.

In another embodiment, provided herein is a polypeptide, or pharmaceutically acceptable salt thereof, for use in therapy.

In another embodiment, provided herein is a polypeptide, or a pharmaceutically acceptable salt thereof, for use in treating a disease or condition selected from the group consisting of diabetes mellitus, obesity, chronic weight management, non-alcoholic fatty liver disease (NAFLD), non-alcoholic steatohepatitis (NASH), dyslipidemia, metabolic syndrome, chronic kidney disease (CKD), osteoarthritis (OA), obesity-related sleep apnea (OSA), sarcopenia, cachexia, sarcopenic obesity (SO), and polycystic ovarian syndrome (PCOS).

In an embodiment, the present disclosure provides for the use of a polypeptide, or a pharmaceutically acceptable salt thereof, in the manufacture of a medicament for treatment of a disease or condition selected from the group consisting of diabetes mellitus, obesity, chronic weight management, non-alcoholic fatty liver disease (NAFLD), non-alcoholic steatohepatitis (NASH), dyslipidemia, metabolic syndrome, chronic kidney disease (CKD), osteoarthritis (OA), obesity-related sleep apnea (OSA), sarcopenia, cachexia, sarcopenic obesity (SO), and polycystic ovarian syndrome (PCOS).

In an embodiment, provided herein is a method of treating a disease or condition, selected from the group consisting of diabetes mellitus, obesity, chronic weight management, non-alcoholic fatty liver disease (NAFLD), non-alcoholic steatohepatitis (NASH), dyslipidemia, metabolic syndrome, chronic kidney disease (CKD), osteoarthritis (OA), obesity-related sleep apnea (OSA), sarcopenia, cachexia, sarcopenic obesity (SO), and polycystic ovarian syndrome (PCOS), the method comprising administering to an individual in need thereof an effective amount of a polypeptide, or a pharmaceutically salt thereof, and an effective amount of an additional therapeutic agent.

In an embodiment, provided herein is a polypeptide, or a pharmaceutically acceptable salt thereof, that may be used in simultaneous, separate, or sequential combinations with one or more additional therapeutic agents.

In an embodiment, the additional therapeutic agent is a dual agonist of the GIP and GLP-1 receptors. In a specific embodiment, the dual agonist of the GIP and GLP-1 receptors is a compound comprising SEQ ID NO:55. In an embodiment, the additional therapeutic agent is an agonist of the GLP-1 receptor. In an embodiment, the GLP-1 receptor agonist is a compound comprising SEQ ID NO:79, SEQ ID NO:80, or SEQ ID NO:81.

### DETAILED DESCRIPTION

Amino acids comprise an amino group and a carboxylic acid group. In α-amino acids, a group comprising the 20 canonical amino acids, the amino group and the carboxylic acid group are covalently linked to the same carbon, defined as the α-carbon. The α-carbon may also be substituted with additional functional groups, and these functional groups may have defined stereochemistry. The 20 canonical amino acids are most often found as the L-stereoisomer, and so it is assumed that unless otherwise specified, references to amino acids are specifically to the L-stereoisomer.

Polypeptides are polymers of amino acids covalently linked via amide bonds between a carboxylic acid group on one monomer and an amino group on the next monomer. Most commonly, these are the α-carboxylic acid group and the α-amino group, respectively, and thus unless otherwise specified, polypeptides written using standard one-letter or three-letter amino acid codes are assumed to be α-amino acid polymers.

Typically, amino acids are drawn with the amino group on the left and the carboxylic acid group on the right, and polypeptide sequences are written such that when they are unmodified linear alpha-amino acid chains, the first amino acid is assumed to have a free amino group to the left of the chain (H₂N-polypeptide, also written as H-polypeptide or polypeptide), and the final amino acid is assumed to have a free carboxylic acid group at the far right end of the chain (polypeptide-COOH, typically written as polypeptide or polypeptide-OH). Thus, the left end of a polypeptide is also known as the N-terminus (due to the nitrogen in the amine group), and the right end of a polypeptide is also known as the C-terminus (due to the carboxylic acid group). In the absence of additional description, if a polypeptide is written solely as standard one-letter or three-letter amino acid codes, it is assumed that the polypeptide begins with a free amino group on the N-terminus and ends with a free carboxylic acid on the C-terminus.

Polypeptides may further comprise N-terminal modifications (e.g., acetylation, Ac; methylation, Me; acylation, or other modifications). Polypeptides may also have C-terminal modifications; most commonly, the C-terminal group is an amide (—CONH₂, usually written as polypeptide-NH₂) instead of a carboxylic acid, although other C-terminal modifications are known in the art.

The amino acid sequences of polypeptides described herein incorporate naturally occurring amino acids, typically depicted herein using standard one letter codes (e.g., L=leucine), as well as α-methyl substituted residues of natural amino acids (e.g., α-methyl-leucine (αMeL), and non-natural amino acids, such as α-amino isobutyric acid (Aib). The structures of these amino acids and other monomers and modifications are depicted below.

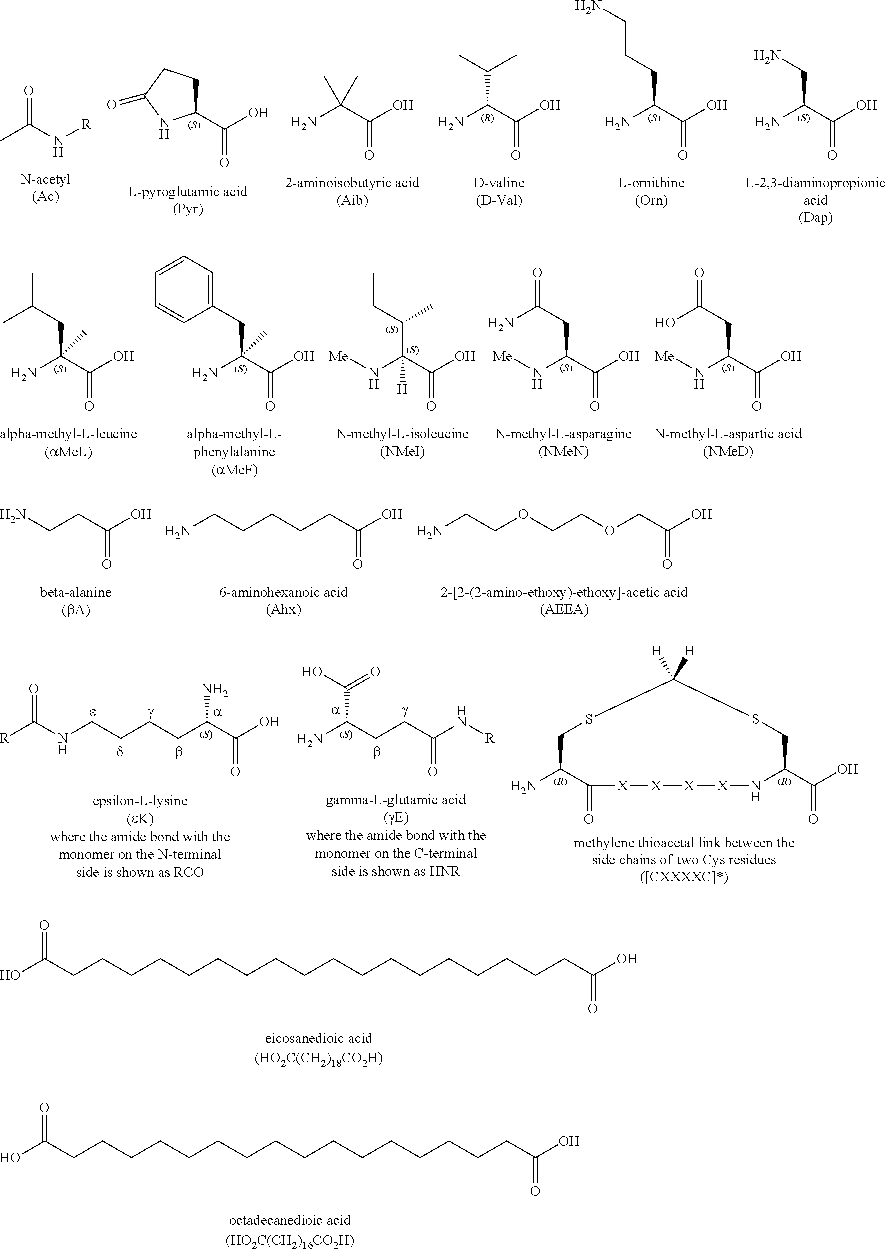

Consequently, as used herein “Ac” means N-acetyl. As used herein “Pyr” means L-pyroglutamic acid. As used herein “Aib” means 2-aminoisobutyric acid. As used herein “D-Val” means D-valine. As used herein “Orn” means L-ornithine. As used herein “Dap” means L-2,3-diaminopropionic acid. As used herein “αMeL” means alpha-methyl-L-leucine. As used herein “αMeF” means alpha-methyl-L-phenylalanine. As used herein “NMeI” means N-methyl-L-isoleucine. As used herein “NMeN” means N-methyl-L-asparagine. As used herein “NMeD” means N-methyl-L-aspartic acid. As used herein “βA” means beta-alanine. As used herein “Ahx” means 6-aminohexanoic acid. As used herein “AEEA” means 2-[2-(2-amino-ethoxy)-ethoxy]-acetic acid. As used herein “εK” means epsilon-L-lysine. As used herein “γE” means gamma-L-glutamic acid.

“N-term” may refer to N-terminus or N-terminal (if used as a modifier); “C-term” may refer to C-terminus or C-terminal (if used as a modifier).

It should be noted that the combination of beneficial characteristics of exemplary analogs described herein is not the result of any single modification in isolation but is instead achieved through the novel combinations of structural features described herein.

In some embodiments, the present disclosure provides a polypeptide, or a pharmaceutically acceptable salt thereof, of Formula I comprising:

(SEQ ID NO: 68)
X₁X₂X₃X₄X₅X₆IVTSX₁₁DX₁₃PX₁₅X₁₆X₁₇LX₁₉
X₂₀X₂₁X₂₂EQEX₂₆X₂₇EKX₃₀X₃₁QQAX₃₅E
X₃₇X₃₈EILAQV,

wherein
X₁ is Pyr, E, γE, or absent,
X₂ is G or absent,
X₃ is G, S, or absent,
X₄ is S, P, G, or absent,
X₅ is S, P, or absent,
X₆ is G, S, P, or absent,
X₁₁ is L or αMeL,
X₁₃ is V or D-Val,
X₁₅ is T or I,
X₁₆ is Aib or G,
X₁₇ is L or αMeL,
X₁₉ is Q or E,
X₂₀ is K or I,
X₂₁ is I, K, Aib, or L,
X₂₂ is I or L,
X₂₆ is R or K,
X₂₇ is A or Q,
X₃₀ is A or E,
X₃₁ is R or K,
X₃₅ is K or T,
X₃₇ is A or N,
X₃₈ is A or T,
wherein the N-term amino acid is optionally N-acylated, N-acetylated or N-methylated, and wherein the C-term amino acid is optionally amidated.

In some embodiments of Formula I, the V at position 44 is the C-term amino acid. The C-term may comprise an amide, carboxylic acid, or other modifications. The N-term amino acid depends on the embodiment but will always be the first amino acid present in the sequence which indicates the start of the polypeptide. Therefore, in embodiments of Formula I, the N-term amino acid may be at position X₁, X₂, X₃, X₄, X₅, X₆, or the I at position 7. Further, in some embodiments of Formula I, the N-term may comprise additional modifications (e.g., acetylation, Ac; methylation, Me; acylation, or other modifications). Further, if any amino acids at positions X₁—X₆ are indicated as “absent”, the next available amino acid will be conjugated to the previously available amino acid. Thus, various embodiments of Formula I result in polypeptides comprising varying lengths, e.g., 38 to 44 amino acids.

Further, in addition to the sequences described herein, the polypeptides described herein may include one or more conservative amino acid substitutions, provided, however, that the polypeptides remain capable of having activity at the CRHR2.

In some embodiments, the polypeptide, or a pharmaceutically acceptable salt thereof, further comprises a means for increasing the half-life of the polypeptide. In some embodiments, the polypeptide, or a pharmaceutically acceptable salt thereof, further comprises a means for increasing the half-life of a polypeptide via any amino acid on the polypeptide with a suitable functional group for conjugation. In some embodiments, the amino acid with a suitable functional group for conjugation is γE, E, or K.

In some embodiments, the functional group available for conjugation is conjugated to a phosphonate, tetrazole, sulfonate, bifurcated fatty acid, peptide, protein (such as a VHH, Fc, or monoclonal antibody), or protein fragment (such as, Fab, scFv, Fv, or scFab (single chain Fab). In some embodiments, the fatty acid, linker, protein, or protein fragment may further act as an albumin binder and provide a potential to further generate long-acting compounds.

In some embodiments, the means is a fatty acid.

In some embodiments wherein the amino acid with a functional group for conjugation is K, the conjugation is to an epsilon-amino group of the K. In some embodiments, the polypeptide further comprises a fatty acid conjugated to K at X₃₅, wherein the fatty acid is conjugation to the epsilon-amino group of K via a direct bond or via a linker between K and the fatty acid. In some embodiments wherein the amino acid with a functional group for conjugation is γE at X₁, the conjugation is via an amide bond between the alpha amino group of the γE and the carboxylic group of the fatty acid. In some embodiments, the means for increasing the half-life of the polypeptide is a fatty acid, wherein the fatty acid is conjugated to the polypeptide at an amino acid with a functional group available for conjugation, via a direct bond or via a linker between the amino acid and the fatty acid.

In some embodiments, the N-term amino acid is N-acylated via a fatty acid via a fatty acid conjugated to the N-term amino acid, via a direct bond or via a linker between the amino acid and the fatty acid. In some such embodiments, the linker-fatty acid is conjugated to the peptide after synthesis of the peptide. In other such embodiments, the linker-fatty acid is conjugated to an amino acid (for example, in some embodiments to an E or γE) prior to addition to the peptide.

In some embodiments, the polypeptide is conjugated to a fatty acid optionally via a linker between the amino acid and the fatty acid. Thus, in some embodiments, the polypeptide is conjugated to a fatty acid via a direct bond between the amino acid and the fatty acid. In other embodiments, the polypeptide is conjugated to a fatty acid via a linker between the amino acid and the fatty acid. In some embodiments, the fatty acid is a C₁₆-C₂₂ fatty acid. It some embodiments, the conjugation of a linker to a C₁₆-C₂₂ fatty acid is referred to as a linker-fatty acid.

Examples of saturated C₁₆-C₂₂ fatty acids for use herein include, but are not limited to, palmitic acid (hexadecenoic acid)(C₁₆ monoacid), hexadecanedioic acid (C₁₆ diacid), margaric acid (heptadecanoic acid) (C₁₇ monoacid), heptadecanedioic acid (C₁₇ diacid), stearic acid (Cis monoacid), octadecanedioic acid (C₁₈ diacid), nonadecylic acid (nonadecanoic acid) (C₁₉ monoacid), nonadecanedioic acid (C₁₉ diacid), arachidic acid (eicosanoic acid) (C₂₀ monoacid), eicosanedioic acid (C₂₀ diacid), heneicosylic acid (heneicosanoic acid) (C₂₁ monoacid), henicosanedioic acid (C₂₁ diacid), behenic acid (docosanoic acid)(C₂₂ monoacid), docosanedioic acid (C₂₂ diacid), including branched and substituted derivatives thereof.

In certain instances, the linker can have one or more (2-[2-(2-amino-ethoxy)-ethoxy]-acetyl moieties or εK, optionally in combination with one or more amino acids.

In instances where the linker includes at least one amino acid, the amino acid can be one to five E or γE amino acid residues. In some instances, the linker can include one or two or three or four or five E or γE amino acid residues, including the D-forms thereof. For example, the linker can include either one or two or three or four γE amino acid residues. Alternatively, the linker can include one to five amino acid residues (e.g., E or γE amino acids) used in combination with one to five (2-[2-(2-amino-ethoxy)-ethoxy]-acetyl) (“AEEA”) or one to five εK moieties. Specifically, the linker can be combinations of one to five E or γE amino acids and one to five (2-[2-(2-amino-ethoxy)-ethoxy]-acetyl) (“AEEA”) or one to five εK moieties, or one to five Glu or γGlu amino acids and one to five εK moieties. In some instances, the linker can be combinations of one or two or three γGlu amino acids and one or two (2-[2-(2-amino-ethoxy)-ethoxy]-acetyl) or εK moieties.

In specific embodiments, the polypeptide, or pharmaceutically acceptable salt thereof, further comprises a linker-fatty acid, wherein the linker-fatty acid is selected from the group consisting of: (2-[2-(2-amino-ethoxy)-ethoxy]-acetyl)-PKE-γE-CO—(CH₂)₁₈—CO₂H, Ahx-(2-[2-(2-amino-ethoxy)-ethoxy]-acetyl)-γE-CO—(CH₂)₁₈—CO₂H, γE-(2-[2-(2-amino-ethoxy)-ethoxy]-acetyl)-Ahx-CO—(CH₂)₁₈—CO₂H, γE-GKEKEKE-CO—(CH₂)₁₈—CO₂H, G-(βA)₃-γE-CO—(CH₂)₁₈—CO₂H, G-(βA)₄-γE-CO—(CH₂)₁₈—CO₂H, (2-[2-(2-amino-ethoxy)-ethoxy]-acetyl)-KEKEKE-γE-CO—(CH₂)₁₈—CO₂H, βA-S-βA-S-γE-CO—(CH₂)₁₈—CO₂H, γE-PKE-Ahx-CO—(CH₂)₁₈—CO₂H, γE-PKE-εK—CO—(CH₂)₁₈—CO₂H, γE-PPP—εK—CO—(CH₂)₁₈—CO₂H, γE-PPPPPP-CO—(CH₂)₁₈—CO₂H, γE-PEPEPE-CO—(CH₂)₁₈—CO₂H, γE-(2-[2-(2-amino-ethoxy)-ethoxy]-acetyl)₂-εK—CO—(CH₂)₁₈—CO₂H, γE-εK-γE-εK—CO—(CH₂)₁₈—CO₂H, γE-εK-(2-[2-(2-amino-ethoxy)-ethoxy]-acetyl)-εK—CO—(CH₂)₁₈—CO₂H, γE-(2-[2-(2-amino-ethoxy)-ethoxy]-acetyl)-εK—CO—(CH₂)₁₆—CO₂H, 2-[2-(2-amino-ethoxy)-ethoxy]-acetyl)-εK—CO—(CH₂)₁₈—CO₂H, (2-[2-(2-amino-ethoxy)-ethoxy]-acetyl)-K—CO—(CH₂)₁₈—CO₂H, (2-[2-(2-amino-ethoxy)-ethoxy]-acetyl)-εK—CO—(CH₂)₁₆—CO₂H, (2-[2-(2-amino-ethoxy)-ethoxy]-acetyl)-Dap-CO—(CH₂)₁₈—CO₂H, (2-[2-(2-amino-ethoxy)-ethoxy]-acetyl)₂-γE-CO—(CH₂)₁₈—CO₂H, γE-(2-[2-(2-amino-ethoxy)-ethoxy]-acetyl)-γE-CO—(CH₂)₁₈—CO₂H, γE₂-(2-[2-(2-amino-ethoxy)-ethoxy]-acetyl)-CO—(CH₂)₁₈—CO₂H, 2-[2-(2-amino-ethoxy)-ethoxy]-acetyl)-εK-γE-CO—(CH₂)₁₈—CO₂, 2-[2-(2-amino-ethoxy)-ethoxy]-acetyl)-γE-εK—CO—(CH₂)₁₈—CO₂H, γK-(2-[2-(2-amino-ethoxy)-ethoxy]-acetyl)-γE-CO—(CH₂)₁₈—CO₂H, γK-γE-(2-[2-(2-amino-ethoxy)-ethoxy]-acetyl)-CO—(CH₂)₁₈—CO₂H, γE-εK-(2-[2-(2-amino-ethoxy)-ethoxy]-acetyl)-CO—(CH₂)₁₈—CO₂H, γE-γE-εK—CO—(CH₂)₁₈—CO₂H, γE-εK-γE-CO—(CH₂)₁₈—CO₂H, εK-γE-γE-CO—(CH₂)₁₈—CO₂H, K(GKEKEKE-CO—(CH₂)₁₈—CO₂H), 2-[2-(2-amino-ethoxy)-ethoxy]-acetyl)-γE₂-CO—(CH₂)₁₈—CO₂H, γE-(2-[2-(2-amino-ethoxy)-ethoxy]-acetyl)-εK—CO—(CH₂)₁₈—CO₂H, 2-[2-(2-amino-ethoxy)-ethoxy]-acetyl)₂γE-C20-OH, and γE-(2-[2-(2-amino-ethoxy)-ethoxy]-acetyl)-εK—CO—(CH₂)₁₈—CO₂H.

In some embodiments, the N-term amino acid is N-acetylated. In some embodiments, the N-term amino acid is N-methylated. In some embodiments, the C-term amino acid is methylated.

In one embodiment of Formula I, X₁ is absent, X₂ is absent, X₃ is G, X₄ is S, X₅ is P, X₆ is S, X₁₁ is αMeL, X₁₃ is V, X₁₅ is T, X₁₆ is Aib, X₁₇ is αMeL, X₁₉ is Q, X₂₀ is K, X₂₁ is I or L, X₂₂ is L, X₂₆ is R, X₂₇ is A, X₃₀ is A, X₃₁ is R, X₃₅ is K, X₃₇ is A, and X₃₈ is A. In such embodiments, the polypeptide comprises a 42 amino acid sequence.

Thus, in some embodiments, provided herein is a polypeptide, or a pharmaceutically acceptable salt thereof, of Formula Ia comprising:

(SEQ ID NO: 69)
GSPSIVTS αMeL DVPT Aib αMeL
LQKX₂₁LEQERAEKARQQAKEAAEILAQV,

wherein,
X₂₁ is I or L[wherein when X₁—X₂ are absent, X₂₁ is at position 19 of the polypeptide], wherein the N-term amino acid is optionally N-acetylated, and wherein the C-term amino acid is optionally amidated.

In some embodiments of Formula Ia, X₂₁ is I. In some embodiments, the polypeptide comprises SEQ ID NO:20. In another embodiment, the polypeptide is SEQ ID NO:20. Alternatively, the polypeptide can have at least about 90 to 97% sequence similarity to SEQ ID NO:20. Alternatively, the polypeptide can have at least 90%, 91%, 92%, 93%, 94%, 95%, 96%, or 97% sequence similarity to SEQ ID NO:20. An exemplary embodiment comprising SEQ ID NO:20 is shown.

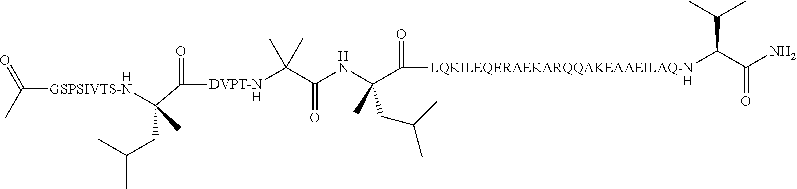

In a specific embodiment of Formula Ia comprising the polypeptide, or a pharmaceutically acceptable salt thereof, X₂₁ is I and the polypeptide further comprises a linker-fatty acid. In a particular embodiment, the linker-fatty acid is γE-(2-[2-(2-amino-ethoxy)-ethoxy]-acetyl)-εK—CO—(CH₂)₁₈—CO₂H. In a specific embodiment of Formula Ia comprising the polypeptide, or a pharmaceutically acceptable salt thereof, the K at X₃₅ is conjugated to the linker-fatty acid. In another embodiment, the polypeptide comprises SEQ ID NO:1. In another embodiment, the polypeptide is SEQ ID NO:1. Alternatively, the polypeptide can have at least about 90 to 97% sequence similarity to SEQ ID NO: 1. Alternatively, the polypeptide can have at least 90%, 91%, 92%, 93%, 94%, 95%, 96%, or 97% sequence similarity to SEQ ID NO:1. An exemplary embodiment comprising SEQ ID NO:1 is shown.

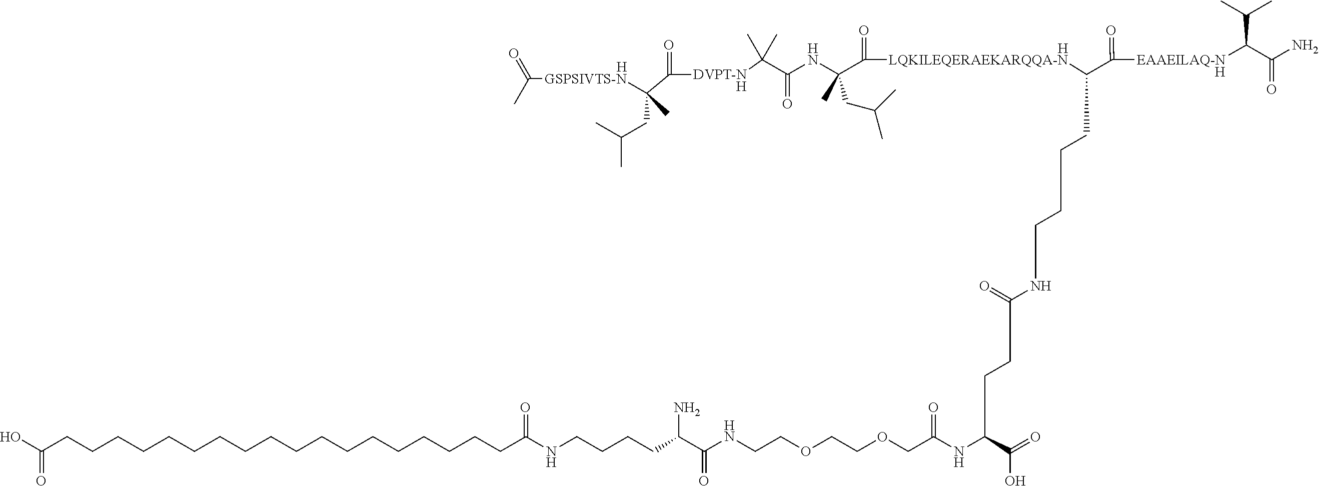

In a specific embodiment of Formula Ia comprising the polypeptide, or a pharmaceutically acceptable salt thereof, X₂₁ is L and the polypeptide further comprises a linker-fatty acid. In a particular embodiment, the linker-fatty acid is γE-(2-[2-(2-amino-ethoxy)-ethoxy]-acetyl)-εK—CO—(CH₂)₁₈—CO₂H. In a specific embodiment of Formula Ia comprising the polypeptide, or a pharmaceutically acceptable salt thereof, the K at X₃₅ is conjugated to the linker-fatty acid. In another embodiment, the polypeptide comprises SEQ ID NO:2. In another embodiment, the polypeptide is SEQ ID NO:2. Alternatively, the polypeptide can have at least about 90 to 97% sequence similarity to SEQ ID NO:2. Alternatively, the polypeptide can have at least 90%, 91%, 92%, 93%, 94%, 95%, 96%, or 97% sequence similarity to SEQ ID NO:2. An exemplary embodiment comprising SEQ ID NO:2 is shown.

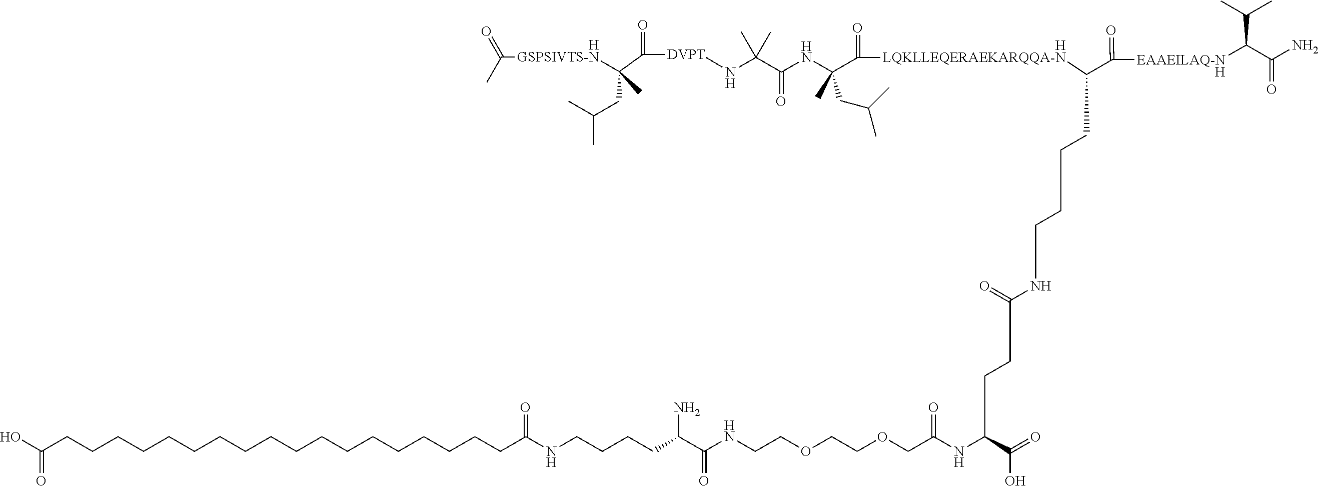

In a specific embodiment of Formula Ia comprising the polypeptide, or a pharmaceutically acceptable salt thereof, X₂₁ is I and the polypeptide further comprises a linker-fatty acid. In a particular embodiment, the linker-fatty acid is 2-[2-(2-amino-ethoxy)-ethoxy]-acetyl)-PKE-γE-CO—(CH₂)₁₈—CO₂H. In a specific embodiment of Formula Ia comprising the polypeptide, or a pharmaceutically acceptable salt thereof, the K at X₃₅ is conjugated to the linker-fatty acid. In another embodiment, the polypeptide comprises SEQ ID NO:3. In another embodiment, the polypeptide is SEQ ID NO:3. Alternatively, the polypeptide can have at least about 90 to 97% sequence similarity to SEQ ID NO:3. Alternatively, the polypeptide can have at least 90%, 91%, 92%, 93%, 94%, 95%, 96%, or 97% sequence similarity to SEQ ID NO:3. An exemplary embodiment comprising SEQ ID NO:3 is shown.

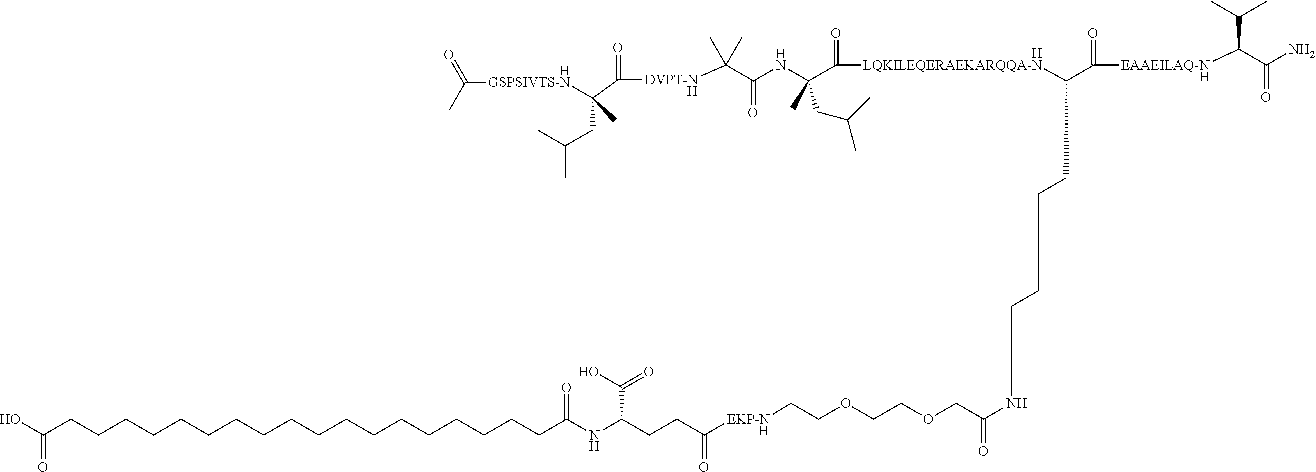

In a specific embodiment of Formula Ia comprising the polypeptide, or a pharmaceutically acceptable salt thereof, X₂₁ is I and the polypeptide further comprises a linker-fatty acid. In a particular embodiment, the linker-fatty acid is Ahx-(2-[2-(2-amino-ethoxy)-ethoxy]-acetyl)-γE-CO—(CH₂)₁₈—CO₂H. In a specific embodiment of Formula Ia comprising the polypeptide, or a pharmaceutically acceptable salt thereof, the K at X₃₅ is conjugated to the linker-fatty acid. In another embodiment, the polypeptide comprises SEQ ID NO:4. In another embodiment, the polypeptide is SEQ ID NO:4. Alternatively, the polypeptide can have at least about 90 to 97% sequence similarity to SEQ ID NO:4. Alternatively, the polypeptide can have at least 90%, 91%, 92%, 93%, 94%, 95%, 96%, or 97% sequence similarity to SEQ ID NO:4. An exemplary embodiment comprising SEQ ID NO:4 is shown.

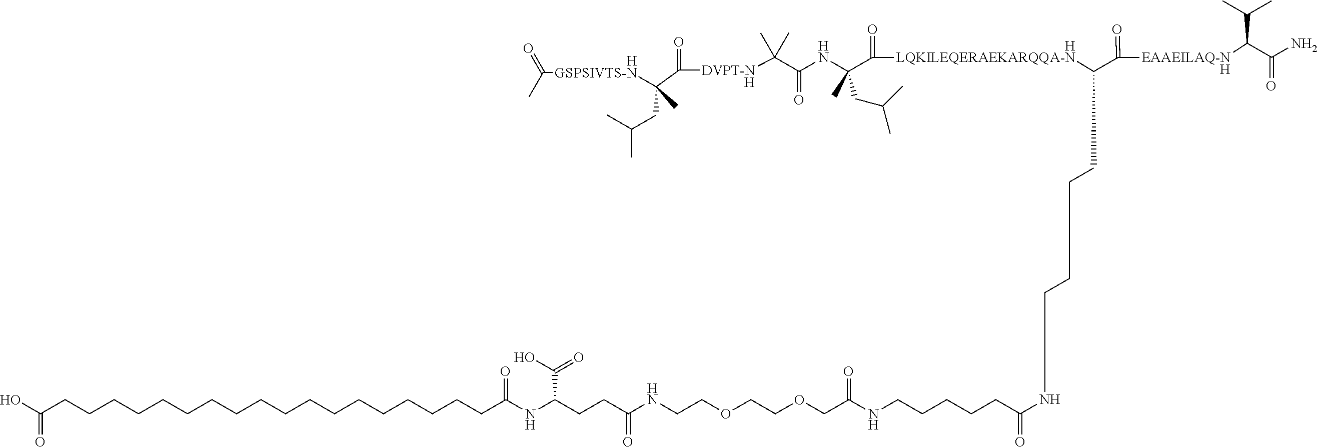

In a specific embodiment of Formula Ia comprising the polypeptide, or a pharmaceutically acceptable salt thereof, X₂₁ is I and the polypeptide further comprises a linker-fatty acid. In a particular embodiment, the linker-fatty-acid is γE-(2-[2-(2-amino-ethoxy)-ethoxy]-acetyl)-Ahx-CO—(CH₂)₁₈—CO₂H. In a specific embodiment of Formula Ia comprising the polypeptide, or a pharmaceutically acceptable salt thereof, the K at X₃₅ is conjugated to the linker-fatty acid. In another embodiment, the polypeptide comprises SEQ ID NO:5. In another embodiment, the polypeptide is SEQ ID NO:5. Alternatively, the polypeptide can have at least about 90 to 97% sequence similarity to SEQ ID NO:5. Alternatively, the polypeptide can have at least 90%, 91%, 92%, 93%, 94%, 95%, 96%, or 97% sequence similarity to SEQ ID NO:5. An exemplary embodiment comprising SEQ ID NO:5 is shown.

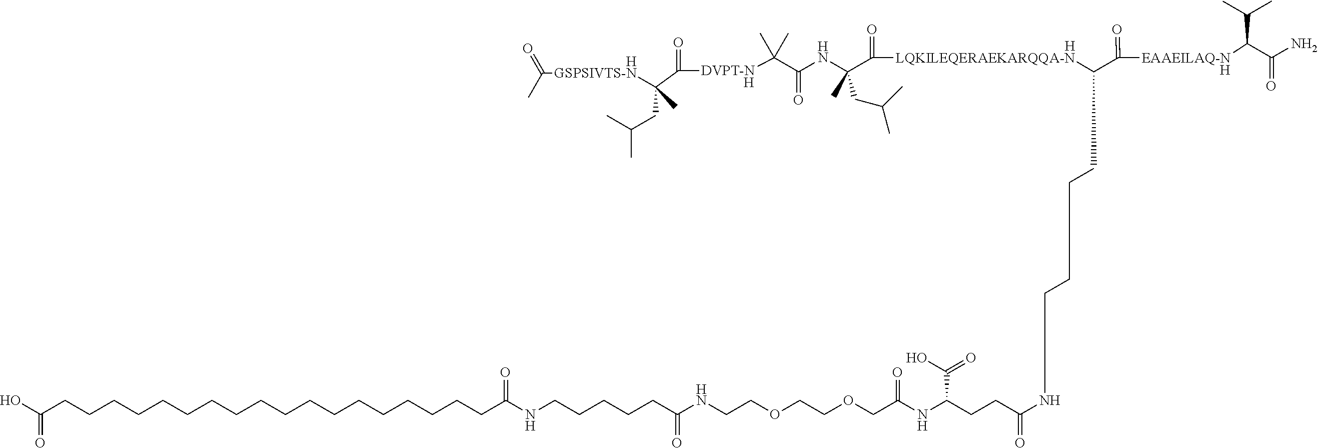

In a specific embodiment of Formula Ia comprising the polypeptide, or a pharmaceutically acceptable salt thereof, X₂₁ is I and the polypeptide further comprises a linker-fatty acid. In a particular embodiment, the linker-fatty acid is γE-GKEKEKE-CO—(CH₂)₁₈—CO₂H. In a specific embodiment of Formula Ia comprising polypeptide or a pharmaceutically acceptable salt thereof, the K at X₃₅ is conjugated to the linker-fatty acid. In another embodiment, the polypeptide comprises SEQ ID NO:6. In another embodiment, the polypeptide is SEQ ID NO:6. Alternatively, the polypeptide can have at least about 90 to 97% sequence similarity to SEQ ID NO:6. Alternatively, the polypeptide can have at least 90%, 91%, 92%, 93%, 94%, 95%, 96%, or 97% sequence similarity to SEQ ID NO:6. An exemplary embodiment comprising SEQ ID NO:6 is shown.

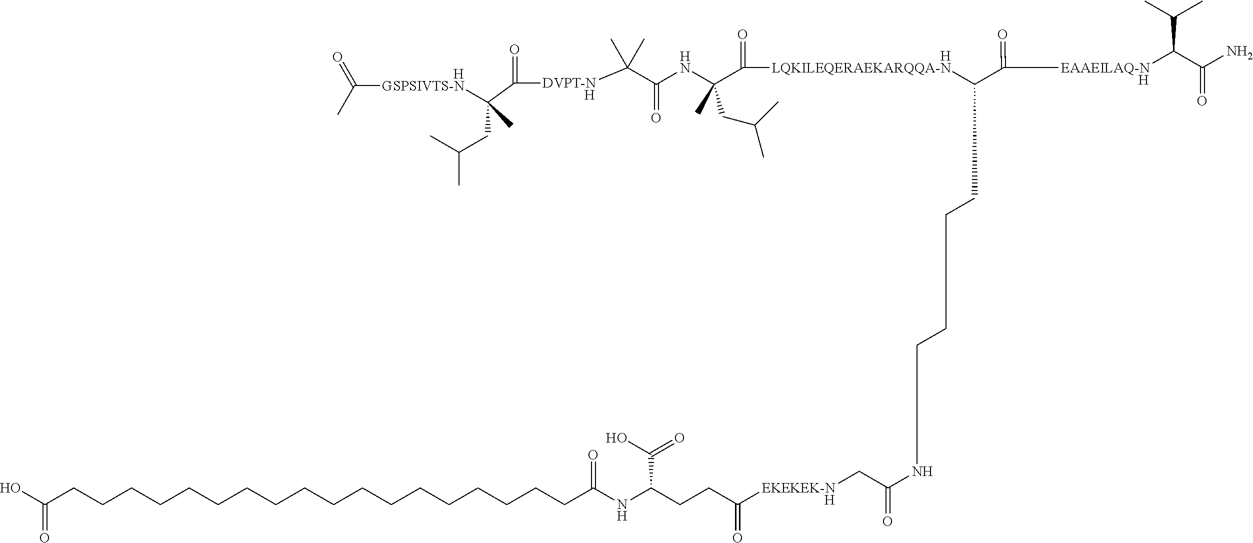

In a specific embodiment of Formula Ia comprising the polypeptide, or a pharmaceutically acceptable salt thereof, X₂₁ is I and the polypeptide further comprises a linker-fatty acid. In a particular embodiment, the linker-fatty acid is G-(βA)₃-γE-CO—(CH₂)₁₈—CO₂H. In a specific embodiment of Formula Ia comprising the polypeptide, or a pharmaceutically acceptable salt thereof, the K at X₃₅ is conjugated to the linker-fatty acid. In another embodiment, the polypeptide comprises SEQ ID NO:7. In another embodiment, the polypeptide is SEQ ID NO:7. Alternatively, the polypeptide can have at least about 90 to 97% sequence similarity to SEQ ID NO:7. Alternatively, the polypeptide can have at least 90%, 91%, 92%, 93%, 94%, 95%, 96%, or 97% sequence similarity to SEQ ID NO:7. An exemplary embodiment comprising SEQ ID NO:7 is shown.

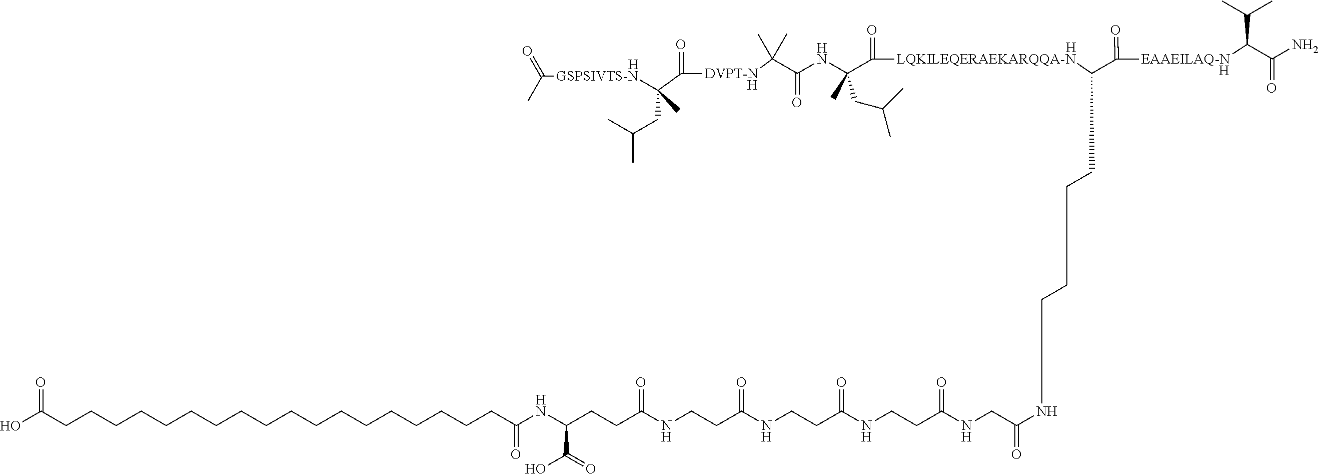

In a specific embodiment of Formula Ia comprising the polypeptide, or a pharmaceutically acceptable salt thereof, X₂₁ is I and the polypeptide further comprises a linker-fatty acid. In a particular embodiment, the linker-fatty acid is G-(βA)₄-γE-CO—(CH₂)₁₈—CO₂H. In a specific embodiment of Formula Ia comprising the polypeptide, or a pharmaceutically acceptable salt thereof, the K at X₃₅ is conjugated to the linker-fatty acid. In another embodiment, the polypeptide comprises SEQ ID NO:8. In another embodiment the polypeptide is SEQ ID NO:8. Alternatively, the polypeptide can have at least about 90 to 97% sequence similarity to SEQ ID NO:8. Alternatively, the polypeptide can have at least 90%, 91%, 92%, 93%, 94%, 95%, 96%, or 97% sequence similarity to SEQ ID NO:8. An exemplary embodiment comprising SEQ ID NO:8 is shown.

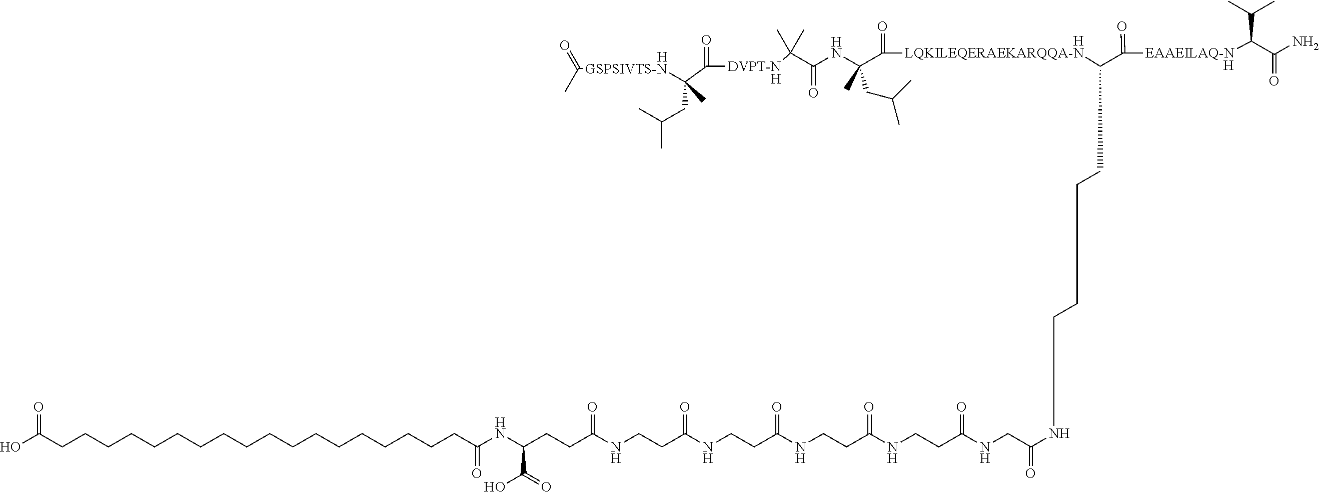

In a specific embodiment of Formula Ia comprising the polypeptide, or a pharmaceutically acceptable salt thereof, X₂₁ is I and the polypeptide further comprises a linker-fatty acid. In a particular embodiment, the linker-fatty acid is 2-[2-(2-amino-ethoxy)-ethoxy]-acetyl)-KEKEKE-γE-CO—(CH₂)₁₈—CO₂H. In a specific embodiment of Formula Ia comprising the polypeptide, or a pharmaceutically acceptable salt thereof, the K at X₃₅ is conjugated to the linker-fatty acid. In another embodiment, the polypeptide comprises SEQ ID NO:9. In another embodiment, the polypeptide is SEQ ID NO:9. Alternatively, the polypeptide can have at least about 90 to 97% sequence similarity to SEQ ID NO:9. Alternatively, the polypeptide can have at least 90%, 91%, 92%, 93%, 94%, 95%, 96%, or 97% sequence similarity to SEQ ID NO:9. An exemplary embodiment comprising SEQ ID NO:9 is shown.

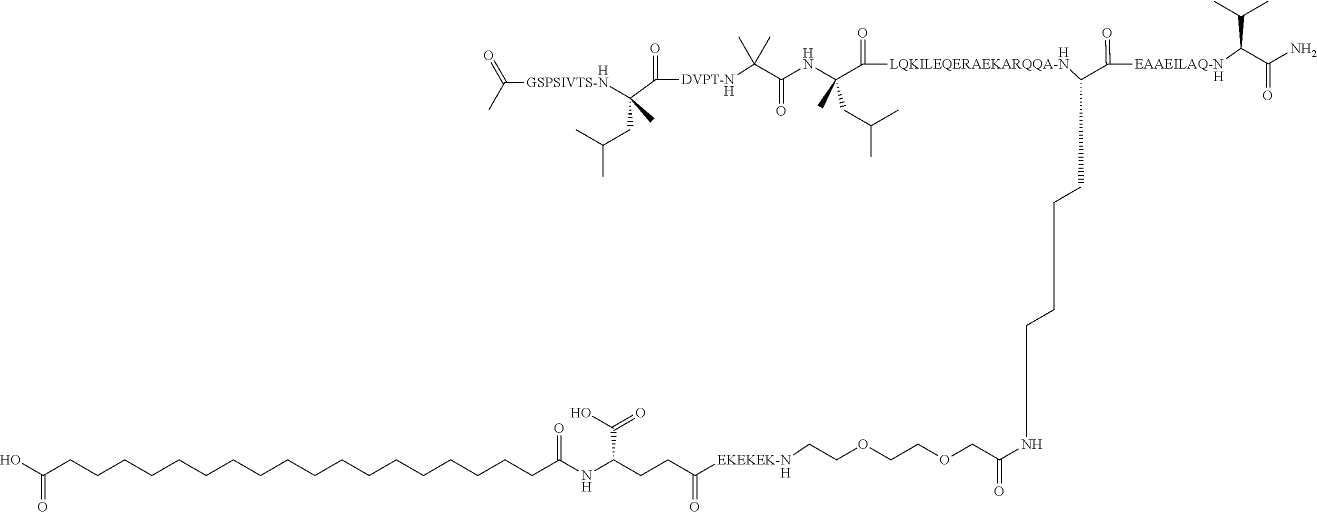

In a specific embodiment of Formula Ia comprising the polypeptide, or a pharmaceutically acceptable salt thereof, X₂₁ is I and the polypeptide further comprises a linker-fatty acid. In a particular embodiment, the linker-fatty acid is βA-S-βA-S-γE-CO—(CH₂)₁₈—CO₂H. In a specific embodiment of Formula Ia comprising the polypeptide, or a pharmaceutically acceptable salt thereof, the K at X₃₅ is conjugated to the linker-fatty acid. In another embodiment, the polypeptide comprises SEQ ID NO:10. In another embodiment, the polypeptide is SEQ ID NO:10. Alternatively, the polypeptide can have at least about 90 to 97% sequence similarity to SEQ ID NO:10. Alternatively, the polypeptide can have at least 90%, 91%, 92%, 93%, 94%, 95%, 96%, or 97% sequence similarity to SEQ ID NO:10. An exemplary embodiment comprising SEQ ID NO:10 is shown.

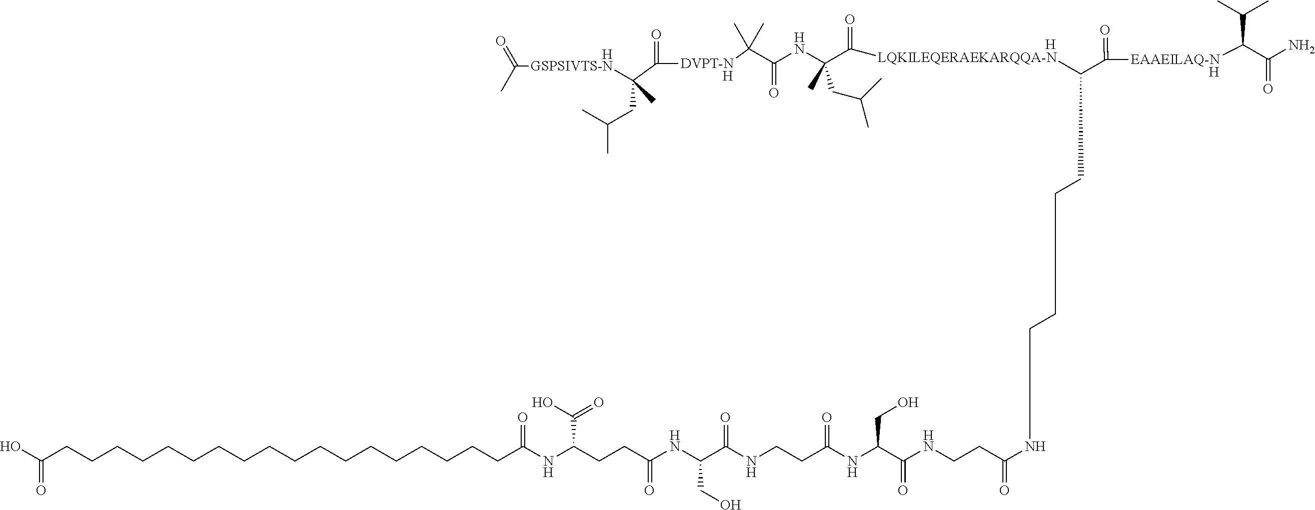

In a specific embodiment of Formula Ia comprising the polypeptide, or a pharmaceutically acceptable salt thereof, X₂₁ is I and the polypeptide further comprises a linker-fatty acid. In a particular embodiment, the linker-fatty acid is γE-PKE-Ahx-CO—(CH₂)₁₈—CO₂H. In a specific embodiment of Formula Ia comprising the polypeptide, or a pharmaceutically acceptable salt thereof, the K at X₃₅ is conjugated to the linker-fatty acid. In another embodiment, the polypeptide comprises SEQ ID NO:11. In another embodiment, the polypeptide is SEQ ID NO:11. Alternatively, the polypeptide can have at least about 90 to 97% sequence similarity to SEQ ID NO:11. Alternatively, the polypeptide can have at least 90%, 91%, 92%, 93%, 94%, 95%, 96%, or 97% sequence similarity to SEQ ID NO:11. An exemplary embodiment comprising SEQ ID NO:11 is shown.

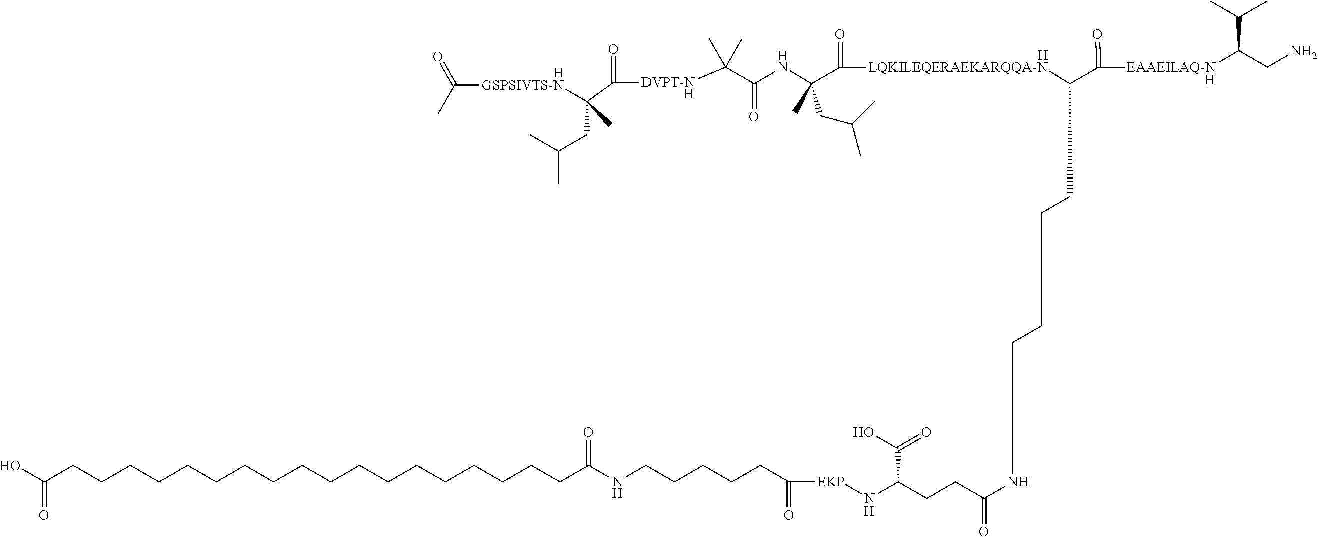

In a specific embodiment of Formula Ia comprising the polypeptide, or a pharmaceutically acceptable salt thereof, X₂₁ is L and the polypeptide further comprises a linker-fatty acid. In a particular embodiment, the linker-fatty acid is γE-(2-[2-(2-amino-ethoxy)-ethoxy]-acetyl)-εK—CO—(CH₂)₁₆—CO₂H. In a specific embodiment of Formula Ia comprising the polypeptide, or a pharmaceutically acceptable salt thereof, the K at X₃₅ is conjugated to the linker-fatty acid. In another embodiment, the polypeptide comprises SEQ ID NO:12. In another embodiment, the polypeptide is SEQ ID NO:12. Alternatively, the polypeptide can have at least about 90 to 97% sequence similarity to SEQ ID NO: 12. Alternatively, the polypeptide can have at least 90%, 91%, 92%, 93%, 94%, 95%, 96%, or 97% sequence similarity to SEQ ID NO:12. An exemplary embodiment comprising SEQ ID NO:12 is shown.

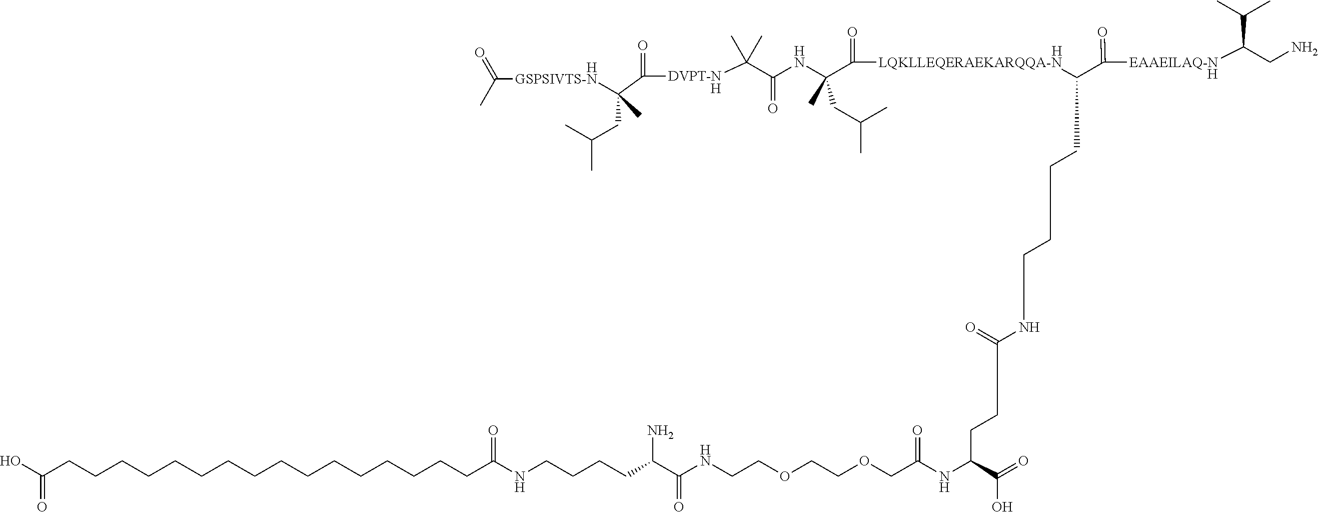

In a specific embodiment of Formula Ia comprising the polypeptide, or a pharmaceutically acceptable salt thereof, X₂₁ is I and the polypeptide further comprises a linker-fatty acid. In a particular embodiment, the linker-fatty acid is γE-PKE-εK—CO—(CH₂)₁₈—CO₂H. In a specific embodiment of Formula Ia comprising the polypeptide, or a pharmaceutically acceptable salt thereof, the K at X₃₅ is conjugated to the linker-fatty acid. Alternatively, the polypeptide can have at least about 90 to 97% sequence similarity to SEQ ID NO:13. Alternatively, the polypeptide can have at least 90%, 91%, 92%, 93%, 94%, 95%, 96%, or 97% sequence similarity to SEQ ID NO: 13. An exemplary embodiment comprising SEQ ID NO:13 is shown.

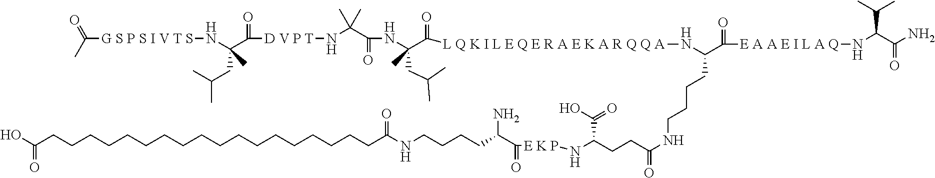

In a specific embodiment of Formula Ia comprising the polypeptide, or a pharmaceutically acceptable salt thereof, X₂₁ is I and the polypeptide further comprises a linker-fatty acid. In a particular embodiment, the linker-fatty acid is γE-PPP—εK—CO—(CH₂)₁₈—CO₂H. In a specific embodiment of Formula Ia comprising the polypeptide, or a pharmaceutically acceptable salt thereof, the K at X₃₅ is conjugated to the linker-fatty acid. In another embodiment, the polypeptide comprises SEQ ID NO:14. In another embodiment, the polypeptide is SEQ ID NO:14. Alternatively, the polypeptide can have at least about 90 to 97% sequence similarity to SEQ ID NO:14. Alternatively, the polypeptide can have at least 90%, 91%, 92%, 93%, 94%, 95%, 96%, or 97% sequence similarity to SEQ ID NO:14. An exemplary embodiment comprising SEQ ID NO:14 is shown.

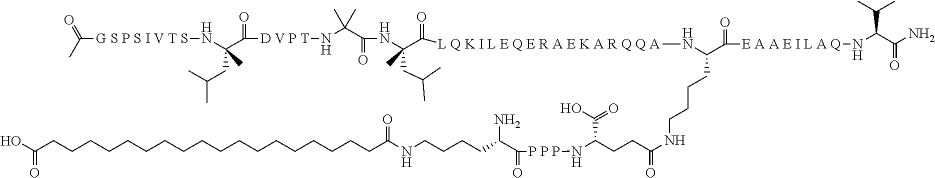

In a specific embodiment of Formula Ia comprising the polypeptide, or a pharmaceutically acceptable salt thereof, X₂₁ is I and the polypeptide further comprises a linker-fatty acid. In a particular embodiment, the linker-fatty acid is γE-PPPPPP-CO—(CH₂)₁₈—CO₂H. In a specific embodiment of the Formula Ia comprising the polypeptide, or a pharmaceutically acceptable salt thereof, the K at X₃₅ is conjugated to the linker-fatty acid. In another embodiment the polypeptide comprises SEQ ID NO:15. In another embodiment, the polypeptide is SEQ ID NO:15. Alternatively, the polypeptide can have at least about 90 to 97% sequence similarity to SEQ ID NO:15. Alternatively, the polypeptide can have at least 90%, 91%, 92%, 93%, 94%, 95%, 96%, or 97% sequence similarity to SEQ ID NO:15. An exemplary embodiment comprising SEQ ID NO:15 is shown.

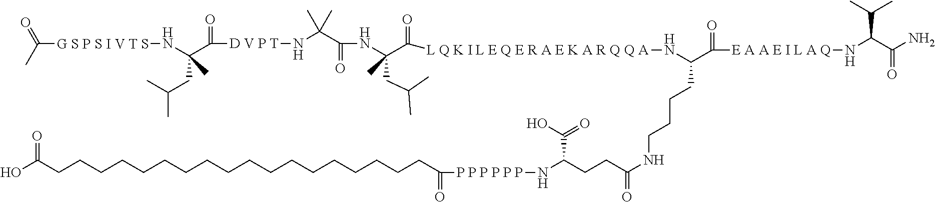

In a specific embodiment of Formula Ia comprising the polypeptide, or a pharmaceutically acceptable salt thereof, X₂₁ is I and the polypeptide further comprises a linker-fatty acid. In a particular embodiment, the linker-fatty acid is γE-PEPEPE-CO—(CH₂)₁₈—CO₂H. In a specific embodiment of Formula Ia comprising the polypeptide, or a pharmaceutically acceptable salt thereof, the K at X₃₅ is conjugated to the linker-fatty acid. In another embodiment, the polypeptide comprises SEQ ID NO:16. In another embodiment, the polypeptide is SEQ ID NO:16. Alternatively, the polypeptide can have at least about 90 to 97% sequence similarity to SEQ ID NO:16. Alternatively, the polypeptide can have at least 90%, 91%, 92%, 93%, 94%, 95%, 96%, or 97% sequence similarity to SEQ ID NO:16. An exemplary embodiment comprising SEQ ID NO:16 is shown.

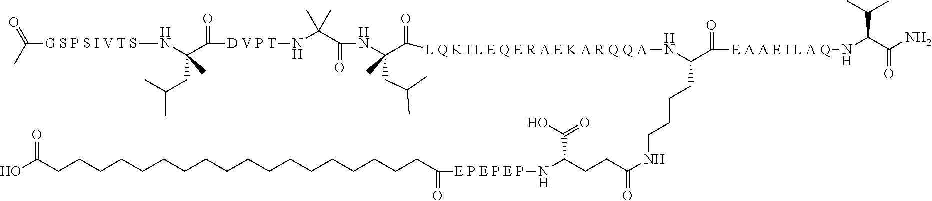

In a specific embodiment of Formula Ia comprising the polypeptide, or a pharmaceutically acceptable salt thereof, X₂₁ is I and the polypeptide further comprises a linker-fatty acid. In a particular embodiment, the linker-fatty acid is γE-(2-[2-(2-amino-ethoxy)-ethoxy]-acetyl)₂-εK—CO—(CH₂)₁₈—CO₂H. In a specific embodiment of Formula Ia comprising the polypeptide, or a pharmaceutically acceptable salt thereof, the K at X₃₅ is conjugated to the linker-fatty acid. In another embodiment, the polypeptide comprises SEQ ID NO:17. In another embodiment, the polypeptide is SEQ ID NO:17. Alternatively, the polypeptide can have at least about 90 to 97% sequence similarity to SEQ ID NO: 17. Alternatively, the polypeptide can have at least 90%, 91%, 92%, 93%, 94%, 95%, 96%, or 97% sequence similarity to SEQ ID NO:17. An exemplary embodiment comprising SEQ ID NO:17 is shown.

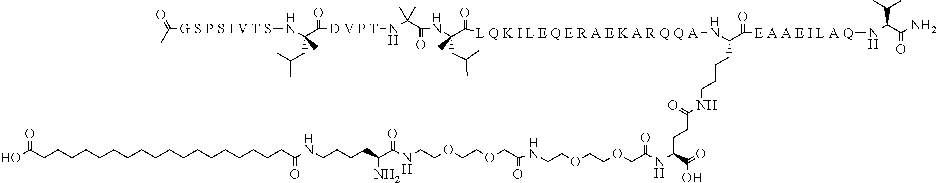

In a specific embodiment of Formula Ia comprising the polypeptide, or a pharmaceutically acceptable salt thereof, X₂₁ is I and the polypeptide further comprises a linker-fatty acid. In a particular embodiment, the linker-fatty acid is γE-εK-γE-εK—CO—(CH₂)₁₈—CO₂H. In a specific embodiment of Formula Ia comprising the polypeptide, or a pharmaceutically acceptable salt thereof, the K at X₃₅ is conjugated to the linker-fatty acid. Alternatively, the polypeptide can have at least about 90 to 99% sequence similarity to SEQ ID NO:18. Alternatively, the polypeptide can have at least 90%, 91%, 92%, 93%, 94%, 95%, 96%, or 97% sequence similarity to SEQ ID NO:18. An exemplary embodiment comprising SEQ ID NO:18 is shown.

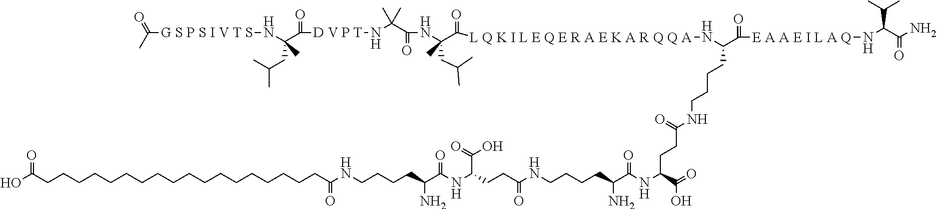

In a specific embodiment of Formula Ia comprising the polypeptide, or a pharmaceutically acceptable salt thereof, X₂₁ is I and the polypeptide further comprises a linker-fatty acid. In a particular embodiment, the linker-fatty acid is γE-εK-(2-[2-(2-amino-ethoxy)-ethoxy]-acetyl)-εK—CO—(CH₂)₁₈—CO₂H. In a specific embodiment of Formula Ia comprising the polypeptide, or a pharmaceutically acceptable salt thereof, the K at X₃₅ is conjugated to the linker-fatty acid. In another embodiment, the polypeptide comprises SEQ ID NO:19. In another embodiment, the polypeptide is SEQ ID NO:19. Alternatively, the polypeptide can have at least about 90 to 97% sequence similarity to SEQ ID NO:19. Alternatively, the polypeptide can have at least 90%, 91%, 92%, 93%, 94%, 95%, 96%, or 97% sequence similarity to SEQ ID NO: 19. An exemplary embodiment comprising SEQ ID NO:19 is shown.

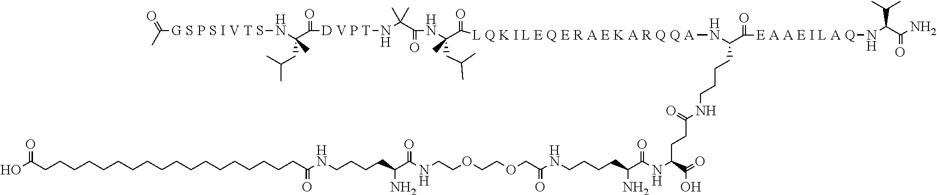

In a specific embodiment of Formula Ia comprising the polypeptide, or a pharmaceutically acceptable salt thereof, X₂₁ is I and the polypeptide further comprises a linker-fatty acid. In a particular embodiment, the linker-fatty acid is γE-(2-[2-(2-amino-ethoxy)-ethoxy]-acetyl)-εK—CO—(CH₂)₁₆—CO₂H. In a specific embodiment of Formula Ia comprising the polypeptide, or a pharmaceutically acceptable salt thereof, the K at X₃₅ is conjugated to the linker-fatty acid. In another embodiment, the polypeptide comprises SEQ ID NO:21. In another embodiment, the polypeptide is SEQ ID NO:21. Alternatively, the polypeptide can have at least about 90 to 97% sequence similarity to SEQ ID NO:21. Alternatively, the polypeptide can have at least 90%, 91%, 92%, 93%, 94%, 95%, 96%, or 97% sequence similarity to SEQ ID NO:21. An exemplary embodiment comprising SEQ ID NO:21 is shown.

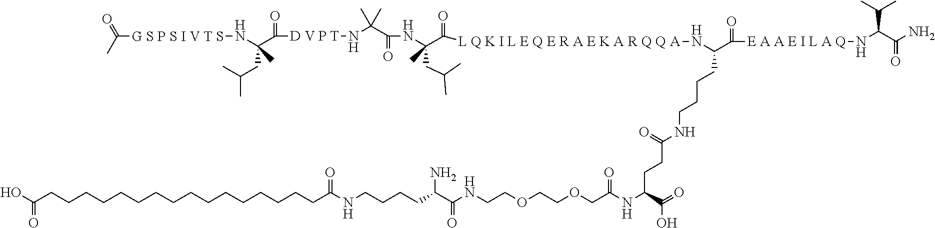

In another embodiment of Formula I, X₁ is Pyr, X₂ is G, X₃ is S, X₄ is P, X₅ is S, X₆ is G, X₁₁ is αMeL, X₁₃ is V, X₁₅ is T, X₁₆ is Aib, X₁₇ is αMeL, X₁₉ is Q, X₂₀ is K, X₂₁ is I, X₂₂ is L, X₂₆ is R, X₂₇ is A, X₃₀ is A, X₃₁ is R, X₃₅ is K, X₃₇ is A, and X₃₈ is A. In such embodiments, the polypeptide comprises a 44 amino acid sequence.

Thus, in some embodiments, the present disclosure provides a polypeptide, or a pharmaceutically acceptable salt thereof, of Formula Ib comprising:

(SEQ ID NO: 70)
Pyr GSPSGIVTS αMeL DVPT Aib αMeL
LQKILEQERAEKARQQAKEAAEILAQV,

or a pharmaceutically acceptable salt thereof,
wherein the C-term amino acid optionally amidated.

In another embodiment the polypeptide comprises SEQ ID NO:23. In another embodiment, the polypeptide is SEQ ID NO:23. Alternatively, the polypeptide can have at least about 90 to 97% sequence similarity to SEQ ID NO:23. Alternatively, the polypeptide can have at least 90%, 91%, 92%, 93%, 94%, 95%, 96%, or 97% sequence similarity to SEQ ID NO:23. An exemplary embodiment comprising SEQ ID NO:23 is shown.

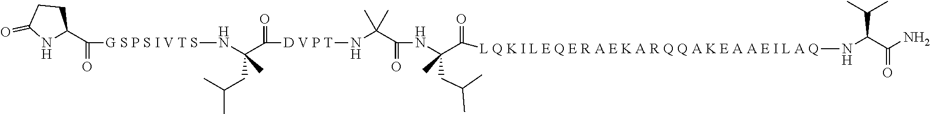

In a specific embodiment of Formula Ib comprising the polypeptide, or a pharmaceutically acceptable salt thereof, the polypeptide further comprises a linker-fatty acid. In a particular embodiment, the linker-fatty acid is γE-(2-[2-(2-amino-ethoxy)-ethoxy]-acetyl)-εK—CO—(CH₂)₁₈—CO₂H. In a specific embodiment of Formula Ib comprising the polypeptide, or a pharmaceutically acceptable salt thereof, the K at X₃₅ is conjugated to the linker-fatty acid. In another embodiment, the polypeptide comprises SEQ ID NO:22. In another embodiment, the polypeptide is SEQ ID NO:22. Alternatively, the polypeptide can have at least about 90 to 97% sequence similarity to SEQ ID NO:22. Alternatively, the polypeptide can have at least 90%, 91%, 92%, 93%, 94%, 95%, 96%, or 97% sequence similarity to SEQ ID NO:22. An exemplary embodiment comprising SEQ ID NO:22 is shown.

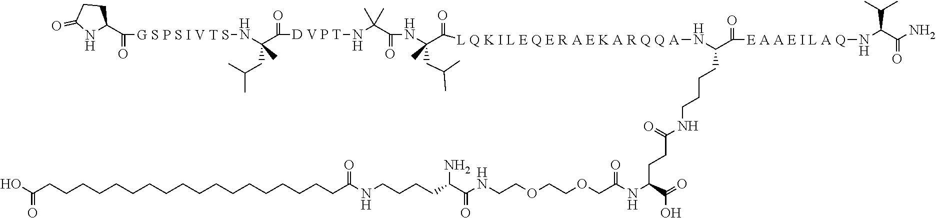

In a specific embodiment of Formula Ib comprising the polypeptide, or a pharmaceutically acceptable salt thereof, the polypeptide further comprises a linker-fatty acid. In a particular embodiment, the linker-fatty acid is γE-(2-[2-(2-amino-ethoxy)-ethoxy]-acetyl)-εK—CO—(CH₂)₁₆—CO₂H. In a specific embodiment of Formula Ib comprising the polypeptide, or a pharmaceutically acceptable salt thereof, the K at X₃₅ is conjugated to the linker-fatty acid. In another embodiment, the polypeptide comprises SEQ ID NO:24. In another embodiment the polypeptide is SEQ ID NO:24. Alternatively, the polypeptide can have at least about 90 to 97% sequence similarity to SEQ ID NO:24. Alternatively, the polypeptide can have at least 90%, 91%, 92%, 93%, 94%, 95%, 96%, or 97% sequence similarity to SEQ ID NO:24. An exemplary embodiment comprising SEQ ID NO:24 is shown.

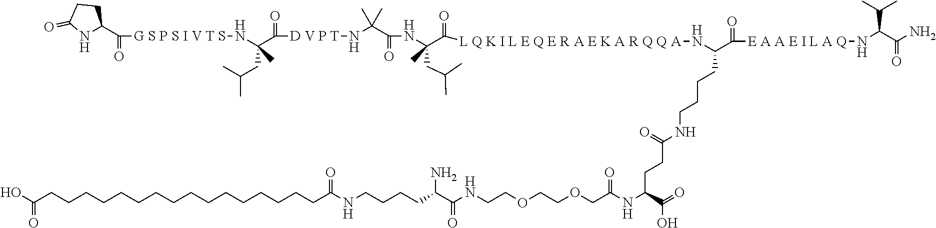

In another embodiment of Formula I, X₁ is γE, E, or absent, X₂ is absent, X₃ is G, X₄ is S, X₅ is P, X₆ is S, X₁₁ is αMeL, X₁₃ is V, X₁₅ is T, X₁₆ is Aib, X₁₇ is αMeL, X₁₉ is Q or E, X₂₀ is K, X₂₁ is I, X₂₂ is L, X₂₆ is R, X₂₇ is A, X₃₀ is A, X₃₁ is R, X₃₅ is T, X₃₇ is A, and X₃₈ is A.

In another embodiment of Formula I, X₁ is absent, X₂ is absent, X₃ is G, X₄ is S, X₅ is P, X₆ is S, X₁₁ is αMeL, X₁₃ is V, X₁₅ is T, X₁₆ is Aib, X₁₇ is αMeL, X₁₉ is Q, X₂₀ is K, X₂₁ is I, X₂₂ is L, X₂₆ is R, X₂₇ is A, X₃₀ is A, X₃₁ is R, X₃₅ is T, X₃₇ is A, and X₃₈ is A. In such embodiments, the polypeptide comprises a 42 amino acid sequence.

In another embodiment, the polypeptide comprises SEQ ID NO:27. In another embodiment, the polypeptide is SEQ ID NO:27. Alternatively, the polypeptide can have at least about 90 to 97% sequence similarity to SEQ ID NO:27. Alternatively, the polypeptide can have at least 90%, 91%, 92%, 93%, 94%, 95%, 96%, or 97% sequence similarity to SEQ ID NO:27. An exemplary embodiment comprising SEQ ID NO:27 is shown.

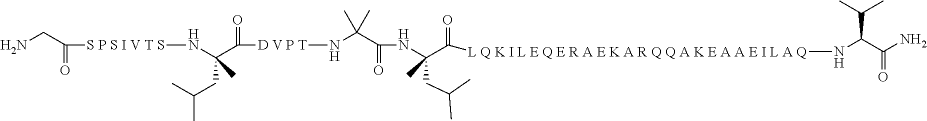

In another embodiment of Formula I, X₁ is γE or E, X₂ is absent, X₃ is G, X₄ is S, X₅ is P, X₆ is S, X₁₁ is αMeL, X₁₃ is V, X₁₅ is T, X₁₆ is Aib, X₁₇ is αMeL, X₁₉ is Q or E, X₂₀ is K, X₂₁ is I, X₂₂ is L, X₂₆ is R, X₂₇ is A, X₃₀ is A, X₃₁ is R, X₃₅ is T, X₃₇ is A, and X₃₈ is A. In such embodiments, the polypeptide comprises a 43 amino acid sequence.

In a specific embodiment of Formula Ic comprising the polypeptide, or a pharmaceutically acceptable salt thereof, X₁ is γE and the polypeptide further comprises a linker-fatty acid. In a specific embodiment, the polypeptide further comprises a fatty acid conjugated to γE at position X₁ via an amide bond between the alpha-amino group of the γE and the carboxylic acid of a (2-{2-(2-amino-ethoxy)-ethoxy]acetic monomer of a linker-fatty acid.

Thus, in some embodiments, the present disclosure provides a polypeptide, or a pharmaceutically acceptable salt thereof, of Formula Ic, comprising

(SEQ ID NO: 71)
X₁GSPSIVTS αMeL DVPT Aib αMeLLX₁₉
KILEQERAEKARQQATEAAEILAQV,

wherein
X₁ is γE or E,
X₁₉ is Q or E [wherein when X₂ is absent, X₁₉ is at position 18 of the polypeptide], wherein the C-term amino acid is optionally amidated.

In a specific embodiment of the polypeptide, or a pharmaceutically acceptable salt thereof wherein X₁ is γE, the γE is conjugated to the G at X₃ via an amide bond between the gamma-carboxylic acid group of the γE and the amino group of the G.

In a specific embodiment of Formula Ic comprising the polypeptide, or a pharmaceutically acceptable salt thereof, X₁ is γE, X₁₉ is Q, and the linker-fatty acid is (2-[2-(2-amino-ethoxy)-ethoxy]-acetyl)-εK—CO—(CH₂)₁₈—CO₂H. In another embodiment, the polypeptide comprises SEQ ID NO:25. In another embodiment, the polypeptide is SEQ ID NO:25. Alternatively, the polypeptide can have at least about 90 to 97% sequence similarity to SEQ ID NO:25. Alternatively, the polypeptide can have at least 90%, 91%, 92%, 93%, 94%, 95%, 96%, or 97% sequence similarity to SEQ ID NO:25. An exemplary embodiment comprising SEQ ID NO:25 is shown.

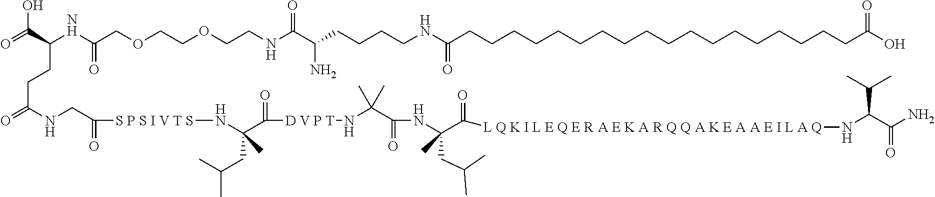

In a specific embodiment of Formula Ic comprising the polypeptide, or a pharmaceutically acceptable salt thereof, X₁ is γE, X₁₉ is Q, and the linker-fatty acid is (2-[2-(2-amino-ethoxy)-ethoxy]-acetyl)-K—CO—(CH₂)₁₈—CO₂H. In another embodiment, the polypeptide comprises SEQ ID NO:26. In another embodiment the polypeptide is SEQ ID NO:26. Alternatively, the polypeptide can have at least about 90 to 97% sequence similarity to SEQ ID NO:26. Alternatively, the polypeptide can have at least 90%, 91%, 92%, 93%, 94%, 95%, 96%, or 97% sequence similarity to SEQ ID NO:26. An exemplary embodiment comprising SEQ ID NO:26 is shown.

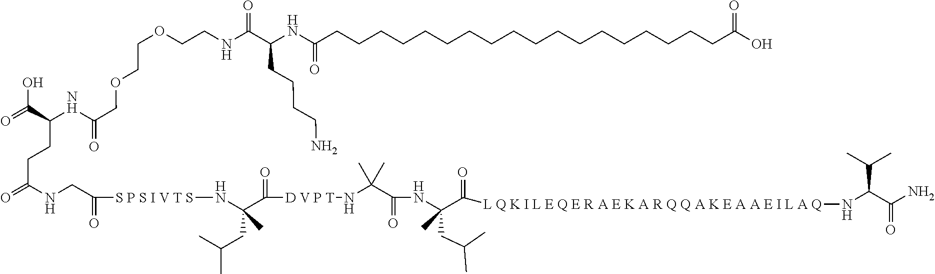

In a specific embodiment of Formula Ic comprising the polypeptide, or a pharmaceutically acceptable salt thereof, X₁ is γE, X₁₉ is Q and the linker-fatty acid is (2-[2-(2-amino-ethoxy)-ethoxy]-acetyl)-εK—CO—(CH₂)₁₆—CO₂H. In another embodiment, the polypeptide comprises SEQ ID NO:28. In another embodiment, the polypeptide is SEQ ID NO:28. Alternatively, the polypeptide can have at least about 90 to 97% sequence similarity to SEQ ID NO:28. Alternatively, the polypeptide can have at least 90%, 91%, 92%, 93%, 94%, 95%, 96%, or 97% sequence similarity to SEQ ID NO:28. An exemplary embodiment comprising SEQ ID NO:28 is shown.

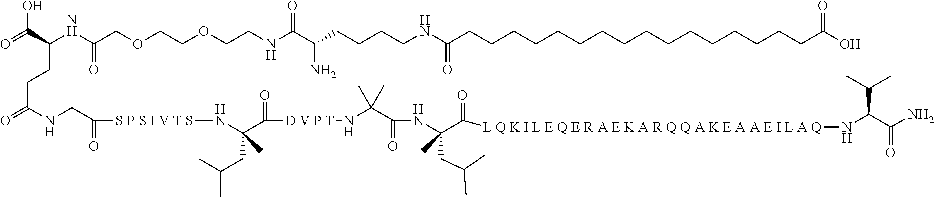

In a specific embodiment of Formula Ic comprising the polypeptide, or a pharmaceutically acceptable salt thereof, X₁ is γE, X₁₉ is E, and the linker-fatty acid is (2-[2-(2-amino-ethoxy)-ethoxy]-acetyl)-Dap-CO—(CH₂)₁₈—CO₂H. In another embodiment, the polypeptide comprises SEQ ID NO:52. In another embodiment, the polypeptide is SEQ ID NO:52. Alternatively, the polypeptide can have at least about 90 to 97% sequence similarity to SEQ ID NO:52. Alternatively, the polypeptide can have at least 90%, 91%, 92%, 93%, 94%, 95%, 96%, or 97% sequence similarity to SEQ ID NO:52. An exemplary embodiment comprising SEQ ID NO:52 is shown.

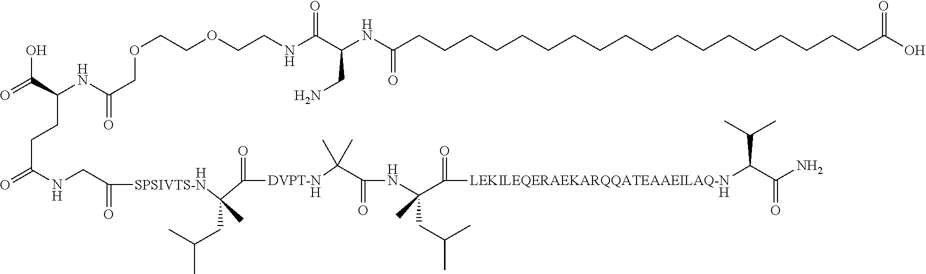

In another embodiment of Formula I, X₁ is absent, X₂ is absent, X₃ is absent, X₄ is absent, X₅ is absent, X₆ is absent, X₁₁ is αMeL, X₁₃ is D-Val, X₁₅ is T or I, X₁₆ is Aib or G, X₁₇ is αMeL, X₁₉ is Q, X₂₀ is I, X₂₁ is K, X₂₂ is L, X₂₆ is R, X₂₇ is A, X₃₀ is A, X₃₁ is R, X₃₅ is K, X₃₇ is N, and X₃₈ is T. In such embodiments, the polypeptide comprises a 38 amino acid sequence.

In some embodiments, the present disclosure provides a compound, or a pharmaceutically acceptable salt thereof, of Formula Id, comprising

(SEQ ID NO: 72)
IVTS αMeL D D-Val PX₁₅X₁₆ αMeL L
QIKLEQERAEKARQQAKENTEILAQV,

wherein
X₁₅ is T or I [wherein when X₁—X₆ are absent, X₁₅ is at position 9 of the polypeptide],
X₁₆ is G or Aib [wherein when X₁—X₆ are absent, X₁₆ is at position 10 of the polypeptide], wherein the N-term is amino acid is optionally N-acetylated, and wherein the C-term amino acid is optionally amidated.

In a specific embodiment of Formula Id comprising the polypeptide, or a pharmaceutically acceptable salt thereof, X₁₅ is T, X₁₆ is G, and the polypeptide further comprises a linker-fatty acid. In a particular embodiment, the linker-fatty acid is 2-[2-(2-amino-ethoxy)-ethoxy]-acetyl)₂-γE-CO—(CH₂)₁₈—CO₂H. In a specific embodiment of Formula Id comprising the polypeptide, or a pharmaceutically acceptable salt thereof, the K at X₃₅ is conjugated to the linker-fatty acid. In another embodiment, the polypeptide is SEQ ID NO:29. Alternatively, the polypeptide can have at least about 90 to 97% sequence similarity to SEQ ID NO:29. Alternatively, the polypeptide can have at least 90%, 91%, 92%, 93%, 94%, 95%, 96%, or 97% sequence similarity to SEQ ID NO:29. An exemplary embodiment comprising SEQ ID NO:29 is shown.

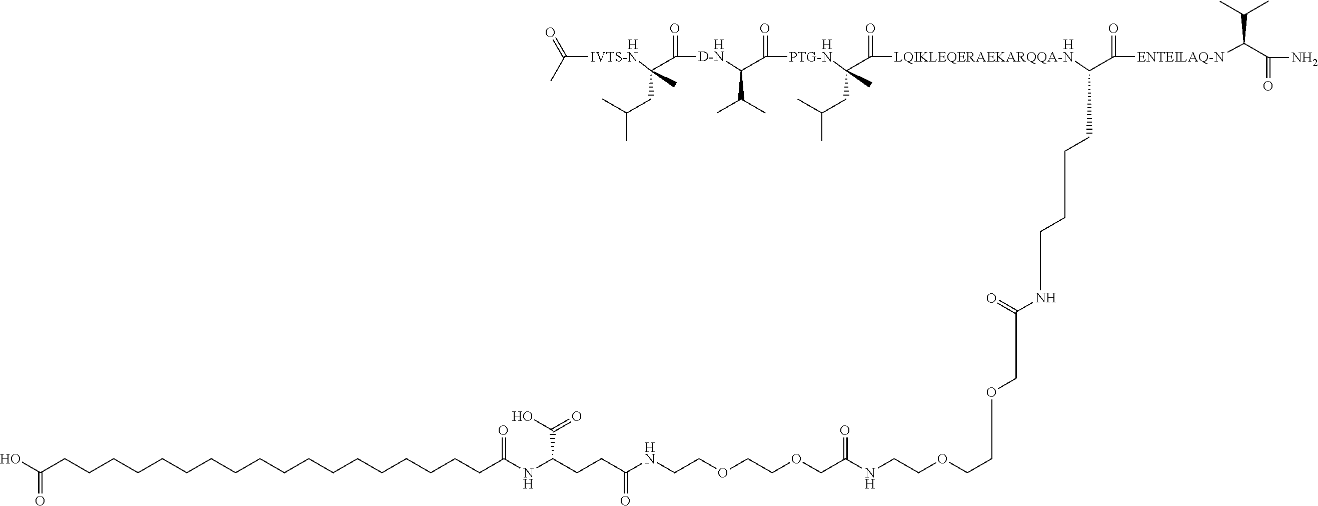

In a specific embodiment of Formula Id comprising the polypeptide, or a pharmaceutically acceptable salt thereof, X₁₅ is T, X₁₆ is G, and the polypeptide further comprises a linker-fatty acid. In a particular embodiment, the linker-fatty acid is γE-(2-[2-(2-amino-ethoxy)-ethoxy]-acetyl)-γE-CO—(CH₂)₁₈—CO₂H. In a specific embodiment of Formula Id comprising the polypeptide, or a pharmaceutically acceptable salt thereof, the K at X₃₅ is conjugated to the linker-fatty acid. In another embodiment, the polypeptide comprises SEQ ID NO:30. In another embodiment, the polypeptide is SEQ ID NO:30. Alternatively, the polypeptide can have at least about 90 to 97% sequence similarity to SEQ ID NO:30. Alternatively, the polypeptide can have at least 90%, 91%, 92%, 93%, 94%, 95%, 96%, or 97% sequence similarity to SEQ ID NO:30. An exemplary embodiment comprising SEQ ID NO:30 is shown.

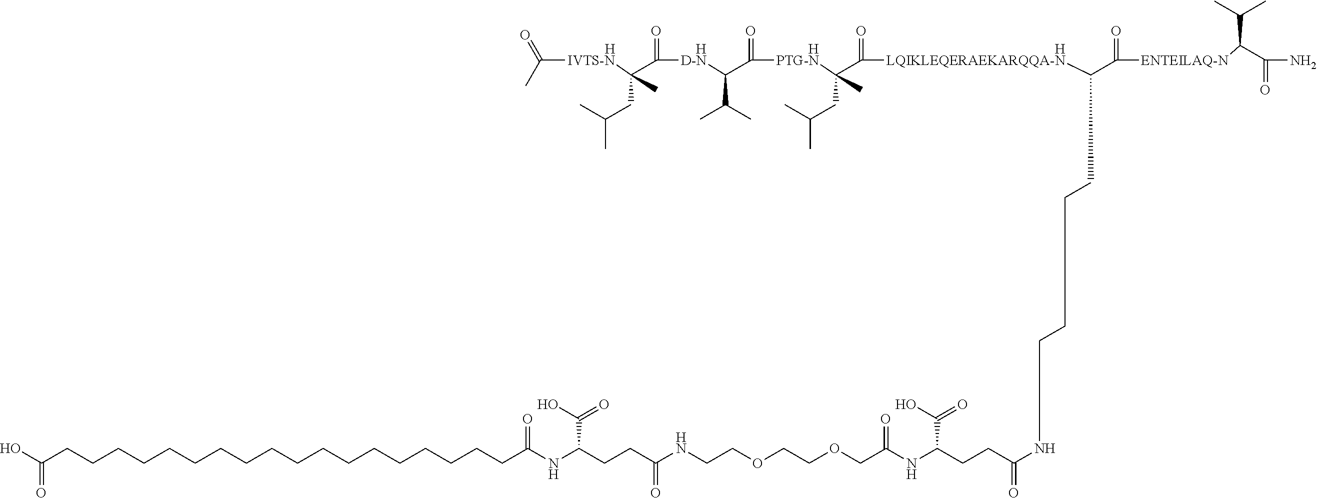

In a specific embodiment of Formula Id comprising the polypeptide, or a pharmaceutically acceptable salt thereof, X₁₅ is T, X₁₆ is G, and the polypeptide further comprises a linker-fatty acid. In a particular embodiment, the linker-fatty acid is γE₂-(2-[2-(2-amino-ethoxy)-ethoxy]-acetyl)-CO—(CH₂)₁₈—CO₂H. In a specific embodiment of Formula Id comprising the polypeptide, or pharmaceutically acceptable salt thereof, the K at X₃₅ is conjugated to the linker-fatty acid. In another embodiment, the polypeptide comprises SEQ ID NO:31. In another embodiment, the polypeptide is SEQ ID NO:31. Alternatively, the polypeptide can have at least about 90 to 97% sequence similarity to SEQ ID NO:31. Alternatively, the polypeptide can have at least 90%, 91%, 92%, 93%, 94%, 95%, 96%, or 97% sequence similarity to SEQ ID NO:31. An exemplary embodiment comprising SEQ ID NO:31 is shown.

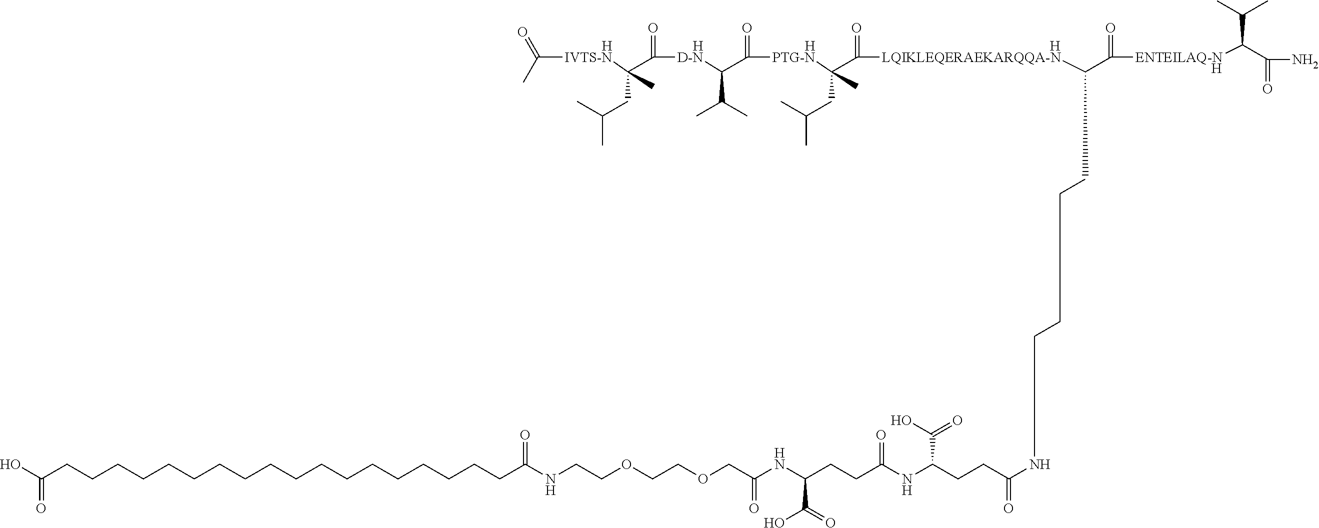

In a specific embodiment of Formula Id comprising the polypeptide, or a pharmaceutically acceptable salt thereof, X₁₅ is T, X₁₆ is G, and the polypeptide further comprises a linker-fatty acid. In a particular embodiment, the linker-fatty acid is 2-[2-(2-amino-ethoxy)-ethoxy]-acetyl)-εK-γE-CO—(CH₂)₁₈—CO₂. In a specific embodiment of Formula Id comprising the polypeptide, or pharmaceutically acceptable salt thereof, the K at X₃₅ is conjugated to the linker-fatty acid. In another embodiment, the polypeptide comprises SEQ ID NO:32. In another embodiment, the polypeptide is SEQ ID NO:32. Alternatively, the polypeptide can have at least about 90 to 97% sequence similarity to SEQ ID NO:32. Alternatively, the polypeptide can have at least 90%, 91%, 92%, 93%, 94%, 95%, 96%, or 97% sequence similarity to SEQ ID NO:32. An exemplary embodiment comprising SEQ ID NO:32 is shown.

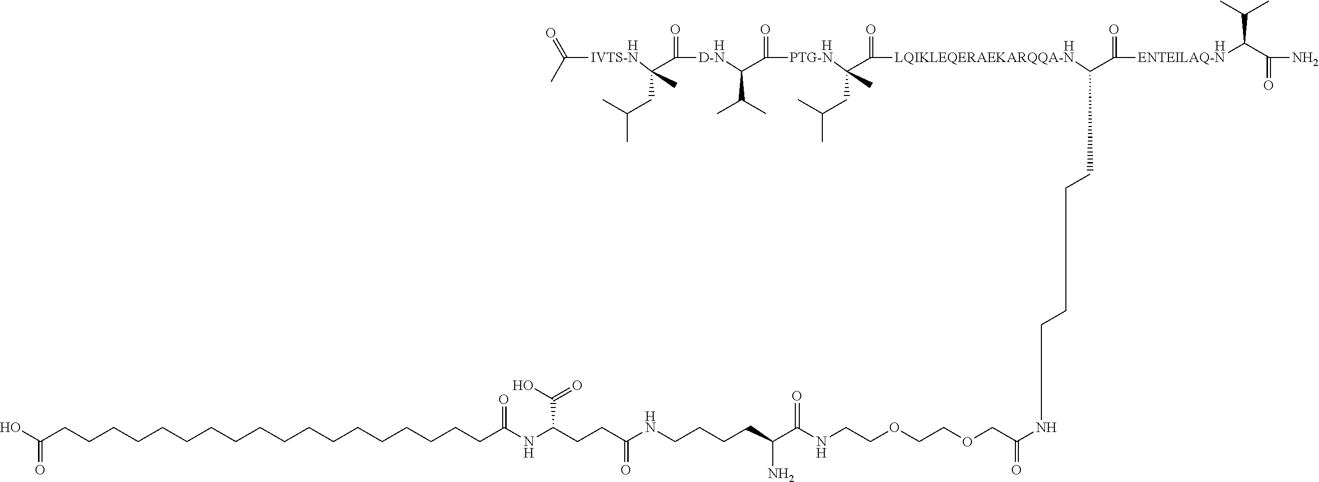

In a specific embodiment of Formula Id comprising the polypeptide, or a pharmaceutically acceptable salt thereof, X₁₅ is T, X₁₆ is G, and the polypeptide further comprises a linker-fatty acid. In a particular embodiment, linker-fatty acid is 2-[2-(2-amino-ethoxy)-ethoxy]-acetyl)-γE-εK—CO—(CH₂)₁₈—CO₂H. In a specific embodiment of the Formula Id comprising the polypeptide, or a pharmaceutically acceptable salt thereof, the K at X₃₅ is conjugated to the linker-fatty acid. In another embodiment, the polypeptide comprises SEQ ID NO:33. In another embodiment, the polypeptide is SEQ ID NO:33. Alternatively, the polypeptide can have at least about 90 to 97% sequence similarity to SEQ ID NO:33. Alternatively, the polypeptide can have at least 90%, 91%, 92%, 93%, 94%, 95%, 96%, or 97% sequence similarity to SEQ ID NO:33. An exemplary embodiment comprising SEQ ID NO:33 is shown.

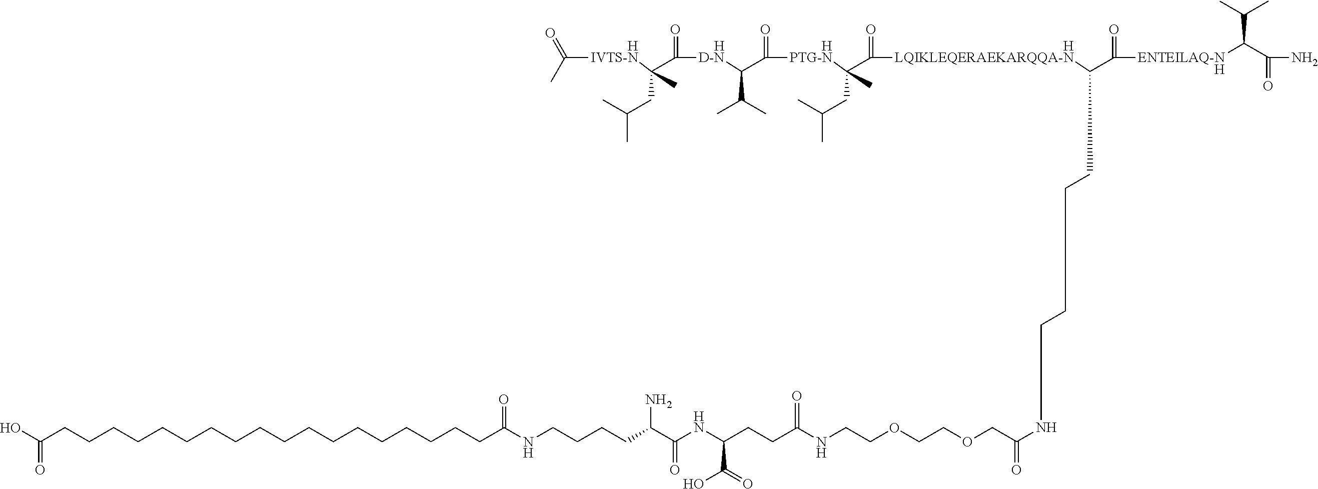

In a specific embodiment of Formula Id comprising the polypeptide, or a pharmaceutically acceptable salt thereof, X₁₅ is T, X₁₆ is G, and the polypeptide further comprises a linker-fatty acid. In a particular embodiment, the linker-fatty acid is εK-(2-[2-(2-amino-ethoxy)-ethoxy]-acetyl)-γE-CO—(CH₂)₁₈—CO₂H. In a specific embodiment of the polypeptide, or a pharmaceutically acceptable salt thereof, the K at X₃₅ is conjugated to the linker-fatty acid. In another embodiment, the polypeptide comprises SEQ ID NO:34. In another embodiment, the polypeptide is SEQ ID NO:34. Alternatively, the polypeptide can have at least about 90 to 97% sequence similarity to SEQ ID NO:34. Alternatively, the polypeptide can have at least 90%, 91%, 92%, 93%, 94%, 95%, 96%, or 97% sequence similarity to SEQ ID NO:34. An exemplary embodiment comprising SEQ ID NO:34 is shown.

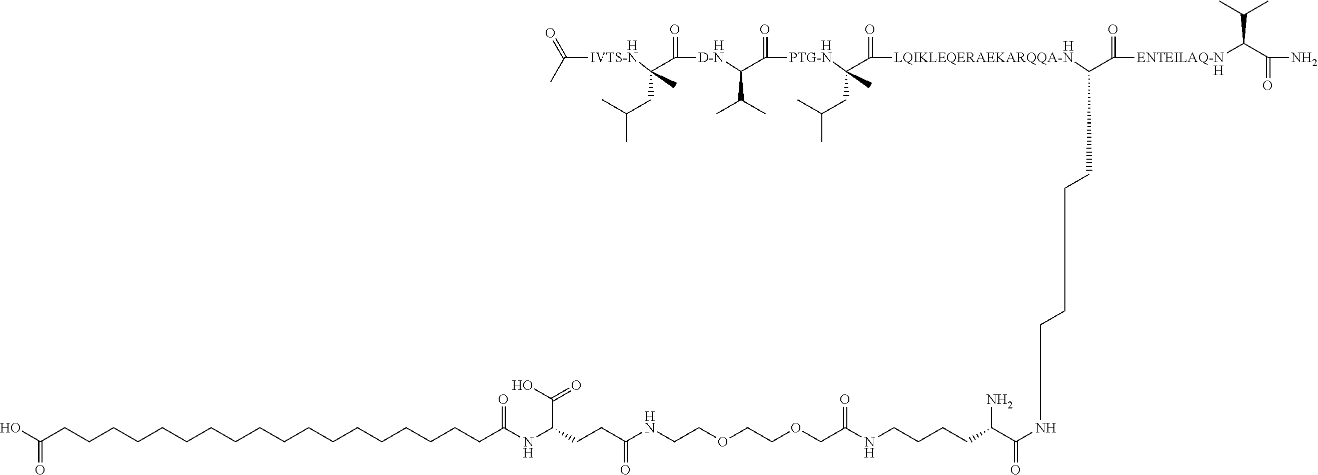

In a specific embodiment of Formula Id comprising the polypeptide, or a pharmaceutically acceptable salt thereof, X₁₅ is T, X₁₆ is G, and the polypeptide further comprises a linker-fatty acid. In a particular embodiment, the linker-fatty acid is εK-γE-(2-[2-(2-amino-ethoxy)-ethoxy]-acetyl)-CO—(CH₂)₁₈—CO₂H. In a specific embodiment of the Formula Id comprising the polypeptide, or a pharmaceutically acceptable salt thereof, the K at X₃₅ is conjugated to the linker-fatty acid. In another embodiment, the polypeptide comprises SEQ ID NO:35. In another embodiment, the polypeptide is SEQ ID NO:35. Alternatively, the polypeptide can have at least about 90 to 97% sequence similarity to SEQ ID NO:35. Alternatively, the polypeptide can have at least 90%, 91%, 92%, 93%, 94%, 95%, 96%, or 97% sequence similarity to SEQ ID NO:35. An exemplary embodiment comprising SEQ ID NO:35 is shown.

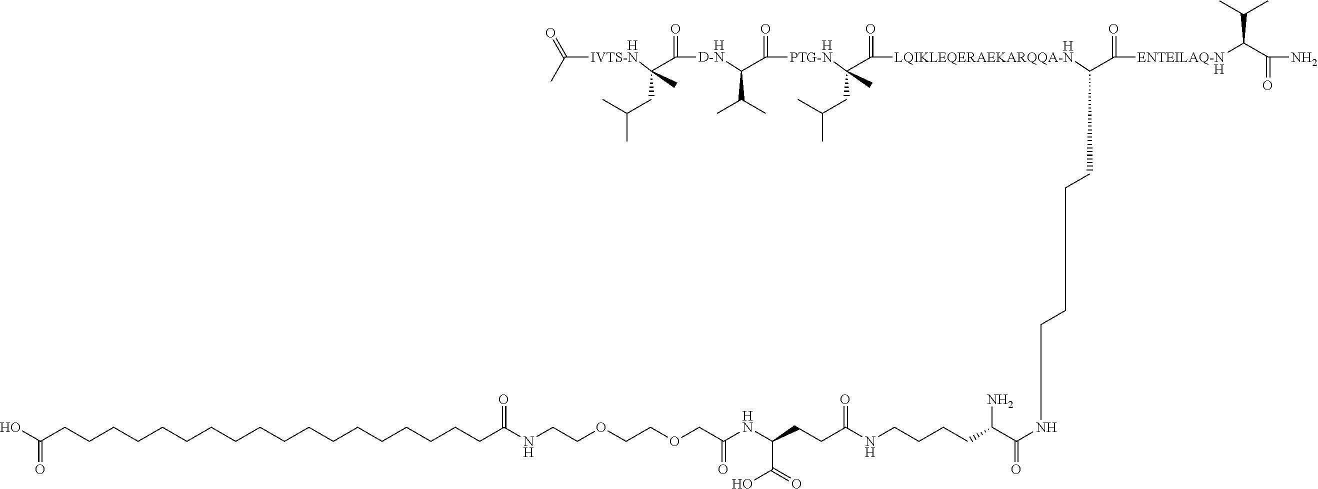

In a specific embodiment of Formula Id comprising the polypeptide, or a pharmaceutically acceptable salt thereof, X₁₅ is T, X₁₆ is G, and the polypeptide further comprises a linker-fatty acid. In a particular embodiment, the linker-fatty acid is γE-(2-[2-(2-amino-ethoxy)-ethoxy]-acetyl)-εK—CO—(CH₂)₁₈—CO₂H. In a specific embodiment of Formula Id comprising the polypeptide, or a pharmaceutically acceptable salt thereof, the K at X₃₅ is conjugated to the linker-fatty acid. In another embodiment, the polypeptide comprises SEQ ID NO:36. In another embodiment, the polypeptide is SEQ ID NO:36. Alternatively, the polypeptide can have at least about 90 to 97% sequence similarity to SEQ ID NO:36. Alternatively, the polypeptide can have at least 90%, 91%, 92%, 93%, 94%, 95%, 96%, or 97% sequence similarity to SEQ ID NO:36. An exemplary sequence comprising SEQ ID NO:36 is shown.

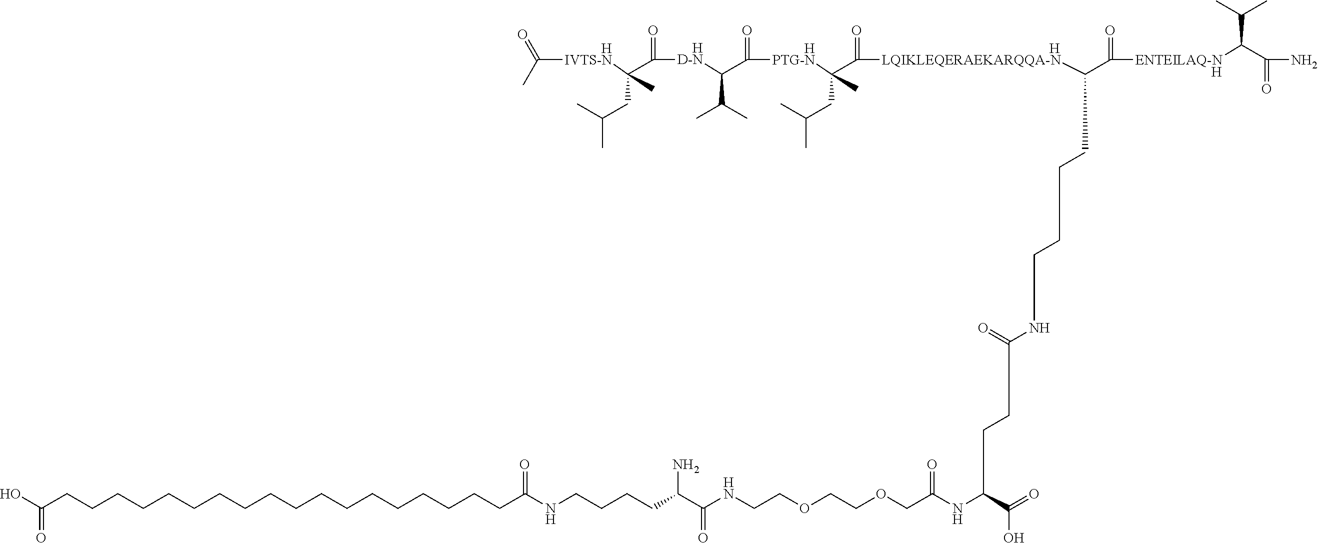

In a specific embodiment of Formula Id comprising the polypeptide, or a pharmaceutically acceptable salt thereof, X₁₅ is T, X₁₆ is G, and the polypeptide further comprises a linker-fatty acid. In a particular embodiment, the linker-fatty acid is γE-εK-(2-[2-(2-amino-ethoxy)-ethoxy]-acetyl)-CO—(CH₂)₁₈—CO₂H. In a specific embodiment of Formula Id comprising the polypeptide, or a pharmaceutically acceptable salt thereof, the K at X₃₅ is conjugated to the linker-fatty acid. In another embodiment, the polypeptide comprises SEQ ID NO:37. In another embodiment the polypeptide is SEQ ID NO:37. Alternatively, the polypeptide can have at least about 90 to 97% sequence similarity to SEQ ID NO:37. Alternatively, the polypeptide can have at least 90%, 91%, 92%, 93%, 94%, 95%, 96%, or 97% sequence similarity to SEQ ID NO:37. An exemplary sequence comprising SEQ ID NO:37 is shown.

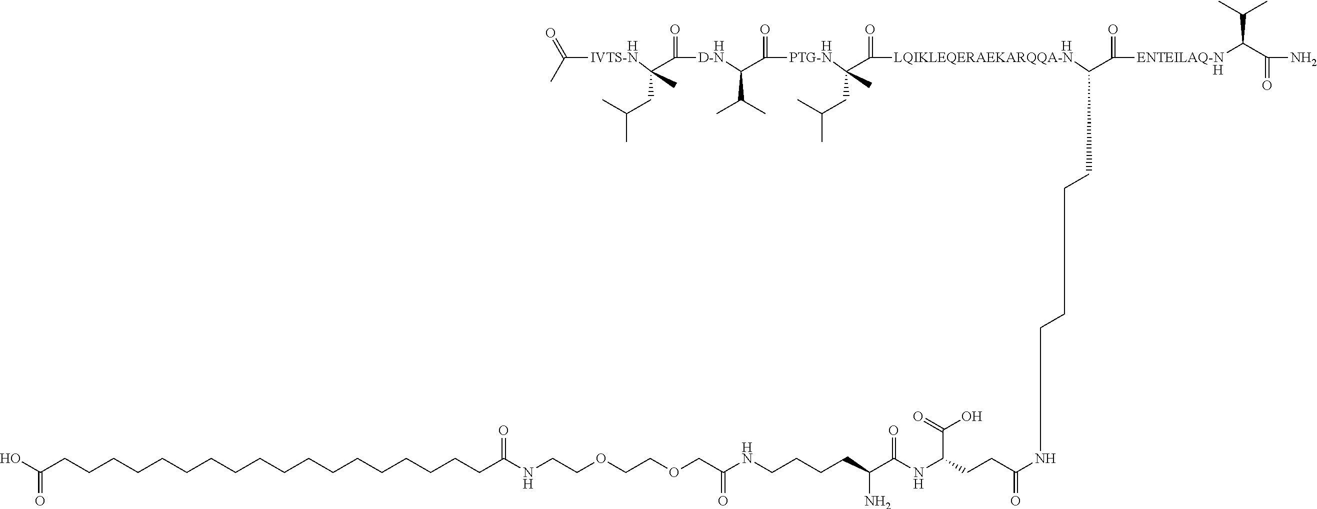

In a specific embodiment of Formula Id comprising the polypeptide, or a pharmaceutically acceptable salt thereof, X₁₅ is T, X₁₆ is G, and the polypeptide further comprises a linker-fatty acid. In a particular embodiment, the linker-fatty acid is γE-γE-εK—CO—(CH₂)₁₈—CO₂H. In a specific embodiment of Formula Id comprising the polypeptide, or a pharmaceutically acceptable salt thereof, the K at X₃₅ is conjugated to the linker-fatty acid. In another embodiment, the polypeptide comprises SEQ ID NO:38. In another embodiment, the polypeptide is SEQ ID NO:38. Alternatively, the polypeptide can have at least about 90 to 97% sequence similarity to SEQ ID NO:38. Alternatively, the polypeptide can have at least 90%, 91%, 92%, 93%, 94%, 95%, 96%, or 97% sequence similarity to SEQ ID NO:38. An exemplary embodiment comprising SEQ ID NO:38 is shown.

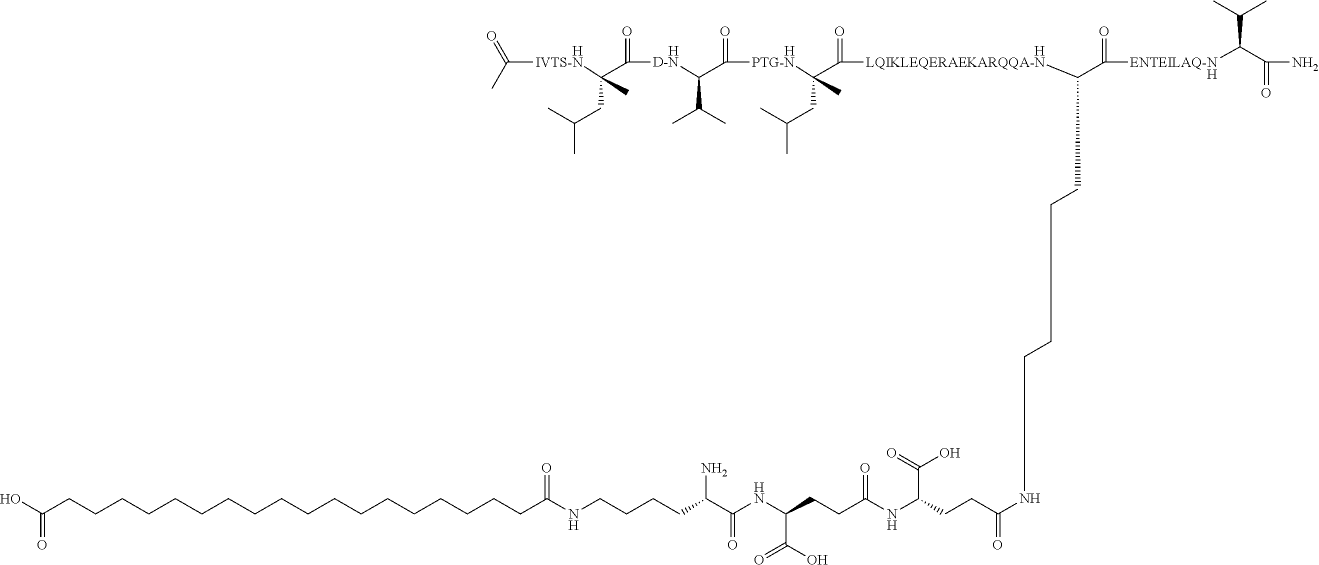

In a specific embodiment of Formula Id comprising the polypeptide, or a pharmaceutically acceptable salt thereof, X₁₅ is T, X₁₆ is G, and the polypeptide further comprises a linker-fatty acid. In a particular embodiment, the linker-fatty acid is γE-εK-γE-CO—(CH₂)₁₈—CO₂H. In a specific embodiment of Formula Id comprising the polypeptide, or a pharmaceutically acceptable salt thereof, the K at X₃₅ is conjugated to the linker-fatty acid. In another embodiment, the polypeptide comprises SEQ ID NO:39. In another embodiment the polypeptide is SEQ ID NO:39. Alternatively, the polypeptide can have at least about 90 to 97% sequence similarity to SEQ ID NO:39. Alternatively, the polypeptide can have at least 90%, 91%, 92%, 93%, 94%, 95%, 96%, or 97% sequence similarity to SEQ ID NO:39. An exemplary embodiment comprising SEQ ID NO:39 is shown.

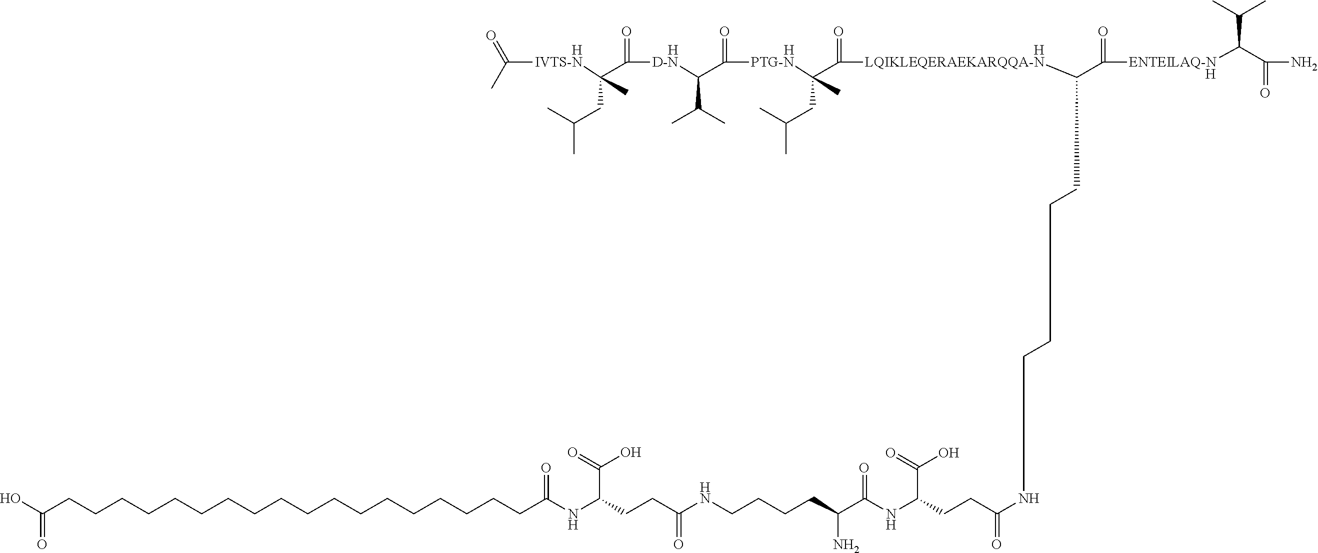

In a specific embodiment of Formula Id comprising the polypeptide, or a pharmaceutically acceptable salt thereof, X₁₅ is T, X₁₆ is G, and the polypeptide further comprises a linker-fatty acid. In a particular embodiment, the linker-fatty acid is εK-γE-γE-CO—(CH₂)₁₈—CO₂H. In a specific embodiment of Formula Id comprising the polypeptide, or a pharmaceutically acceptable salt thereof, the K at X₃₅ is conjugated to the linker-fatty acid. In another embodiment, the polypeptide comprises SEQ ID NO:40. In another embodiment, the polypeptide is SEQ ID NO:40. Alternatively, the polypeptide can have at least about 90 to 97% sequence similarity to SEQ ID NO:40. Alternatively, the polypeptide can have at least 90%, 91%, 92%, 93%, 94%, 95%, 96%, or 97% sequence similarity to SEQ ID NO:40. An exemplary embodiment comprising SEQ ID NO:40 is shown.

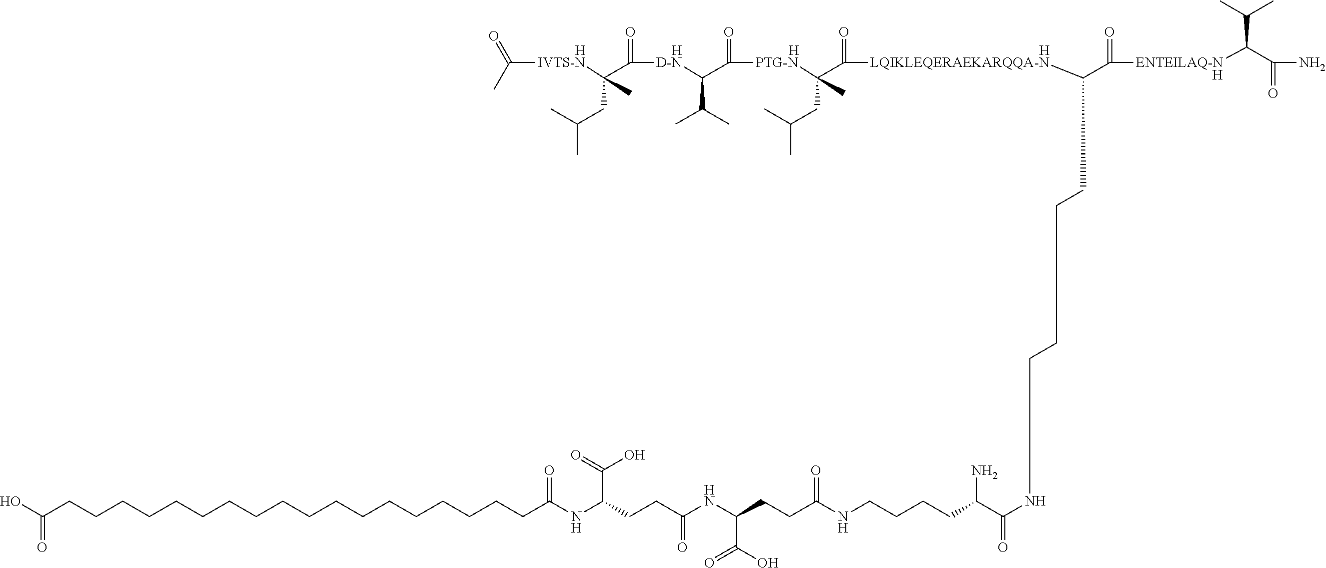

In a specific embodiment of Formula Id comprising the polypeptide, or a pharmaceutically acceptable salt thereof, X₁₅ is T, X₁₆ is Aib, and the polypeptide further comprises a linker-fatty acid. In a particular embodiment, the linker-fatty acid is 2-[2-(2-amino-ethoxy)-ethoxy]-acetyl)₂-γE-CO—(CH₂)₁₈—CO₂H. In a specific embodiment of Formula Id comprising the polypeptide, or a pharmaceutically acceptable salt thereof, the K at X₃₅ is conjugated to the linker-fatty acid. In another embodiment, the polypeptide comprises SEQ ID NO:41. In another embodiment, the polypeptide is SEQ ID NO:41. Alternatively, the polypeptide can have at least about 90 to 97% sequence similarity to SEQ ID NO:41. Alternatively, the polypeptide can have at least 90%, 91%, 92%, 93%, 94%, 95%, 96%, or 97% sequence similarity to SEQ ID NO:41. An exemplary embodiment comprising SEQ ID NO:41 is shown.

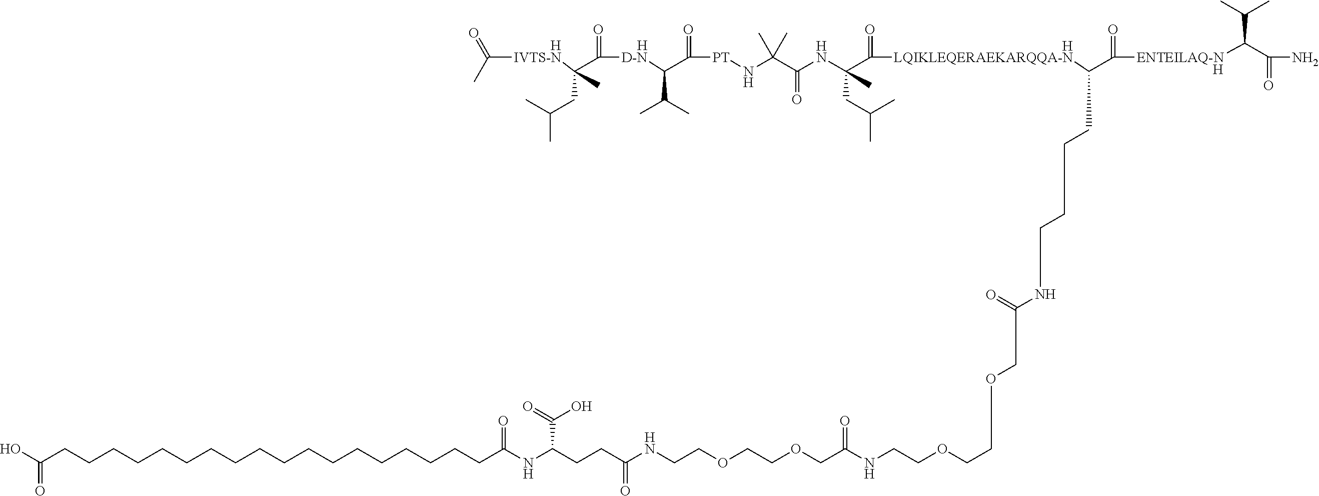

In a specific embodiment of Formula Id comprising the polypeptide, or a pharmaceutically acceptable salt thereof, X₁₅ is I, X₁₆ is Aib, and the polypeptide further comprises a linker-fatty acid moiety. In a particular embodiment, the linker-fatty acid is 2-[2-(2-amino-ethoxy)-ethoxy]-acetyl)₂-γE-CO—(CH₂)₁₈—CO₂H. In a specific embodiment of Formula Id comprising the polypeptide, or pharmaceutically acceptable salt thereof, the K at X₃₅ is conjugated to the linker-fatty acid. In another embodiment, the polypeptide comprises SEQ ID NO:42. In another embodiment, the polypeptide is SEQ ID NO:42. Alternatively, the polypeptide can have at least about 90 to 97% sequence similarity to SEQ ID NO:42. Alternatively, the polypeptide can have at least 90%, 91%, 92%, 93%, 94%, 95%, 96%, or 97% sequence similarity to SEQ ID NO:42. An exemplary embodiment comprising SEQ ID NO:42 is shown.

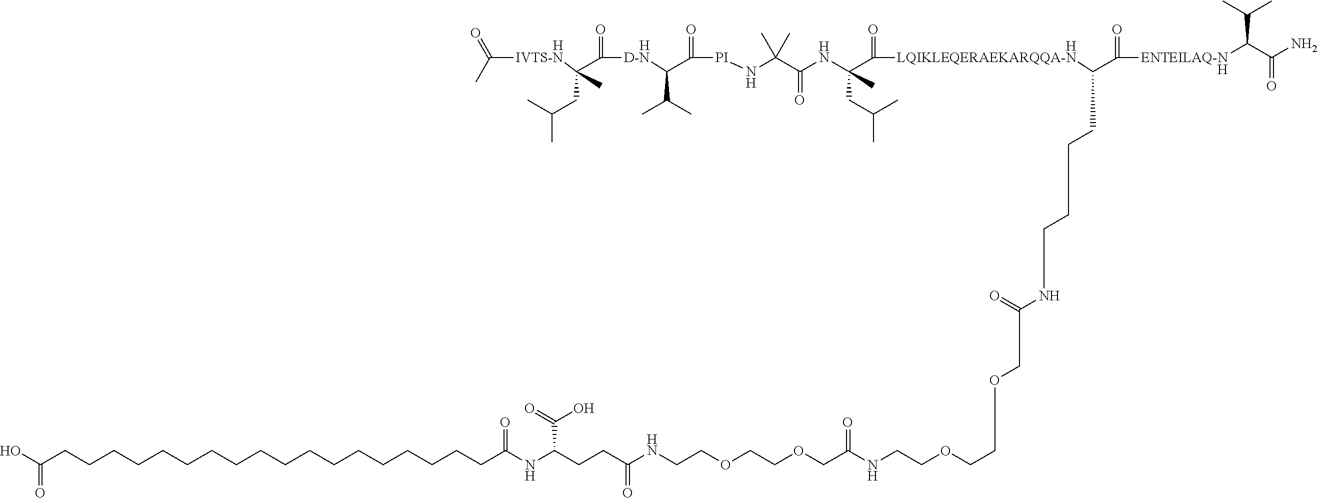

In a specific embodiment of Formula Id comprising the polypeptide, or a pharmaceutically acceptable salt thereof, X₁₅ is T, X₁₆ is G, and the polypeptide further comprises a linker-fatty acid. In a particular embodiment, the linker-fatty acid is GKEKEKE-CO—(CH₂)₁₈—CO₂H. In a specific embodiment of Formula Id comprising the polypeptide, or a pharmaceutically acceptable salt thereof, the K at X₃₅ is conjugated to the linker-fatty acid. In another embodiment, the polypeptide comprises SEQ ID NO:43. In another embodiment, the polypeptide is SEQ ID NO:43. Alternatively, the polypeptide can have at least about 90 to 97% sequence similarity to SEQ ID NO:43. Alternatively, the polypeptide can have at least 90%, 91%, 92%, 93%, 94%, 95%, 96%, or 97% sequence similarity to SEQ ID NO:43. An exemplary embodiment comprising SEQ ID NO:43 is shown.

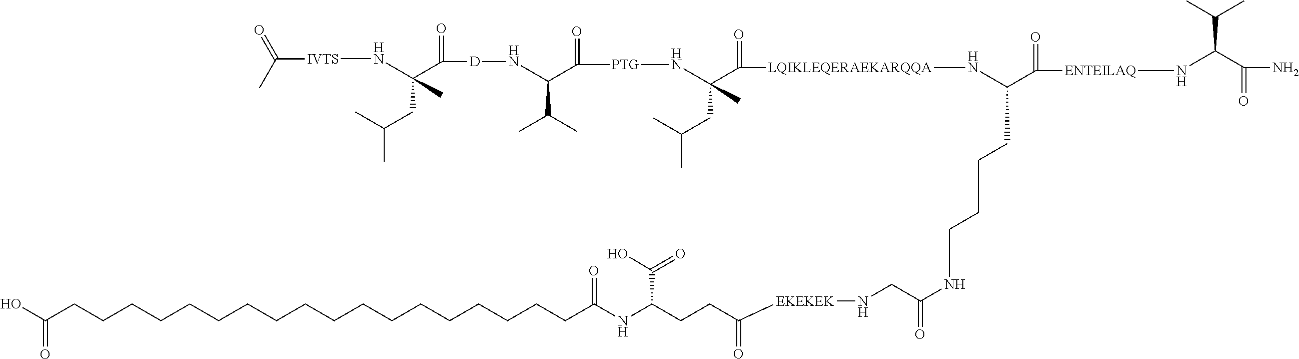

In a specific embodiment of Formula Id comprising the polypeptide, or a pharmaceutically acceptable salt thereof, X₁₅ is T, X₁₆ is G, and the polypeptide further comprises a linker-fatty acid. In a particular embodiment, the linker-fatty acid is 2-[2-(2-amino-ethoxy)-ethoxy]-acetyl)-γE₂-CO—(CH₂)₁₈—CO₂H. In a specific embodiment of Formula Id comprising the polypeptide, or a pharmaceutically acceptable salt thereof, the K at X₃₅ is conjugated to the linker-fatty acid. In another embodiment, the polypeptide comprises SEQ ID NO:44. In another embodiment, the polypeptide is SEQ ID NO:44. Alternatively, the polypeptide can have at least about 90 to 97% sequence similarity to SEQ ID NO:44. Alternatively, the polypeptide can have at least 90%, 91%, 92%, 93%, 94%, 95%, 96%, or 97% sequence similarity to SEQ ID NO:44. An exemplary embodiment comprising SEQ ID NO:44 is shown.

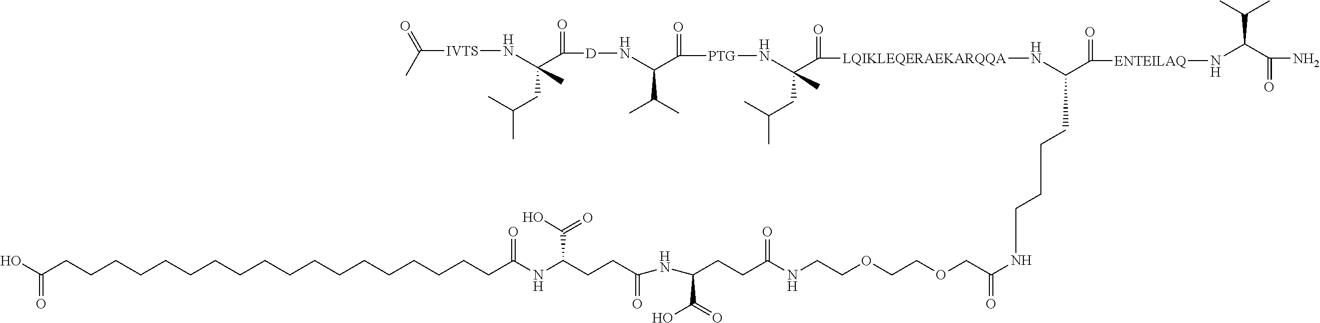

In a specific embodiment of Formula Id comprising the polypeptide, or a pharmaceutically acceptable salt thereof, X₁₅ is I, X₁₆ is Aib, and the polypeptide further comprises a linker-fatty acid. In a particular embodiment, the linker-fatty acid is γE₂-CO—(CH₂)₁₈—CO₂H). In a specific embodiment of the Formula Id comprising the polypeptide, or a pharmaceutically acceptable salt thereof, the K at X₃₅ is conjugated to the linker-fatty acid. In another embodiment, the polypeptide comprises SEQ ID NO:51. In another embodiment, the polypeptide is SEQ ID NO:51. Alternatively, the polypeptide can have at least about 90 to 97% sequence similarity to SEQ ID NO:51. Alternatively, the polypeptide can have at least 90%, 91%, 92%, 93%, 94%, 95%, 96%, or 97% sequence similarity to SEQ ID NO:51. An exemplary embodiment comprising SEQ ID NO:51 is shown.

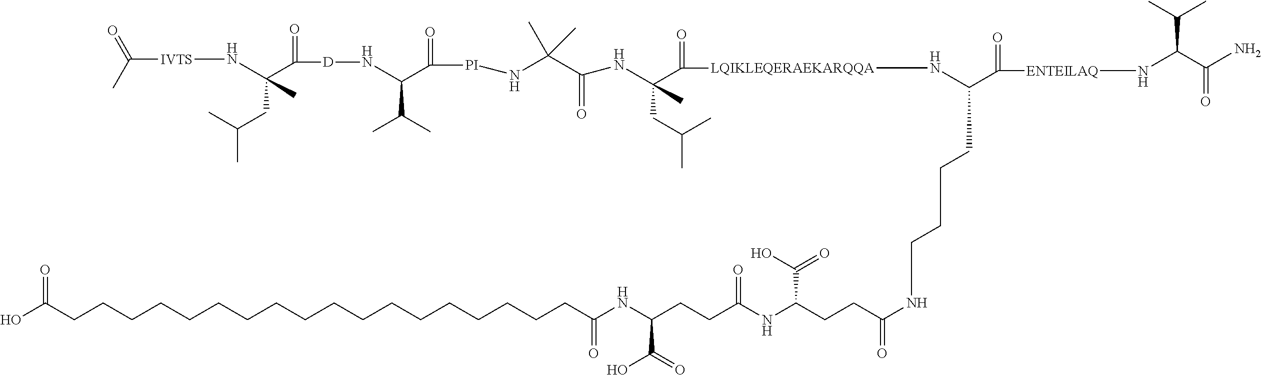

In another embodiment of Formula I, X₁ is absent, X₂ is absent, X₃ is absent, X₄ is P, X₅ is P, X₆ is P, X₁₁ is αMeL, X₁₃ is V or D-Val, X₁₅ is T, X₁₆ is Aib, X₁₇ is αMeL, X₁₉ is Q, X₂₀ is K, X₂₁ is L or Aib, X₂₂ is L, X₂₆ is R, X₂₇ is A, X₃₀ is A, X₃₁ is R, X₃₅ is K, X₃₇ is A or N, X₃₈ is A or T. In such embodiments, the polypeptide comprises a 41 amino acid sequence.

In some embodiments of the present disclosure, the polypeptide, or a pharmaceutically acceptable salt thereof, comprises Formula Ie, comprising

(SEQ ID NO: 73)
PPPIVTS αMeL DX₁₃PTAib αMeL LQKX₂₁
LEQERAEKARQQAKEX₃₇X₃₈EILAQV,

wherein,
X₁₃ is V or D-Val [wherein when X₁—X₃ are absent, X₁₃ is at position 10 of the polypeptide],
X₂₁ is L or Aib [wherein when X₁—X₃ are absent, X₂₁ is at position 18 of the polypeptide],
X₃₇ is A or N [wherein when X₁—X₃ are absent, X₃₇ is at position 34 of the polypeptide],
X₃₈ is A or T [wherein when X₁—X₃ are absent, X₃₈ is at position 35 of the polypeptide],
wherein the N-term amino acid is optionally N-acetylated, and wherein the C-term amino acid is optionally amidated.

In a specific embodiment of Formula Ie comprising the polypeptide, or a pharmaceutically acceptable salt thereof, X₁₃ is D-Val, X₂₁ is Aib, X₃₇ is N, X₃₈ is T, and the polypeptide further comprises a linker-fatty acid. In a particular embodiment, the linker-fatty acid is γE-(2-[2-(2-amino-ethoxy)-ethoxy]-acetyl)-εK—CO—(CH₂)₁₈—CO₂H. In a specific embodiment, the K at X₃₅ is conjugated to the linker-fatty acid. In some embodiments, the polypeptide comprises SEQ ID NO:46. In some embodiments, the polypeptide is SEQ ID NO:46. Alternatively, the polypeptide can have at least about 90 to 97% sequence similarity to SEQ ID NO:46. Alternatively, the polypeptide can have at least 90%, 91%, 92%, 93%, 94%, 95%, 96%, or 97% sequence similarity to SEQ ID NO:46. An exemplary embodiment comprising SEQ ID NO:46 is shown.

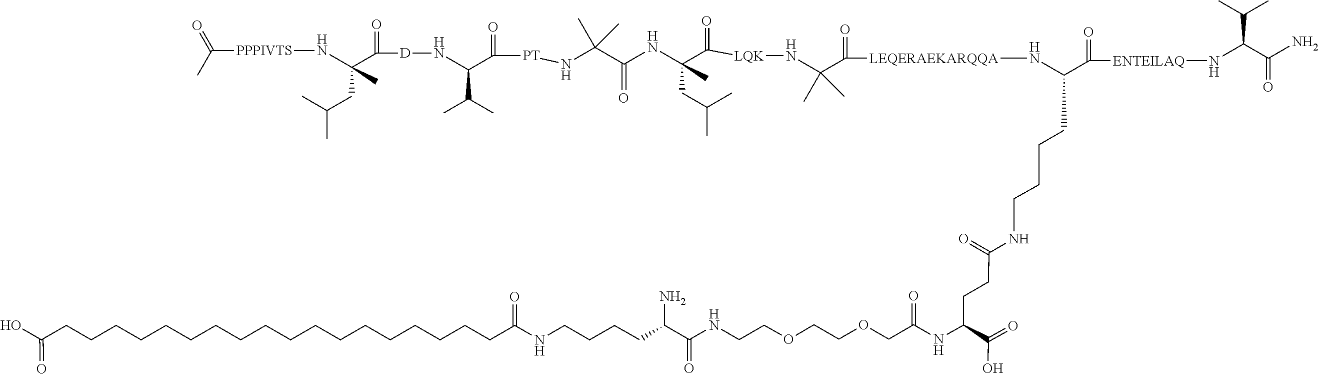

In a specific embodiment of Formula Id comprising the polypeptide, or a pharmaceutically acceptable salt thereof, X₁₃ is D-Val, X₂₁ is L, X₃₇ is A, X₃₈ is A, and the polypeptide further comprises a linker-fatty acid. In a particular embodiment, the linker-fatty acid is γE-(2-[2-(2-amino-ethoxy)-ethoxy]-acetyl)-εK—CO—(CH₂)₁₈—CO₂H. In a specific embodiment of Formula Id comprising the polypeptide, or a pharmaceutically acceptable salt thereof, the K at X₃₅ is conjugated to the linker-fatty acid. In some embodiments, the polypeptide comprises SEQ ID NO:47. In some embodiments, the polypeptide is SEQ ID NO:47. Alternatively, the polypeptide can have at least about 90 to 97% sequence similarity to SEQ ID NO:47. Alternatively, the polypeptide can have at least 90%, 91%, 92%, 93%, 94%, 95%, 96%, or 97% sequence similarity to SEQ ID NO:47. An exemplary embodiment comprising SEQ ID NO:47 is shown.

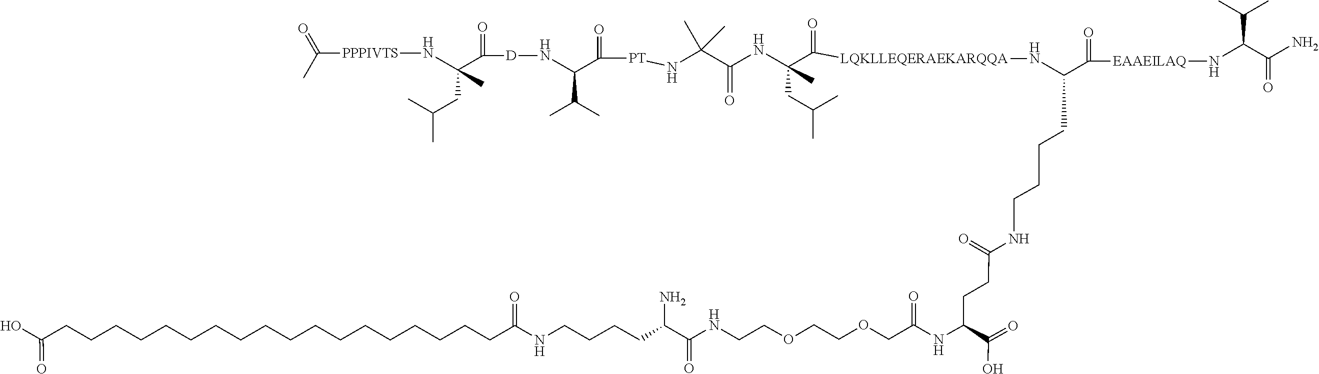

In a specific embodiment of Formula Id comprising the polypeptide, or a pharmaceutically acceptable salt thereof, X₁₃ is V, X₂₁ is L, X₃₇ is A, X₃₈ is A, and the polypeptide further comprises a linker-fatty acid. In a particular embodiment, the linker-fatty acid is (γE-(2-[2-(2-amino-ethoxy)-ethoxy]-acetyl)-εK—CO—(CH₂)₁₈—CO₂H. In a specific embodiment of Formula Id comprising the polypeptide, or a pharmaceutically acceptable salt thereof, the K at X₃₅ is conjugated to the linker-fatty acid. In some embodiments, the polypeptide comprises SEQ ID NO:48. In some embodiments, the polypeptide is SEQ ID NO:48. Alternatively, the polypeptide can have at least about 90 to 97% sequence similarity to SEQ ID NO:48. Alternatively, the polypeptide can have at least 90%, 91%, 92%, 93%, 94%, 95%, 96%, or 97% sequence similarity to SEQ ID NO:48. An exemplary embodiment comprising SEQ ID NO:48 is shown.

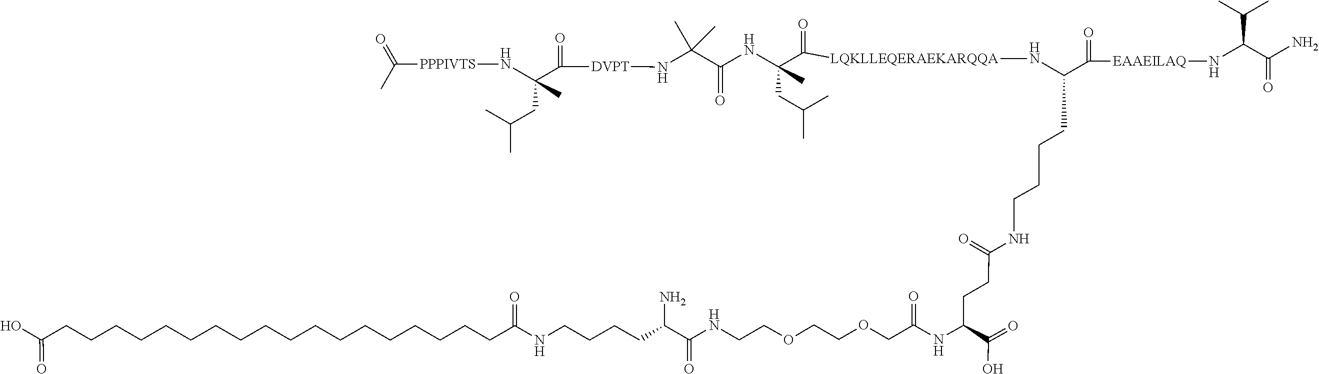

In another embodiment of Formula I, X₁ is γE, X₂ is absent, X₃ is absent; X₄ is G; X₅ is P; X₆ is S; X₁₁ is αMeL, X₁₃ is D-Val, X₁₅ is I, X₁₆ is Aib, X₁₇ is αMeL, X₁₉ is Q, X₂₀ is I, X₂₁ is K, X₂₂ is L, X₂₆ is R, X₂₇ is A, X₃₀ is A, X₃₁ is R, X₃₅ is K, X₃₇ is N, X₃₈ is T. In such embodiments, the polypeptide comprises a 42 amino acid sequence. In a specific embodiment, the polypeptide further comprises a linker-fatty acid. In a particular embodiment, the linker fatty acid is (2-[2-(2-Amino-ethoxy)-ethoxy]-acetyl)₂-γE-CO—(CH₂)₁₈—CO₂H. In a specific embodiment comprising the polypeptide, or a pharmaceutically acceptable salt thereof, the K at X₃₅ is conjugated to the linker-fatty acid.

In some embodiments, the polypeptide comprises SEQ ID NO:45. In some embodiments, the polypeptide is SEQ ID NO:45. Alternatively, the polypeptide can have at least about 90 to 97% sequence similarity to SEQ ID NO:45. Alternatively, the polypeptide can have at least 90%, 91%, 92%, 93%, 94%, 95%, 96%, or 97%, sequence similarity to SEQ ID NO:45. An exemplary embodiment comprising SEQ ID NO:45 is shown.

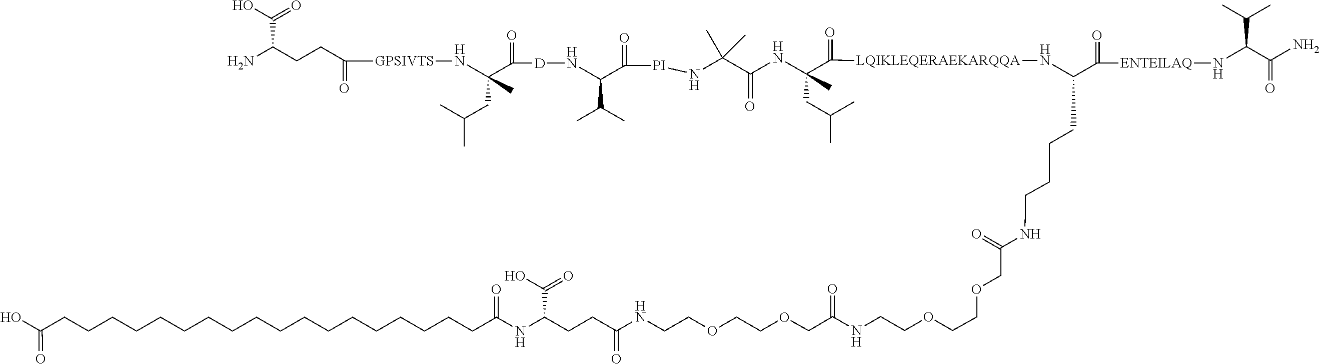

In another embodiment of Formula I, X₁ is absent, X₂ is absent, X₃ is absent, X₄ is absent, X₅ is absent, X₆ is absent, X₁₁ is L, X₁₃ is V, X₁₅ is I, X₁₆ is G, X₁₇ is αMeL, X₁₉ is Q, X₂₀ is K, X₂₁ is L, X₂₂ is I, X₂₆ is K, X₂₇ is Q, X₃₀ is E, X₃₁ is K, X₃₅ is K, X₃₇ is N, X₃₈ is T. In such embodiments, the polypeptide comprises a 38 amino acid sequence.

In a specific embodiment of the polypeptide, or a pharmaceutically acceptable salt thereof, the polypeptide further comprises a linker-fatty acid. In a particular embodiment, the linker-fatty acid is (2-[2-(2-amino-ethoxy)-ethoxy]-acetyl)₂γE-C20-OH)). In a specific embodiment comprising the polypeptide, or a pharmaceutically acceptable salt thereof, the K at X₃₅ is conjugated to the linker-fatty acid.

In some embodiments, the polypeptide comprises SEQ ID NO:49. In some embodiments, the polypeptide is SEQ ID NO:49. Alternatively, the polypeptide can have at least about 90 to 97% sequence similarity to SEQ ID NO:49. Alternatively, the polypeptide can have at least 90%, 91%, 92%, 93%, 94%, 95%, 96%, or 97% sequence similarity to SEQ ID NO:49. An exemplary embodiment comprising SEQ ID NO:49 is shown.

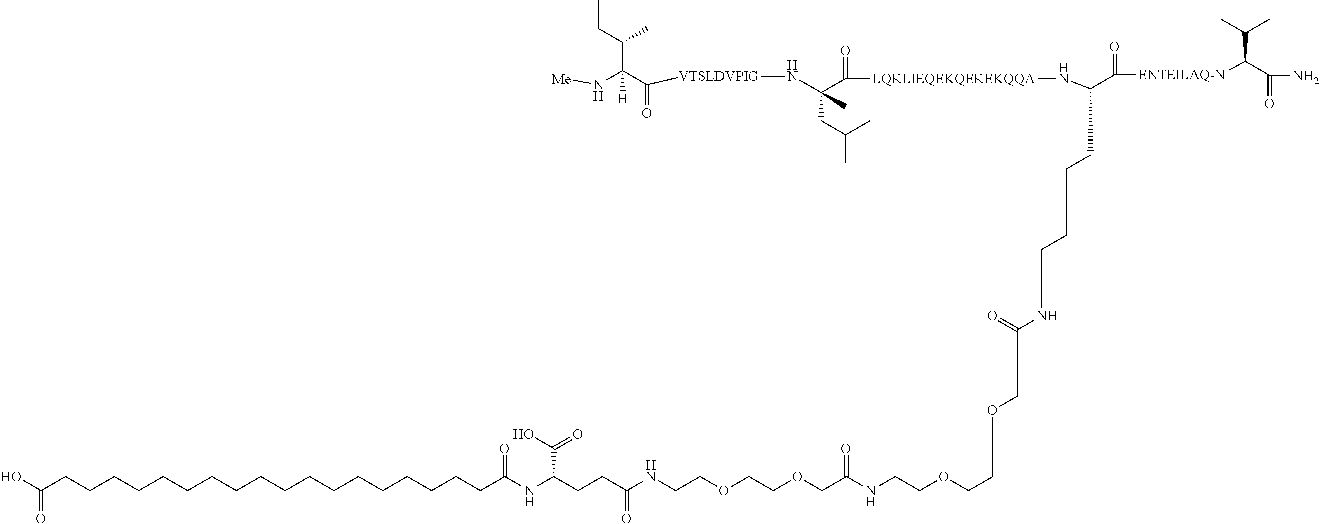

In another embodiment of Formula I, X₁ is absent, X₂ is absent, X₃ is absent, X₄ is absent, X₅ is absent, X₆ is absent, X₁₁ is L, X₁₃ is V, X₁₅ is I, X₁₆ is G, X₁₇ is αMeL, X₁₉ is Q, X₂₀ is K, X₂₁ is L, X₂₂ is L, X₂₆ is R, X₂₇ is A, X₃₀ is A, X₃₁ is R, X₃₅ is K, X₃₇ is A, X₃₈ is A. In such embodiments, the polypeptide comprises a 38 amino acid sequence.

In a specific embodiment of the polypeptide, or a pharmaceutically acceptable salt thereof, the polypeptide further comprises a linker-fatty acid. In a particular embodiment, the linker-fatty acid is 2-[2-(2-Amino-ethoxy)-ethoxy]-acetyl)₂-γE-CO—(CH₂)₁₈—CO₂H. In a specific embodiment comprising the polypeptide, or a pharmaceutically acceptable salt thereof, the K at X₃₅ is conjugated to the linker-fatty acid.

In some embodiments, the polypeptide comprises SEQ ID NO:50. In other embodiments, the polypeptide is SEQ ID NO:50. Alternatively, the polypeptide can have at least about 90 to 97% sequence similarity to SEQ ID NO:50. Alternatively, the polypeptide can have at least 90%, 91%, 92%, 93%, 94%, 95%, 96%, or 97% sequence similarity to SEQ ID NO:50. An exemplary embodiment comprising SEQ ID NO:50 is shown.

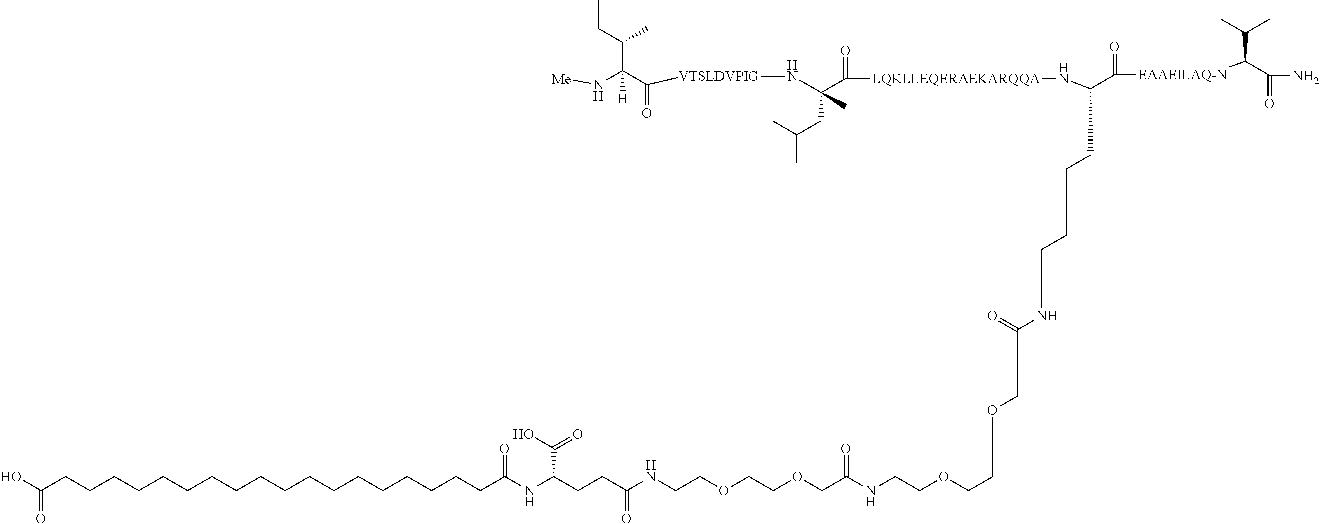

In one aspect, provided herein are polypeptides, or pharmaceutically acceptable salts thereof, comprising sequences with at least eight amino acid modifications from human urocortin-2 (SEQ ID NO:53). In some embodiments, the polypeptides provided herein comprise sequences containing at least one amino acid modification at position: 3, 19, 22, 23, 26, 30, 33, or 37 from human urocortin-2 (SEQ ID NO:53). In some embodiments, the polypeptides provided herein comprise sequences containing at least one of the following amino acid modifications from human urocortin-2 (SEQ ID NO:53): L3T, A19E, R22E, A23K, E26Q, T30E, R33E, R37Q, or any combination thereof.

In some embodiments, the present disclosure provides a polypeptide comprising any one of SEQ ID NOs: 1-52, or a pharmaceutically acceptable salt thereof. Alternatively, the polypeptide can have at least about 90 to 99% sequence similarity to any one of SEQ ID NOs:1-52, or a pharmaceutically acceptable salt thereof. Alternatively, the polypeptide can have at least 90%, 91%, 92%, 93%, 94%, 95%, 96%, 97%, 98%, or 99% sequence similarity to any one of SEQ ID NOs:1-52.

In a specific embodiment, the polypeptide comprises SEQ ID NO:1, or a pharmaceutically acceptable salt thereof. In a specific embodiment, the polypeptide comprises SEQ ID NO:2, or a pharmaceutically acceptable salt thereof. In a specific embodiment, the polypeptide comprises SEQ ID NO:22, or a pharmaceutically acceptable salt thereof. In a specific embodiment, the polypeptide comprises SEQ ID NO:25, or a pharmaceutically acceptable salt thereof. In a specific embodiment, the polypeptide comprises SEQ ID NO:29, or a pharmaceutically acceptable salt thereof. In a specific embodiment, the polypeptide comprises SEQ ID NO:41, or a pharmaceutically acceptable salt thereof. In a specific embodiment, the polypeptide comprises SEQ ID NO:42, or a pharmaceutically acceptable salt thereof. In a specific embodiment, the polypeptide comprises SEQ ID NO:45, or a pharmaceutically acceptable salt thereof. In a specific embodiment, the polypeptide comprises SEQ ID NO:46, or a pharmaceutically acceptable salt thereof. In a specific embodiment, the polypeptide comprises SEQ ID NO:48, or a pharmaceutically acceptable salt thereof. In a specific embodiment, the polypeptide comprises SEQ ID NO:49, or a pharmaceutically acceptable salt thereof. In a specific embodiment, the polypeptide comprises SEQ ID NO:50, or a pharmaceutically acceptable salt thereof.

In a specific embodiment, the polypeptide, or a pharmaceutically acceptable salt thereof, is selected from the group consisting of SEQ ID NO:1, SEQ ID NO:2, SEQ ID NO:22, SEQ ID NO:25, SEQ ID NO:29, SEQ ID NO:41, SEQ ID NO:42, SEQ ID NO:45, SEQ ID NO:46, SEQ ID NO:48, SEQ ID NO:49, an SEQ ID NO:50.

In specific embodiments of the present disclosure, the pharmaceutically acceptable salt is selected from sodium, potassium, trifluoroacetate, hydrochloride, or acetate.

In some embodiments, the present disclosure provides a pharmaceutical composition comprising the polypeptide or a pharmaceutically acceptable salt thereof, and at least one pharmaceutically acceptable carrier, diluent, or excipient.

In some embodiments, the present disclosure provides a pharmaceutical composition, wherein the composition is formulated for oral administration. In other embodiments, the composition is formulated for subcutaneous administration.

In some embodiments, the present disclosure provides a method of treating a disease or condition selected from the group consisting of diabetes mellitus, obesity, chronic weight management, non-alcoholic fatty liver disease (NAFLD), non-alcoholic steatohepatitis (NASH), dyslipidemia, metabolic syndrome, chronic kidney disease (CKD), osteoarthritis (OA), obesity-related sleep apnea (OSA), sarcopenia, cachexia, sarcopenic obesity (SO), and polycystic ovarian syndrome (PCOS), the method comprising administering to an individual in need thereof an effective amount of a polypeptide, or a pharmaceutically acceptable salt thereof.

In some embodiments, the present disclosure provides a polypeptide, or a pharmaceutically acceptable salt thereof, for use in therapy.

In some embodiments, the present disclosure provides a polypeptide, or a pharmaceutically acceptable salt thereof, for use in treating a disease or condition selected from the group consisting of diabetes mellitus, obesity, chronic weight management, non-alcoholic fatty liver disease (NAFLD), non-alcoholic steatohepatitis (NASH), dyslipidemia, metabolic syndrome, chronic kidney disease (CKD), osteoarthritis (OA), obesity-related sleep apnea (OSA), sarcopenia, cachexia, sarcopenic obesity (SO), and polycystic ovarian syndrome (PCOS).

In some embodiments, the present disclosure provides for use of a polypeptide, or a pharmaceutically acceptable salt thereof, in the manufacture of a medicament for treatment a disease or condition selected from the group consisting of diabetes mellitus, obesity, chronic weight management, non-alcoholic fatty liver disease (NAFLD), non-alcoholic steatohepatitis (NASH), dyslipidemia, metabolic syndrome, chronic kidney disease (CKD), osteoarthritis (OA), obesity-related sleep apnea (OSA), sarcopenia, cachexia, sarcopenic obesity (SO), and polycystic ovarian syndrome (PCOS).

In some embodiments, the present disclosure provides a method of treating a disease or condition i, selected from the group consisting of: diabetes mellitus, obesity, chronic weight management, non-alcoholic fatty liver disease (NAFLD), non-alcoholic steatohepatitis (NASH), dyslipidemia, metabolic syndrome, chronic kidney disease (CKD), osteoarthritis (OA), obesity-related sleep apnea (OSA), sarcopenia, cachexia, sarcopenic obesity (SO), and polycystic ovarian syndrome (PCOS), comprising administering to the patient an effective amount of the polypeptide and an effective amount of an additional therapeutic agent.

In an embodiment, provided herein is a polypeptide, or a pharmaceutically acceptable salt thereof, that may be used in simultaneous, separate, or sequential combinations with one or more additional therapeutic agents.

In some embodiments, the present disclosure provides a method of increasing exercise capacity or VO2 max.

In some embodiments, the additional therapeutic agent is a dual agonist of the GIP and GLP-1 receptors. In a specific embodiment, the dual agonist of the GIP and GLP-1 receptors is a compound comprising SEQ ID NO:55.

In some embodiments, the additional therapeutic agent is a tri-agonist of the GIP, GLP-1, and glucagon receptors. In a specific embodiment, the tri-agonist of the GIP, GLP-1, and glucagon receptors is a compound comprising SEQ ID NO:56, SEQ ID NO:57, or SEQ ID NO:58, or a pharmaceutically acceptable salt thereof.

In some embodiments, the present disclosure provides a method wherein the additional therapeutic agent is a dual agonist of the glucagon and GLP-1 receptors. In a specific embodiment, the dual agonist of the glucagon and GLP-1 receptors is a compound comprising SEQ ID NO:59, SEQ ID NO:60, SEQ ID NO:61, or SEQ ID NO:62, or a pharmaceutically acceptable salt thereof.

In some embodiments, the additional therapeutic agent is an agonist selective for the amylin receptor. In a specific embodiment, the agonist selective for the amylin receptor is a compound comprising SEQ ID NO:63. SEQ ID NO:64, SEQ ID NO:65, or SEQ ID NO:66.

In some embodiments, the additional therapeutic agent is a dual agonist of both the calcitonin and amylin receptors. In a specific embodiment, the dual agonist of both the calcitonin and amylin receptors is a compound comprising SEQ ID NO:67.

In some embodiments, the additional therapeutic agent is an agonist of the GLP-1 receptor. In a specific embodiment, the agonist of the GLP-1 receptor is a compound comprising SEQ ID NO:79, SEQ ID NO:80, or SEQ ID NO:81.

Unless defined otherwise, all technical and scientific terms used herein have the same meaning as commonly understood to one of skill in the art to which the disclosure pertains. Although any methods and materials similar to or equivalent to those described herein can be used in the practice or testing of the polypeptides, pharmaceutical compositions, and methods, the preferred methods and materials are described herein.

Moreover, reference to an element by the indefinite article “a” or “an” does not exclude the possibility that more than one element is present, unless the context clearly requires that there be one and only one element. The indefinite article “a” or “an” thus usually means “at least one.”

As used herein, “about” means within a statistically meaningful range of a value or values such as, for example, a stated concentration, length, molecular weight, pH, sequence identity, time frame, temperature or volume. Such a value or range can be within an order of magnitude typically within 20%, more typically within 10%, and even more typically within 5% of a given value or range. The allowable variation encompassed by “about” will depend upon the particular system under study, and can be readily appreciated by one of skill in the art.

As used herein, “additional therapeutic agent” refers to a substance used to treat, manage, or prevent diseases that is different than a polypeptide with activity at the CRH2 receptor. Herein, an additional therapeutic agent may include, but is not limited to, a dual agonist of the GIP and GLP-1 receptors, a triagonist of the GIP, GLP-1, and glucagon receptors, a dual agonist of the glucagon and GLP-1 receptors, an agonist selective for the amylin receptor, a dual agonist for both the calcitonin and amylin receptors, an agonist of the GLP-1 receptor, or a leptin receptor antibody.

As used herein, and in reference to one or more of the CRH (e.g., CRHR1 or CRHR2) receptors, “activity,” “activate,” “activating” and the like means a capacity of a compound, such as the polypeptides described herein, to bind to and induce a response at the receptor(s), as measured using assays known in the art, such as the in vitro assays described below.

As used herein, “amino acid with a functional group available for conjugation” means any natural (coded) or non-natural (non-coded) amino acid with a functional group that may be conjugated to fatty acid directly or by way of, for example, a linker. Examples of such functional groups include, but are not limited to, alkynyl, alkenyl, amino, azido, bromo, carboxyl, chloro, iodo, and thiol groups. Examples of natural amino acids including such functional groups include K (amino), C (thiol), E (carboxyl) and D (carboxyl).

As used herein, “conservative amino acid substitution” means substitution of an amino acid with an amino acid having similar characteristics (e.g., charge, sidechain size, hydrophobicity/hydrophilicity, backbone conformation and rigidity, etc.) and having minimal impact on the biological activity of the resulting substituted peptide or polypeptide. Conservative substitutions of functionally similar amino acids are well known in the art and thus need not be exhaustively described herein.

As used herein, “at least” means not less than; at the minimum.

As used herein, body mass index (BMI) refers to a person's weight in kilograms divided by the square of a person's height in meters.

As used herein, “cachexia” refers to a complex syndrome associated with an underlying illness, causing ongoing muscle loss that is not entirely reversed with nutritional supplementation. A range of diseases can cause cachexia, including but not limited to cancer, congestive heart failure, chronic obstructive pulmonary disease, chronic kidney disease, and AIDS.

As used herein, “chronic weight management” refers to the long-term management of body weight in individuals who are overweight or have obesity. Chronic weight management may be managed by polypeptides of the present disclosure. Chronic weight management may also be managed by polypeptides of the present disclosure in addition to healthy lifestyle changes, including but not limited to, a balanced diet and regular physical activity.

As used herein, “chronic kidney disease” (CKD) refers to the loss of kidney function over time. A person with CKD may be affected by one or more of the following signs or symptoms due to loss of kidney function: nausea, vomiting, loss of appetite, fatigue, weakness, sleep problems, urinating more than normal, muscle cramps, swelling of feet or ankles, high blood pressure, or shortness of breath.

As used herein, “conservative amino acid substitution” means substitution of an amino acid with an amino acid having similar characteristics (e.g., charge, sidechain size, hydrophobicity/hydrophilicity, backbone conformation and rigidity, etc.) and having minimal impact on the biological activity of the resulting substituted peptide or polypeptide. Conservative substitutions of functionally similar amino acids are well known in the art and thus need not be exhaustively described herein.

As used herein, “C₁₆-C₂₂ fatty acid” means a carboxylic acid having between 16 and 22 carbon atoms. The C₁₆-C₂₂ fatty acid suitable for use herein can be a saturated monoacid or saturated diacid. As used herein, “saturated” means the fatty acid contains no carbon-carbon double or triple bonds.

As used herein, “diabetes” or “diabetes mellitus” refers to a group of endocrine diseases characterized by high blood sugar levels. Diabetes herein refers either the pancreas of the body not producing enough insulin, or the cells of the body becoming unresponsive to the hormone's effects. Diabetes herein also refers to type 2 diabetes mellitus (T2DM), which is characterized by high blood sugar, insulin resistance, and/or relative lack of insulin. As used herein, diabetes also refers to high blood sugar levels developed by a woman during pregnancy, known as gestational diabetes.

As used herein, “dyslipidemia” refers to abnormally high levels of lipids (fats) in the blood. Dyslipidemia also refers to high levels of cholesterol in the blood.

As used herein, “effective amount” means an amount, concentration or dose of one or more polypeptides described herein, or a pharmaceutically acceptable salt thereof which, upon single or multiple dose administration to an individual in need thereof, provides a desired effect in such an individual under diagnosis or treatment. An effective amount can be readily determined by one of skill in the art through the use of known techniques and by observing results obtained under analogous circumstances. In determining the effective amount for an individual, a number of factors are considered including, but not limited to, the species of mammal; its size, age, and general health; the specific disease or disorder involved; the degree of or involvement of or the severity of the disease or disorder; the response of the individual patient; the particular polypeptide administered; the mode of administration; the bioavailability characteristics of the preparation administered; the dose regimen selected; the use of concomitant medication; and other relevant circumstances.

As used herein, “extended duration of action” means that binding affinity and activity for polypeptide continues for a period of time greater than native human urocortin, allowing for dosing at least as infrequently as once daily or even thrice-weekly, twice-weekly or once-weekly. The time action profile of the polypeptide may be measured using known pharmacokinetic test methods such as those utilized in the examples below.

As used herein, “metabolic syndrome” refers to a group of health conditions leading to increased risk of heart disease and related problems. A person with metabolic syndrome has three or more of the following health conditions (1) high blood sugar levels, (2) low levels of HDL cholesterol, (3) high levels of triglycerides in the blood, (4) large waist circumference, and (5) high blood pressure.

As used herein, “NASH” refers to nonalcoholic steatohepatitis, aka fatty liver disease. “NASH” also refers to liver inflammation and damage caused by a buildup of fat in the liver. “NASH” also refers to a subtype of nonalcoholic fatty liver disease (“NAFLD”). In some embodiments, “NASH” may be synonymous with “NAFLD.”

As used herein “obesity” refers to a disorder involving excess body fat that increases the risk of health problems. The term “obesity” also refers to weight that is higher than what is considered healthy weight for a given height. The term “obesity” also refers to a BMI greater than 30.0 or a BMI of 27.0 or greater (overweight) with at least one weight-related comorbid condition (e.g., hypertension, type 2 diabetes mellitus, or dyslipidemia).

As used herein, “osteoarthritis” (OA) is a form of arthritis causing inflammation of one or more joints. “Osteoarthritis” may refer to arthritis that affects joints in the hand, spine knees, and hips.

As used herein, “obstructive sleep apnea” (OSA) refers to a sleep-related breathing disorder and is characterized by recurrent episodes of complete or partial obstruction of the upper airway leading to reduced or absent breathing during sleep.

As used herein, “patient” and “individual” are used interchangeably, and mean a mammal, and preferably a human being. In certain embodiments, the patient, preferably a human, is further characterized with a disease, disorder, or condition that would benefit from administration of a polypeptide of the present disclosure.

As used herein, “polypeptide” or “peptide” means a polymer of amino acid residues. The term applies to polymers comprising naturally occurring amino acids and polymers comprising one or more non-naturally occurring amino acids.

As used herein, “polycystic ovarian syndrome” (PCOS) refers to an endocrine disorder in women of reproductive age. PCOS also refers to a woman who may have one or more of the following symptoms: cysts on the ovaries, irregular menstrual periods, heavy periods, excess hair, difficulty getting pregnant, or insulin resistance.

As used herein, “individual in need thereof” means a mammal, such as a human, with a condition, disease, disorder, or symptom requiring treatment or therapy, included for example, those listed herein.

As used herein, “quality of weight loss” refers to a decrease in fat mass but preservation of lean mass.

As used herein, “sarcopenia” refers to a progressive and generalized skeletal muscle disorder involving the accelerated loss of muscle mass and function.

As used herein, “sarcopenic obesity” refers to a person who has both sarcopenia (loss of muscle) and obesity. Sarcopenic obesity also refer to the presence of low muscle mass or strength along with a high fat mass.

As used herein, the term “sequence similarity” refers to the degree of similarity between two sequences. The degree of sequence similarity between two polypeptides may be expressed as a percentage, calculated as follows:

% sequence identity=100% (number of identical amino acids)/(length of the shortest common sequence)

As used herein, “treat,” “treating,” “to treat” and the like mean restraining, slowing, stopping or reversing the progression or severity of an existing condition, disease, disorder, or symptom.

Additional non-limiting embodiments are set forth below.

1. A polypeptide, or pharmaceutically acceptable salt thereof, comprising:

(SEQ ID NO: 68)
X₁X₂X₃X₄X₅X₆IVTSX₁₁DX₁₃PX₁₅X₁₆X₁₇
LX₁₉X₂₀X₂₁X₂₂EQEX₂₆X₂₇EKX₃₀X₃₁QQA
X₃₅EX₃₇X₃₈EILAQV,

wherein
X₁ is Pyr, γE, or absent,
X₂ is G or absent,
X₃ is G, S, or absent,
X₄ is S, P, G, or absent,
X₅ is S, P, or absent,
X₆ is G, S, P, or absent,
X₁₁ is L or αMeL,
X₁₃ is V or D-Val,
X₁₅ is T or I,
X₁₆ is Aib or G,
X₁₇ is αMeL,
X₁₉ is Q or E,
X₂₀ is K or I,
X₂₁ is I, K, Aib, or L,
X₂₂ is I or L,
X₂₆ is R or K,
X₂₇ is A or Q,
X₃₀ is A or E,
X₃₁ is R or K,
X₃₅ is K or T,
X₃₇ is A or N, and
X₃₈ is A or T;
   2. The polypeptide of embodiment 1, or a pharmaceutically acceptable salt thereof, comprising:

(SEQ ID NO: 69)
GSPSIVTS αMeL DVPT Aib αMeL L
QKX₂₁LEQERAEKARQQAKEAAEILAQV,

wherein,
X₂₁ is I or L,
   wherein the N-term amino acid is the G at X₃, and wherein the G is N-acetylated, and wherein the C-term amino acid is V, and wherein the V is amidated.
   3. The polypeptide, or pharmaceutically acceptable salt thereof, of embodiment 2 further comprising a linker-fatty acid conjugated to the epsilon-amino group of the side chain at the K at X₃₅, wherein the K conjugated to the linker-fatty acid is selected from the group consisting of K((2-[2-(2-amino-ethoxy)-ethoxy]-acetyl)-PKE-γE-CO—(CH₂)₁₈—CO₂H), K(Ahx-(2-[2-(2-amino-ethoxy)-ethoxy]-acetyl)-γE-CO—(CH₂)₁₈—CO₂H), K(γE-(2-[2-(2-amino-ethoxy)-ethoxy]-acetyl)-Ahx-CO—(CH₂)₁₈—CO₂H), K(γE-GKEKEKE-CO—(CH₂)₁₈—CO₂H), K(G-(βA)₃-γE-CO—(CH₂)₁₈—CO₂H), K(G-(βA)₄-γE-CO—(CH₂)₁₈—CO₂H), K((2-[2-(2-amino-ethoxy)-ethoxy]-acetyl)-KEKEKE-γE-CO—(CH₂)₁₈—CO₂H), K(βA-S-βA-S-γE-CO—(CH₂)₁₈—CO₂H), K(γE-PKE-Ahx-CO—(CH₂)₁₈—CO₂H), K(γE-PKE-εK—CO—(CH₂)₁₈—CO₂H), K(γE-PPP—εK—CO—(CH₂)₁₈—CO₂H), K(γE-PPPPPP-CO—(CH₂)₁₈—CO₂H), K(γE-PEPEPE-CO—(CH₂)₁₈—CO₂H), K(γE-(2-[2-(2-amino-ethoxy)-ethoxy]-acetyl)₂-εK—CO—(CH₂)₁₈—CO₂H), K(γE-εK-γE-εK—CO—(CH₂)₁₈—CO₂H), K(γE-εK-(2-[2-(2-amino-ethoxy)-ethoxy]-acetyl)-εK—CO—(CH₂)₁₈—CO₂H), K(γE-(2-[2-(2-amino-ethoxy)-ethoxy]-acetyl)-εK—CO—(CH₂)₁₈—CO₂H), and K(γE-(2-[2-(2-amino-ethoxy)-ethoxy]-acetyl)-εK—CO—(CH₂)₁₆—CO₂H).
   4. The polypeptide, or a pharmaceutically acceptable salt thereof, of embodiment 3 wherein X₂₁ is I and the K at X₃₅ is conjugated to the fatty acid according to the formula K(γE-(2-[2-(2-amino-ethoxy)-ethoxy]-acetyl)-εK—CO—(CH₂)₁₈—CO₂H).
   5. The polypeptide, or a pharmaceutically acceptable salt thereof, of embodiment 3 wherein X₂₁ is L and the K at X₃₅ is conjugated to the fatty acid according to the formula K(γE-(2-[2-(2-amino-ethoxy)-ethoxy]-acetyl)-εK—CO—(CH₂)₁₈—CO₂H).
   6. The polypeptide, or a pharmaceutically acceptable salt thereof, of embodiment 3 wherein X₂₁ is I and the K at X₃₅ is conjugated to the fatty acid according to the formula K((2-[2-(2-amino-ethoxy)-ethoxy]-acetyl)-PKE-γE-CO—(CH₂)₁₈—CO₂H).
   7. The polypeptide, or a pharmaceutically acceptable salt thereof, of embodiment 3 wherein X₂₁ is I and the K at X₃₅ is conjugated to the fatty acid according to the formula K(Ahx-(2-[2-(2-amino-ethoxy)-ethoxy]-acetyl)-γE-CO—(CH₂)₁₈—CO₂H).
   8. The polypeptide, or a pharmaceutically acceptable salt thereof, of embodiment 3 wherein X₂₁ is I and the K at X₃₅ is conjugated to the fatty acid according to the formula K(γE-(2-[2-(2-amino-ethoxy)-ethoxy]-acetyl)-Ahx-CO—(CH₂)₁₈—CO₂H).
   9. The polypeptide, or a pharmaceutically acceptable salt thereof, of embodiment 3 wherein X₂₁ is I and the K at X₃₅ is conjugated to the fatty acid according to the formula K(γE-GKEKEKE-CO—(CH₂)₁₈—CO₂H).
   10. The polypeptide, or a pharmaceutically acceptable thereof, of embodiment 3 wherein X₂₁ is I and the K at X₃₅ is conjugated to the fatty acid according to the formula K(G-(βA)₃-γE-CO—(CH₂)₁₈—CO₂H).
   11. The polypeptide, or a pharmaceutically acceptable salt thereof, of embodiment 3 wherein X₂₁ is I and the K at X₃₅ is conjugated to the fatty acid according to the formula K(G-(βA)₄-γE-CO—(CH₂)₁₈—CO₂H).
   12. The polypeptide, or a pharmaceutically acceptable salt thereof, of embodiment 3 wherein X₂₁ is I and the K at X₃₅ is conjugated to the fatty acid according to the formula K((2-[2-(2-amino-ethoxy)-ethoxy]-acetyl)-KEKEKE-γE-CO—(CH₂)₁₈—CO₂H).
   13. The polypeptide, or a pharmaceutically acceptable salt thereof, of embodiment 3 wherein X₂₁ is I and the K at X₃₅ is conjugated to the fatty acid according to the formula K(βA-S-βA-S-γE-CO—(CH₂)₁₈—CO₂H).
   14. The polypeptide, or a pharmaceutically acceptable salt thereof, of embodiment 3 wherein X₂₁ is I and the K at X₃₅ is conjugated to the fatty acid according to the formula K(γE-PKE-Ahx-CO—(CH₂)₁₈—CO₂H).
   15. The polypeptide, or a pharmaceutically acceptable salt thereof, of embodiment 3 wherein X₂₁ is L and the K at X₃₅ is conjugated to the fatty acid according to the formula K(γE-(2-[2-(2-amino-ethoxy)-ethoxy]-acetyl)-εK—CO—(CH₂)₁₆—CO₂H).
   16. The polypeptide, or a pharmaceutically acceptable salt thereof, of embodiment 3 wherein X₂₁ is I and the K at X₃₅ is conjugated to the fatty acid according to the formula K(γE-PKE-εK—CO—(CH₂)₁₈—CO₂H).
   17. The polypeptide, or a pharmaceutically acceptable salt thereof, of embodiment 3 wherein X₂₁ is I and the K at X₃₅ is conjugated to the fatty acid according to the formula K(γE-PPP—εK—CO—(CH₂)₁₈—CO₂H).
   18. The polypeptide, or a pharmaceutically acceptable salt thereof, of embodiment 3 wherein X₂₁ is I and the K at X₃₅ is conjugated to the fatty acid according to the formula K(γE-PPPPPP-CO—(CH₂)₁₈—CO₂H).
   19. The polypeptide, or a pharmaceutically acceptable salt thereof, of embodiment 3 wherein X₂₁ is I and the K at X₃₅ is conjugated to the fatty acid according to the formula K(γE-PEPEPE-CO—(CH₂)₁₈—CO₂H).
   20. The polypeptide, or a pharmaceutically acceptable salt thereof, of embodiment 3 wherein X₂₁ is I and the K at X₃₅ is conjugated to the fatty acid according to the formula K(γE-(2-[2-(2-amino-ethoxy)-ethoxy]-acetyl)₂-εK—CO—(CH₂)₁₈—CO₂H).
   21. The polypeptide, or pharmaceutically acceptable salt thereof, of embodiment 3 wherein X₂₁ is I and the K at X₃₅ is conjugated to the fatty acid according to the formula K(γE-εK-γE-εK—CO—(CH₂)₁₈—CO₂H).
   22. The polypeptide, or a pharmaceutically acceptable salt thereof, of embodiment 3 wherein X₂₁ is I and the K at X₃₅ is conjugated to the fatty acid according to the formula K(γE-εK-(2-[2-(2-amino-ethoxy)-ethoxy]-acetyl)-εK—CO—(CH₂)₁₈—CO₂H).
   23. The polypeptide of embodiment 2, or a pharmaceutically acceptable salt thereof, wherein X₂₁ is I.
   24. The polypeptide, or a pharmaceutically acceptable salt thereof, of embodiment 3 wherein X₂₁ is I, and the K at X₃₅ is conjugated to the fatty acid according to the formula K(γE-(2-[2-(2-amino-ethoxy)-ethoxy]-acetyl)-εK—CO—(CH₂)₁₆—CO₂H).
   25. The polypeptide, or a pharmaceutically acceptable salt thereof, of embodiment 1 comprising:
   Pyr GSPSGIVTS αMeL DVPT Aib αMeL LQKILEQERAEKARQQAKEAAEILAQV (SEQ ID NO:70), or a pharmaceutically acceptable salt thereof, wherein the C-term V is amidated.
   26. The polypeptide, or pharmaceutically acceptable salt thereof, of embodiment 25 further comprising a linker-fatty acid conjugated to the epsilon-amino group of the side chain at K at X₃₅, wherein the K conjugated to the linker-fatty acid is selected from the group consisting of K(γE-(2-[2-(2-amino-ethoxy)-ethoxy]-acetyl)-εK—CO—(CH₂)₁₈—CO₂H) and K(γE-(2-[2-(2-amino-ethoxy)-ethoxy]-acetyl)-εK—CO—(CH₂)₁₆—CO₂H).
   27. The polypeptide of embodiment 26 wherein the K at X₃₅ is conjugated to the fatty acid according to the formula K(γE-(2-[2-(2-amino-ethoxy)-ethoxy]-acetyl)-εK—CO—(CH₂)₁₈—CO₂H).
   28. The polypeptide of embodiment 26 wherein the K at X₃₅ is conjugated to the fatty acid according to the formula K(γE-(2-[2-(2-amino-ethoxy)-ethoxy]-acetyl)-εK—CO—(CH₂)₁₆—CO₂H).
   29. The polypeptide, or a pharmaceutically acceptable salt thereof, of embodiment 1 comprising:

(SEQ ID NO: 71)
X₁GSPSIVTS αMeL DVPT Aib αMeL L
X₁₉KILEQERAEKARQQATEAAEILAQV,

wherein
X₁ is γE or absent,
X₂ is absent,
X₁₉ is Q or E,
and wherein the C-term amino acid is V at position X₄₄, and the V at X₄₄ is amidated.
   30. The polypeptide of embodiment 29 wherein X₁ is γE and the γE at X₁ is conjugated to the G at X₃ via an amide bond between the γ-carboxylic acid group of the γE and the amino group of the G.
   31. The polypeptide of embodiment 30 further comprising a fatty acid conjugated to the γE at position X₁ via an amide bond between the alpha-amino group of the γE and the carboxylic acid group of the (2-[2-(2-amino-ethoxy)-ethoxy]acetic acid monomer, wherein the linker-fatty acid is selected from the group consisting of (2-[2-(2-amino-ethoxy)-ethoxy]-acetyl)-εK—CO—(CH₂)₁₈—CO₂H, (2-[2-(2-amino-ethoxy)-ethoxy]-acetyl)-K—CO—(CH₂)₁₈—CO₂H, (2-[2-(2-amino-ethoxy)-ethoxy]-acetyl)-Dap-CO—(CH₂)₁₈—CO₂H, and (2-[2-(2-amino-ethoxy)-ethoxy]-acetyl)-εK—CO—(CH₂)₁₆—CO₂H.
   32. The polypeptide of embodiment 31 wherein the linker-fatty acid conjugated to the γE at X₁ is (2-[2-(2-amino-ethoxy)-ethoxy]-acetyl)-εK—CO—(CH₂)₁₈—CO₂H and X₁₉ is Q.
   33. The polypeptide of embodiment 31 wherein the linker-fatty acid conjugated to the γE at X₁ is (2-[2-(2-amino-ethoxy)-ethoxy]-acetyl)-K—CO—(CH₂)₁₈—CO₂H and X₁₉ is Q.
   34. The polypeptide of embodiment 31 wherein X₁ is absent and X₁₉ is Q.
   35. The polypeptide of embodiment 31 wherein the linker-fatty acid conjugated to the γE at X₁ is (2-[2-(2-amino-ethoxy)-ethoxy]-acetyl)-εK—CO—(CH₂)₁₆—CO₂H and X₁₉ is Q.
   36. The polypeptide of embodiment 31 wherein the linker-fatty acid conjugated to the γE at X₁ is (2-[2-(2-amino-ethoxy)-ethoxy]-acetyl)-Dap-CO—(CH₂)₁₈—CO₂H and X₁₉ is E.
   37. The polypeptide of embodiment 1, or a pharmaceutically acceptable salt thereof, comprising:

(SEQ ID NO: 72)
IVTS αMeL D D-Val PX₁₅X₁₆ αMeL L
QIKLEQERAEKARQQAKENTEILAQV,

or a pharmaceutically acceptable salt thereof, wherein
X₁₅ is T or I,
X₁₆ is G or Aib, and
wherein the N-term amino acid is I at position X₇, and wherein the I at X₇ is N-acetylated, and wherein the C-term amino acid is V at position X₄₄, and wherein the V at X₄₄ is amidated.
   38. The polypeptide of embodiment 37 further comprising a linker-fatty acid conjugated to the epsilon-amino group of the side chain at the K at X₃₅, wherein the K conjugated to the linker-fatty acid is selected from the group consisting of K((2-[2-(2-amino-ethoxy)-ethoxy]-acetyl)₂-γE-CO—(CH₂)₁₈—CO₂H), K(γE-(2-[2-(2-amino-ethoxy)-ethoxy]-acetyl)-γE-CO—(CH₂)₁₈—CO₂H), K(γE₂-CO—(CH₂)₁₈—CO₂H), K((2-[2-(2-amino-ethoxy)-ethoxy]-acetyl)-εK-γE-CO—(CH₂)₁₈—CO₂H), K((2-[2-(2-amino-ethoxy)-ethoxy]-acetyl)-γE-εK—CO—(CH₂)₁₈—CO₂H), K(γE₂-(2-[2-(2-amino-ethoxy)-ethoxy]-acetyl)-CO—(CH₂)₁₈—CO₂H), K(K-γE-(2-[2-(2-amino-ethoxy)-ethoxy]-acetyl)-CO—(CH₂)₁₈—CO₂H), K(K-γE-γE-CO—(CH₂)₁₈—CO₂H), K(K-(2-[2-(2-amino-ethoxy)-ethoxy]-acetyl)-γE-CO—(CH₂)₁₈—CO₂H), K(γE-(2-[2-(2-amino-ethoxy)-ethoxy]-acetyl)-εK—CO—(CH₂)₁₈—CO₂H), K(γE-εK-(2-[2-(2-amino-ethoxy)-ethoxy]-acetyl)-CO—(CH₂)₁₈—CO₂H), K(γE-γE-εK—CO—(CH₂)₁₈—CO₂H), K(γE-εK-γE-CO—(CH₂)₁₈—CO₂H), K(GKEKEKE-CO—(CH₂)₁₈—CO₂H), K(γE₂-CO—(CH₂)₁₈—CO₂H), and K((2-[2-(2-amino-ethoxy)-ethoxy]-acetyl)-E₂-CO—(CH₂)₁₈—CO₂H).
   39. The polypeptide of embodiment 38 wherein X₁₅ is T, X₁₆ is G, and the K at X₃₅ is conjugated to the fatty acid according to the formula K((2-[2-(2-amino-ethoxy)-ethoxy]-acetyl)₂-γE-CO—(CH₂)₁₈—CO₂H.
   40. The polypeptide of embodiment 38 wherein X₁₅ is T, X₁₆ is G, and the K at X₃₅ is conjugated to the fatty acid according to the formula K(γE-(2-[2-(2-amino-ethoxy)-ethoxy]-acetyl)-γE-CO—(CH₂)₁₈—CO₂H).
   41. The polypeptide of embodiment 38 wherein X₁₅ is T, X₁₆ is G, and the K at X₃₅ is conjugated to the fatty acid according to the formula K(γE2-(2-[2-(2-amino-ethoxy)-ethoxy]-acetyl)-CO—(CH₂)₁₈—CO₂H).
   42. The polypeptide of embodiment 38 wherein X₁₅ is T, X₁₆ is G, and the K at X₃₅ is conjugated to the fatty acid according to the formula K((2-[2-(2-amino-ethoxy)-ethoxy]-acetyl)-εK-γE-CO—(CH₂)₁₈—CO₂H).
   43. The polypeptide of embodiment 38 wherein X₁₅ is T, X₁₆ is G, and the K at X₃₅ is conjugated to the fatty acid according to the formula K((2-[2-(2-amino-ethoxy)-ethoxy]-acetyl)-γE-εK—CO—(CH₂)₁₈—CO₂H).
   44. The polypeptide of embodiment 38 wherein X₁₅ is T, X₁₆ is G, and the K at X₃₅ is conjugated to the fatty acid according to the formula K(K-(2-[2-(2-amino-ethoxy)-ethoxy]-acetyl)-γE-CO—(CH₂)₁₈—CO₂H).
   45. The polypeptide of embodiment 38 wherein X₁₅ is T, X₁₆ is G, and the K at X₃₅ is conjugated to the fatty acid according to the formula K(K-γE-(2-[2-(2-amino-ethoxy)-ethoxy]-acetyl)-CO—(CH₂)₁₈—CO₂H).
   46. The polypeptide of embodiment 38 wherein X₁₅ is T, X₁₆ is G, and the K at X₃₅ is conjugated to the fatty acid according to the formula K(γE-(2-[2-(2-amino-ethoxy)-ethoxy]-acetyl)-εK—CO—(CH₂)₁₈—CO₂H).
   47. The polypeptide of embodiment 38 wherein X₁₅ is T, X₁₆ is G, and the K at X₃₅ is conjugated to the fatty acid according to the formula K(γE-εK-(2-[2-(2-amino-ethoxy)-ethoxy]-acetyl)-CO—(CH₂)₁₈—CO₂H.
   48. The polypeptide of embodiment 38 wherein X₁₅ is T, X₁₆ is G, and the K at X₃₅ is conjugated to the fatty acid according to the formula K(γE-γE-εK—CO—(CH₂)₁₈—CO₂H).
   49. The polypeptide of embodiment 38 wherein X₁₅ is T, X₁₆ is G, and the K at X₃₅ is conjugated to the fatty acid according to the formula K(γE-εK-γE-CO—(CH₂)₁₈—CO₂H).
   50. The polypeptide of embodiment 38 wherein X₁₅ is T, X₁₆ is G, and the K at X₃₅ is conjugated to the fatty acid according to the formula K(K-γE-γE-CO—(CH₂)₁₈—CO₂H).
   51. The polypeptide of embodiment 38 wherein X₁₅ is T, X₁₆ is Aib, and the K at X₃₅ is conjugated to the fatty acid according to the formula K((2-[2-(2-amino-ethoxy)-ethoxy]-acetyl)₂-γE-CO—(CH₂)₁₈—CO₂H).
   52. The polypeptide of embodiment 38 wherein X₁₅ is I, X₁₆ is Aib, and the K at X₃₅ is conjugated to the fatty acid according to the formula K((2-[2-(2-amino-ethoxy)-ethoxy]-acetyl)₂-γE-CO—(CH₂)₁₈—CO₂H).
   53. The polypeptide of embodiment 38 wherein X₁₅ is T, X₁₆ is G, and the K at X₃₅ is conjugated to the fatty acid according to the formula K(GKEKEKE-CO—(CH₂)₁₈—CO₂H).
   54. The polypeptide of embodiment 38 wherein X₁₅ is T, X₁₆ is G, and the K at X₃₅ is conjugated to the fatty acid according to the formula K((2-[2-(2-amino-ethoxy)-ethoxy]-acetyl)-γE₂-CO—(CH₂)₁₈—CO₂H).
   55. The polypeptide of embodiment 38 wherein X₁₅ is I, X₁₆ is Aib, and the K at X₃₅ is conjugated to the fatty acid according to the formula K(γE₂-CO—(CH₂)₁₈—CO₂H).
   56. The polypeptide of embodiment 1, or a pharmaceutically acceptable salt thereof, comprising:

(SEQ ID NO: 73)
PPPIVTS αMeL DX₁₃PTAib αMeL LQKX₂₁
LEQERAEKARQQAKEX₃₇X₃₈EILAQV,

wherein,
X₁₃ is V or D-Val,
X₂₁ is L or Aib,
X₃₇ is A or N,
X₃₈ is A or T,
wherein the N-term amino acid is the P at X₄ and wherein the P at X₄ is N-acetylated, and wherein the C-term amino acid is V at position X₄₄, and wherein the V at X₄₄ is amidated.
   57. The polypeptide of embodiment 1, or a pharmaceutically acceptable salt thereof, wherein,
X₁ is γE,
X₂ is absent,
X₃ is absent,
X₄ is G,
X₅ is P,
X₆ is S,
X₁₁ is αMeL,
X₁₃ is D-Val,
X₁₅ is I,
X₁₆ is Aib,
X₁₉ is Q,
X₂₀ is I,
X₂₁ is K,
X₂₂ is L,
X₂₆ is R,
X₂₇ is A,
X₃₀ is A,
X₃₁ is R,
X₃₅ is K,
X₃₇ is N,
X₃₈ is T,
and wherein the C-term amino acid is V at position X₄₄, and wherein the V at X₄₄ is amidated.
   58. The polypeptide, or a pharmaceutically acceptable salt thereof, of embodiment 56 further comprising a linker-fatty acid conjugated to the epsilon-amino group of the side chain at the K at X₃₅, wherein K conjugated to the linker-fatty acid is selected from the group consisting of K((2-[2-(2-amino-ethoxy)-ethoxy]-acetyl)₂-γE-CO—(CH₂)₁₈—CO₂H) and K(γE-(2-[2-(2-amino-ethoxy)-ethoxy]-acetyl)-εK—CO—(CH₂)₁₈—CO₂H).
   59. The polypeptide of embodiment 58 wherein X₁₃ is D-Val, X₂₁ is Aib, the K at X₃₅ is conjugated to the fatty acid according to the formula K(γE-(2-[2-(2-amino-ethoxy)-ethoxy]-acetyl)-εK—CO—(CH₂)₁₈—CO₂H), X₃₇ is N, and X₃₈ is T.
   60. The polypeptide of embodiment 58 wherein X₁₃ is D-Val, X₂₁ is L, the K at X₃₅ is conjugated to the fatty acid according to the formula K(γE-(2-[2-(2-amino-ethoxy)-ethoxy]-acetyl)-εK—CO—(CH₂)₁₈—CO₂H), X₃₇ is A, and X₃₈ is A.
   61. The polypeptide of embodiment 58 wherein X₁₃ is V, X₂₁ is L, the K at X₃₅ is conjugated to the fatty acid according to the formula K(γE-(2-[2-(2-amino-ethoxy)-ethoxy]-acetyl)-εK—CO—(CH₂)₁₈—CO₂H), X₃₇ is A, and X₃₈ is A.
   62. The polypeptide of embodiment 1, or a pharmaceutically acceptable salt thereof, wherein
X₁ is absent,
X₂ is absent,
X₃ is absent,
X₄ is absent,
X₅ is absent,
X₆ is absent,
X₁₁ is L,
X₁₃ is V,
X₁₅ is I,
X₁₆ is G,
X₁₉ is Q,
X₂₀ is K,
X₂₁ is L,
X₂₂ is L,
X₂₆ is K,
X₂₇ is Q,
X₃₀ is E,
X₃₁ K,
X₃₅ is K,
X₃₇ is N,
X₃₈ is T,
wherein the N-term amino acid is the I at X₇, and wherein the I at X₇ is N-methylated,
and wherein the C-term amino acid is V at position X₄₄, and wherein the V at X₄₄ is amidated.
   63. The polypeptide of embodiment 62, or a pharmaceutically acceptable salt thereof, further comprising a linker-fatty acid conjugated to the epsilon-amino group of the side chain at the K at X₃₅, wherein the K conjugated to the linker-fatty acid is of the formula K((2-[2-(2-amino-ethoxy)-ethoxy]-acetyl)₂-γE-C20-OH).
   64. The polypeptide of embodiment 1, or a pharmaceutically acceptable salt thereof, wherein
X₁ is absent,
X₂ is absent,
X₃ is absent,
X₄ is absent,
X₅ is absent,
X₆ is absent,
X₁₁ is L,
X₁₃ is V,
X₁₅ is I,
X₁₆ is G,
X₁₉ is Q,
X₂₀ is K,
X₂₁ is L,
X₂₂ is L,
X₂₆ is R,
X₂₇ is A,
X₃₀ is A,
X₃₁ is R,
X₃₅ is K,
X₃₇ is A,
X₃₈ is A,
wherein the N-term amino acid is the I at X₇, and wherein the I at X₇ is N-methylated,
and wherein the C-term amino acid is V at position X₄₄, and wherein the V at X₄₄ is amidated.
   65. The polypeptide of embodiment 64, or a pharmaceutically acceptable salt thereof, further comprising a linker-fatty acid conjugated to epsilon-amino group of the side chain at the K at X₃₅, wherein the K conjugated to the linker-fatty acid is of the formula K((2-[2-(2-Amino-ethoxy)-ethoxy]-acetyl)₂-γE-CO—(CH₂)₁₈—CO₂H).
   66. A polypeptide comprising any one of SEQ ID NOs: 1-52, or a pharmaceutically acceptable salt thereof.
   67. The polypeptide of any one of embodiments 1-66, or a pharmaceutically acceptable salt thereof, wherein the pharmaceutically acceptable salt is selected from sodium, potassium, trifluoroacetate, hydrochloride, or acetate.
   68. A pharmaceutical composition comprising the polypeptide or a pharmaceutically acceptable salt thereof of any one of embodiments 1-66 and at least one pharmaceutically acceptable carrier, diluent, or excipient.
   69. The pharmaceutical composition of embodiment 68, wherein the composition is formulated for oral administration.
   70. The pharmaceutical composition of embodiment 68, wherein the composition is formulated for subcutaneous administration.
   71. A method of treating a disease or condition selected from the group consisting of diabetes mellitus, obesity, chronic weight management, non-alcoholic fatty liver disease (NAFLD), non-alcoholic steatohepatitis (NASH), dyslipidemia, metabolic syndrome, chronic kidney disease (CKD), osteoarthritis (OA), obesity-related sleep apnea (OSA), sarcopenia, cachexia, sarcopenic obesity (SO), and polycystic ovarian syndrome (PCOS), the method comprising administering to an individual in need thereof an effective amount of a polypeptide, or a pharmaceutically acceptable salt thereof, of any one of embodiments 1 to 66.
   72. The polypeptide, or a pharmaceutically acceptable salt thereof, as claimed by any one of embodiments 1 to 66 for use in therapy.
   73. The polypeptide, or a pharmaceutically acceptable salt thereof, as claimed by any one of embodiments 1 to 66 for use in treating a disease or condition selected from the group consisting of diabetes mellitus, obesity, chronic weight management, non-alcoholic fatty liver disease (NAFLD), non-alcoholic steatohepatitis (NASH), dyslipidemia, metabolic syndrome, chronic kidney disease (CKD), osteoarthritis (OA), obesity-related sleep apnea (OSA), sarcopenia, cachexia, sarcopenic obesity (SO), and polycystic ovarian syndrome (PCOS).
   74. Use of a polypeptide, or a pharmaceutically acceptable salt thereof, as claimed by any one of embodiments 1-66 in the manufacture of a medicament for treatment of a disease or condition selected from the group consisting of diabetes mellitus, obesity, chronic weight management, non-alcoholic fatty liver disease (NAFLD), non-alcoholic steatohepatitis (NASH), dyslipidemia, metabolic syndrome, chronic kidney disease (CKD), osteoarthritis (OA), obesity-related sleep apnea (OSA), sarcopenia, cachexia, sarcopenic obesity (SO), and polycystic ovarian syndrome (PCOS).
   75. A method of treating a disease or condition in a patient in need thereof, selected from the group consisting of: diabetes mellitus, obesity, chronic weight management, non-alcoholic fatty liver disease (NAFLD), non-alcoholic steatohepatitis (NASH), dyslipidemia, metabolic syndrome, chronic kidney disease (CKD), osteoarthritis (OA), obesity-related sleep apnea (OSA), sarcopenia, cachexia, sarcopenic obesity (SO), and polycystic ovarian syndrome (PCOS), comprising administering to the patient an effective amount of the polypeptide in any one of embodiments 1-66 and an effective amount of an additional therapeutic agent.
   76. The method of treatment according to embodiment 75 wherein the additional therapeutic agent is a dual agonist of the GIP and GLP-1 receptors.
   77. The method of embodiment 76 wherein the dual agonist of the GIP and GLP-1 receptors is a compound comprising SEQ ID NO:55.
   78. The method of treatment according to embodiment 75 wherein the additional therapeutic agonist is a tri-agonist of the GIP, GLP-1, and glucagon receptors.
   79. The method of embodiment 78 wherein the tri-agonist of the GIP, GLP-1, and glucagon receptors is a compound comprising SEQ ID NO:56, SEQ ID NO:57, or SEQ ID NO:58, or a pharmaceutically acceptable salt thereof.
   80. The method of treatment according to embodiment 75 wherein the additional therapeutic agent is a dual agonist of the glucagon and GLP-1 receptors.
   81. The method of embodiment 80 wherein the dual agonist of the glucagon and GLP-1 receptors is a compound comprising SEQ ID NO:59, SEQ ID NO:60, SEQ ID NO:61, or SEQ ID NO:62, or a pharmaceutically acceptable salt thereof.
   82. The method of treatment according to embodiment 75 wherein the additional therapeutic agent is an agonist selective for the amylin receptor.
   83. The method of embodiment 82 wherein the agonist selective for the amylin receptor is a compound comprising SEQ ID NO:63. SEQ ID NO:64, SEQ ID NO:65, or SEQ ID NO:66.
   84. The method of treatment according to embodiment 75 wherein the additional therapeutic agent is a dual agonist of both the calcitonin and amylin receptors.
   85. The method of embodiment 84 wherein the dual agonist of both the calcitonin and amylin receptors is a compound comprising SEQ ID NO:67.
   86. The method of treatment according to embodiment 75 wherein the additional therapeutic agent is a GLP-1 receptor agonist.
   87. The method of embodiment 86 wherein the GLP-1 receptor agonists is a compound comprising SEQ ID NO:79, SEQ ID NO:80, or SEQ ID NO:81.

### EXAMPLES

### Example 1: Preparation and Purification of Polypeptides Comprising SEQ ID NO:20 and SEQ ID NO:1

The polypeptides comprising SEQ ID NO:20 and SEQ ID NO:1, were made according to the following steps. First, the polypeptide comprising SEQ ID NO:20 was synthesized using Fluorenylmethyloxycarbonyl (Fmoc)/tert-Butyl (t-Bu) chemistry on a Symphony 12-channel multiplex peptide synthesizer (Protein Technologies, Inc. Tucson, AZ). During solid-phase synthesis, glass reaction vessels were wrapped with heat tape (BriskHeat controller: model SDC-120JC-A; tape: model BS0051080); the heating tape temperature was set to 60° C. for all reactions except removal of the Mtt protecting group, which proceeded at ambient temperature.

Polystyrene Rink Amide MBHA resin LL resin (Novabiochem, sub: 0.39 meq/g, 100-200 mesh, Cat #855045) was used for the synthesis at 0.13 mmol scale. Standard sidechain protecting groups were used unless otherwise noted. Fmoc-Lys(Mtt)-OH was used for the lysine to be acylated. Fmoc groups were removed prior to each coupling step (2×5 minutes) using 20% piperidine in DMF. All amino acid couplings were performed for 30 minutes using an equimolar ratio of Fmoc amino acid (0.3M), diisopropylcarbodiimide (0.9M) and Oxyma (0.9M), at a 7.7-fold molar excess over the theoretical peptide loading. The amino acid couplings of and following αMeL were performed for 6 h. After the synthesis of the backbone, the N-terminal amino acid was deprotected and then mixed with DCM for 30 minutes and drained. Then, N-terminal acetylation was performed: a solution of 100 μL acetic anhydride, 150 μL DIPEA, and 3 mL DMF was added and mixed for 30 minutes. This step was repeated once. Below is a schematic of the polypeptide comprising SEQ ID NO:20, before the addition of the linker-fatty acid moiety, using the standard single letter amino acid codes with the exception of the N-terminal acetyl group, non-coded residues (αMeL and Aib) and C-terminal valine amide, where the structures of these modifications and amino acid residues have been expanded:

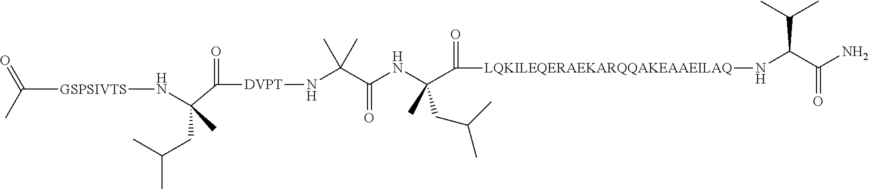

Following N-terminal acetylation, the Mtt protecting group on the lysine to be acylated was selectively removed from the resin-bound peptide using six treatments of 30% hexafluoroisopropanol (HFIP; Oakwood Chemical) in DCM (6×15-minute treatment). A DMF wash was performed to wash out residual HFIP before the next step.

For all acylated polypeptides described herein, subsequent attachment of the linker-fatty acid moiety is accomplished by performing the Fmoc/tBu chemistry steps described above to achieve the desired order and combination of amino acid monomers with standard protecting groups as well as the following non-standard monomers: N^{α}-Boc-N^{ε}-Fmoc-L-Lysine (Boc-L-Lys(Fmoc)-OH, Chem-Impex Int'l Inc), N^{α}-Fmoc-N^{β}-Boc-L-2,3-diaminopropionic acid (Fmoc-Dap(Boc)-OH, Chem-Impex Int'l Inc), Fmoc-β-alanine (Fmoc-β-Ala-OH, Orpegen), Fmoc-6-aminohexanoic acid (Fmoc-Ahx-OH, described as Fmoc-ε-Acp-OH by vendor Chem-Impex Int'l Inc), Fmoc-L-glutamic acid α-t-butyl ester (Fmoc-L-Glu-OtBu, Ark Pharm, Inc.), 1-(9H-fluoren-9-yl)-3-oxo-2,7,10-trioxa-4-azadodecan-12-oic acid (Fmoc-AEEA-OH, ChemShuttle), mono-OtBu-octadecanedioic acid (WuXi AppTec, Shanghai, China), and mono-OtBu-eicosanedioic acid (WuXi AppTec, Shanghai, China). For SEQ ID NO: 1, the order was Fmoc-Glu-OtBu, Fmoc-AEEA-OH, Boc-L-Lys(Fmoc)-OH, mono-OtBu-eicosanedioic acid. 3-Fold excess of reagents (equimolar AA: DIC: Oxyma) was used for each 6-hour long coupling.

After the synthesis was complete, the peptide resin was washed with DCM and then thoroughly air-dried. The dry resin was treated with 10 mL of cleavage cocktail (trifluoroacetic acid/TFA: 1,2-ethanedithiol:triisopropylsilane:water=89:3:3:5 v/v) for 2 hours at room temperature. The resin was collected by filtration and the filtrate was treated with 4-fold cold diethyl ether (−20° C.) to precipitate the crude peptide. The peptide/ether suspension was then centrifuged at 3200 RCF for 1.5 min to form a solid pellet, the supernatant was decanted, and the solid pellet was triturated with ether two additional times. The pellet after air drying was dissolved in acetic acid (5 mL) and water (10 mL).

The crude peptide was purified by RP-HPLC on a Phenomenex PhenylHexyl column (5 um, 100 A, 250×21.2 mm, Part Number:OOG-4257-PO-AX) with a linear gradient using a 100% acetonitrile and 0.05% TFA/water buffer system. The purity of the peptide was assessed using analytical RP-HPLC using a Waters Acuity CSH C18 column (1.7 um, 2.1×100 mm, Part 186005572) and pooling criteria was >90%. The main pool purity of SEQ ID NO:20 was found to be >92.0%. Subsequent lyophilization of the final main product pool yields the lyophilized peptide TFA salt. The molecular weight of SEQ ID NO:20 was determined by LC/MS (Found: [M+3H]³⁺=1540.1; Calc. [M+3H]³⁺=1539.1; Found MW (avg)=4617.3; Calc. MW (avg)=4614.3). The molecular weight of SEQ ID NO:1 was determined by LC/MS (Found: [M+3H]³⁺=1781.1; Calc. [M+3H]³⁺=1781.4; Found MW (avg)=5340.3; Calc. MW (avg)=5341.3). Below is a schematic of the polypeptide comprising SEQ ID NO:1 using the standard single letter amino acid codes with the exception of the N-terminal acetyl group, non-coded residues (αMeL and Aib), the acylated lysine at position 35, and C-terminal valine amide, where the structures of these modifications and amino acid residues have been expanded:

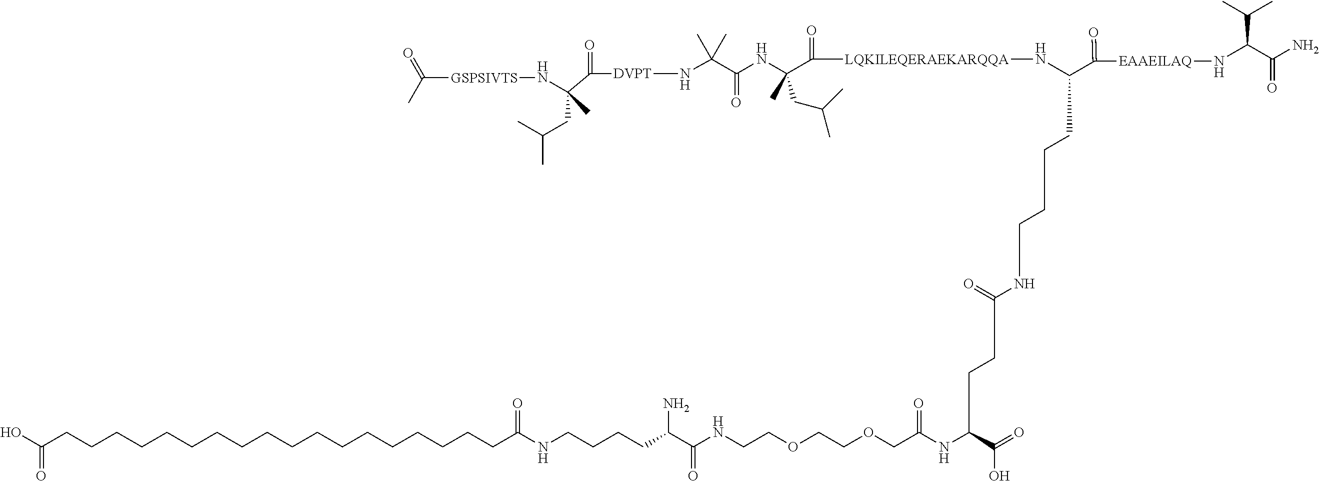

Similar processes to those described above and known to those of skill in the art may be used to synthesize the peptide backbone, conjugate the linker-fatty acid moiety, examine the purity, and confirm the molecular weight of the inventive polypeptides described herein. The compounds according to SEQ ID NO: 2-19, 21, 29-44, 46-48 and 51 were prepared substantially as described by the procedures of Example 1.

### Example 2: Preparation and Purification of Polypeptides Comprising SEQ ID NO:23 and SEQ ID NO:22

The solid-phase synthesis was performed analogously to Example 1, except instead of deprotecting the N-terminal amino acid and treating with acetic anhydride to acetylate the N-terminus, the monomer Boc-L-pyroglutamic acid (Boc-L-Pyr-OH, Combi-Blocks) was coupled with standard conditions as described in Example 1.

Sequence ID NO:45 was prepared analogously to the N-terminal Pyr, but with Boc-Glu-OtBu (Synthonix). Sequence ID NO: 49 and 50 were prepared analogously to the N-terminal Pyr, but with Boc-NMeI (Combi-Blocks).

Below is a schematic of SEQ ID NO:23 using the standard single letter amino acid codes with the exception of N-terminal L-Pyr-OH, non-coded residues (αMeL and Aib), and C-terminal valine amide, where the structures of these modifications and amino acid residues have been expanded:

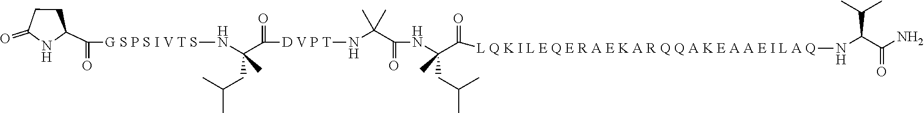

The molecular weight of SEQ ID NO:23 was determined by LC/MS (Found: [M+3H]³⁺=1582.1; Calc. [M+3H]³⁺=1581.2; Found MW (avg)=4743.3; Calc. MW (avg)=4740.5).

Below is a schematic of SEQ ID NO:22 using the standard single letter amino acid codes with the exception of N-terminal L-Pyr-OH, non-coded residues (αMeL and Aib), acylated lysine at position 35, and C-terminal valine amide, where the structures of these modifications and amino acid residues have been expanded:

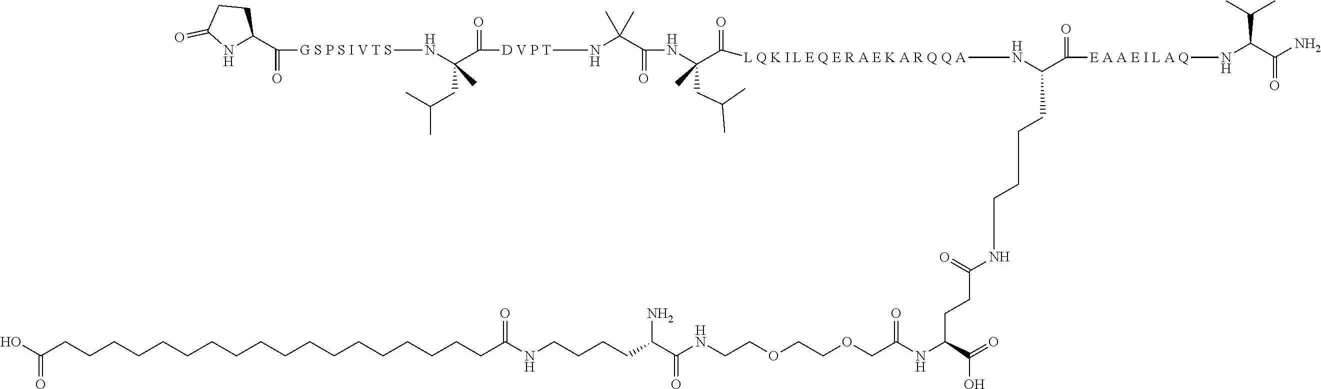

The molecular weight is determined by LC/MS (Found: [M+3H]³⁺=1823.4; Calc. [M+3H]³⁺=1823.5; Found MW (avg)=5467.2; Calc. MW (avg)=5467.4). The compound according to SEQ ID NO: 24 was prepared substantially as described by the procedures of Example 2.

### Example 3: Preparation and Purification of Polypeptides Comprising SEQ ID NO:27 and SEQ ID NO:25

For this and other polypeptides that were eventually N-acylated with a linker-fatty acid group, N-terminal acetylation was not appropriate; the synthesis did not include the acetic anhydride step described in Example 1. For SEQ ID NO:27, the synthesis concluded with the removal of the Fmoc protecting group on the N-terminal glycine residue.

Below is a schematic of SEQ ID NO:27 using the standard single letter amino acid codes with the exception of N-terminal glycine, non-coded residues (αMeL and Aib), and C-terminal valine amide, where the structures of these modifications and amino acid residues have been expanded:

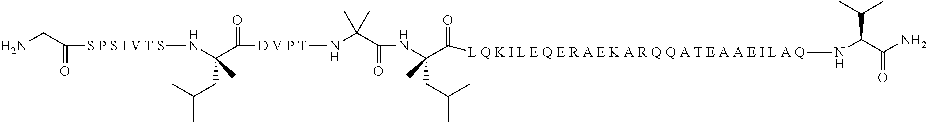

The molecular weight of SEQ ID NO:27 was determined by LC/MS (Found: [M+3H]³⁺=1516.9; Calc. [M+3H]³⁺=1516.1; Found MW (avg)=4547.7; Calc. MW (avg)=4545.2).
For SEQ ID NO:25 and other polypeptides that were N-acylated with a linker-fatty acid group (SEQ ID NO:26, 28, 52), Fmoc-Lys(Mtt)-OH was not used in the sequence; the synthesis was continued without the 30% HFIP deprotection step described in Example 1. For SEQ ID NO:25, after the final Fmoc deprotection step of the SEQ ID NO:27, synthesis, Fmoc/tBu chemistry was used as described in Example 1 to finish the synthesis, with coupling/Fmoc deprotection steps performed for monomers added in the following order: Fmoc-Glu-OtBu, Fmoc-AEEA-OH, Boc-L-Lys(Fmoc)-OH, mono-OtBu-eicosanedioic acid.

Below is a schematic of SEQ ID NO:25, using the standard single letter amino acid codes with the exception of N-terminal acylated glycine, non-coded residues (αMeL and Aib), and C-terminal valine amide, where the structures of these modifications and amino acid residues have been expanded:

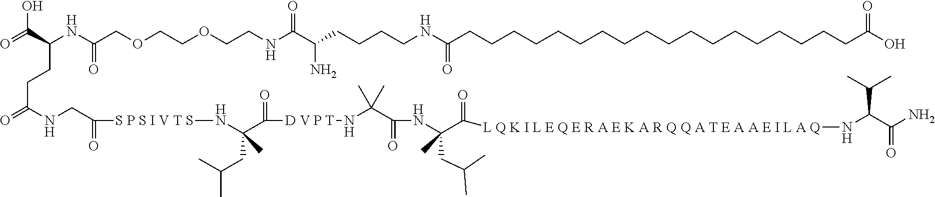

The molecular weight was determined by LC/MS (Found: [M+3H]³⁺=1758.1; Calc. [M+3H]³⁺=1758.4; Found MW (avg)=5271.3; Calc. MW (avg)=5272.2). The compounds according to SEQ ID NO: 26, 28, 52 were prepared substantially as described by the procedures of Example 3.

TABLE 1
Found and calculated [M + 3H]³⁺ m/z ions and MW(avg)
for polypeptides described herein.

| | | | Found | |
|---|---|---|---|---|
| SEQ | | | MW | Calc. MW |
| ID | Found | Calc | (avg, | (avg, |
| NO | [M + 3H]³⁺ | [M + 3H]³⁺ | g/mol) | g/mol) |
| 1 | 1781.0 | 1781.4 | 5340.0 | 5341.3 |
| 2 | 1781.0 | 1781.4 | 5340.0 | 5341.2 |
| 3 | 1856.5 | 1856.8 | 5566.5 | 5567.5 |
| 4 | 1776.2 | 1776.4 | 5325.6 | 5326.3 |
| 5 | 1776.2 | 1776.4 | 5325.6 | 5326.3 |
| 6 | 1966.2 | 1966.6 | 5895.6 | 5896.9 |
| 7 | 1780.5 | 1780.4 | 5338.5 | 5338.2 |
| 8 | 1804.0 | 1804.1 | 5409.0 | 5409.3 |
| 9 | 1995.8 | 1996.0 | 5984.4 | 5985.0 |
| 10 | 1795.5 | 1795.8 | 5383.5 | 5384.3 |
| 11 | 1845.9 | 1846.2 | 5534.7 | 5535.5 |
| 12 | 1773.2 | 1772.1 | 5316.6 | 5313.2 |
| 13 | 1850.9 | 1851.2 | 5549.7 | 5550.5 |
| 14 | 1830.0 | 1830.2 | 5487.0 | 5487.5 |
| 15 | 1884.3 | 1884.6 | 5649.9 | 5650.7 |
| 16 | 1916.3 | 1916.6 | 5745.9 | 5746.7 |
| 17 | 1830.6 | 1829.8 | 5488.8 | 5486.4 |
| 18 | 1819.7 | 1818.8 | 5456.1 | 5453.4 |
| 19 | 1825.1 | 1824.2 | 5472.3 | 5469.5 |
| 20 | 1540.1 | 1539.1 | 4617.3 | 4614.3 |
| 21 | 1773.1 | 1772.4 | 5316.3 | 5313.2 |
| 22 | 1823.4 | 1823.5 | 5467.2 | 5467.4 |
| 23 | 1582.1 | 1581.2 | 4743.3 | 4740.5 |
| 24 | 1815.3 | 1814.4 | 5442.9 | 5439.4 |
| 25 | 1758.1 | 1758.4 | 5271.3 | 5272.2 |
| 26 | 1758.0 | 1758.4 | 5271.0 | 5272.2 |
| 27 | 1516.9 | 1516.1 | 4547.7 | 4545.2 |
| 28 | 1750.2 | 1749.0 | 5247.6 | 5244.1 |
| 29 | 1692.8 | 1692.6 | 5075.4 | 5074.9 |
| 30 | 1686.8 | 1687.3 | 5057.4 | 5058.8 |
| 31 | 1686.9 | 1687.3 | 5057.7 | 5058.8 |
| 32 | 1686.7 | 1687.0 | 5057.1 | 5057.9 |
| 33 | 1686.6 | 1687.0 | 5056.8 | 5057.9 |
| 34 | 1686.4 | 1687.0 | 5056.2 | 5057.9 |
| 35 | 1686.6 | 1687.0 | 5056.8 | 5057.9 |
| 36 | 1686.4 | 1687.0 | 5056.2 | 5057.9 |
| 37 | 1686.6 | 1687.0 | 5056.8 | 5057.9 |
| 38 | 1681.2 | 1681.6 | 5040.6 | 5041.8 |
| 39 | 1681.3 | 1681.6 | 5040.9 | 5041.8 |
| 40 | 1681.3 | 1681.6 | 5040.9 | 5041.8 |
| 41 | 1702.9 | 1702.0 | 5105.7 | 5102.9 |
| 42 | 1707.1 | 1706.0 | 5118.3 | 5115.0 |
| 43 | 1871.8 | 1872.8 | 5612.4 | 5613.6 |
| 44 | 1686.8 | 1687.3 | 5057.4 | 5058.8 |
| 45 | 1815.9 | 1815.4 | 5444.7 | 5443.3 |
| 46 | 1785.2 | 1784.1 | 5352.6 | 5349.2 |
| 47 | 1770.1 | 1769.1 | 5307.3 | 5304.2 |
| 48 | 1770.2 | 1769.1 | 5307.6 | 5304.2 |
| 49 | 1701.6 | 1702.3 | 5101.8 | 5103.9 |
| 50 | 1657.4 | 1658.3 | 4969.2 | 4971.8 |
| 51 | 1653.2 | 1652.3 | 4956.6 | 4953.8 |
| 52 | 1744.4 | 1744.7 | 5230.2 | 5231.1 |

### Example 4. In Vitro CRHR1 and CRHR2 Activity Assessment Via Intracellular cAMP Production

The corticotropin-releasing hormone (CRH) receptor 1 and 2 (CRHR1 and CRHR2) are GPCRs that were functionally coupled to Gₛ proteins. Stimulation of these receptors results in an increased production of intracellular cAMP, which can be detected using standard in vitro technologies. In vitro activity of peptides was measured by the amount of cAMP formed in human CRHR1 and CRHR2 overexpressed cells.

### Human CRHR1 and CRHR2 Expressing Cell Lines

Human CRHR1 and CRHR2 receptors were stably expressed in human embryonic kidney cells (HEK293) under control of a pcDNA expression vector. The HEK293 cell line was cultured in DMEM 1× (Gibco, 93-0152DK) supplemented with 10% FBS, 1 mM sodium pyruvate, 1×MEM NEAA, 1× GlutaMAX and 10 mM HEPES. The plasmids of human CRHR1-pQCXIP (T7364) and human CRHR2b-R2683puro (T16761) DNA were transfected into HEK293 cells using Lipo fectAMINE 3000 Transfection reagent (Invitrogen, L3000001). After 20 days under selection, the mRNA levels from different clones were measured to confirm the expression of human CRHR1 and CRHR2 genes. To determine the function of over-expressed CRHR cells, the intracellular cAMP levels in response to human UCN1 and UCN2 were measured and compared to the expression of CRHR1 and CRHR2 mRNA in each clone.

Human CRHR1 cells were cultured in DMEM 1× (Gibco, 93-0152DK) supplemented with 10% FBS, 1 mM sodium pyruvate, 1×MEM NEAA, 1×GlutaMAX, 10 mM HEPES, and 800 μg/ml G418. Human CRHR2 cells were cultured at the same media (DMEM 1×, 10% FBS, 1 mM sodium pyruvate, 1×MEM NEAA, 1×GlutaMAX, 10 mM HEPES) in the presence of 0.5 ug/mL puromycin. Cultured cells were grown to 90% confluency and harvested. The cells were frozen at 10×10e6/ml in a mixture of 100% fetal bovine serum plus 7% DMSO and stored in liquid nitrogen until use.

Native Cell Lines that Express Endogenous Human CRHR1 and CRHR2

Y79 cells, a human retinoblastoma cell line, express a high level of human CRHR1 with no detectable CRHR2. A7r5 cells, a rat aortic smooth muscle cell line, only express CRHR2 receptor with no detectable CRHR1. The rat CRHR2 gene shares 93% homology of nucleotides sequences with human CRHR2 and responds to human UCN2. Therefore, these two native cell lines were used for additional characterization of polypeptide selectivity.

Y79 cells were grown in a suspension culture as recommended by ATCC in the media containing RPMI (Gibco Cat #11875093) and 20% FBS (Gibco dialyzed Cat #26400044). Y79 cells were incubated at 37° C. with 5% CO₂. A subcultivation was performed by removing the supernatant after centrifugation and suspending the cell pellet in fresh medium (A subcultivation ratio was 1:5). Adherent A7r5 cells were cultured in tissue culture flasks in DMEM with 10% FBS, 1 mM pyruvate, and 5 mM HEPES at 37° C. and 5% CO₂. Cultured Y79 and A7r5 cells were harvested and frozen at 10×10e6/mL in a mixture of 100% fetal bovine serum plus 7% DMSO and stored in liquid nitrogen until use.

Human CRHR1 and CRHR2 cAMP Assay

On the day of the assay, the frozen cells were thawed and diluted in assay buffer (Phenol Red free MEM (Corning, 17-305-CV, lot #32121010), 0.1% Casein (Sigma C4765, lot #SLCG2120), 5 mM HEPES pH 7.4 containing 0.25 mM IBMX). 10 ul of human CRHR1 cells (25,000 cells) or 10 μL of human CRHR2 cells (5,000 cells) were dispensed into each well of white poly-D-lysine coated 384-well plates (Corning cat #354661). Test compounds diluted in DMSO were added (200 nL/well) in a 1:3 dilution series using ECHO acoustic liquid handler (Beckman). The mixture of cells and test compounds were incubated at 37° C. for 1 hour for the hCRHR1 plate, while the CRHR2 plates were incubated at room temperature for 30 min.

The amount of intracellular cAMP was quantitated using HTRF technology (Homogeneous Time Resolved Fluorescence; Cisbio) as per vendor instructions. Briefly, 10 uL CAMP-d2 conjugate and 10 uL anti-CAMP-cryptate conjugate in lysis buffer were incubated with the treated cells at room temperature for 60 min. The HTRF signal was immediately detected using an Envision plate reader (Perkin-Elmer) to calculate the ratio of fluorescence at 665 to 620 nm.

Human Y79 and Rat A7r5 cAMP Assay

The cAMP assays for Y79 and A7r5 cells were conducted similarly to the hCRHR1 and hCRR2 assays. Briefly, on the day of the assay, the frozen cells were thawed and diluted in the assay buffer. 10 μL of human Y79 cells (5,000 cells) or 10 μL of rat A7r5 cells (5,000 cells) were dispensed into each well of white 384-well plates. Test compounds diluted in DMSO were added (200 nL/well) in a 1:3 dilution series using ECHO acoustic liquid handler (Beckman). The mixture of cells and test compounds were incubated at 37° C. for 1 hour for both the Y79 cells and A7r5 cells.

### Data Analysis

Raw fluorescence data for concentration curves of the peptides in Table 2 and Table 3 were converted to cAMP amount (pmol/well) using a cAMP standard curve generated for each experiment. Relative EC50 values were calculated from the top-bottom range of the concentration response curve defined using 10 nM human UCN2 as the maximum for the CRHR2 cells and 66 nM human UCN1 for the CRHR1 cells and buffer alone as the minimum with a four-parameter logistic curve fitting program (Genedata Screener® v12.0.4).

### Results

TABLE 2
Comparison of Functional Activity Data at human
CRHR2 and CRHR1 Receptor*

| SEQUENCE | hCRHR2 cAMP EC₅₀ | hCRHR1 cAMP EC₅₀ |
|---|---|---|
| IDS | (pM, ± SE) | (pM, ± SE) |
| SEQ ID NO: 1 | 9.3 ± 1.1 (n = 11) | >1.0e+7 (n = 10) |
| SEQ ID NO: 2 | 12.3 ± 1.8 (n = 8) | >1.0e+7 (n = 7) |
| SEQ ID NO: 3 | 18.3 ± 2.1 (n = 3) | >1.0e+7 (n = 3) |
| SEQ ID NO: 4 | 20.3 ± 2.9 (n = 3) | >1.0e+7 (n = 3) |
| SEQ ID NO: 5 | 10.7 ± 0.8 (n = 3) | >1.0e+7 (n = 3) |
| SEQ ID NO: 6 | 10.1 ± 2.5 (n = 3) | >1.0e+7 (n = 3) |
| SEQ ID NO: 7 | 10.6 ± 1.4 (n = 3) | >1.0e+7 (n = 3) |
| SEQ ID NO: 8 | 11.0 ± 0.9 (n = 3) | >1.0e+7 (n = 3) |
| SEQ ID NO: 9 | 15.5 ± 3.3 (n = 3) | >1.0e+7 (n = 3) |
| SEQ ID NO: 10 | 10.1 ± 1.1 (n = 3) | >1.0e+7 (n = 3) |
| SEQ ID NO: 11 | 8.3 ± 1.4 (n = 4) | >1.0e+7 (n = 3) |
| SEQ ID NO: 12 | 23.8 ± 1.6 (n = 3) | >1.0e+7 (n = 3) |
| SEQ ID NO: 13 | 7.9 ± 0.3 (n = 3) | >1.0e+7 (n = 3) |
| SEQ ID NO: 14 | 8.3 ± 0.5 (n = 3) | >1.0e+7 (n = 3) |
| SEQ ID NO: 15 | 11.3 ± 0.4 (n = 3) | >1.0e+7 (n = 3) |
| SEQ ID NO: 16 | 12.5 ± 1.1 (n = 3) | >1.0e+7 (n = 3) |
| SEQ ID NO: 17 | 9.5 ± 0.9 (n = 3) | >1.0e+7 (n = 3) |
| SEQ ID NO: 18 | 10.9 ± 1.3 (n = 3) | >1.0e+7 (n = 3) |
| SEQ ID NO: 19 | 11.1 ± 0.5 (n = 3) | >1.0e+7 (n = 3) |
| SEQ ID NO: 20 | 15.3 ± 1.4 (n = 4) | >1.0e+7 (n = 4) |
| SEQ ID NO: 21 | 16.3 ± 1.3 (n = 3) | >1.0e+7 (n = 3) |
| SEQ ID NO: 22 | 11.3 ± 3.0 (n = 8) | >1.0e+7 (n = 7) |
| SEQ ID NO: 23 | 28.9 ± 1.2 (n = 3) | >1.0e+7 (n = 3) |
| SEQ ID NO: 24 | 21.7 ± 3.6 (n = 3) | >1.0e+7 (n = 3) |
| SEQ ID NO: 25 | 6.9 ± 0.5 (n = 12) | >1.0e+7 (n = 11) |
| SEQ ID NO: 26 | 6.9 ± 0.7 (n = 3) | >1.0e+7 (n = 3) |
| SEQ ID NO: 27 | 23.6 ± 2.9 (n = 3) | >1.0e+7 (n = 3) |
| SEQ ID NO: 28 | 12.8 ± 2.0 (n = 3) | >1.0e+7 (n = 3) |
| SEQ ID NO: 29 | 5.6 ± 0.4 (n = 17) | >1.0e+7 (n = 11) |
| SEQ ID NO: 30 | 5.7 ± 0.1 (n = 4) | >1.0e+7 (n = 3) |
| SEQ ID NO: 31 | 4.5 ± 0.7 (n = 4) | >1.0e+7 (n = 3) |
| SEQ ID NO: 32 | 5.4 ± 0.7 (n = 5) | >1.0e+7 (n = 3) |
| SEQ ID NO: 33 | 4.6 ± 0.6 (n = 5) | 1.1e+6 ± 6.1e+4 (n = 3) |
| SEQ ID NO: 34 | 4.9 ± 0.7 (n = 5) | >1.0e+7 (n = 3) |
| SEQ ID NO: 35 | 5.3 ± 0.9 (n = 5) | 3.0e+6 ± 4.6e+5 (n = 3) |
| SEQ ID NO: 36 | 4.4 ± 0.4 (n = 6) | 1.8e+6 ± 3.2e+5 (n = 3/4) |
| SEQ ID NO: 37 | 5.0 ± 0.2 (n = 6) | 1.7e+6 ± 2.8e+5 (n = 3) |
| SEQ ID NO: 38 | 6.3 ± 2.1 (n = 4) | 1.1e+6 ± 2.9e+5 (n = 2) |
| SEQ ID NO: 39 | 7.3 ± 0.8 (n = 4) | 3.7e+6 (n = 1/2) |
| SEQ ID NO: 40 | 10.3 ± 1.9 (n = 4) | >1.0e+7 (n = 1/2) |
| SEQ ID NO: 41 | 7.0 ± 0.9 (n = 7) | 3.7e+6 ± 3.1e+5 (n = 6) |
| SEQ ID NO: 42 | 6.6 ± 0.8 (n = 10) | 2.3e+6 ± 1.9e+5, (n = 9) |
| SEQ ID NO: 43 | 7.9 ± 1.3 (n = 3) | >1.0e+7 (n = 3) |
| SEQ ID NO: 44 | 5.4 ± 0.5 (n = 4) | >1.0e+7 (n = 3) |
| SEQ ID NO: 45 | 9.0 ± 3.6 (n = 3) | 3.7e+6 ± 5e+5 (n = 3) |
| SEQ ID NO: 46 | 13.0 ± 1.1 (n = 9) | >1.0e+7 (n = 7) |
| SEQ ID NO: 47 | 17.5 ± 2.2 (n = 9) | 2.3e+6 ± 1.3e+5 (n = 3/8) |
| SEQ ID NO: 48 | 26.7 ± 3.8 (n = 10) | >1.0e+7 (n = 9) |
| SEQ ID NO: 49 | 6.6 ± 0.9 (n = 19) | >1.0e+7 (n = 7) |
| SEQ ID NO: 50 | 22.4 ± 3.6 (n = 24) | >1.0e+7 (n = 14) |
| SEQ ID NO: 51 | 8.6 ± 3.2 (n = 3) | 4.3e+6 ± 1.4e+5 (n = 3) |
| SEQ ID NO: 52 | 6.6 ± 0.5 (n = 3) | 1.5e+6 (n = 1/3) |
| SEQ ID NO: 53 | 24.5 ± 1.6 (n = 124) | >1.0e+7 (n = 65) |
| SEQ ID NO: 54 | 29.8 ± 2.5 (n = 127) | >1.0e+7 (n = 64) |

This table shows that all polypeptides of the present disclosure have specific activity at the hCRTIR2 receptor and little to no activity at the hCRH1 receptor.

TABLE 3
Comparison of Functional Activity Data in human Y79 and rat A7r5 cells

| | A7r5 cAMP EC₅₀ | Y79 cAMP EC₅₀ |
|---|---|---|
| SEQ ID | (pM, ± SE) | (pM, ± SE) |
| SEQ ID NO: 1 | 157 ± 8 (n = 5) | 1.4e+6 ± 2.8e+5 (n = 3) |
| SEQ ID NO: 2 | 544 ± 133 (n = 2) | 2.1e+6 ± 3.3e+5 (n = 2) |
| SEQ ID NO: 22 | 314 ± 24 (n = 5) | 2.4e+6 ± 3.1e+5 (n = 3) |
| SEQ ID NO: 25 | 112 ± 5 (n = 5) | 6.2e+5 ± 1.8e+5 (n = 3) |
| SEQ ID NO: 29 | 123 ± 2 (n = 4) | 1.0e+6 ± 1.1e+5 (n = 2) |
| SEQ ID NO: 46 | 313 ± 24 (n = 4) | 4.0e+6 ± 4.5e+5 (n = 2) |
| SEQ ID NO: 47 | 1061 ± 176 (n = 2) | 7.2e+5 ± 3.0e+5 (n = 2) |
| SEQ ID NO: 48 | 1156 ± 29 (n = 2) | 4.5e+6 ± 1.4e+5 (n = 2) |
| SEQ ID NO: 49 | 137 ± 14 (n = 4) | >1e+7 (n = 2) |
| SEQ ID NO: 50 | 967 ± 347 (n = 4) | >1e+7 (n = 2) |
| SEQ ID NO: 53 | 102 ± 11 (n = 5) | >1e+7 (n = 3) |
| SEQ ID NO: 54 | 471 ± 85 (n = 5) | 6.0e+6 ± 2.6e+6 (n = 3) |

Similar to the selectivity of the polypeptides seen in hCRTIR2 over hCRH1 cells, the data shown in this table indicates that all polypeptides of the present disclosure demonstrate significantly higher potency in A7r5 cells (CRHR2 receptor) than in Y79 cells (CRHIR1 receptor).

### Example 5: Association State Determination by Sedimentation Velocity Analytical UltraCentrifugation (SV-AUC)

SV-AUC was performed at 20° C. using a Beckman Optima with AN 60 Ti rotor and final rotor speed of 60,000 rpm. Peptides were dialyzed into 5 mM phosphate pH 7 or 1× phosphate buffered saline (PBS) pH 7.2. Samples were adjusted to the desired concentration (0.1 mg/mL, 1 mg/mL, or 10 mg/mL) using relevant buffer as needed. Both samples and buffer were passed through 0.22 m PVDF centrifugal filter units. The filtered samples were loaded into the sample sectors of the cells; filtered buffer alone was loaded in the reference sector of the cells. A Wavelength Scan was performed as the first step to choose a suitable lambda (k) value for the given compound with 1-1.2 OD at the concentration(s) tested; this value was generally between 225 nm and 254 nm and used for Method Scan in the next step. The λ for each compound is indicated in Table 1. In the Method Scan, both absorbance and interference detection modules were used. Temperature equilibration was set as first stage with 0 rpm rotor speed for 3 hours. Sedimentation velocity was set as second stage with rotor speed at 60,000 rpm. 300 scans with frequency of 60 seconds or 500 scan counts with frequency of 120 seconds were performed at the designated wavelength. Data were analyzed with SEDFIT software wherein a continuous c(s) distribution model was applied; the setting of relevant parameters is shown in Table 4 and Table 5; sedimentation coefficient (s) values were reported in Svedherg units (S).

TABLE 4
Summary of the concentrations, buffer, wavelength, sedimentation coefficient(s),
molecular weight, and apparent molecular weight(s) by compound.

| | | | | Sedimentation | | |
|---|---|---|---|---|---|---|
| SEQ | Concentration | | λ | Coefficient | MW | Mw |
| ID NO. | (mg/mL) | Buffer | (nm) | (% of total) | (g/mol) | (SEDFIT) |
| 54 | 0.1 | 1x PBS, pH 7.2 | 225 | 3.5 S (89%) | 5157.1 | 38787 Da |
| | 1 | | | 0.9 S (5%), | | 5608 Da, |
| | | | | 2.5 S (2.5%), | | 27636 Da, |
| | | | | 3.7 S (88.5%) | | 50313 Da |
| | 10 | | | 0.9 S (25.3%), | | 5654 Da, |
| | | | | 2.9 S (12.4%), | | 34479 Da, |
| | | | | 3.9 S (51.2%) | | 53507 Da |
| 49 | 10 | 1x PBS, pH 7.2 | 252 | 0.9 S (33.2%), | 5104 | 5103 Da, |
| | | | | 1.7 S (73.7%) | | 13921 Da |
| 50 | 10 | 1x PBS, pH 7.2 | 253 | 0.9 S (13%), | 4971.8 | 5236 Da, |
| | | | | 2.1 S (72.8%) | | 17851 Da |
| 46 | 1 | 5 mM phosphate, | 239 | 0.7 S (35.9%), | 5349.3 | 6642 Da, |
| | | pH 7 | | 1.4 S (62%) | | 19213 Da |
| 47 | 1 | 5 mM phosphate, | 237 | 0.8 S (11.8%), | 5304.3 | 7027 Da, |
| | | pH 7 | | 1.4 S (89.3%) | | 17800 Da |
| 48 | 1 | 5 mM phosphate, | 238 | 0.9 S (11%), | 5304.3 | 7278 Da, |
| | | pH 7 | | 1.5 S (87.2%) | | 16252 Da |
| 2 | 1 | 5 mM phosphate, | 237 | 0.9 S (10%), | 5341.3 | 8092 Da, |
| | | PH 7 | | 1.5 S (84.6%) | | 17142 Da |
| 1 | 1 | 5 mM phosphate, | 234 | 0.9 S (13.7%), | 5341.3 | |
| | | pH 7 | | 1.5 S (87.5%) | | |
| 1 | 1 | 5 mM phosphate, | 234 | 1.25 S (8.9%), | 5341.3 | |
| | | pH 7 + 150 mM | | 1.82 S (90.2%) | | |
| | | NaCl | | | | |
| 22 | 1 | 5 mM phosphate, | 237 | 0.8 S (13.3%), | 5467.4 | |
| | | pH 7 | | 1.5 S (86.4%) | | |
| 22 | 1 | 5 mM phosphate, | 237 | 1.33 S (8.3%), | 5467.4 | |
| | | pH 7 + 150 mM | | 1.82 S (90.6%) | | |
| | | NaCl | | | | |
| 25 | 1 | 5 mM phosphate, | 237 | 0.7 S (8.6%), | 5272.2 | |
| | | pH 7 | | 1.4 S (87.3%) | | |
| 25 | 1 | 5 mM phosphate, | 237 | 0.7 S (2.9%), | 5272.2 | |
| | | pH 7 + 150 mM | | 1.75 S (85.4%) | | |
| | | NaCl | | | | |

The experiment to characterize a compound comprising SEQ ID NO:54 was performed at 300 scan counts with frequency of 60 seconds; for all other tested polypeptides, the experiments were performed at 500 scan counts with frequency of 120 seconds.

TABLE 5
The setting of relevant parameters for SEDFIT software
using a continuous c(s) distribution model.

| Parameter | Value |
|---|---|
| (a) Parameters used for SEQ ID NO: 54 continuous | |
| sedimentation coefficient distribution c(s) | |
| resolution | 50 |
| s min | 0.0 |
| s max | 20.0 |
| frictional ratio | 1.2 |
| baseline | 0.00000 |
| Fit RI Noise | Yes |
| Fit Time Independent Noise | No |
| Meniscus | 5.8002 |
| Bottom | Not fixed |
| Confidence level (F-ratio) | 0.67000 |
| Partial spec. volume | 0.73000 |
| Buffer density | 1.00000 |
| Buffer viscosity (Poise) | 0.01002 |
| S grad from file | No |
| Log spaced s grid | No |
| (b) Parameters used for the other | |
| polypeptides in this disclosure. | |
| resolution | 50 |
| s min | 0.0 |
| s max | 5.0 |
| frictional ratio | 1.2 |
| baseline | 0.00000 |
| Fit RI Noise | Yes |
| Fit Time Independent Noise | Yes |
| Meniscus | 6.1357 |
| Bottom | Not fixed |
| Confidence level (F-ratio) | 0.95000 |
| Partial spec. volume | 0.73000 |
| Buffer density | 1.00000 |
| Buffer viscosity (Poise) | 0.01002 |
| S grad from file | No |
| Log spaced s grid | No |

Reference compound SEQ ID NO:54 had sedimentation coefficients indicating that higher order oligomers were the major species at all three concentrations tested (0.1 mg/mL, 1 mg/mL, 10 mg/mL). In contrast, the polypeptides of the present disclosure were measured as lower self-association state compared with SEQ ID NO:54.

### Example 6. In Vivo Diet Induced Obesity (DIO) Model-Chronic Dose Administration

The DIO model represents a pre-diabetic state that is obese. These animals, although not diabetic, display insulin resistance, dyslipidemia, and hepatic steatosis, all characteristics of metabolic syndrome, after being placed on a high fat (60% Kcal from fat) diet for 12 weeks (Surwit R S et al., Diet-induced type II diabetes in C₅₇BL/6J mice. Diabetes 37(9): 1163-7 (1988)). Assessment of body weight change from start of study, fat mass change from start of study, lean mass change from start of study, and fasting glucose or glucose change from vehicle in same study substantially as set forth below. In the case of SEQ ID NO:55 combined with SEQ ID NO:25, tibialis anterior muscle weight at the end of the study is also reported as a measure of effect on muscle weight.

Male C57BL6 mice 22-24 weeks old (on high fat diet since 6 weeks of age, Jackson Laboratories 3800050; Bar Harbor, ME) are housed 1 per cage and maintained on D12492 chow (60% lard high fat diet: Research diets New Brunswick NJ) for 2 weeks in the vivarium and on a normal light cycle prior to experiment start. Animals were randomized by body weight to treatment groups using block randomization. On day 1 of experiment animals and food were weighed and recorded. Animals were separated in to two equal groups and started on separate days (data combined) to simplify the logistics of the study. Animals are given a single subcutaneous injection (s.c.) of the indicated treatment in 40 mM Tris HCl pH8.0+50 mM D-mannitol+0.02% polysorbate 80 on days 1(start), 4, 7, 10, and 13 of experiment at a volume of 10 ml/kg. Vehicle control animals were injected with a similar volume of this solution. The solutions were kept in sterile capped vials stored at 4° C. for the duration of the study. Each treatment arm has an n of 5-6 mice per group.

### Body Weight

From study day 1 to study day 14 the animals were weighed daily prior to dose administration. These data were used to calculate body weight change. The animals were placed in a weigh pan and the balance is allowed to stabilize. The weight was recorded.

### Fasting Glucose

On Study Day 14, the animals were fasted overnight (approximately 16-18 hours) by placing them in a clean cage with a clean wire rack without food but allowed access to water, and on day 15 animals were subjected to fasting blood glucose measurement as follows; the tail of the animal was resected and a blood samples was collected and blood glucose was measured using an Accu-Chek Aviva glucose meter (Roche; Indianapolis, IN). Statistical significance (*=p>0.05 vs. 0 dose; one way ANOVA Dunnett's post hoc) was calculated using GraphPad Prism software (La Jolla, Ca).

### Body Composition Analysis

On study day 1 and study day 14 body composition was analyzed using Quantitative Nuclear Magnetic Resonance EchoMRI analyzer (EMR-166-s, EchoMRI; Houston Tx). After calibrating the analyzer with a known amount of canola oil, the animals were placed in the analyzer which measures fat and non-fat (lean) mass in grams. Change in mass is calculated by subtraction of the day 14 value from the day 1 value.

### Tibialis Anterior Weights

On study day 15, following tail bleed for blood glucose, animals were sacrificed by C₀₂ asphyxiation and the right tibialis anterior muscle was dissected. Muscles were trimmed of fascia and tendons and weighted. Weight was recorded and, in the case of relative weight, Tibialis anterior weight in milligrams was divided by body weight in grams.

Tables 6-11 below show data corresponding to each of the above measurements. The data are represented as the arithmetic mean plus or minus the standard error of the mean (SEM). Tables 6-9 show ED 50s for the listed parameters as appropriate. ED 50 was calculated using the log(agonist) vs. response—Find ECanything function in GraphPad Prism software (La Jolla, Ca) with F=50 and the top fixed to vehicle mean.

TABLE 6
Dose response data for a polypeptide comprising SEQ ID NO: 29

| | 0 | 0.3 | 1 | 3 | 10 | 30 | 100 | 300 | |
|---|---|---|---|---|---|---|---|---|---|
| | nmol/kg | nmol/kg | nmol/kg | nmol/kg | nmol/kg | nmol/kg | nmol/kg | nmol/kg | ED 50 |
| | (n = 5) | (n = 6) | (n = 6) | n = 6 | (n = 6) | (n = 6) | (n = 6) | (n = 6) | (nmol/kg) |
| body | 1.5 ± | 0.88 ± | 0.30 ± | −0.72 ± | −5.43 ± | −6.51 ± | −8.12 ± | −10.42 ± | 10.56 |
| weight | 0.21 | 0.57 | 0.36 | 0.38* | 0.54* | 0.75* | 0.40* | 0.65* | |
| change | | | | | | | | | |
| (g) | | | | | | | | | |
| Fat | 1.20 ± | 1.09 ± | −1.33 ± | −2.40 ± | −5.76 ± | −5.86 ± | −7.57 ± | −9.29 ± | 6.62 |
| mass | 0.23 | 0.86 | .041 | 0.68* | 0.43* | 0.93* | 2.20* | 0.45* | |
| change | | | | | | | | | |
| (g) | | | | | | | | | |
| lean | −0.52 ± | 0.43 ± | 0.70 ± | 1.76 ± | 0.64 ± | −0.80 ± | −0.56 ± | −1.30 ± | Ambiguous |
| mass | 0.46 | 0.74 | 0.37 | 0.62 | 0.39 | 0.63 | 0.82 | 0.55 | |
| change | | | | | | | | | |
| (g) | | | | | | | | | |
| fasting | 129.6 ± | 111.7 ± | 95.78 ± | 91.67 ± | 89.28 ± | 92.61 ± | 90.22 ± | 84.67 ± | 0.35 |
| blood | 10.14 | 5.04 | 3.28* | 3.09* | 1.70* | 2.50* | 3.50* | 3.10* | |
| glucose | | | | | | | | | |
| (mg/dL) | | | | | | | | | |

*= p < 0.05 vs. vehicle (one way ANOVA, Dunnett's post hoc

TABLE 7
Dose response data for a polypeptide comprising SEQ ID NO: 1

| | 0 | 0.3 | 1 | 3 | 10 | 30 | 100 | 300 | |
|---|---|---|---|---|---|---|---|---|---|
| | nmol/kg | nmol/kg | nmol/kg | nmol/kg | nmol/kg | nmol/kg | nmol/kg | nmol/kg | ED 50 |
| | (n = 6) | (n = 6) | (n = 6) | (n = 6) | (n = 6) | (n = 6) | (n = 6) | (n = 6) | (nmol/kg) |
| body | 0.85 ± | 1.63 ± | −0.05 ± | −1.27 ± | −4.23 ± | −7.05 ± | −9.60 ± | −9.50 ± | 11.27 |
| weight | 0.79 | 0.59 | 0.7 | 0.72 | 0.85* | 0.83* | 0.63* | 0.78* | |
| change | | | | | | | | | |
| (g) | | | | | | | | | |
| Fat | 0.44 ± | −0.23 ± | −1.69 ± | −3.02 ± | −5.56 ± | −7.19 ± | −9.20 ± | −8.97 ± | 6.45 |
| mass | 0.63 | 0.45 | 0.67 | 0.71* | 0.52* | 0.66* | 0.53* | 0.55* | |
| change | | | | | | | | | |
| (g) | | | | | | | | | |
| lean | −0.19 ± | 1.09 ± | 0.86 ± | 1.42 ± | 1.00 ± | 0.47 ± | 0.15 ± | −0.62 ± | Ambiguous |
| mass | 0.30 | 0.10* | 0.30 | 0.20* | 0.46* | 0.24 | 0.27 | 0.32 | |
| change | | | | | | | | | |
| (g) | | | | | | | | | |
| fasting | 132.2 ± | 115.8 ± | 97.00 ± | 90.33 ± | 86.00 ± | 89.00 ± | 86.40 ± | 84.44 ± | 0.45 |
| blood | 4.79 | 5.43 | 4.50* | 2.89* | 3.96* | 1.53* | 5.54* | 2.22* | |
| glucose | | | | | | | | | |
| (mg/dL) | | | | | | | | | |

*= p < 0.05 vs. vehicle (one way ANOVA, Dunnett's post hoc)

TABLE 8
Dose response data for a Polypeptide comprising SEQ ID NO: 22

| | 0 | 0.3 | 1 | 3 | 10 | 30 | 100 | 300 | ED 50 |
|---|---|---|---|---|---|---|---|---|---|
| | nmol/kg | nmol/kg | nmol/kg | nmol/kg | nmol/kg | nmol/kg | nmol/kg | nmol/kg | (nmol/kg) |
| body | 1.23 ± | 0.15 ± | 0.67 ± | −1.87 ± | −4.75 ± | −8.82 ± | −9.72 ± | −12.27 ± | 12.50 |
| weight | 0.49 | 0.51 | 0.34 | 0.71* | 0.52* | 0.48* | 0.62* | 0.84* | |
| change | | | | | | | | | |
| (g) | | | | | | | | | |
| Fat | 0.93 ± | −0.92 ± | −0.96 ± | −2.90 ± | −5.56 ± | −8.34 ± | −8.86 ± | −10.95 ± | 9.80 |
| mass | 0.37 | 0.35* | 0.35* | 0.51* | 0.45* | 0.31* | 0.52* | 0.50* | |
| change | | | | | | | | | |
| (g) | | | | | | | | | |
| lean | −0.96 ± | −0.03 ± | 0.42 ± | 0.47 ± | 0.11 ± | −0.56 ± | −0.67 ± | −0.91 ± | Ambiguous |
| mass | 0.24 | 0.08 | 0.14* | 0.21* | 0.16* | 0.29 | 0.12 | 0.35 | |
| change | | | | | | | | | |
| (g) | | | | | | | | | |
| fasting | 162.4 ± | 105.70 ± | 105.20 ± | 93.92 ± | 93.50 ± | 87.42 ± | 91.58 ± | 89.25 ± | 0.03 |
| blood | 9.54 | 5.74 | 6.01 | 2.91 | 4.89* | 3.17 | 2.14* | 2.90* | |
| glucose | | | | | | | | | |
| (mg/dL) | | | | | | | | | |

*= p < 0.05 vs. vehicle (one way ANOVA, Dunnett's post hoc)

TABLE 9
Dose response data for a polypeptide comprising SEQ ID NO: 25

| | 0 | 0.3 | 1 | 3 | 10 | 30 | 100 | 300 | |
|---|---|---|---|---|---|---|---|---|---|
| | nmol/kg | nmol/kg | nmol/kg | nmol/kg | nmol/kg | nmol/kg | nmol/kg | nmol/kg | ED 50 |
| | (n = 6) | (n = 6) | (n = 6) | (n = 6) | (n = 6) | (n = 6) | (n = 6) | (n = 6) | (nmol/kg) |
| body | 1.78 ± | 1.47 ± | −0.62 ± | 0.33 ± | −5.03 ± | −6.67 ± | −9.07 ± | −10.10 ± | 11.11 |
| weight | 0.72 | 1.39 | 0.47 | 0.97 | 0.68* | 0.53* | 0.76* | 0.74* | |
| change | | | | | | | | | |
| (g) | | | | | | | | | |
| Fat | 1.15 ± | 0.05 ± | −2.01 ± | −1.60 ± | −5.45 ± | −6.71 ± | −8.57 ± | −9.16 ± | 8.46 |
| mass | 0.40 | 1.06 | 0.49* | 0.69* | 0.26* | 0.49* | 0.50* | 0.64* | |
| change | | | | | | | | | |
| (g) | | | | | | | | | |
| lean | 0.04 ± | 0.85 ± | 0.81 ± | 1.19 ± | 0.57 ± | 0.36 ± | −0.16 ± | −0.46 ± | Ambiguous |
| mass | 0.26 | 0.32 | 0.28 | 0.37 | 0.64 | 0.17 | 0.28 | 0.10 | |
| change | | | | | | | | | |
| (g) | | | | | | | | | |
| fasting | 146.20 ± | 113.6 ± | 105.60 ± | 92.67 ± | 90.33 ± | 90.42 ± | 86.58 ± | 87.08 ± | 0.25 |
| blood | 13.74 | 5.14* | 6.32* | 2.94* | 3.77* | 1.70* | 3.18* | 2.86* | |
| glucose | | | | | | | | | |
| (mg/dL) | | | | | | | | | |

*= p < 0.05 vs. vehicle (one way ANOVA, Dunnett's post hoc)

TABLE 10
Single dose (30 nmol/kg) data for polypeptides comprising SEQ ID NO:
50, SEQ ID NO: 41, SEQ ID NO: 42, SEQ ID NO: 45, SEQ ID NO: 46, SEQ
ID NO: 47, SEQ ID NO: 2, and reference compound SEQ ID NO: 54.

| | | | | | | | | | SEQ ID |
|---|---|---|---|---|---|---|---|---|---|
| | | | | | | | | | NO: 54 |
| | SEQ ID | SEQ ID | SEQ ID | SEQ ID | SEQ ID | SEQ ID | SEQ ID | SEDQ ID | 30 nmol/kg |
| | NO: 50 | NO: 41 | NO: 42 | NO: 45 | NO: 46 | NO: 47 | NO: 48 | NO: 2 | (pooled |
| | 30 | 30 | 30 | 30 | 30 | 30 | 30 | 30 | data from |
| | nmol/kg | nmol/kg | nmol/kg | nmol/kg | nmol/kg | nmol/kg | nmol/kg | nmol/kg | four studies) |
| body | −7.20 ± | −6.42 ± | −4.88 ± | −4.93 ± | −5.80 ± | −4.87 ± | −6.16 ± | −4.87 ± | −3.75 ± |
| weight | 0.57* | 0.70* | 0.45* | 0.80* | 0.62* | 0.68* | +0.65* | 0.68* | 0.32 |
| change | | | | | | | | | |
| (g) | | | | | | | | | |
| Fat | −7.18 ± | −6.59 ± | −5.33 ± | −5.40 ± | −5.63 ± | −4.80 ± | −5.73 ± | −5.76 ± | −4.05 ± |
| mass | 0.47* | 0.54* | 0.40* | 0.63* | 0.42* | 0.54* | 0.41* | 0.96* | 0.22 |
| change | | | | | | | | | |
| (g) | | | | | | | | | |
| lean | −0.02 ± | 0.40 ± | 0.48 ± | 0.56 ± | −0.19 ± | 0.26 ± | −0.19 ± | −0.35 ± | 0.60 ± |
| mass | 0.16 | 0.23 | 0.13 | 0.16 | 0.45 | 0.45 | 0.37 | 0.56 | 0.17 |
| change | | | | | | | | | |
| (g) | | | | | | | | | |
| fasting | 40.16 ± | 42.83 ± | 29.41 ± | 40.88 ± | 35.44 ± | 27.62 ± | 32.53 ± | 37.33 ± | 37.01 ± |
| blood | 3.15* | 3.28* | 3.43* | 1.70* | 1.98* | 4.06* | 3.35* | 1.68* | 1.50 |
| glucose | | | | | | | | | |
| (% reduction | | | | | | | | | |
| from | | | | | | | | | |
| vehicle) | | | | | | | | | |

*= p < 0.05 vs. vehicle from same study (one way ANOVA, Dunnett's post hoc)

TABLE 11
Data for a single dose (10 nmol/kg) of a compound comprising SEQ ID NO: 55 by itself
and in combination with a range of doses of a compound comprising SEQ ID NO: 25

| | SEQ ID NO: 55 | | | | | | | | |
|---|---|---|---|---|---|---|---|---|---|
| | 0 | 10 | 10 | 10 | 10 | 10 | 10 | 10 | 10 |
| | nmol/kg | nmol/kg | nmol/kg | nmol/kg | nmol/kg | nmol/kg | nmol/kg | nmol/kg | nmol/kg |
| | SEQ ID NO: 25 | | | | | | | | |
| | 0 | 0 | 0.3 | 1 | 3 | 10 | 30 | 100 | 300 |
| | nmol/kg | nmol/kg | nmol/kg | nmol/kg | nmol/kg | nmol/kg | nmol/kg | nmol/kg | nmol/kg |
| | (n = 6) | (n = 6) | (n = 6) | (n = 6) | (n = 6) | (n = 6) | (n = 6) | (n = 6) | (n = 6) |
| body | 1.23 ± | −10.33 ± | −9.43 ± | −12.45 ± | −11.15 ± | −12.55 ± | −14.77 ± | −16.37 ± | −17.08 ± |
| weight | 0.49 | 0.86* | 0.74* | 1.25* | 0.54* | 0.66* | 0.93*# | 1.36*# | 0.89*# |
| change (g) | | | | | | | | | |
| Fat mass | 0.93 ± | −7.55 ± | −7.61 ± | −10.45 ± | −9.60 ± | −11.14 ± | −12.32 ± | −13.36 ± | −13.55 ± |
| change (g) | 0.37 | 0.63* | 0.73* | 0.98*# | 0.49* | 0.55*# | 0.72*# | 0.72*# | 0.65*# |
| lean mass | −0.96 ± | −2.31 ± | −1.74 ± | −1.81 ± | −1.42 ± | −1.07 ± | −2.02 ± | −2.87 ± | −3.02 ± |
| change (g) | 0.24 | 0.31 | 0.16 | 0.21 | 0.28 | 0.24 | 0.31 | 0.65* | 0.35* |
| fasting | 162.4 ± | 77.67 ± | 82.33 ± | 76.83 ± | 76.08 ± | 81.25 ± | 76.50 ± | 71.08 ± | 79.42 ± |
| blood | 9.54 | 3.52* | 2.36* | 2.95* | 6.31* | 4.57* | 2.75* | 5.54* | 2.00* |
| glucose | | | | | | | | | |
| (mg/dL) | | | | | | | | | |
| Tibialis | 70.32 ± | 55.45 ± | 68.95 ± | 75.88 ± | 78.32 ± | 80.72 ± | 83.23 ± | 76.22 ± | 69.72 ± |
| weights | 2.72 | 3.21 | 3.90 | 3.68# | 5.10# | 6.63# | 5.49# | 6.86# | 2.45 |
| absolute | | | | | | | | | |
| (mg) | | | | | | | | | |
| Tibialis | 1.49 ± | 1.62 ± | 1.92 ± | 2.33 ± | 2.30 ± | 2.49 ± | 2.76 ± | 2.63 ± | 2.55 ± |
| weights | 0.06 | 0.15 | 0.12 | 0.16*# | 0.15*# | 0.16*# | 0.19*# | 0.14*# | 0.18*# |
| relative | | | | | | | | | |
| (mg/g | | | | | | | | | |
| body | | | | | | | | | |
| weight) | | | | | | | | | |

*= p < 0.05 vs. vehicle,
#= p < 0.05 vs SEQ ID NO: 55 Tirzepatide 10 nmol/kg (one way ANOVA, Dunnett's post hoc)

When the tested polypeptides were injected every three days s.c. for 14 consecutive days, the following significant differences were observed: (1) reduction in body weight, (2) reduction in fat mass and (3) reduction in fasting blood glucose (4) in the absence of significant changes in lean mass. Dose response experiments for SEQ ID NO:29, SEQ ID NO:1, SEQ ID NO:22, and SEQ ID NO:25 demonstrate improvement in efficacy over previous polypeptide SEQ ID NO:54 for (1) body weight change and (2) fat mass change as measured by ED 50 (Tables 6-9). At a single dose level of 30 nmol/kg, polypeptides comprising SEQ ID NO:50, SEQ ID NO:41, SEQ ID NO:42, SEQ ID NO:45, SEQ ID NO:46, SEQ ID NO:47, SEQ ID NO:48, SEQ ID NO:2 also demonstrated superiority in efficacy over previous polypeptide SEQ ID NO:54 for (1) body weight change and (2) fat mass change (Table 10). As shown in Table 11, polypeptide comprising SEQ ID NO:25, when combined at different dose levels with 10 nmol/kg of SEQ ID NO:55 enhanced both the body weight loss and fat mass loss induced by SEQ ID NO:55, with statistically significant differences at doses as low as 30 nmol/kg SEQ ID NO:25 (for total body weight change) and 1 nmol/kg SEQ ID NO:25 (for fat mass change). Relative to 10 nmol/kg SEQ ID NO:55 alone, the polypeptide comprising SEQ ID NO:25 also increased both relative and absolute tibialis anterior weights when doses as low as 1 nmol/kg were combined with 10 nmol/kg of SEQ ID NO:55 (Table 11).

### Example 7: Pharmacokinetics

### Bioanalytical Method

Plasma concentrations of compounds were determined by LC/MS methods. For each assay, the compound and an internal standard (IS), were extracted from 100% mouse, rat, pig, or monkey plasma (70 μL) using [50-50] Isopropanol-Methanol. Two distinct layers were formed upon centrifugation with the compound and the IS located in the supernatant layer. An aliquot of the supernatant (150 μL) was transferred to a Waters Sirocco Protein Precipitation Plate. The supernatant was eluted using a positive pressure manifold. The supernatant was mixed with 100-2 Water-Formic acid (400 μL) in a pretreated Sep-Pak tC18 SPE plate and eluted to waste. The peptide and IS were eluted with [100-2] Acetonitrile-Formic acid (100 μL) in 2 steps (50 μL each step). The elution (40 μL) from SPE plate was mixed with diluent solution (1.875 μL 5× invitrosol and 3.125 μL 1% formic acid in water, 80 μL). A final sample (10 μl) was loaded onto a Xselect CSH C18 IS column (3.5 μm 2.1×20 mm). The column effluent was directed into a Thermo Fisher Scientific Orbitrap (Exploris 240) mass spectrometer for detection and quantitation.

### Pharmacokinetic Studies

Male and female Cynomolgus monkeys were administered a single subcutaneous dose or intravenous dose (183 to 190 nmol/kg) of compound in 40 mM Tris-HCl 5% D-mannitol (pH 8) at a volume of 0.5 mL/kg. Blood was collected from each animal at 0.5, 3, 6, 24, 48, 72, 96, 168, 240, 336, 408, and 504 hours postdose for pharmacokinetic characterization.

Male Yucatan mini-pigs were administered a single subcutaneous dose or intravenous dose (19 or 99 nmol/kg) of compound in 40 mM Tris-HCl 5% D-mannitol (pH 8) at a volume of 0.01 or 0.05 mL/kg. Blood was collected from each animal at 0.083 (IV only), 0.5, 3, 6, 24, 48, 72, 96, 168, 240, 336, 408, and 504 hours postdose for pharmacokinetic characterization.

Male Sprague Dawley rats were administered a single subcutaneous dose (183 to 201 nmol/kg) of compound in 40 mM Tris-HCl 5% D-mannitol (pH 8) at a volume of 0.5 or 2 mL/kg. Blood was collected from each animal at 0.5, 3, 6, 24, 48, 72, 96, 120, and 168 hours postdose for pharmacokinetic characterization.

Male Sprague Dawley rats were administered a single subcutaneous dose (10, 30 or 100 nmol/kg) of compound in 40 mM Tris-HCl 5% D-mannitol (pH 8) at a volume of 1 mL/kg. Blood was collected from each animal at 3, 6, 24, 48, 72, 96, 120, and 168 hours postdose for pharmacokinetic characterization.

Male CD-1 mice were administered a single subcutaneous dose (100 nmol/kg) of compound in 40 mM Tris-HCl 5% D-mannitol (pH 8) at a volume of 5 mL/kg. Blood was collected at 6, 24, 48, 96, 168, 240, 288, and 336 hours postdose for pharmacokinetic characterization.

TABLE 12
Individual and Mean Pharmacokinetic Parameters Following a Single
Subcutaneous Dose to Male and Female Cynomolgus Monkeys

| SEQ ID | | T_{1/2} | Tmax | Cmax | AUC_{0-inf} | CL/F |
|---|---|---|---|---|---|---|
| (Dose) | Animal_ID | (hr) | (hr) | (nmole/L) | (hr*nmole/L) | (mL/hr/kg) |
| SEQ ID NO: 1 | PM214419 | 72.0 | 48 | 1100 | 221000 | 0.867 |
| (187 nmol/kg) | PM220568 | 91.7 | 48 | 1260 | 255000 | 0.755 |
| | Mean | 81.9 | 48 | 1180 | 238000 | 0.811 |
| SEQ ID NO: 22 | PM220212 | 182 | 72 | 1720 | 387000 | 0.522 |
| (183 nmol/kg) | PM220229 | 90.2 | 48 | 2280 | 342000 | 0.552 |
| | Mean | 136 | 60 | 2000 | 365000 | 0.537 |
| SEQ ID NO: 25 | PM220563 | 184 | 24 | 1540 | 364000 | 0.599 |
| (190 nmol/kg) | PM220223 | 214 | 24 | 1680 | 551000 | 0.423 |
| | Mean | 199 | 24 | 1610 | 458000 | 0.511 |

Abbreviations: AUC_{0-inf} = area under the curve from time 0 hours to infinity, CL/F = clearance/bioavailability, Tmax = time to maximum concentration, Cmax = maximum observed plasma concentration, T½ = half-life.

TABLE 13
Individual and Mean Pharmacokinetic Parameters
Following a Single Intravenous Dose to Male and
Female Cynomolgus Monkeys

| | | | C₀ | AUC_{0-inf} | CL |
|---|---|---|---|---|---|
| SEQ ID | | T_{1/2} | (nmole/ | (hr*nmole/ | (mL/ |
| (Dose) | Animal_ID | (hr) | L) | L) | hr/kg) |
| SEQ ID NO: 1 | PM222982 | 91.3 | 4150 | 261000 | 0.717 |
| (187 nmol/kg) | PM203046 | 91.9 | 5040 | 369000 | 0.507 |
| | Mean | 91.6 | 4600 | 315000 | 0.612 |
| SEQ ID NO: 22 | PM210594 | 83.1 | 4160 | 467000 | 0.392 |
| (183 nmol/kg) | PM210588 | 113 | 5320 | 424000 | 0.431 |
| | Mean | 98.1 | 4740 | 446000 | 0.412 |
| SEQ ID NO: 25 | PM214424 | 100 | 3040 | 355000 | 0.534 |
| (190 nmol/kg) | PM220569 | 126 | 3960 | 449000 | 0.422 |
| | Mean | 113 | 3500 | 402000 | 0.478 |

Abbreviations: AUC_{0-inf} = area under the curve from time 0 hours to infinity, CL = clearance, C₀ = estimated plasma concentration at time zero, T_{1/2} = half-life.

TABLE 14
Individual and Mean Pharmacokinetic Parameters Following
a Single Subcutaneous Dose to Male Yucatan Pigs

| SEQ ID NO: | | T_{1/2} | Tmax | Cmax | AUC_{0-inf} | CL/F |
|---|---|---|---|---|---|---|
| (Dose) | Animal_ID | (hr) | (hr) | (nmole/L) | (hr*nmole/L) | (mL/hr/kg) |
| SEQ ID NO: 29 | 2001 | 166 | 24 | 497 | 128000 | 1.03 |
| (99 nmol/kg) | 2002 | 292 | 24 | 201 | 67500 | 2.06 |
| | 2003 | 185 | 168 | 79.6 | 33800 | 3.56 |
| | Mean | 214 | 72 | 259 | 76400 | 2.22 |
| | SD | 67.9 | 83 | 215 | 47700 | 1.27 |
| SEQ ID NO: 48 | 1519 | 70.1 | 48 | 140 | 29300 | 0.653 |
| (19 nmol/kg) | 1523 | 66.8 | 6.0 | 157 | 35600 | 0.565 |
| | Mean | 68.5 | 27 | 149 | 32500 | 0.609 |
| SEQ ID NO: 2 | 1578 | 70.7 | 6 | 154 | 24400 | 0.788 |
| (19 nmol/kg) | 1857 | 90.8 | 96 | 60.4 | 11700 | 1.63 |
| | Mean | 80.8 | 51 | 107 | 18100 | 1.21 |
| SEQ ID NO: 1 | 1572 | 91.6 | 6 | 171 | 22400 | 0.848 |
| (19 nmol/kg) | 1579 | 60.2 | 6 | 122 | 13100 | 1.46 |
| | Mean | 75.9 | 6 | 147 | 17800 | 1.15 |
| SEQ ID NO: 22 | 1944 | 79.1 | 24 | 165 | 23900 | 0.777 |
| (18 nmol/kg) | 1947 | 85.8 | 6.0 | 143 | 21000 | 0.886 |
| | Mean | 82.5 | 15 | 154 | 22500 | 0.832 |
| SEQ ID NO: 25 | 1954 | 80.8 | 24 | 159 | 28300 | 0.681 |
| (19 nmol/kg) | 1988 | 70.4 | 24 | 181 | 26800 | 0.717 |
| | Mean | 75.6 | 24 | 170 | 27600 | 0.699 |

Abbreviations: AUC_{0-inf} = area under the curve from time 0 hours to infinity, CL/F = clearance/bioavailability, Tmax = time to maximum concentration, Cmax = maximum observed plasma concentration, T½ = half-life.

TABLE 15
Individual and Mean Pharmacokinetic Parameters
Following a Single Intravenous Dose to Male Yucatan Pigs

| | | | C₀ | AUC_{0-inf} | CL |
|---|---|---|---|---|---|
| Compound | | T_{1/2} | (nmole/ | (hr*nmole/ | (mL/ |
| (Dose) | Animal_ID | (hr) | L) | L) | hr/kg) |
| SEQ ID NO: 29 | 1001 | 126 | 2560 | 220000 | 0.448 |
| (99 nmol/kg) | 1002 | 263 | 2380 | 329000 | 0.299 |
| | 1003 | 109 | 2530 | 307000 | 0.321 |
| | Mean | 166 | 2490 | 285000 | 0.356 |
| | SD | 84.4 | 96.4 | 57600 | 0.080 |
| SEQ ID NO: 48 | 1570 | 86.7 | 899 | 32500 | 0.580 |
| (19 nmol/kg) | 2711 | 89.2 | 987 | 41200 | 0.458 |
| | Mean | 88.0 | 943 | 36900 | 0.519 |
| SEQ ID NO: 2 | 1530 | 95.4 | 809 | 28900 | 0.648 |
| (19 nmol/kg) | 1568 | 75.1 | 578 | 32800 | 0.571 |
| | Mean | 85.3 | 694 | 30900 | 0.610 |
| SEQ ID NO: 1 | 1561 | 87.0 | 591 | 32100 | 0.583 |
| (19 nmol/kg) | 1565 | 55.9 | 646 | 20200 | 0.927 |
| | Mean | 71.5 | 619 | 26200 | 0.755 |
| SEQ ID NO: 22 | 1933 | 90.1 | 373 | 22300 | 0.820 |
| (18 nmol/kg) | 1938 | 101 | 462 | 28300 | 0.646 |
| | Mean | 95.6 | 418 | 25300 | 0.733 |
| SEQ ID NO: 25 | 1950 | 106 | 522 | 41900 | 0.453 |
| (19 nmol/kg) | 1963 | 86.8 | 545 | 37200 | 0.510 |
| | Mean | 96.4 | 534 | 39600 | 0.482 |

Abbreviations: AUC_{0-inf} = area under the curve from time 0 hours to infinity, CL = clearance, C₀ = estimated plasma concentration at time zero, T_{1/2} = half-life.

TABLE 16
Individual and Mean Pharmacokinetic Parameters Following
a Single Subcutaneous Dose to Male Sprague Dawley Rats

| Compound | | T_{1/2} | Tmax | Cmax | AUC_{0-inf} | CL/F |
|---|---|---|---|---|---|---|
| (Dose) | Animal_ID | (hr) | (hr) | (nmole/L) | (hr*nmole/L) | (mL/hr/kg) |
| SEQ ID NO: 49 | R0101 | 19.1 | 24 | 340 | 17100 | 11.5 |
| (196 nmol/kg) | R0102 | 16.0 | 6 | 474 | 22400 | 8.76 |
| | R0103 | 20.2 | 24 | 367 | 17300 | 11.4 |
| | Mean | 18.4 | 18 | 394 | 18900 | 10.6 |
| | SD | 2.18 | 10 | 70.9 | 3000 | 1.55 |
| SEQ ID NO: 50 | R0301 | 19.7 | 24 | 577 | 26700 | 7.55 |
| (201 nmol/kg) | R0302 | 14.9 | 24 | 460 | 25100 | 8.03 |
| | R0303 | 16.7 | 24 | 618 | 28700 | 7.01 |
| | Mean | 17.1 | 24 | 552 | 26800 | 7.53 |
| | SD | 2.42 | 0.0 | 82.0 | 1800 | 0.510 |
| SEQ ID NO: 29 | R0501 | 52.8 | 24 | 881 | 96800 | 2.35 |
| (197 nmol/kg) | R0502 | 41.1 | 24 | 749 | 82900 | 2.61 |
| | Mean | 47.0 | 24 | 815 | 89900 | 2.48 |
| SEQ ID NO: 41 | R0101 | 43.8 | 24 | 467 | 43100 | 5.04 |
| (196 nmol/kg) | R0102 | 36.5 | 24 | 645 | 56200 | 3.73 |
| | R0103 | 37.7 | 24 | 539 | 45000 | 4.63 |
| | Mean | 39.3 | 24 | 550 | 48100 | 4.47 |
| | SD | 3.91 | 0.0 | 89.5 | 7080 | 0.670 |
| SEQ ID NO: 42 | R0301 | 33.2 | 48 | 399 | 38100 | 5.41 |
| (196 nmol/kg) | R0302 | 39.1 | 24 | 559 | 43200 | 4.80 |
| | R0303 | 54.2 | 48 | 460 | 51500 | 4.35 |
| | Mean | 42.2 | 40 | 473 | 44300 | 4.85 |
| | SD | 10.8 | 14 | 80.7 | 6760 | 0.532 |

Abbreviations: AUC_{0-inf} = area under the curve from time 0 hours to infinity, CL/F = clearance/bioavailability, Tmax = time to maximum concentration, Cmax = maximum observed plasma concentration, T½ = half-life.

TABLE 17
Individual and Mean Pharmacokinetic Parameters Following
a Single Subcutaneous Dose to Male Sprague Dawley Rats

| Compound | | T_{1/2} | Tmax | Cmax | AUC_{0-inf} | CL/F |
|---|---|---|---|---|---|---|
| (Dose) | Animal_ID | (hr) | (hr) | (nmole/L) | (hr*nmole/L) | (mL/hr/kg) |
| SEQ ID NO: 1 | R0101 | 37.7 | 24 | 804 | 53900 | 3.67 |
| (187 nmol/kg) | R0102 | 38.2 | 24 | 511 | 41200 | 4.77 |
| | R0103 | 31.2 | 24 | 657 | 51800 | 3.77 |
| | Mean | 35.7 | 24 | 657 | 49000 | 4.07 |
| | SD | 3.91 | 0.0 | 147 | 6810 | 0.608 |
| SEQ ID NO: 22 | R0301 | 26.2 | 24 | 783 | 38900 | 4.75 |
| (183 nmol/kg) | R0302 | 21.8 | 24 | 439 | 34300 | 5.39 |
| | R0303 | 23.5 | 48 | 527 | 37100 | 4.99 |
| | Mean | 23.8 | 32 | 583 | 36800 | 5.04 |
| | SD | 2.22 | 14 | 179 | 2320 | 0.323 |
| SEQ ID NO: 25 | R0101 | 14.8 | 24 | 466 | 23400 | 8.13 |
| (190 nmol/kg) | R0102 | 13.9 | 24 | 475 | 21600 | 8.80 |
| | R0103 | 14.5 | 24 | 582 | 24700 | 7.69 |
| | Mean | 14.4 | 24 | 508 | 23200 | 8.21 |
| | SD | 0.458 | 0.0 | 64.5 | 1560 | 0.559 |

Abbreviations: AUC_{0-inf} = area under the curve from time 0 hours to infinity, CL/F = clearance/bioavailability, Tmax = time to maximum concentration, Cmax = maximum observed plasma concentration, T½ = half-life.

TABLE 18
Individual and Mean Pharmacokinetic Parameters Following
a Single Intravenous Dose to Male Sprague Dawley Rats

| | | | C₀ | AUC_{0-inf} | CL |
|---|---|---|---|---|---|
| Compound | | T_{1/2} | (nmole/ | (hr*nmole/ | (mL/ |
| (Dose) | Animal_ID | (hr) | L) | L) | hr/kg) |
| SEQ ID NO: 49 | R0001 | 21.7 | 2610 | 53100 | 3.69 |
| (196 nmol/kg) | R0002 | 21.6 | 3110 | 48400 | 4.05 |
| | R0003 | 17.8 | 2790 | 41500 | 4.72 |
| | Mean | 20.4 | 2840 | 47700 | 4.15 |
| | SD | 2.22 | 253 | 5830 | 0.523 |
| SEQ ID NO: 50 | R0201 | 20.3 | 5560 | 59600 | 3.37 |
| (201 nmol/kg) | R0202 | 18.3 | 4360 | 74200 | 2.71 |
| | R0203 | 19.0 | 5960 | 73900 | 2.72 |
| | Mean | 19.2 | 5290 | 69200 | 2.93 |
| | SD | 1.01 | 833 | 8340 | 0.378 |
| SEQ ID NO: 29 | R0401 | 41.7 | 6910 | 119000 | 1.66 |
| (197 nmol/kg) | R0402 | 45.2 | 4840 | 152000 | 1.30 |
| | R0403 | 49.6 | 1050 | 136000 | 1.45 |
| | Mean | 45.5 | 4270 | 136000 | 1.47 |
| | SD | 3.96 | 2970 | 16500 | 0.181 |
| SEQ ID NO: 41 | R0001 | 48.4 | 1350 | 50100 | 3.91 |
| (196 nmol/kg) | R0002 | 49.8 | 1620 | 60600 | 3.23 |
| | R0003 | 39.0 | 586 | 45500 | 4.31 |
| | Mean | 45.7 | 1190 | 52100 | 3.82 |
| | SD | 5.87 | 536 | 7740 | 0.546 |
| SEQ ID NO: 42 | R0201 | 36.1 | 2760 | 81500 | 2.40 |
| (196 nmol/kg) | R0202 | 35.8 | 2250 | 77900 | 2.51 |
| | R0204 | 46.0 | 2260 | 82500 | 2.37 |
| | Mean | 39.3 | 2420 | 80600 | 2.43 |
| | SD | 5.80 | 292 | 2420 | 0.074 |

Abbreviations: AUC_{0-inf} = area under the curve from time 0 hours to infinity, CL/F = clearance/bioavailability, C₀ = estimated plasma concentration at time zero, T_{1/2} = half-life.

TABLE 19
Individual and Mean Pharmacokinetic Parameters Following
a Single Intravenous Dose to Male Sprague Dawley Rats

| | | | C₀ | AUC_{0-inf} | CL |
|---|---|---|---|---|---|
| Compound | | T_{1/2} | (nmole/ | (hr*nmole/ | (mL/ |
| (Dose) | Animal_ID | (hr) | L) | L) | hr/kg) |
| SEQ ID NO: 1 | R0001 | 23.2 | 6950 | 91900 | 2.04 |
| (187 nmol/kg) | R0003 | 31.0 | 2400 | 77300 | 2.42 |
| | R0004 | 47.5 | 5970 | 111000 | 1.69 |
| | Mean | 33.9 | 5110 | 93400 | 2.05 |
| | SD | 12.4 | 2390 | 16900 | 0.365 |
| SEQ ID NO: 22 | R0201 | 24.7 | 3240 | 88400 | 2.07 |
| (183 nmol/kg) | R0202 | 21.3 | 5390 | 79600 | 2.30 |
| | R0203 | 22.9 | 5200 | 92300 | 1.98 |
| | Mean | 23.0 | 4610 | 86800 | 2.12 |
| | SD | 1.70 | 1190 | 6510 | 0.165 |
| SEQ ID NO: 25 | R0001 | 17.4 | 1610 | 41300 | 4.59 |
| (190 nmol/kg) | R0002 | 16.9 | 3540 | 46900 | 4.04 |
| | R0003 | 18.2 | 3610 | 61800 | 3.07 |
| | Mean | 17.5 | 2920 | 50000 | 3.90 |
| | SD | 0.656 | 1140 | 10600 | 0.770 |

Abbreviations: AUC_{0-inf} = area under the curve from time 0 hours to infinity, CL/F = clearance/bioavailability, C₀ = estimated plasma concentration at time zero, T_{1/2} = half-life.

TABLE 20
Individual and Mean Pharmacokinetic Parameters Following a Single 10, 30 or
100 nmol/kg Subcutaneous Dose to Male Sprague Dawley Rats (Dose Response)

| Compound | | T_{1/2} | Tmax | Cmax | AUC_{0-inf} | CL/F |
|---|---|---|---|---|---|---|
| (Dose) | Animal_ID | (hr) | (hr) | (nmole/L) | (hr*nmole/L) | (mL/hr/kg) |
| SEQ ID NO: 1 | R0001 | 26.0 | 24 | 37.7 | 2070 | 5.10 |
| (10 nmol/kg) | R0002 | 41.7 | 24 | 26.4 | 1860 | 6.10 |
| | R0003 | 31.1 | 24 | 38.5 | 2210 | 4.63 |
| | Mean | 32.9 | 24 | 34.2 | 2050 | 5.28 |
| | SD | 8.01 | 0.0 | 6.77 | 176 | 0.751 |
| SEQ ID NO: 1 | R0101 | 31.5 | 24 | 102 | 6420 | 4.80 |
| (30 nmol/kg) | R0102 | 37.1 | 24 | 110 | 6920 | 4.55 |
| | R0103 | 36.1 | 48 | 104 | 7690 | 4.19 |
| | Mean | 34.9 | 32 | 105 | 7010 | 4.51 |
| | SD | 2.99 | 14 | 4.16 | 640 | 0.307 |
| SEQ ID NO: 1 | R0201 | 30.8 | 24 | 348 | 18800 | 5.46 |
| (100 nmol/kg) | R0202 | 26.0 | 24 | 332 | 21100 | 4.86 |
| | R0203 | 24.6 | 24 | 321 | 20700 | 4.91 |
| | Mean | 27.1 | 24 | 334 | 20200 | 5.08 |
| | SD | 3.25 | 0.0 | 13.6 | 1230 | 0.333 |
| SEQ ID NO: 22 | R0301 | 30.4 | 24 | 49.5 | 2730 | 3.75 |
| (10 nmol/kg) | R0302 | 23.1 | 24 | 51.3 | 2810 | 3.60 |
| | R0303 | 41.5 | 24 | 48.6 | 3020 | 3.54 |
| | Mean | 31.7 | 24 | 49.8 | 2850 | 3.63 |
| | SD | 9.27 | 0.0 | 1.37 | 150 | 0.108 |
| SEQ ID NO: 22 | R0401 | 27.4 | 24 | 126 | 7520 | 4.07 |
| (30 nmol/kg) | R0402 | 33.6 | 24 | 132 | 8600 | 3.62 |
| | R0403 | 24.1 | 24 | 128 | 7160 | 4.25 |
| | Mean | 28.4 | 24 | 129 | 7760 | 3.98 |
| | SD | 4.82 | 0.0 | 3.06 | 749 | 0.324 |
| SEQ ID NO: 22 | R0501 | 28.6 | 24 | 372 | 26700 | 3.85 |
| (100 nmol/kg) | R0502 | 25.9 | 24 | 416 | 26000 | 3.91 |
| | R0503 | 30.7 | 24 | 369 | 26300 | 3.91 |
| | Mean | 28.4 | 24 | 386 | 26300 | 3.89 |
| | SD | 2.41 | 0.0 | 26.3 | 351 | 0.035 |
| SEQ ID NO: 25 | R0601 | 19.9 | 24 | 20.8 | 1050 | 9.70 |
| (10 nmol/kg) | R0602 | 18.6 | 24 | 21.9 | 972 | 10.5 |
| | R0603 | 31.5 | 6 | 26.8 | 1110 | 9.09 |
| | Mean | 23.3 | 18 | 23.2 | 1040 | 9.76 |
| | SD | 7.10 | 10 | 3.19 | 69.2 | 0.707 |
| SEQ ID NO: 25 | R0701 | 19.2 | 24 | 55.0 | 2640 | 11.4 |
| (30 nmol/kg) | R0702 | 19.6 | 24 | 68.0 | 2910 | 10.4 |
| | R0703 | 20.8 | 6 | 59.1 | 2960 | 10.2 |
| | Mean | 19.9 | 18 | 60.7 | 2840 | 10.7 |
| | SD | 0.833 | 10 | 6.65 | 172 | 0.643 |
| SEQ ID NO: 25 | R0801 | 16.0 | 24 | 239 | 12500 | 7.99 |
| (100 nmol/kg) | R0802 | 17.4 | 24 | 316 | 14000 | 7.15 |
| | R0803 | 15.7 | 24 | 328 | 13400 | 7.47 |
| | Mean | 16.4 | 24 | 294 | 13300 | 7.54 |
| | SD | 0.907 | 0.0 | 48.3 | 755 | 0.424 |

Abbreviations: AUC_{0-inf} = area under the curve from time 0 hours to infinity, CL/F = clearance/bioavailability, Tmax = time to maximum concentration, Cmax = maximum observed plasma concentration, T½ = half-life.

TABLE 21
Mean Pharmacokinetic Parameters Following a Single 100 nmol/kg
Subcutaneous Dose to Male CD-1 Mice

| Compound | T_{1/2} | Tmax | Cmax | AUC_{0-inf} | CL/F |
|---|---|---|---|---|---|
| (Dose) | (hr) | (hr) | (nmole/L) | (hr*nmole/L) | (mL/hr/kg) |
| SEQ ID NO: 1 | 22.8 | 24 | 327 | 25700 | 3.94 |
| (100 nmol/kg) | | | | | |
| SEQ ID NO: 22 | 25.1 | 24 | 270 | 15000 | 6.76 |
| (100 nmol/kg) | | | | | |
| SEQ ID NO: 25 | 32.5 | 24 | 366 | 25000 | 4.01 |
| (100 nmol/kg) | | | | | |

Abbreviations: AUC_{0-inf} = area under the curve from time 0 hours to infinity, CL/F = clearance/bioavailability, Tmax = time to maximum concentration, Cmax = maximum observed concentration, T1/2 = half-life.

### PK Conclusions

Across the different species, the PK profiles support a prolonged elimination half-life and decreased clearance compared to wildtype UCN2. These data demonstrate that the above compounds have a pharmacokinetic profile suitable for once weekly administration.

### Example 8: Immunogenicity Risk Assessment

### MAPPS Assay (MHC-Associated Peptide Proteomics)

MAPPS profiles the MHC-II presented peptides on human dendritic cells treated with tested molecules. CD14+ cells, isolated from the PBMCs of normal human donors, were cultured and differentiated into immature DCs by incubation with IL-4 and GM-CSF. On day 4, culture media was replaced with fresh media containing tested molecules. On day 5, LPS was added to transform the cells into mature DCs. On day 6, cells were lysed in RIPA buffer with protease inhibitors. Immunoprecipitation of MHC-II complexes were performed using biotinylated anti-MHC-II antibody coupled to streptavidin beads. The bound complex was eluted and filtered. The isolated MHC-II peptides were analyzed by a mass spectrometer. Peptide identifications were generated by an internal proteomics pipeline using search algorithms with no enzyme and a bovine/human database with test sequences appended to the database. A KNIME workflow was used to process the identification files from the samples. Peptides identified from the test articles were aligned against the parent sequence of the test molecule. The output was used to determine the percentage of donors displaying MHC-II peptides from regions of the test molecule. Polypeptides comprising SEQ ID NOs:54, 49, 50, 29, 41, 45, 1, 22, 25 were tested in the MAPPs assay. Polypeptides comprising SEQ ID NOs: 41, 45, 25 showed no peptides displayed on MHC-II complexes in the assay. Compounds comprising SEQ ID NO:54, 49, 50, 29, 1, 22 displayed at least one peptide cluster on MHC-II complexes in a subset of donors. Depending on the donors, up to three major clusters were observed, each slightly shifted from the others, altogether spanning residues 1-33.

### T Cell Proliferation Assay

This assay assessed the ability of tested antibodies or tested MAPPS peptides to activate CD4+ T cells by inducing cellular proliferation. CD8+ T cell depleted PBMC's were prepared and labeled with CFSE. Each sample was tested with media control, keyhole limpet haemocyanin (KLH; positive clinical benchmark control), tested antibodies, or tested MAPPS peptides. Cultures were incubated for 7 days. On day 7, samples were analyzed by flow cytometry. Reference analog comprising SEQ ID NO:54 elicited a response in four out of ten donors, whereas the analogs of this disclosure did not elicit a response.

### Pre-Existing Reactivity (ACE Assay Format)

This assay assessed the presence of pre-existing antibodies (ADA) against the tested molecules in treatment-naive normal human serum (NHS). Diluted NHS was incubated overnight on a Pierce Streptavidin plate coated with biotinylated tested molecules. On the following day, the captured binding proteins were acid eluted, hard-coated to a Mesoscale (MSD) plate, and detected with a combination of biotin-labeled molecule and ruthenium-labeled streptavidin. If anti-drug antibodies were present, they would bind the labeled drug and the resulting signal was referred to as Tier 1 signal (expressed as electrochemiluminescence). This signal was confirmed in Tier 2 by adding excess unlabeled tested molecule in the detection step, which resulted in the suppression of the Tier 1 signal. The presence of pre-existing anti-drug antibodies was expressed as magnitude of the 90^{th} percentile of Tier 2 inhibition. The 90^{th} percentile of Tier 2 inhibition is a statistical tool to assess the magnitude of specificity of Tier 1 reactivity. This 90^{th} percentile was used to rank molecules in terms of ADA risk. The polypeptide comprising SEQ ID NO:1 had higher 90^{th} percentile Tier 2 inhibition than polypeptides comprising SEQ ID NOs: 22 and 25.

TABLE 22
Immunogenicity Risk Assessment Summary

| | MAPPS | T Cell Proliferation | Pre-existing |
|---|---|---|---|
| | Assay | Assay: protein | Reactivity |
| SEQ ID | >10 donors with | 7-10 donors with | 90^{th} percentile T2 |
| NO | diverse MHC | diverse MHC | inhibition >50 donors |
| 54 | 7/10 (70%) | 4/10 | ND |
| 49 | 6/26 (23%) | ND | ND |
| 50 | 3/10 (30%) | ND | ND |
| 29 | 8/30 (27%) | ND | ND |
| 41 | 0/10 (0%) | ND | ND |
| 45 | 0/20 (0%) | ND | ND |
| 1 | 3/20 (15%) | 0/9 | 37.9% |
| 22 | 1/10 (10%) | 0/9 | 14.3% |
| 25 | 0/10 (0%) | 0/9 | 4.5% |

Abbreviations: ACE = acid capture elution; ADA = anti-drug antibodies; CDR = complementarity determining region; DC = dendritic cell; H1 = VH CDR1; H2 = VH CDR2; H3 = VH CDR3; L1 = VL CDR1; L2 = VL CDR2; MAPPs = MHC-associated peptide proteomics; MHC = major histocompatibility complex; MS = mass spectrometry; ND = no data; T2 = Tier 2; TCEM = T cell exposed motif; VH = variable heavy; VL = variable light; VHFR3 = variable heavy framework 3

Polypeptides comprising SEQ ID NO:49, 50, 29. 41, 45, 1, 22, and 25 demonstrated substantial improvements in immunogenicity over a compound comprising SEQ ID NO:54 in the MAPPs assay and/or the T cell proliferation assay.

SEQUENCES
SEQ ID NO: 1
Ac-GSPSIVTS αMeL DVPT Aib αMeL LQKILEQERAEKARQQAK(γE-(2-[2-(2-
Amino-ethoxy)-ethoxy]-acetyl)-ϵK-CO-(CH₂)₁₈-CO₂H)EAAEILAQV-NH₂
SEQ ID NO: 2
Ac-GSPSIVTS αMeL DVPT Aib αMeL LQKLLEQERAEKARQQAK(γE-(2-[2-(2-
Amino-ethoxy)-ethoxy]-acetyl)-ϵK-CO-(CH₂)₁₈-CO₂H)EAAEILAQV-NH₂
SEQ ID NO: 3
Ac-GSPSIVTS αMeL DVPT Aib αMeL LQKILEQERAEKARQQAK((2-[2-(2-Amino-
ethoxy)-ethoxy]-acetyl)-PKE-γE-CO-(CH₂)₁₈-CO₂H)EAAEILAQV-NH₂
SEQ ID NO: 4
Ac-GSPSIVTS αMeL DVPT Aib αMeL LQKILEQERAEKARQQAK(Ahx-(2-[2-(2-
Amino-ethoxy)-ethoxy]-acetyl)-γE-CO-(CH₂)₁₈-CO₂H)EAAEILAQV-NH₂
SEQ ID NO: 5
Ac-GSPSIVTS αMeL DVPT Aib αMeL LQKILEQERAEKARQQAK(γE-(2-[2-(2-
Amino-ethoxy)-ethoxy]-acetyl)-Ahx-CO-(CH₂)₁₈-CO₂H)EAAEILAQV-NH₂
SEQ ID NO: 6
Ac-GSPSIVTS αMeL DVPT Aib αMeL LQKILEQERAEKARQQAK(γE-GKEKEKE-
CO-(CH₂)₁₈-CO₂H)EAAEILAQV-NH₂
SEQ ID NO: 7
Ac-GSPSIVTS αMeL DVPT Aib αMeL LQKILEQERAEKARQQAK(G-(βA)₃-γE-
CO-(CH₂)₁₈-CO₂H)EAAEILAQV-NH₂
SEQ ID NO: 8
Ac-GSPSIVTS αMeL DVPT Aib αMeL LQKILEQERAEKARQQAK(G-(βA)₄-γE-
CO-(CH₂)₁₈-CO₂H)EAAEILAQV-NH₂
SEQ ID NO: 9
Ac-GSPSIVTS αMeL DVPT Aib αMeL LQKILEQERAEKARQQAK((2-[2-(2-Amino-
ethoxy)-ethoxy]-acetyl)-KEKEKE-γE-CO-(CH₂)₁₈-CO₂H)EAAEILAQV-NH₂
SEQ ID NO: 10
Ac-GSPSIVTS αMeL DVPT Aib αMeL LQKILEQERAEKARQQAK((βA)-S-(βA)-S-
γE-CO-(CH₂)₁₈-CO₂H)EAAEILAQV-NH₂
SEQ ID NO: 11
Ac-GSPSIVTS αMeL DVPT Aib αMeL LQKILEQERAEKARQQAK(γE-PKE-Ahx-CO-
(CH₂)₁₈-CO₂H)EAAEILAQV-NH₂
SEQ ID NO: 12
Ac-GSPSIVTS αMeL DVPT Aib αMeL LQKLLEQERAEKARQQAK(γE-(2-[2-(2-
Amino-ethoxy)-ethoxy]-acetyl)-K-CO-(CH₂)₁₆-CO₂H)EAAEILAQV-NH₂
SEQ ID NO: 13
Ac-GSPSIVTS αMeL DVPT Aib αMeL LQKILEQERAEKARQQAK(γE-PKE-ϵK-CO-
(CH₂)₁₈-CO₂H)EAAEILAQV-NH₂
SEQ ID NO: 14
Ac-GSPSIVTS αMeL DVPT Aib αMeL LQKILEQERAEKARQQAK(γE-PPP-EK-CO-
(CH₂)₁₈-CO₂H)EAAEILAQV-NH₂
SEQ ID NO: 15
Ac-GSPSIVTS αMeL DVPT Aib αMeL LQKILEQERAEKARQQAK(γE-PPPPPP-CO-
(CH₂)₁₈-CO₂H)EAAEILAQV-NH₂
SEQ ID NO: 16
Ac-GSPSIVTS αMeL DVPT Aib αMeL LQKILEQERAEKARQQAK(γE-PEPEPE-CO-
(CH₂)₁₈-CO₂H)EAAEILAQV-NH₂
SEQ ID NO: 17
Ac-GSPSIVTS αMeL DVPT Aib αMeL LQKILEQERAEKARQQAK(γE-(2-[2-(2-
Amino-ethoxy)-ethoxy]-acetyl)₂-ϵK-CO-(CH₂)₁₈-CO₂H)EAAEILAQV-NH₂
SEQ ID NO: 18
Ac-GSPSIVTS αMeL DVPT Aib αMeL LQKILEQERAEKARQQAK(γE-ϵK-γE-ϵK-
CO-(CH₂)₁₈-CO₂H)EAAEILAQV-NH₂
SEQ ID NO: 19
Ac-GSPSIVTS αMeL DVPT Aib αMeL LQKILEQERAEKARQQAK(γE-EK-(2-[2-
(2-Amino-ethoxy)-ethoxy]-acetyl)-ϵK-CO-(CH₂)₁₈-CO₂H)EAAEILAQV-NH₂
SEQ ID NO: 20
Ac-GSPSIVTS αMeL DVPT Aib αMeL LQKILEQERAEKARQQAKEAAEILAQV-
NH₂
SEQ ID NO: 21
Ac-GSPSIVTS αMeL DVPT Aib αMeL LQKILEQERAEKARQQAK(γE-(2-[2-(2-
Amino-ethoxy)-ethoxy]-acetyl)-ϵK-CO-(CH₂)₁₆-CO₂H)EAAEILAQV-NH₂
SEQ ID NO: 22
Pyr-GSPSGIVTS αMeL DVPT Aib αMeL LQKILEQERAEKARQQAK(γE-(2-[2-(2-
Amino-ethoxy)-ethoxy]-acetyl)-ϵK-CO-(CH₂)₁₈-CO₂H)EAAEILAQV-NH₂
SEQ ID NO: 23
Pyr-GSPSGIVTS αMeL DVPT Aib αMeL LQKILEQERAEKARQQAKEAAEILAQV-
NH₂
SEQ ID NO: 24
Pyr-GSPSGIVTS αMeL DVPT Aib αMeL LQKILEQERAEKARQQAK(γE-(2-[2-(2-
Amino-ethoxy)-ethoxy]-acetyl)-ϵK-CO-(CH₂)₁₆-CO₂H)EAAEILAQV-NH₂
SEQ ID NO: 25
HO₂C-(CH₂)₁₈-CO-ϵK-(2-[2-(2-Amino-ethoxy)-ethoxy]-acetyl)-γE-GSPSIVTS αMeL
DVPT Aib αMeL LQKILEQERAEKARQQATEAAEILAQV-NH₂
SEQ ID NO: 26
HO₂C-(CH₂)₁₈-CO-K-(2-[2-(2-Amino-ethoxy)-ethoxy]-acetyl)-γE-GSPSIVTS αMeL
DVPT Aib αMeL LQKILEQERAEKARQQATEAAEILAQV-NH₂
SEQ ID NO: 27
GSPSIVTS αMeL DVPT Aib αMeL LQKILEQERAEKARQQATEAAEILAQV-NH₂
SEQ ID NO: 28
HO₂C-(CH₂)₁₆-CO-ϵK-(2-[2-(2-Amino-ethoxy)-ethoxy]-acetyl)-γE-GSPSIVTS αMeL
DVPT Aib αMeL LQKILEQERAEKARQQATEAAEILAQV-NH₂
SEQ ID NO: 29
Ac-IVTS αMeL D (D-Val) PTG αMeL LQIKLEQERAEKARQ QAK((2-[2-(2-Amino-
ethoxy)-ethoxy]-acetyl)₂-γE-CO-(CH₂)₁₈-CO₂H)ENTEILAQV-NH₂
SEQ ID NO: 30
Ac-IVTS αMeL D (D-Val) PTG αMeL LQIKLEQERAEKARQQAK(γE-(2-[2-(2-Amino-
ethoxy)-ethoxy]-acetyl)-γE-CO-(CH₂)₁₈-CO₂H)ENTEILAQV-NH₂
SEQ ID NO: 31
Ac-IVTS αMeL D (D-Val) PTG αMeL LQIKLEQERAEKARQQAK((γE)₂-(2-[2-(2-
Amino-ethoxy)-ethoxy]-acetyl)-CO-(CH₂)₁₈-CO₂H)ENTEILAQV-NH₂
SEQ ID NO: 32
Ac-IVTS αMeL D (D-Val) PTG αMeL LQIKLEQERAEKARQQAK((2-[2-(2-Amino-
ethoxy)-ethoxy]-acetyl)-ϵK-γE-CO-(CH₂)₁₈-CO₂H)ENTEILAQV-NH₂
SEQ ID NO: 33
Ac-IVTS αMeL D (D-Val) PTG αMeL LQIKLEQERAEKARQQAK((2-[2-(2-Amino-
ethoxy)-ethoxy]-acetyl)-γE-ϵK-CO-(CH₂)₁₈-CO₂H)ENTEILAQV-NH₂
SEQ ID NO: 34
Ac-IVTS αMeL D (D-Val) PTG αMeL LQIKLEQERAEKARQQAK(ϵK-(2-[2-(2-
Amino-ethoxy)-ethoxy]-acetyl)-γE-CO-(CH₂)₁₈-CO₂H)ENTEILAQV-NH₂
SEQ ID NO: 35
Ac-IVTS αMeL D (D-Val) PTG αMeL LQIKLEQERAEKARQQAK(ϵK-γE-(2-[2-(2-
Amino-ethoxy)-ethoxy]-acetyl)-CO-(CH₂)₁₈-CO₂H)ENTEILAQV-NH₂
SEQ ID NO: 36
Ac-IVTS αMeL D (D-Val) PTG αMeL LQIKLEQERAEKARQQAK(γE-(2-[2-(2-Amino-
ethoxy)-ethoxy]-acetyl)-ϵK-CO-(CH₂)₁₈-CO₂H)ENTEILAQV-NH₂
SEQ ID NO: 37
Ac-IVTS αMeL D (D-Val) PTG αMeL LQIKLEQERAEKARQQAK(γE-ϵK-(2-[2-(2-
Amino-ethoxy)-ethoxy]-acetyl)-CO-(CH₂)₁₈-CO₂H)ENTEILAQV-NH₂
SEQ ID NO: 38
Ac-IVTS αMeL D (D-Val) PTG αMeL LQIKLEQERAEKARQQAK(γE-γE-ϵK-CO-
(CH₂)₁₈-CO₂H)ENTEILAQV-NH₂
SEQ ID NO: 39
Ac-IVTS αMeL D (D-Val) PTG αMeL LQIKLEQERAEKARQQAK(γE-ϵK-γE-CO-
(CH₂)₁₈-CO₂H)ENTEILAQV-NH₂
SEQ ID NO: 40
Ac-IVTS αMeL D (D-Val) PTG αMeL LQIKLEQERAEKARQQAK(ϵK-(γE)₂-CO-
(CH₂)₁₈-CO₂H)ENTEILAQV-NH₂
SEQ ID NO: 41
Ac-IVTS αMeL D (D-Val) PT Aib αMeL LQIKLEQERAEKARQQAK((2-[2-(2-Amino-
ethoxy)-ethoxy]-acetyl)₂-γE-CO-(CH₂)₁₈-CO₂H)ENTEILAQV-NH₂
SEQ ID NO: 42
Ac-IVTS αMeL D (D-Val) PI Aib αMeL LQIKLEQERAEKARQQAK((2-[2-(2-Amino-
ethoxy)-ethoxy]-acetyl)₂-γE-CO-(CH₂)₁₈-CO₂H)ENTEILAQV-NH₂
SEQ ID NO: 43
Ac-IVTS αMeL D (D-Val) PTG αMeL LQIKLEQERAEKARQQAK(GKEKEKE-γE-
CO-(CH₂)₁₈-CO₂H)ENTEILAQV-NH₂
SEQ ID NO: 44
Ac-IVTS αMeL D (D-Val) PTG αMeL LQIKLEQERAEKARQQAK((2-[2-(2-Amino-
ethoxy)-ethoxy]-acetyl)-(γE)₂-CO-(CH₂)₁₈-CO₂H)ENTEILAQV-NH₂
SEQ ID NO: 45
γE-GPSIVTS αMeL D (D-Val) PI Aib αMeL LQIKLEQERAEKARQQAK((2-[2-(2-
Amino-ethoxy)-ethoxy]-acetyl)₂-γE-CO-(CH₂)₁₈-CO₂H)ENTEILAQV-NH₂
SEQ ID NO: 46
Ac-PPPIVTS αMeL D (D-Val) PT Aib αMeL LQK Aib LEQERAEKARQQAK(γE-(2-
[2-(2-Amino-ethoxy)-ethoxy]-acetyl)-ϵK-CO-(CH₂)₁₈-CO₂H)ENTEILAQV-NH₂
SEQ ID NO: 47
Ac-PPPIVTS αMeL D (D-Val) PT Aib αMeL LQKLLEQERAEKARQQAK(γE-(2-[2-(2-
Amino-ethoxy)-ethoxy]-acetyl)-ϵK-CO-(CH₂)₁₈-CO₂H)EAAEILAQV-NH₂
SEQ ID NO: 48
Ac-PPPIVTS αMeL DVPT Aib αMeL LQKLLEQERAEKARQQAK(γE-(2-[2-(2-
Amino-ethoxy)-ethoxy]-acetyl)-ϵK-CO-(CH₂)₁₈-CO₂H)EAAEILAQV-NH₂
SEQ ID NO: 49
(NMeI) VTSLDVPIG αMeL LQKLIEQEKQEKEKQQAK((2-[2-(2-Amino-ethoxy)-
ethoxy]-acetyl)₂-γE-CO-(CH₂)₁₈-CO₂H)ENTEILAQV-NH₂
SEQ ID NO: 50
(NMeI) VTSLDVPIG αMeL LQKLLEQERAEKARQQAK((2-[2-(2-Amino-ethoxy)-
ethoxy]-acetyl)₂-γE-CO-(CH₂)₁₈-CO₂H)EAAEILAQV-NH₂
SEQ ID NO: 51
Ac-IVTS αMeL D (D-Val) PI Aib αMeL LQIKLEQERAEKARQQAK((E)₂-CO-
(CH₂)₁₈-CO₂H)ENTEILAQV-NH₂
SEQ ID NO: 52
HO₂C-(CH₂)₁₈-CO-Dap-(2-[2-(2-Amino-ethoxy)-ethoxy]-acetyl)-γE-GSPSIVTS αMeL
DVPT Aib αMeL LEKILEQERAEKARQQATEAAEILAQV-NH₂
SEQ ID NO: 53
IVLSLDVPIGLLQILLEQARARAAREQATTNARILARV-NH₂
SEQ ID NO: 54
(NMeI) VLSLDVPIGLLQILLEQEKQEKEKQQAK((2-[2-(2-Amino-ethoxy)-ethoxy]-
acetyl)₂-(γE)₂-CO-(CH₂)₁₈-CO₂H)TNAQILAQV-NH₂
SEQ ID NO: 55
Y Aib EGTFTSDYSI Aib LDKIAQK((2-[2-(2-Amino-ethoxy)-ethoxy]-acetyl)₂-γE-CO-
(CH₂)₁₈-CO₂H)AFVQWLIAGGPSSGAPPPS-NH₂
SEQ ID NO: 56
Y Aib QGTFTSDYSI αMeL LDKK((2-[2-(2-Amino-ethoxy)-ethoxy]-acetyl)₂-γE-CO-
(CH₂)₁₈-CO₂H)AQ Aib AFIEYLLEGGPSSGAPPPS-NH₂
SEQ ID NO: 57
Y Aib QGTFTSDYSI αMeL LDKK((2-[2-(2-Amino-ethoxy)-ethoxy]-acetyl)-γE-CO-
(CH₂)₁₈-CO₂H)AQ Aib AFIEYLLEGGPSSGAPPPS-NH₂
SEQ ID NO: 58
Y Aib QGTFTSDYSILLDKK((2-[2-(2-Amino-ethoxy)-ethoxy]-acetyl)₂-γE-CO-(CH₂)₁₈-
CO₂H)AQ Aib AFIEYLLEGGPSSGAPPPS-NH₂
SEQ ID NO: 59
H Aib QGTFTSDYSKYLDEKKAK((2-[2-(2-Amino-ethoxy)-ethoxy]-acetyl)₂-γE-CO-
(CH₂)₁₆-CO₂H)EFVEWLLEGGPSSG-NH₂
SEQ ID NO: 60
H Aib QGTFTSDYSKYLDEKKAK((2-[2-(2-Amino-ethoxy)-ethoxy]-acetyl)₂-(γE)₂-CO-
(CH₂)₁₆-CO₂H)EFVEWLLSGGPSSG-NH₂
SEQ ID NO: 61
H Aib QGTFTSDYSKYLDEKKAK((2-[2-(2-Amino-ethoxy)-ethoxy]-acetyl)₂-γE-CO-
(CH₂)₁₈-CO₂H)EFVEWLLEGGPSSG-NH₂
SEQ ID NO: 62
H Aib QGTFTSDYSKYLDEKKAK((2-[2-(2-Amino-ethoxy)-ethoxy]-acetyl)₂-(γE)₂-CO-
(CH₂)₁₈-CO₂H)EFVEWLLSGGPSSG-NH₂
SEQ ID NO: 63
γE-[CNTATC]*ATG Orn LAE aMeF LVRSSN (NMeN) FGPK((γE)₂-CO-(CH₂)₁₈-
CO₂H)LPPTEVGSNTY-NH₂
SEQ ID NO: 64
γE-[CNTATC]*ATG Orn LAE aMeF LVRSSN (NMeD) FGPK((γE)₂-CO-(CH₂)₁₈-
CO₂H)LPPTEVGSNTY-NH₂
SEQ ID NO: 65
K((γE)₂-CO-(CH₂)₁₈-CO₂H)-[CETATC]*ATG Orn LAE aMeF LVRSSN (NMeD)
FGPILPPTEVGSNTY-NH₂
SEQ ID NO: 66
K((γE)₂-CO-(CH₂)₁₈-CO₂H)-[CETATC]*ATG Orn αMeL
AEFL VRSSHNFGPILPPTEVGSNTY-NH₂
SEQ ID NO: 67
Ac-ASHLSTAVLGK((2-[2-(2-Amino-ethoxy)-ethoxy]-acetyl)₂-γE-CO-(CH₂)₁₈-CO₂H)
LS Aib ELHKLEDYPRTDVGAESP-NH₂
SEQ ID NO: 68
X₁X₂X₃X₄X₅X₆IVTSX₁₁DX₁₃PX₁₅X₁₆X₁₇LX₁₉X₂₀X₂₁X₂₂EQEX₂₆X₂₇EKX₃₀X₃₁QQAX₃₅E
X₃₇X₃₈EILAQV wherein,
X₁ is Pyr, E, γE, or absent,
X₂ is G or absent,
X₃ is G, S, or absent,
X₄ is S, P, G, or absent,
X₅ is S, P, or absent,
X₆ is G, S, P, or absent,
X₁₁ is L or αMeL,
X₁₃ is V or D-Val,
X₁₅ is T or I,
X₁₆ is Aib or G,
X₁₇ is L or αMeL,
X₁₉ is Q or E,
X₂₀ is K or I,
X₂₁ is I, K, Aib, or L,
X₂₂ is I or L,
X₂₆ is R or K,
X₂₇ is A or Q,
X₃₀ is A or E,
X₃₁ is R or K,
X₃₅ is K or T,
X₃₇ is A or N,
X₃₈ is A or T,
wherein the N-term amino acid is optionally N-acylated, N-acetylated or N-
methylated, and wherein the C-term amino acid is optionally amidated.
SEQ ID NO: 69
GSPSIVTS αMeL DVPT Aib αMeL LQKX₂₁LEQERAEKARQQAKEAAEILAQV
wherein,
X₂₁ is I or L,
wherein the N-term amino acid is optionally N-acetylated, and wherein the C-term
amino acid is optionally amidated.
SEQ ID NO: 70
Pyr GSPSGIVTS αMeL DVPT Aib αMeL LQKILEQERAEKARQQAKEAAEILAQV,
wherein the C-term amino acid is optionally amidated
SEQ ID NO: 71
X₁X₂GSPSIVTS αMeL DVPT Aib αMeLLX₁₉KILEQERAEKARQQATEAAEILAQV,
wherein
X₁ is γE or absent,
X₂ is absent,
X₁₉ is Q or E,
wherein the C-term amino acid is optionally amidated
SEQ ID NO: 72
IVTS αMeL D D-Val PX₁₅X₁₆ αMeL LQIKLEQERAEKARQQAKENTEILAQV
(SEQ ID NO: 72), wherein
X₁₅ is T or I,
X₁₆ is G or Aib,
wherein the N-term is amino acid is optionally N-acetylated, and wherein the C-
term amino acid is optionally amidated.
SEQ ID NO: 73
PPPIVTS αMeL DX₁₃PTAib αMeL LQKX₂₁LEQERAEKARQQAKEX₃₇X₃₈EILAQV
wherein,
X₁₃ is V or D-Val,
X₂₁ is L or Aib,
X₃₇ is A or N,
X₃₈ is A or T,
wherein the N-term amino acid is optionally N-acetylated, and wherein the C-term
amino acid is optionally amidated.
SEQ ID NO: 74
γE-GKEKEKE-CO-(CH₂)₁₈-CO₂H
SEQ ID NO: 75
(2-[2-(2-Amino-ethoxy)-ethoxy]-acetyl)-KEKEKE-γE-CO-(CH₂)₁₈-CO₂H
SEQ ID NO: 76
γE-PPPPPP-CO-(CH₂)₁₈-CO₂H
SEQ ID NO: 77
γE-PEPEPE-CO-(CH₂)₁₈-CO₂H
SEQ ID NO: 78
GKEKEKE-γE-CO-(CH₂)₁₈-CO₂H
SEQ ID NO: 79
H Aib EGTFTSDVSSYLEGQAAK((2-[2-(2-Amino-ethoxy)-ethoxy]-acetyl)₂-γE-CO-
(CH₂)₁₆-CO₂H)EFIAWLVRGRG-OH
SEQ ID NO: 80
HAEGTFTSDVSSYLEGQAAK(γE-CO-(CH₂)₁₄-CH₃)EFIAWLVRGRG-OH
Dulaglutide is a human GLP-1 receptor agonist which comprises a dimer of a GLP-1
analog fused at its C-terminus via a peptide linker to the N-terminus of an
analog of an Fc portion of an immunoglobulin, and is identified by CAS registry
number 923950-08-7, which provides the following chemical name:
7-37-Glucagon-like peptide I [8-glycine,22-glutamic acid,36-glycine]
(synthetic human) fusion protein with peptide (synthetic 16-amino acid linker)
fusion protein with immunoglobulin G4 (synthetic human Fc fragment), dimer.
Each monomer of dulaglutide has the amino acid sequence
set forth in SEQ ID NO: 81
SEQ ID NO: 81
HGEGTFTSDVSSYLEEQAAKEFIAWLVKGGGGGGGSGGGGSGGGGSAESKYGPP
CPPCPAPEAAGGPSVFLFPPKPKDTLMISRTPEVTCVVVDVSQEDPEVQFNWYV
DGVEVHNAKTKPREEQFNSTYRVVSVLTVLHQDWLNGKEYKCKVSNKGLPSSIE
KTISKAKGQPREPQVYTLPPSQEEMTKNQVSLTCLVKGFYPSDIAVEWESNGQP
ENNYKTTPPVLDSDGSFFLYSRLTVDKSRWQEGNVFSCSVMHEALHNHYTQKSL
SLSLG

We claim:

1. A polypeptide, or a pharmaceutically acceptable salt thereof, comprising:
X₁X₂X₃X₄X₅X₆IVTSX₁₁DX₁₃PX₁₅X₁₆X₁₇LX₁₉X₂₀X₂₁X₂₂EQEX₂₆X₂₇EKX₃₀X₃₁QQAX₃₅E X₃₇X₃₈EILAQV (SEQ ID NO:68), wherein
X₁ is Pyr, E, γE, or absent,
X₂ is G or absent,
X₃ is G, S, or absent,
X₄ is S, P, G, or absent,
X₅ is S, P, or absent,
X₆ is G, S, P, or absent,
X₁₁ is L or αMeL,
X₁₃ is V or D-Val,
X₁₅ is T or I,
X₁₆ is Aib or G,
X₁₇ is L or αMeL,
X₁₉ is Q or E,
X₂₀ is K or I,
X₂₁ is I, K, Aib, or L,
X₂₂ is I or L,
X₂₆ is R or K,
X₂₇ is A or Q,
X₃₀ is A or E,
X₃₁ is R or K,
X₃₅ is K or T,
X₃₇ is A or N, and
X₃₈ is A or T;
wherein the N-term amino acid is optionally N-acylated, N-acetylated or N-methylated; and wherein the C-term amino acid is optionally amidated.

2. The polypeptide of claim 1, or the pharmaceutically acceptable salt thereof, wherein:
a) X₁ is absent, X₂ is absent, X₃ is G, X₄ is S, X₅ is P, X₆ is S, X₁₁ is αMeL, X₁₃ is V, X₁₅ is T, X₁₆ is Aib, X₁₇ is αMeL, X₁₉ is Q, X₂₀ is K, X₂₁ is I or L, X₂₂ is L, X₂₆ is R, X₂₇ is A, X₃₀ is A, X₃₁ is R, X₃₅ is K, X₃₇ is A, and X₃₈ is A; or
b) X₁ is Pyr, X₂ is G, X₃ is S, X₄ is P, X₅ is S, X₆ is G, X₁₁ is αMeL, X₁₃ is V, X₁₅ is T, X₁₆ is Aib, X₁₇ is αMeL, X₁₉ is Q, X₂₀ is K, X₂₁ is I, X₂₂ is L, X₂₆ is R, X₂₇ is A, X₃₀ is A, X₃₁ is R, X₃₅ is K, X₃₇ is A, and X₃₈ is A; or
c) X₁ is γE or absent, X₂ is absent, X₃ is G, X₄ is S, X₅ is P, X₆ is S, X₁₁ is αMeL, X₁₃ is V, X₁₅ is T, X₁₆ is Aib, X₁₇ is αMeL, X₁₉ is Q or E, X₂₀ is K, X₂₁ is I, X₂₂ is L, X₂₆ is R, X₂₇ is A, X₃₀ is A, X₃₁ is R, X₃₅ is T, X₃₇ is A, and X₃₈ is A; or
d) X₁ is absent, X₂ is absent, X₃ is absent, X₄ is absent, X₅ is absent, X₆ is absent, X₁₁ is αMeL, X₁₃ is D-Val, X₅ is T or I, X₁₁ is Aib or G, X₁₉ is Q, X₂₀ is I, X₂₁ is K, X₂₂ is L, X₂₆ is R, X₂₇ is A, X₃₀ is A, X₃₁ is R, X₃₅ is K, X₃₇ is N, and X₃₈ is T; or
e) X₁ is absent, X₂ is absent, X₃ is absent, X₄ is P, X₅ is P, X₆ is P, X₁₁ is αMeL, X₁₃ is V or D-Val, X₁₅ is T, X₁₆ is Aib, X₁₇ is αMeL, X₁₉ is Q, X₂₀ is K, X₂₁ is L or Aib, X₂₂ is L, X₂₆ is R, X₂₇ is A, X₃₀ is A, X₃₁ is R, X₃₅ is K, X₃₇ is A or N, X₃₈ is A or T; or
f) X₁ is γE, X₂ is absent, X₃ is absent; X₄ is G; X₅ is P; X₆ is S; X₁₁ is αMeL, X₁₃ is D-Val, X₁₅ is I, X₁₆ is Aib, X₁₇ is αMeL, X₁₉ is Q, X₂₀ is I, X₂₁ is K, X₂₂ is L, X₂₆ is R, X₂₇ is A, X₃₀ is A, X₃₁ is R, X₃₅ is K, X₃₇ is N, X₃₈ is T; or
g) X₁ is absent, X₂ is absent, X₃ is absent, X₄ is absent, X₅ is absent, X₆ is absent, X₁₁ is L, X₁₃ is V, X₁₅ is I, X₁₆ is G, X₁₇ is αMeL, X₁₉ is Q, X₂₀ is K, X₂₁ is L, X₂₂ is I, X₂₆ is K, X₂₇ is Q, X₃₀ is E, X₃₁ is K, X₃₅ is K, X₃₇ is N, X₃₈ is T; or
h) X₁ is absent, X₂ is absent, X₃ is absent, X₄ is absent, X₅ is absent, X₆ is absent, X₁₁ is L, X₁₃ is V, X₁₅ is I, X₁₁ is G, X₁₇ is αMeL, X₁₉ is Q, X₂₀ is K, K₂₁ is L, X₂₂ is L, X₂₆ is R, X₂₇ is A, X₃₀ is A, X₃₁ is R, X₃₅ is K, X₃₇ is A, X₃₈ is A.

3. The polypeptide of claim 1, or the pharmaceutically acceptable salt thereof, further comprising a means for increasing the half-life of the polypeptide.

4. The polypeptide of claim 3, or the pharmaceutically acceptable salt thereof, wherein the means for increasing the half-life of the polypeptide is a fatty acid, wherein the fatty acid is conjugated to the polypeptide at an amino acid with a functional group available for conjugation, via a direct bond or via a linker between the amino acid and the fatty acid.

5. The polypeptide of claim 2, or the pharmaceutically acceptable salt thereof, wherein the polypeptide is polypeptide c), and wherein X₁ is γE.

6. The polypeptide of claim 5, or the pharmaceutically acceptable salt thereof, wherein the N-term amino acid is N-acylated via a fatty acid conjugated to the N-term amino acid, via a direct bond or via a linker between the amino acid and the fatty acid.

7. The polypeptide of claim 2, or pharmaceutically acceptable salt thereof, wherein the polypeptide is selected from the group consisting of polypeptide a), b), d), e), f), and g), and wherein the polypeptide further comprises a fatty acid conjugated to K at X₃₅, wherein the fatty acid is conjugated to the epsilon-amino group of K via a direct bond or via a linker between K and the fatty acid.

8. The polypeptide of claim 7, or a pharmaceutically acceptable salt thereof, wherein the N-term amino acid is N-acetylated.

9. The polypeptide of claim 7, or a pharmaceutically acceptable salt thereof, wherein the N-term amino acid is N-methylated.

10. The polypeptide claim 1, or a pharmaceutically acceptable salt thereof, wherein the C-term amino acid is amidated.

11. A polypeptide comprising any one of SEQ ID NOs: 1-52, or a pharmaceutically acceptable salt thereof.

12. The polypeptide of claim 11, or a pharmaceutically acceptable salt thereof, selected from the group consisting of SEQ ID NO:1, SEQ ID NO:2, SEQ ID NO:22, SEQ ID NO:25, SEQ ID NO:29, SEQ ID NO:41, SEQ ID NO:42, SEQ ID NO:45, SEQ ID NO:46, SEQ ID NO:48, SEQ ID NO:49, and SEQ ID NO:50.

13. A pharmaceutical composition comprising the polypeptide of claim 1, or a pharmaceutically acceptable salt thereof, and at least one pharmaceutically acceptable carrier, diluent, or excipient.

14. A method of treating a disease or condition selected from the group consisting of diabetes mellitus, obesity, chronic weight management, non-alcoholic fatty liver disease (NAFLD), non-alcoholic steatohepatitis (NASH), dyslipidemia, metabolic syndrome, chronic kidney disease (CKD), osteoarthritis (OA), obesity-related sleep apnea (OSA), sarcopenia, cachexia, sarcopenic obesity (SO), and polycystic ovarian syndrome (PCOS), the method comprising administering to an individual in need thereof an effective amount of a polypeptide, or a pharmaceutically acceptable salt thereof, of claim 1.

15. A method of treating a disease or condition, selected from the group consisting of: diabetes mellitus, obesity, chronic weight management, non-alcoholic fatty liver disease (NAFLD), non-alcoholic steatohepatitis (NASH), dyslipidemia, metabolic syndrome, chronic kidney disease (CKD), osteoarthritis (OA), obesity-related sleep apnea (OSA), sarcopenia, cachexia, sarcopenic obesity (SO), and polycystic ovarian syndrome (PCOS), the method comprising administering to an individual in need thereof an effective amount of the polypeptide of claim 1, or the pharmaceutically acceptable salt thereof, and an effective amount of an additional therapeutic agent.

16. The method of claim 15 wherein the additional therapeutic agent is a dual agonist of GIP and GLP-1 receptors.

17. The method of claim 16 wherein the dual agonist of the GIP and GLP-1 receptors is a compound comprising SEQ ID NO:55.

18. The method of claim 15 wherein the additional therapeutic agent is a GLP-1 receptor agonist.

19. The method of claim 18 wherein the GLP-1 receptor agonists is a compound comprising SEQ ID NO:79, SEQ ID NO:80, or SEQ ID NO:81.


SEQUENCE LISTING (81 sequences)

SEQ ID NO: 1 - protein, 42 aa, synthetic construct
  SITE 1: note = N-terminal acetylated residue
  SITE 9: note = alpha-methyl-leucine
  SITE 14: note = 2-Aminoisobutyric Acid
  SITE 15: note = alpha-methyl-leucine
  SITE 33: note = Lysine is chemically modified by conjugation of the epsilon-amino group of the Lysine side chain with gamma-Glutamic acid-(2-[2-(2-Amino-ethoxy)-ethoxy]-acetyl) -epsilon-Lysine-CO-(CH2)18-CO2H
  SITE 42: note = Amidated Residue
GSPSIVTSLDVPTXLLQKILEQERAEKARQQAKEAAEILAQV

SEQ ID NO: 2 - protein, 42 aa, synthetic construct
  SITE 1: note = N-terminal acetylated residue
  SITE 9: note = alpha-methyl-leucine
  SITE 14: note = 2-Aminoisobutyric Acid
  SITE 15: note = alpha-methyl-leucine
  SITE 33: note = Lysine is chemically modified by conjugation of the epsilon-amino group of the Lysine side chain with gamma-Glutamic acid-(2-[2-(2-Amino-ethoxy)-ethoxy]-acetyl) -epsilon-Lysine-CO-(CH2)18-CO2H
  SITE 42: note = Amidated Residue
GSPSIVTSLDVPTXLLQKLLEQERAEKARQQAKEAAEILAQV

SEQ ID NO: 3 - protein, 42 aa, synthetic construct; identical to SEQ ID NO: 1
  SITE 1: note = N-terminal acetylated residue
  SITE 9: note = alpha-methyl-leucine
  SITE 14: note = 2-Aminoisobutyric Acid
  SITE 15: note = alpha-methyl-leucine
  SITE 33: note = Lysine is chemically modified by conjugation of the epsilon-amino group of the Lysine side chain with (2-[2-(2-Amino-ethoxy)-ethoxy]-acetyl)-PKE-gamma-Glutamic acid-CO-(CH2)18-CO2H
  SITE 42: note = Amidated Residue

SEQ ID NO: 4 - protein, 42 aa, synthetic construct; identical to SEQ ID NO: 1
  SITE 1: note = N-terminal acetylated residue
  SITE 9: note = alpha-methyl-leucine
  SITE 14: note = 2-Aminoisobutyric Acid
  SITE 15: note = alpha-methyl-leucine
  SITE 33: note = Lysine is chemically modified by conjugation of the epsilon-amino group of the Lysine side chain with 6-amino-hexanoic acid-(2-[2-(2-Amino-ethoxy)-ethoxy]-acetyl)-gamma-Glutamic acid-CO-(CH2)18-CO2H
  SITE 42: note = Amidated Residue

SEQ ID NO: 5 - protein, 42 aa, synthetic construct; identical to SEQ ID NO: 1
  SITE 1: note = N-terminal acetylated residue
  SITE 9: note = alpha-methyl-leucine
  SITE 14: note = 2-Aminoisobutyric Acid
  SITE 15: note = alpha-methyl-leucine
  SITE 33: note = Lysine is chemically modified by conjugation of the epsilon-amino group of the Lysine side chain with gamma-Glutamic acid-(2-[2-(2-Amino-ethoxy)-ethoxy]-acetyl) -6-amino-hexanoic acid-CO-(CH2)18-CO2H
  SITE 42: note = Amidated Residue

SEQ ID NO: 6 - protein, 42 aa, synthetic construct; identical to SEQ ID NO: 1
  SITE 1: note = N-terminal acetylated residue
  SITE 9: note = alpha-methyl-leucine
  SITE 14: note = 2-Aminoisobutyric Acid
  SITE 15: note = alpha-methyl-leucine
  SITE 33: note = Lysine is chemically modified by conjugation of the epsilon-amino group of the Lysine side chain with gamma-Glutamic acid-GKEKEKE-CO-(CH2)18-CO2H
  SITE 42: note = Amidated Residue

SEQ ID NO: 7 - protein, 42 aa, synthetic construct; identical to SEQ ID NO: 1
  SITE 1: note = N-terminal acetylated residue
  SITE 9: note = alpha-methyl-leucine
  SITE 14: note = 2-Aminoisobutyric Acid
  SITE 15: note = alpha-methyl-leucine
  SITE 33: note = Lysine is chemically modified by conjugation of the epsilon-amino group of the Lysine side chain with Glycine-(Beta-Alanine)3-gamma-Glutamic acid-CO-(CH2)18-CO2H
  SITE 42: note = Amidated Residue

SEQ ID NO: 8 - protein, 42 aa, synthetic construct; identical to SEQ ID NO: 1
  SITE 1: note = N-terminal acetylated residue
  SITE 9: note = alpha-methyl-leucine
  SITE 14: note = 2-Aminoisobutyric Acid
  SITE 15: note = alpha-methyl-leucine
  SITE 33: note = Lysine is chemically modified by conjugation of the epsilon-amino group of the Lysine side chain with Glycine-(Beta-Alanine)4-gamma-Glutamic acid-CO-(CH2)18-CO2H
  SITE 42: note = Amidated Residue

SEQ ID NO: 9 - protein, 42 aa, synthetic construct; identical to SEQ ID NO: 1
  SITE 1: note = N-terminal acetylated residue
  SITE 9: note = alpha-methyl-leucine
  SITE 14: note = 2-Aminoisobutyric Acid
  SITE 15: note = alpha-methyl-leucine
  SITE 33: note = Lysine is chemically modified by conjugation of the epsilon-amino group of the Lysine side chain with (2-[2-(2 -Amino-ethoxy)-ethoxy]-acetyl)-KEKEKE-gamma-Glutamic acid-CO-(CH2)18-CO2H
  SITE 42: note = Amidated Residue

SEQ ID NO: 10 - protein, 42 aa, synthetic construct; identical to SEQ ID NO: 1
  SITE 1: note = N-terminal acetylated residue
  SITE 9: note = alpha-methyl-leucine
  SITE 14: note = 2-Aminoisobutyric Acid
  SITE 15: note = alpha-methyl-leucine
  SITE 33: note = Lysine is chemically modified by conjugation of the epsilon-amino group of the Lysine side chain with (Beta-Alanine)-Serine-(Beta-Alanine)-Serine-gamma-Glutamic acid-CO-(CH2)18-CO2H
  SITE 42: note = Amidated Residue

SEQ ID NO: 11 - protein, 42 aa, synthetic construct; identical to SEQ ID NO: 1
  SITE 1: note = N-terminal acetylated residue
  SITE 9: note = alpha-methyl-leucine
  SITE 14: note = 2-Aminoisobutyric Acid
  SITE 15: note = alpha-methyl-leucine
  SITE 33: note = Lysine is chemically modified by conjugation of the epsilon-amino group of the Lysine side chain with gamma-Glutamic acid-PKE-6-amino-hexanoic acid-CO-(CH2)18-CO2H
  SITE 42: note = Amidated Residue

SEQ ID NO: 12 - protein, 42 aa, synthetic construct; identical to SEQ ID NO: 2
  SITE 1: note = N-terminal acetylated residue
  SITE 9: note = alpha-methyl-leucine
  SITE 14: note = 2-Aminoisobutyric Acid
  SITE 15: note = alpha-methyl-leucine
  SITE 33: note = Lysine is chemically modified by conjugation of the epsilon-amino group of the Lysine side chain with gamma-Glutamic acid-(2-[2-(2-Amino-ethoxy)-ethoxy]-acetyl) -epsilon-Lysine-CO-(CH2)16-CO2H
  SITE 42: note = Amidated Residue

SEQ ID NO: 13 - protein, 42 aa, synthetic construct; identical to SEQ ID NO: 1
  SITE 1: note = N-terminal acetylated residue
  SITE 9: note = alpha-methyl-leucine
  SITE 14: note = 2-Aminoisobutyric Acid
  SITE 15: note = alpha-methyl-leucine
  SITE 33: note = Lysine is chemically modified by conjugation of the epsilon-amino group of the Lysine side chain with gamma-Glutamic acid-PKE-epsilon-Lysine-CO-(CH2)18-CO2H
  SITE 42: note = Amidated Residue

SEQ ID NO: 14 - protein, 42 aa, synthetic construct; identical to SEQ ID NO: 1
  SITE 1: note = N-terminal acetylated residue
  SITE 9: note = alpha-methyl-leucine
  SITE 14: note = 2-Aminoisobutyric Acid
  SITE 15: note = alpha-methyl-leucine
  SITE 33: note = Lysine is chemically modified by conjugation of the epsilon-amino group of the Lysine side chain with gamma-Glutamic acid-PPP-epsilon-Lysine-CO-(CH2)18-CO2H
  SITE 42: note = Amidated Residue

SEQ ID NO: 15 - protein, 42 aa, synthetic construct; identical to SEQ ID NO: 1
  SITE 1: note = N-terminal acetylated residue
  SITE 9: note = alpha-methyl-leucine
  SITE 14: note = 2-Aminoisobutyric Acid
  SITE 15: note = alpha-methyl-leucine
  SITE 33: note = Lysine is chemically modified by conjugation of the epsilon-amino group of the Lysine side chain with gamma-Glutamic acid-PPPPPP-CO-(CH2)18-CO2H
  SITE 42: note = Amidated Residue

SEQ ID NO: 16 - protein, 42 aa, synthetic construct; identical to SEQ ID NO: 1
  SITE 1: note = N-terminal acetylated residue
  SITE 9: note = alpha-methyl-leucine
  SITE 14: note = 2-Aminoisobutyric Acid
  SITE 15: note = alpha-methyl-leucine
  SITE 33: note = Lysine is chemically modified by conjugation of the epsilon-amino group of the Lysine side chain with gamma-Glutamic acid-PEPEPE-CO-(CH2)18-CO2H
  SITE 42: note = Amidated Residue

SEQ ID NO: 17 - protein, 42 aa, synthetic construct; identical to SEQ ID NO: 1
  SITE 1: note = N-terminal acetylated residue
  SITE 9: note = alpha-methyl-leucine
  SITE 14: note = 2-Aminoisobutyric Acid
  SITE 15: note = alpha-methyl-leucine
  SITE 33: note = Lysine is chemically modified by conjugation of the epsilon-amino group of the Lysine side chain with gamma-Glutamic acid-(2-[2-(2-Amino-ethoxy)-ethoxy]-acetyl) 2-epsilon-Lysine-CO-(CH2)18-CO2H
  SITE 42: note = Amidated Residue

SEQ ID NO: 18 - protein, 42 aa, synthetic construct; identical to SEQ ID NO: 1
  SITE 1: note = N-terminal acetylated residue
  SITE 9: note = alpha-methyl-leucine
  SITE 14: note = 2-Aminoisobutyric Acid
  SITE 15: note = alpha-methyl-leucine
  SITE 33: note = Lysine is chemically modified by conjugation of the epsilon-amino group of the Lysine side chain with gamma-Glutamic acid-epsilon-Lysine-gamma-Glutamic acid-epsilon-Lysine-CO-(CH2)18-CO2H
  SITE 42: note = Amidated Residue

SEQ ID NO: 19 - protein, 42 aa, synthetic construct; identical to SEQ ID NO: 1
  SITE 1: note = N-terminal acetylated residue
  SITE 9: note = alpha-methyl-leucine
  SITE 14: note = 2-Aminoisobutyric Acid
  SITE 15: note = alpha-methyl-leucine
  SITE 33: note = Lysine is chemically modified by conjugation of the epsilon-amino group of the Lysine side chain with gamma-Glutamic acid-epsilon-Lysine-(2-[2-(2-Amino-ethoxy)- ethoxy]-acetyl)-epsilon-Lysine-CO-(CH2)18-CO2H
  SITE 42: note = Amidated Residue

SEQ ID NO: 20 - protein, 42 aa, synthetic construct; identical to SEQ ID NO: 1
  SITE 1: note = N-terminal acetylated residue
  SITE 9: note = alpha-methyl-leucine
  SITE 14: note = 2-Aminoisobutyric Acid
  SITE 15: note = alpha-methyl-leucine
  SITE 42: note = Amidated Residue

SEQ ID NO: 21 - protein, 42 aa, synthetic construct; identical to SEQ ID NO: 1
  SITE 1: note = N-terminal acetylated residue
  SITE 9: note = alpha-methyl-leucine
  SITE 14: note = 2-Aminoisobutyric Acid
  SITE 15: note = alpha-methyl-leucine
  SITE 33: note = Lysine is chemically modified by conjugation of the epsilon-amino group of the Lysine side chain with gamma-Glutamic acid-(2-[2-(2-Amino-ethoxy)-ethoxy]-acetyl) -epsilon-Lysine-CO-(CH2)16-CO2H
  SITE 42: note = Amidated Residue

SEQ ID NO: 22 - protein, 44 aa, synthetic construct
  SITE 1: note = N-terminal Pyroglutamic acid
  SITE 11: note = alpha-methyl-leucine
  SITE 16: note = 2-Aminoisobutyric Acid
  SITE 17: note = alpha-methyl-leucine
  SITE 35: note = Lysine is chemically modified by conjugation of the epsilon-amino group of the Lysine side chain with gamma-Glutamic acid-(2-[2-(2-Amino-ethoxy)-ethoxy]-acetyl) -epsilon-Lysine-CO-(CH2)18-CO2H
  SITE 44: note = Amidated Residue
XGSPSGIVTSLDVPTXLLQKILEQERAEKARQQAKEAAEILAQV

SEQ ID NO: 23 - protein, 44 aa, synthetic construct; identical to SEQ ID NO: 22
  SITE 1: note = N-terminal Pyroglutamic acid
  SITE 11: note = alpha-methyl-leucine
  SITE 16: note = 2-Aminoisobutyric Acid
  SITE 17: note = alpha-methyl-leucine
  SITE 44: note = Amidated Residue

SEQ ID NO: 24 - protein, 44 aa, synthetic construct; identical to SEQ ID NO: 22
  SITE 1: note = N-terminal Pyroglutamic acid
  SITE 11: note = alpha-methyl-leucine
  SITE 16: note = 2-Aminoisobutyric Acid
  SITE 17: note = alpha-methyl-leucine
  SITE 35: note = Lysine is chemically modified by conjugation of the epsilon-amino group of the Lysine side chain with gamma-Glutamic acid-(2-[2-(2-Amino-ethoxy)-ethoxy]-acetyl) -epsilon-Lysine-CO-(CH2)16-CO2H
  SITE 44: note = Amidated Residue

SEQ ID NO: 25 - protein, 43 aa, synthetic construct
  SITE 1: note = Gamma-glutamic acid is chemically modified by conjugation of the alpha-amino group of the gamma-glutamic acid side chain with the carboxylic acid group of (2-[2-(2 -Amino-ethoxy)-ethoxy]-acetyl)-epsilon-Lysine-CO-(CH2)18-C O2H
  SITE 10: note = alpha-methyl-leucine
  SITE 15: note = 2-Aminoisobutyric Acid
  SITE 16: note = alpha-methyl-leucine
  SITE 43: note = Amidated Residue
XGSPSIVTSLDVPTXLLQKILEQERAEKARQQATEAAEILAQV

SEQ ID NO: 26 - protein, 43 aa, synthetic construct; identical to SEQ ID NO: 25
  SITE 1: note = Gamma-glutamic acid is chemically modified by conjugation of the alpha-amino group of the gamma-glutamic acid side chain with the carboxylic acid group of (2-[2-(2-Amino-ethoxy)-ethoxy]-acetyl)-K-CO-(CH2)18-CO2H
  SITE 10: note = alpha-methyl-leucine
  SITE 15: note = 2-Aminoisobutyric Acid
  SITE 16: note = alpha-methyl-leucine
  SITE 43: note = Amidated Residue

SEQ ID NO: 27 - protein, 42 aa, synthetic construct
  SITE 9: note = alpha-methyl-leucine
  SITE 14: note = 2-Aminoisobutyric Acid
  SITE 15: note = alpha-methyl-leucine
  SITE 42: note = Amidated Residue
GSPSIVTSLDVPTXLLQKILEQERAEKARQQATEAAEILAQV

SEQ ID NO: 28 - protein, 43 aa, synthetic construct; identical to SEQ ID NO: 25
  SITE 1: note = Gamma-glutamic acid is chemically modified by conjugation of the alpha-amino group of the gamma-glutamic acid side chain with the carboxylic acid group of (2-[2-(2 -Amino-ethoxy)-ethoxy]-acetyl)-epsilon-Lysine-CO-(CH2)16-C O2H
  SITE 10: note = alpha-methyl-leucine
  SITE 15: note = 2-Aminoisobutyric Acid
  SITE 16: note = alpha-methyl-leucine
  SITE 43: note = Amidated Residue

SEQ ID NO: 29 - protein, 38 aa, synthetic construct
  SITE 1: note = N-terminal acetylated residue
  SITE 5: note = alpha-methyl-leucine
  SITE 7: note = d-enantiomer of Valine
  SITE 11: note = alpha-methyl-leucine
  SITE 29: note = Lysine is chemically modified by conjugation of the epsilon-amino group of the Lysine side chain with (2-[2-(2-Amino-ethoxy)-ethoxy]-acetyl)2-gamma-Glutamic acid-CO-(CH2)18-CO2H
  SITE 38: note = Amidated Residue
IVTSLDVPTGLLQIKLEQERAEKARQQAKENTEILAQV

SEQ ID NO: 30 - protein, 38 aa, synthetic construct; identical to SEQ ID NO: 29
  SITE 1: note = N-terminal acetylated residue
  SITE 5: note = alpha-methyl-leucine
  SITE 7: note = d-enantiomer of Valine
  SITE 11: note = alpha-methyl-leucine
  SITE 29: note = Lysine is chemically modified by conjugation of the epsilon-amino group of the Lysine side chain with gamma-Glutamic acid-(2-[2-(2-Amino-ethoxy)-ethoxy]-acetyl)-gamma-Glutamic acid-CO-(CH2)18-CO2H
  SITE 38: note = Amidated Residue

SEQ ID NO: 31 - protein, 38 aa, synthetic construct; identical to SEQ ID NO: 29
  SITE 1: note = N-terminal acetylated residue
  SITE 5: note = alpha-methyl-leucine
  SITE 7: note = d-enantiomer of Valine
  SITE 11: note = alpha-methyl-leucine
  SITE 29: note = Lysine is chemically modified by conjugation of the epsilon-amino group of the Lysine side chain with (gamma-Glutamic acid)2-(2-[2-(2-Amino-ethoxy)-ethoxy]-acet yl)-CO-(CH2)18-CO2H
  SITE 38: note = Amidated Residue

SEQ ID NO: 32 - protein, 38 aa, synthetic construct; identical to SEQ ID NO: 29
  SITE 1: note = N-terminal acetylated residue
  SITE 5: note = alpha-methyl-leucine
  SITE 7: note = d-enantiomer of Valine
  SITE 11: note = alpha-methyl-leucine
  SITE 29: note = Lysine is chemically modified by conjugation of the epsilon-amino group of the Lysine side chain with (2-[2-(2 -Amino-ethoxy)-ethoxy]-acetyl)-epsilon-Lysine-gamma-Glutam ic acid-CO-(CH2)18-CO2H
  SITE 38: note = Amidated Residue

SEQ ID NO: 33 - protein, 38 aa, synthetic construct; identical to SEQ ID NO: 29
  SITE 1: note = N-terminal acetylated residue
  SITE 5: note = alpha-methyl-leucine
  SITE 7: note = d-enantiomer of Valine
  SITE 11: note = alpha-methyl-leucine
  SITE 29: note = Lysine is chemically modified by conjugation of the epsilon-amino group of the Lysine side chain with (2-[2-(2-Amino-ethoxy)-ethoxy]-acetyl)-gamma-Glutamic acid-epsilon-Lysine-CO-(CH2)18-CO2H
  SITE 38: note = Amidated Residue

SEQ ID NO: 34 - protein, 38 aa, synthetic construct; identical to SEQ ID NO: 29
  SITE 1: note = N-terminal acetylated residue
  SITE 5: note = alpha-methyl-leucine
  SITE 7: note = d-enantiomer of Valine
  SITE 11: note = alpha-methyl-leucine
  SITE 29: note = Lysine is chemically modified by conjugation of the epsilon-amino group of the Lysine side chain with epsilon- Lysine-(2-[2-(2-Amino-ethoxy)-ethoxy]-acetyl)-gamma-Glutam ic acid-CO-(CH2)18-CO2H
  SITE 38: note = Amidated Residue

SEQ ID NO: 35 - protein, 38 aa, synthetic construct; identical to SEQ ID NO: 29
  SITE 1: note = N-terminal acetylated residue
  SITE 5: note = alpha-methyl-leucine
  SITE 7: note = d-enantiomer of Valine
  SITE 11: note = alpha-methyl-leucine
  SITE 29: note = Lysine is chemically modified by conjugation of the epsilon-amino group of the Lysine side chain with epsilon-Lysine-gamma-Glutamic acid-(2-[2-(2-Amino-ethoxy)- ethoxy]-acetyl)-CO-(CH2)18-CO2H
  SITE 38: note = Amidated Residue

SEQ ID NO: 36 - protein, 38 aa, synthetic construct; identical to SEQ ID NO: 29
  SITE 1: note = N-terminal acetylated residue
  SITE 5: note = alpha-methyl-leucine
  SITE 7: note = d-enantiomer of Valine
  SITE 11: note = alpha-methyl-leucine
  SITE 29: note = Lysine is chemically modified by conjugation of the epsilon-amino group of the Lysine side chain with gamma-Glutamic acid-(2-[2-(2-Amino-ethoxy)-ethoxy]-acetyl) -epsilon-Lysine-CO-(CH2)18-CO2H
  SITE 38: note = Amidated Residue

SEQ ID NO: 37 - protein, 38 aa, synthetic construct; identical to SEQ ID NO: 29
  SITE 1: note = N-terminal acetylated residue
  SITE 5: note = alpha-methyl-leucine
  SITE 7: note = d-enantiomer of Valine
  SITE 11: note = alpha-methyl-leucine
  SITE 29: note = Lysine is chemically modified by conjugation of the epsilon-amino group of the Lysine side chain with gamma-Glutamic acid-epsilon-Lysine-(2-[2-(2-Amino-ethoxy)- ethoxy]-acetyl)-CO-(CH2)18-CO2H
  SITE 38: note = Amidated Residue

SEQ ID NO: 38 - protein, 38 aa, synthetic construct; identical to SEQ ID NO: 29
  SITE 1: note = N-terminal acetylated residue
  SITE 5: note = alpha-methyl-leucine
  SITE 7: note = d-enantiomer of Valine
  SITE 11: note = alpha-methyl-leucine
  SITE 29: note = Lysine is chemically modified by conjugation of the epsilon-amino group of the Lysine side chain with gamma-Glutamic acid-gamma-Glutamic acid-epsilon-Lysine-CO-(CH2)18-CO2H
  SITE 38: note = Amidated Residue

SEQ ID NO: 39 - protein, 38 aa, synthetic construct; identical to SEQ ID NO: 29
  SITE 1: note = N-terminal acetylated residue
  SITE 5: note = alpha-methyl-leucine
  SITE 7: note = d-enantiomer of Valine
  SITE 11: note = alpha-methyl-leucine
  SITE 29: note = Lysine is chemically modified by conjugation of the epsilon-amino group of the Lysine side chain with gamma-Glutamic acid-epsilon-Lysine-gamma-Glutamic acid-CO-(CH2)18-CO2H
  SITE 38: note = Amidated Residue

SEQ ID NO: 40 - protein, 38 aa, synthetic construct; identical to SEQ ID NO: 29
  SITE 1: note = N-terminal acetylated residue
  SITE 5: note = alpha-methyl-leucine
  SITE 7: note = d-enantiomer of Valine
  SITE 11: note = alpha-methyl-leucine
  SITE 29: note = Lysine is chemically modified by conjugation of the epsilon-amino group of the Lysine side chain with epsilon-Lysine-(gamma-Glutamic acid)2-CO-(CH2)18-CO2H
  SITE 38: note = Amidated Residue

SEQ ID NO: 41 - protein, 38 aa, synthetic construct
  SITE 1: note = N-terminal acetylated residue
  SITE 5: note = alpha-methyl-leucine
  SITE 7: note = d-enantiomer of Valine
  SITE 10: note = 2-Aminoisobutyric Acid
  SITE 11: note = alpha-methyl-leucine
  SITE 29: note = Lysine is chemically modified by conjugation of the epsilon-amino group of the Lysine side chain with (2-[2-(2-Amino-ethoxy)-ethoxy]-acetyl)2-gamma-Glutamic acid-CO-(CH2)18-CO2H
  SITE 38: note = Amidated Residue
IVTSLDVPTXLLQIKLEQERAEKARQQAKENTEILAQV

SEQ ID NO: 42 - protein, 38 aa, synthetic construct
  SITE 1: note = N-terminal acetylated residue
  SITE 5: note = alpha-methyl-leucine
  SITE 7: note = d-enantiomer of Valine
  SITE 10: note = 2-Aminoisobutyric Acid
  SITE 11: note = alpha-methyl-leucine
  SITE 29: note = Lysine is chemically modified by conjugation of the epsilon-amino group of the Lysine side chain with (2-[2-(2-Amino-ethoxy)-ethoxy]-acetyl)2-gamma-Glutamic acid-CO-(CH2)18-CO2H
  SITE 38: note = Amidated Residue
IVTSLDVPIXLLQIKLEQERAEKARQQAKENTEILAQV

SEQ ID NO: 43 - protein, 38 aa, synthetic construct; identical to SEQ ID NO: 29
  SITE 1: note = N-terminal acetylated residue
  SITE 5: note = alpha-methyl-leucine
  SITE 7: note = d-enantiomer of Valine
  SITE 11: note = alpha-methyl-leucine
  SITE 29: note = Lysine is chemically modified by conjugation of the epsilon-amino group of the Lysine side chain with GKEKEKE-gamma-Glutamic acid-CO-(CH2)18-CO2H
  SITE 38: note = Amidated Residue

SEQ ID NO: 44 - protein, 38 aa, synthetic construct; identical to SEQ ID NO: 29
  SITE 1: note = N-terminal acetylated residue
  SITE 5: note = alpha-methyl-leucine
  SITE 7: note = d-enantiomer of Valine
  SITE 11: note = alpha-methyl-leucine
  SITE 29: note = Lysine is chemically modified by conjugation of the epsilon-amino group of the Lysine side chain with (2-[2-(2-Amino-ethoxy)-ethoxy]-acetyl)-(gamma-Glutamic acid)2-CO-(CH2)18-CO2H
  SITE 38: note = Amidated Residue

SEQ ID NO: 45 - protein, 42 aa, synthetic construct
  SITE 1: note = gamma-Glutamic acid
  SITE 9: note = alpha-methyl-leucine
  SITE 11: note = d-enantiomer of Valine
  SITE 14: note = 2-Aminoisobutyric Acid
  SITE 15: note = alpha-methyl-leucine
  SITE 33: note = Lysine is chemically modified by conjugation of the epsilon-amino group of the Lysine side chain with (2-[2-(2-Amino-ethoxy)-ethoxy]-acetyl)2-gamma-Glutamic acid-CO-(CH2)18-CO2H
  SITE 42: note = Amidated Residue
XGPSIVTSLDVPIXLLQIKLEQERAEKARQQAKENTEILAQV

SEQ ID NO: 46 - protein, 41 aa, synthetic construct
  SITE 1: note = N-terminal acetylated residue
  SITE 8: note = alpha-methyl-leucine
  SITE 10: note = d-enantiomer of Valine
  SITE 13: note = 2-Aminoisobutyric Acid
  SITE 14: note = alpha-methyl-leucine
  SITE 18: note = 2-Aminoisobutyric Acid
  SITE 32: note = Lysine is chemically modified by conjugation of the epsilon-amino group of the Lysine side chain with gamma-Glutamic acid-(2-[2-(2-Amino-ethoxy)-ethoxy]-acetyl) -epsilon-Lysine-CO-(CH2)18-CO2H
  SITE 41: note = Amidated Residue
PPPIVTSLDVPTXLLQKXLEQERAEKARQQAKENTEILAQV

SEQ ID NO: 47 - protein, 41 aa, synthetic construct
  SITE 1: note = N-terminal acetylated residue
  SITE 8: note = alpha-methyl-leucine
  SITE 10: note = d-enantiomer of Valine
  SITE 13: note = 2-Aminoisobutyric Acid
  SITE 14: note = alpha-methyl-leucine
  SITE 32: note = Lysine is chemically modified by conjugation of the epsilon-amino group of the Lysine side chain with gamma-Glutamic acid-(2-[2-(2-Amino-ethoxy)-ethoxy]-acetyl) -epsilon-Lysine-CO-(CH2)18-CO2H
  SITE 41: note = Amidated Residue
PPPIVTSLDVPTXLLQKLLEQERAEKARQQAKEAAEILAQV

SEQ ID NO: 48 - protein, 41 aa, synthetic construct; identical to SEQ ID NO: 47
  SITE 1: note = N-terminal acetylated residue
  SITE 8: note = alpha-methyl-leucine
  SITE 13: note = 2-Aminoisobutyric Acid
  SITE 14: note = alpha-methyl-leucine
  SITE 32: note = Lysine is chemically modified by conjugation of the epsilon-amino group of the Lysine side chain with gamma-Glutamic acid-(2-[2-(2-Amino-ethoxy)-ethoxy]-acetyl) -epsilon-Lysine-CO-(CH2)18-CO2H
  SITE 41: note = Amidated Residue

SEQ ID NO: 49 - protein, 38 aa, synthetic construct
  SITE 1: note = N-methylated Isoleucine
  SITE 11: note = alpha-methyl-leucine
  SITE 29: note = Lysine is chemically modified by conjugation of the epsilon-amino group of the Lysine side chain with (2-[2-(2-Amino-ethoxy)-ethoxy]-acetyl)2-gamma-Glutamic acid-CO-(CH2)18-CO2H
  SITE 38: note = Amidated Residue
IVTSLDVPIGLLQKLIEQEKQEKEKQQAKENTEILAQV

SEQ ID NO: 50 - protein, 38 aa, synthetic construct
  SITE 1: note = N-methylated Isoleucine
  SITE 11: note = alpha-methyl-leucine
  SITE 29: note = Lysine is chemically modified by conjugation of the epsilon-amino group of the Lysine side chain with (2-[2-(2-Amino-ethoxy)-ethoxy]-acetyl)2-gamma-Glutamic acid-CO-(CH2)18-CO2H
  SITE 38: note = Amidated Residue
IVTSLDVPIGLLQKLLEQERAEKARQQAKEAAEILAQV

SEQ ID NO: 51 - protein, 38 aa, synthetic construct; identical to SEQ ID NO: 42
  SITE 1: note = N-terminal acetylated residue
  SITE 5: note = alpha-methyl-leucine
  SITE 7: note = d-enantiomer of Valine
  SITE 10: note = 2-Aminoisobutyric Acid
  SITE 11: note = alpha-methyl-leucine
  SITE 29: note = Lysine is chemically modified by conjugation of the epsilon-amino group of the Lysine side chain with (gamma-Glutamic acid)2-CO-(CH2)18-CO2H
  SITE 38: note = Amidated Residue

SEQ ID NO: 52 - protein, 43 aa, synthetic construct
  SITE 1: note = Gamma-glutamic acid is chemically modified by conjugation of the alpha-amino group of the gamma-glutamic acid side chain with the carboxylic acid group of (2-[2-(2 -Amino-ethoxy)-ethoxy]-acetyl)-2,3-Diaminopropionic Acid-CO-(CH2)18-CO2H
  SITE 10: note = alpha-methyl-leucine
  SITE 15: note = 2-Aminoisobutyric Acid
  SITE 16: note = alpha-methyl-leucine
  SITE 43: note = Amidated Residue
XGSPSIVTSLDVPTXLLEKILEQERAEKARQQATEAAEILAQV

SEQ ID NO: 53 - protein, 38 aa, synthetic construct
  SITE 38: note = Amidated Residue
IVLSLDVPIGLLQILLEQARARAAREQATTNARILARV

SEQ ID NO: 54 - protein, 38 aa, synthetic construct
  SITE 1: note = N-methylated Isoleucine
  SITE 29: note = Lysine is chemically modified by conjugation of the epsilon-amino group of the Lysine side chain with (2-[2-(2-Amino-ethoxy)-ethoxy]-acetyl)2-(gamma-Glutamic acid)2-CO-(CH2)18-CO2H
  SITE 38: note = Amidated Residue
IVLSLDVPIGLLQILLEQEKQEKEKQQAKTNAQILAQV

SEQ ID NO: 55 - protein, 39 aa, synthetic construct
  SITE 2: note = 2-Aminoisobutyric Acid
  SITE 13: note = 2-Aminoisobutyric Acid
  SITE 20: note = Lysine is chemically modified by conjugation of the epsilon-amino group of the Lysine side chain with (2-[2-(2-Amino-ethoxy)-ethoxy]-acetyl)2-gamma-Glutamic acid-CO-(CH2)18-CO2H
  SITE 39: note = Amidated Residue
YXEGTFTSDYSIXLDKIAQKAFVQWLIAGGPSSGAPPPS

SEQ ID NO: 56 - protein, 39 aa, synthetic construct
  SITE 2: note = 2-Aminoisobutyric Acid
  SITE 13: note = alpha-methyl-leucine
  SITE 17: note = Lysine is chemically modified by conjugation of the epsilon-amino group of the Lysine side chain with (2-[2-(2-Amino-ethoxy)-ethoxy]-acetyl)2-gamma-Glutamic acid-CO-(CH2)18-CO2H
  SITE 20: note = 2-Aminoisobutyric Acid
  SITE 39: note = Amidated Residue
YXQGTFTSDYSILLDKKAQXAFIEYLLEGGPSSGAPPPS

SEQ ID NO: 57 - protein, 39 aa, synthetic construct; identical to SEQ ID NO: 56
  SITE 2: note = 2-Aminoisobutyric Acid
  SITE 13: note = alpha-methyl-leucine
  SITE 17: note = Lysine is chemically modified by conjugation of the epsilon-amino group of the Lysine side chain with (2-[2-(2-Amino-ethoxy)-ethoxy]-acetyl)-gamma-Glutamic acid-CO-(CH2)18-CO2H
  SITE 20: note = 2-Aminoisobutyric Acid
  SITE 39: note = Amidated Residue

SEQ ID NO: 58 - protein, 39 aa, synthetic construct; identical to SEQ ID NO: 56
  SITE 2: note = 2-Aminoisobutyric Acid
  SITE 17: note = Lysine is chemically modified by conjugation of the epsilon-amino group of the Lysine side chain with (2-[2-(2-Amino-ethoxy)-ethoxy]-acetyl)2-gamma-Glutamic acid-CO-(CH2)18-CO2H
  SITE 20: note = 2-Aminoisobutyric Acid
  SITE 39: note = Amidated Residue

SEQ ID NO: 59 - protein, 34 aa, synthetic construct
  SITE 2: note = 2-Aminoisobutyric Acid
  SITE 20: note = Lysine is chemically modified by conjugation of the epsilon-amino group of the Lysine side chain with (2-[2-(2-Amino-ethoxy)-ethoxy]-acetyl)2-gamma-Glutamic acid-CO-(CH2)16-CO2H
  SITE 34: note = Amidated Residue
HXQGTFTSDYSKYLDEKKAKEFVEWLLEGGPSSG

SEQ ID NO: 60 - protein, 34 aa, synthetic construct
  SITE 2: note = 2-Aminoisobutyric Acid
  SITE 20: note = Lysine is chemically modified by conjugation of the epsilon-amino group of the Lysine side chain with (2-[2-(2-Amino-ethoxy)-ethoxy]-acetyl)2-(gamma-Glutamic acid)2-CO-(CH2)16-CO2H
  SITE 34: note = Amidated Residue
HXQGTFTSDYSKYLDEKKAKEFVEWLLSGGPSSG

SEQ ID NO: 61 - protein, 34 aa, synthetic construct; identical to SEQ ID NO: 59
  SITE 2: note = 2-Aminoisobutyric Acid
  SITE 20: note = Lysine is chemically modified by conjugation of the epsilon-amino group of the Lysine side chain with (2-[2-(2-Amino-ethoxy)-ethoxy]-acetyl)2-gamma-Glutamic acid-CO-(CH2)18-CO2H
  SITE 34: note = Amidated Residue

SEQ ID NO: 62 - protein, 34 aa, synthetic construct; identical to SEQ ID NO: 60
  SITE 2: note = 2-Aminoisobutyric Acid
  SITE 20: note = Lysine is chemically modified by conjugation of the epsilon-amino group of the Lysine side chain with (2-[2-(2-Amino-ethoxy)-ethoxy]-acetyl)2-(gamma-Glutamic acid)2-CO-(CH2)18-CO2H
  SITE 34: note = Amidated Residue

SEQ ID NO: 63 - protein, 37 aa, synthetic construct
  SITE 1: note = gamma-Glutamic acid
  CROSSLNK 2..7: note = methylene thioacetal linkage (Cys-Cys)
  SITE 11: note = L-ornithine
  SITE 15: note = alpha-methyl-phenylalanine
  SITE 22: note = N-methylated asparagine
  SITE 26: note = Lysine is chemically modified by conjugation of the epsilon-amino group of the Lysine side chain with (gamma-Glutamic acid)2-CO-(CH2)18-CO2H
  SITE 37: note = Amidated Residue
XCNTATCATGXLAEFLVRSSNNFGPKLPPTEVGSNTY

SEQ ID NO: 64 - protein, 37 aa, synthetic construct
  SITE 1: note = gamma-Glutamic acid
  CROSSLNK 2..7: note = methylene thioacetal linkage (Cys-Cys)
  SITE 11: note = L-ornithine
  SITE 15: note = alpha-methyl-phenylalanine
  SITE 22: note = N-methylated aspartic acid
  SITE 26: note = Lysine is chemically modified by conjugation of the epsilon-amino group of the Lysine side chain with (gamma-Glutamic acid)2-CO-(CH2)18-CO2H
  SITE 37: note = Amidated Residue
XCNTATCATGXLAEFLVRSSNDFGPKLPPTEVGSNTY

SEQ ID NO: 65 - protein, 37 aa, synthetic construct
  SITE 1: note = Lysine is chemically modified by conjugation of the epsilon-amino group of the Lysine side chain with (gamma-Glutamic acid)2-CO-(CH2)18-CO2H
  CROSSLNK 2..7: note = methylene thioacetal linkage (Cys-Cys)
  SITE 11: note = L-ornithine
  SITE 15: note = alpha-methyl-phenylalanine
  SITE 22: note = N-methylated aspartic acid
  SITE 37: note = Amidated Residue
KCETATCATGXLAEFLVRSSNDFGPILPPTEVGSNTY

SEQ ID NO: 66 - protein, 37 aa, synthetic construct
  SITE 1: note = Lysine is chemically modified by conjugation of the epsilon-amino group of the Lysine side chain with (gamma-Glutamic acid)2-CO-(CH2)18-CO2H
  CROSSLNK 2..7: note = methylene thioacetal linkage (Cys-Cys)
  SITE 11: note = L-ornithine
  SITE 12: note = alpha-methyl-leucine
  SITE 37: note = Amidated Residue
KCETATCATGXLAEFLVRSSHNFGPILPPTEVGSNTY

SEQ ID NO: 67 - protein, 32 aa, synthetic construct
  SITE 1: note = N-terminal acetylated residue
  SITE 11: note = Lysine is chemically modified by conjugation of the epsilon-amino group of the Lysine side chain with (2-[2-(2-Amino-ethoxy)-ethoxy]-acetyl)2-gamma-Glutamic acid-CO-(CH2)18-CO2H
  SITE 14: note = 2-Aminoisobutyric Acid
  SITE 32: note = Amidated Residue
ASHLSTAVLGKLSXELHKLEDYPRTDVGAESP

SEQ ID NO: 68 - protein, 44 aa, synthetic construct
  VARIANT 1: note = N-terminal Pyroglutamic acid, gamma-Glutamic acid, E, or may be absent wherein this position may be N-acetylated, N-methylated or N-acylated
  VARIANT 2: note = G or absent, wherein this position may be N-acetylated, N-acylated or N-methylated if position 1 is absent
  VARIANT 3: note = G, S, or absent, wherein this position may be N-acetylated, N-acylated or N-methylated if positions 1-2 are absent
  VARIANT 4: note = S, P, G, or absent, wherein this position may be N-acetylated, N-acylated or N-methylated if positions 1-3 are absent
  VARIANT 5: note = S, P, or absent, wherein this position may be N-acetylated, N-acylated or N-methylated if positions 1-4 are absent
  VARIANT 6: note = G, S, P, or absent, wherein this position may be N-acetylated, N-acylated or N-methylated if positions 1-5 are absent
  VARIANT 7: note = This position may be N-acetylated, or N-acylated or N-methylated if positions 1-6 are absent
  VARIANT 11: note = L or alpha-methyl-leucine
  VARIANT 13: note = V or d-enantiomer of Valine
  VARIANT 15: note = T or I
  VARIANT 16: note = 2-Aminoisobutyric Acid or G
  VARIANT 17: note = alpha-methyl-leucine or L
  VARIANT 19: note = Q or E
  VARIANT 20: note = K or I
  VARIANT 21: note = I, K, 2-Aminoisobutyric Acid, or L
  VARIANT 22: note = I or L
  VARIANT 26: note = R or K
  VARIANT 27: note = A or Q
  VARIANT 30: note = A or E
  VARIANT 31: note = R or K
  VARIANT 35: note = K or T
  VARIANT 37: note = A or N
  VARIANT 38: note = A or T
  VARIANT 44: note = This residue may be amidated
XXXXXXIVTSXDXPXXLLXXXXEQEXXEKXXQQAXEXXEILAQV

SEQ ID NO: 69 - protein, 42 aa, synthetic construct
  VARIANT 1: note = This residue may be N-acetylated
  SITE 9: note = alpha-methyl-leucine
  SITE 14: note = 2-Aminoisobutyric Acid
  SITE 15: note = alpha-methyl-leucine
  VARIANT 19: note = I or L
  VARIANT 42: note = This residue may be amidated
GSPSIVTSLDVPTXLLQKXLEQERAEKARQQAKEAAEILAQV

SEQ ID NO: 70 - protein, 44 aa, synthetic construct; identical to SEQ ID NO: 22
  SITE 1: note = N-terminal Pyroglutamic acid
  SITE 11: note = alpha-methyl-leucine
  SITE 16: note = 2-Aminoisobutyric Acid
  SITE 17: note = alpha-methyl-leucine
  VARIANT 44: note = This residue may be amidated

SEQ ID NO: 71 - protein, 44 aa, synthetic construct
  VARIANT 1: note = gamma-Glutamic acid or absent
  SITE 11: note = alpha-methyl-leucine
  SITE 16: note = 2-Aminoisobutyric Acid
  SITE 17: note = alpha-methyl-leucine
  VARIANT 19: note = Q or E
  VARIANT 43: note = This residue may be amidated
  SITE 2: note = absent
XXGSPSIVTSLDVPTXLLXKILEQERAEKARQQATEAAEILAQV

SEQ ID NO: 72 - protein, 38 aa, synthetic construct
  VARIANT 1: note = Residue may be N-terminal acetylated
  SITE 5: note = alpha-methyl-leucine
  SITE 7: note = d-enantiomer of Valine
  VARIANT 9: note = T or I
  VARIANT 10: note = G or 2-Aminoisobutyric Acid
  SITE 11: note = alpha-methyl-leucine
  VARIANT 38: note = This residue may be amidated
IVTSLDVPXXLLQIKLEQERAEKARQQAKENTEILAQV

SEQ ID NO: 73 - protein, 41 aa, synthetic construct
  VARIANT 1: note = This residue may be N-terminal acetylated
  SITE 8: note = alpha-methyl-leucine
  VARIANT 10: note = V or d-enantiomer of Valine
  SITE 13: note = 2-Aminoisobutyric Acid
  SITE 14: note = alpha-methyl-leucine
  VARIANT 18: note = L or 2-Aminoisobutyric Acid
  VARIANT 34: note = A or N
  VARIANT 35: note = A or T
  VARIANT 41: note = This residue may be amidated
PPPIVTSLDXPTXLLQKXLEQERAEKARQQAKEXXEILAQV

SEQ ID NO: 74 - protein, 8 aa, synthetic construct
  SITE 1: note = gamma-Glutamic acid
  SITE 8: note = Glutamic acid attached to CO-(CH2)18-CO2H
XGKEKEKE

SEQ ID NO: 75 - protein, 7 aa, synthetic construct
  SITE 1: note = (2-[2-(2-Amino-ethoxy)-ethoxy]-acetyl) attached to Lysine
  SITE 7: note = gamma-Glutamic acid attached to CO-(CH2)18-CO2H
KEKEKEX

SEQ ID NO: 76 - protein, 7 aa, synthetic construct
  SITE 1: note = gamma-Glutamic acid
  SITE 7: note = Proline attached to CO-(CH2)18-CO2H
XPPPPPP

SEQ ID NO: 77 - protein, 7 aa, synthetic construct
  SITE 1: note = gamma-Glutamic acid
  SITE 7: note = Glutamic acid attached to CO-(CH2)18-CO2H
XPEPEPE

SEQ ID NO: 78 - protein, 8 aa, synthetic construct
  SITE 8: note = gamma-Glutamic acid attached to CO-(CH2)18-CO2H
GKEKEKEX

SEQ ID NO: 79 - protein, 31 aa, synthetic construct
  SITE 2: note = 2-Aminoisobutyric Acid
  SITE 20: note = Lys is chemically modified by conjugation of the epsilon-amino group of the Lys side chain with (2-[2-(2-Amino-ethoxy)-ethoxy]-acetyl)2-gamma-Glutamic acid-CO-(CH2)16-CO2H
HXEGTFTSDVSSYLEGQAAKEFIAWLVRGRG

SEQ ID NO: 80 - protein, 31 aa, synthetic construct
  SITE 20: note = Lys is chemically modified by conjugation of the epsilon-amino group of the Lys side chain with gamma-Glutamic acid-CO-(CH2)14-CH3
HAEGTFTSDVSSYLEGQAAKEFIAWLVRGRG

SEQ ID NO: 81 - protein, 275 aa, synthetic construct
HGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG